(19) United States
(12) Patent Application Publication
(10) Pub. No.: US 2026/0109690 A1
(43) Pub. Date: Apr. 23, 2026

(54) HDAC6 INHIBITORS AND METHOD OF THEIR USE

(71) Applicant: Baerenkraft Therapeutics, LLC., Lehi, UT (US)

(72) Inventors: Hariprasad Vankayalapati, Sandy, UT (US); Zhaoliang Li, Salt Lake City, UT (US); Chenyu Lin, Lehi, UT (US); Kyle Medley, American Fork, UT (US); David J. Bearss, Alpine, UT (US)

(21) Appl. No.: 19/223,811

(22) Filed: May 30, 2025

Related U.S. Application Data
(60) Provisional application No. 63/654,635, filed on May 31, 2024.

(51) Int. Cl. C07D 413/12; A61K 31/506; A61K 31/5377; A61K 31/5386; A61P 21/00; A61P 25/28; C07D 401/04; C07D 401/12; C07D 403/12; C07D 405/14; C07D 413/04; C07D 413/14; C07D 491/107; C07D 498/08; C07D 498/10

(52) U.S. Cl. CPC C07D 413/12; A61K 31/506; A61K 31/5377; A61K 31/5386; A61P 21/00; A61P 25/28; C07D 401/04; C07D 401/12; C07D 403/12; C07D 405/14; C07D 413/04; C07D 413/14; C07D 491/107; C07D 498/08; C07D 498/10

(57) ABSTRACT

Compounds having activity as inhibitors of HDAC6 are provided. The compounds have Structure (I):

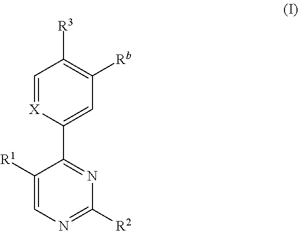

or a pharmaceutically acceptable salt, stereoisomer, or tautomer thereof, wherein X, R¹, R², R³, and R^{b} are as defined herein. Methods associated with preparation and use of such compounds, pharmaceutical compositions comprising such compounds and methods to modulate the activity of HDAC6 are also provided.

### CROSS-REFERENCE TO RELATED APPLICATION

This application claims the benefit of U.S. Provisional Patent Application Ser. No. 63/654,635, filed May 31, 2024, entitled, “HDAC6 INHIBITORS AND METHOD OF THEIR USE,” the entirety of which is hereby incorporated by reference.

### TECHNICAL FIELD

Embodiments of the present disclosure are generally directed to compounds and methods for their preparation and use as therapeutic or prophylactic agents, for example for treatment of neurodegenerative diseases or disorders and/or cancer.

### DESCRIPTION OF THE RELATED ART

Histone deacetylase 6 is an enzyme encoded by the HDAC6 gene in humans. HDAC6 has emerged as a highly promising candidate to selectively inhibit as a therapeutic strategy to combat several types of neurodegenerative disorders specifically neurodegenerative, neurological disorders such as amyotrophic lateral sclerosis (ALS). Alzheimer's disease (AD), Rett syndrome, idiopathic pulmonary fibrosis (IPF) and other conditions related to HDAC6 activity. HDAC6 is also a target for treating cancer particularly non-small cell lung cancer (NSCLC), chronic lymphocytic leukemia (CLL), and multiple myeloma.

ALS is a progressive neurodegenerative disease that gradually and irreversibly affects motor movement due to the death of motor neurons in the brain and spinal cord. Mutations in over 20 genes have been associated with familial ALS, with mutations in 4 genes accounting for most ALS familial cases. ALS disease ultimately leads to paralysis and eventually death, usually because of respiratory failure within 3 to 5 years after symptom onset, typically in spinal, including limb weakness. HDAC6 is an enzyme that regulates the acetylation of proteins, including those involved in transporting important cellular components along the axons of neurons. Studies have shown that HDAC6 plays a role in the clearance of abnormal protein aggregates, such as SOD1 and TDP-43, which are implicated in ALS pathogenesis. Evidence suggests that HDAC6 inhibitors could have potential therapeutic effects in ALS.

Accordingly, there is a need to develop inhibitor compounds that will directly target HDAC6 in several diseases (e.g., neurodegenerative diseases and disorders, cancers, etc.) Embodiments of the present disclosure fulfill this need and provide further related advantages.

### BRIEF SUMMARY

In brief, embodiments of the present disclosure provide compounds, including pharmaceutically acceptable salts, stereoisomers, and tautomers thereof, which are capable of inhibiting HDAC6.

In one aspect, the disclosure provides compounds of Structure (I):

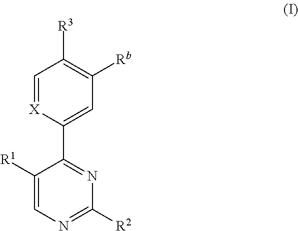

pharmaceutically acceptable salts, stereoisomers, and tautomers thereof, wherein each of X, R¹, R², R³, and R^{b} are as defined below.

In another aspect, pharmaceutical compositions comprising the disclosed compounds, and methods of use of the same for treatment of diseases (e.g., cancer, a neurodegenerative disease, or an autoimmune disease) are also provided.

### BRIEF DESCRIPTION OF THE DRAWINGS

In the figures, identical reference numbers identify similar elements or acts. The sizes and relative positions of elements in the figures are not necessarily drawn to scale. For example, the shapes of various elements and angles are not drawn to scale and some of these elements are enlarged and positioned to improve figure legibility. Further, the shapes of the elements as drawn, are not intended to convey any information regarding the actual shape of the elements and have been solely selected for ease of recognition in the figures. The patterns below are each of solid form(s) of a compound of Structure (I) and are the free forms thereof unless indicated otherwise.

FIG. 1 shows HDAC6 activity of compound I-8 in a 10-dose in vitro test. The EC₅₀ was determined to be 7.319 nM.

FIG. 2 illustrates nanoBRET assay results for target engagement of HEK293 cells transfected with HDAC6. Compound I-8 was tested in comparison to suberoylanilide hydroxamic acid (SAHA) and exhibited on target HDAC6 inhibition with an IC₅₀ of 34.67 nM.

FIG. 3 shows activity of compound I-15 (IC₅₀=16 nM) and compound I-16 (IC₅₀=12.37) against HDAC6. For comparison, Trichostatin A was determined to have an IC₅₀=876.20.

FIG. 4 shows a dose response for representative compounds of Structure (I) tested against HDAC6 via average fluorescence intensity via acetylated α-tubulin in SH-SY5Y neuroblastoma cells.

FIG. 5 shows immunochemistry and Western blotting experimental results for dose response efficacy for compound I-8 in SH-SY5Y neuroblastoma cells. Compound I-8 induces acetylation α-tubulin in SH-SY5Y

FIG. 6 shows the body weight of mice for G1, G2, and G3 for days following dosing (i.e., days are listed along the x-axis).

FIG. 7A shows the number of spontaneous alterations for the Y-Maze test described herein.

FIG. 7B shows the percentage of spontaneous alterations for the Y-Maze test described herein.

FIG. 8 shows the results for the time spent plotted against the time points following each dose. The results are from the elevated plus maze test as described herein.

FIG. 9A shows the results for time spent in a periphery zone for the open filed test as described herein.

FIG. 9B shows the results for the time spent in the central zone for the open field test as described herein.

FIG. 10 shows results for the Morris water maze test (i.e., showing time spent in seconds for each group)

For FIGS. 7A thought 10, data is shown for each timepoint for groups (from left to right) G1, G2, and G3.

FIG. 11 shows the bioanalysis (plasma) results for bleeding timepoints plotted against the concentration of compound I-16 (y-axis; ng/mL).

FIG. 12 shows the effect of compound I-8 on the body weight of animals for groups G1 (vehicle control), G2 (disease control), G3, and G4.

FIG. 13 shows the effect of compound I-8 on the neurological scoring for animals treated in groups G1 (vehicle control), G2 (disease control), G3, and G4.

FIG. 14A shows latency to traverse the beam in the beam walk test as described herein for animals treated in groups G1-G4.

FIG. 14B shows the number of slips in the beam walk test as described herein for animals treated in groups G1-G4.

FIG. 15 shows the effect of compound I-8 on animals subjected to the rotarod test as described herein (i.e., the latency to fall in seconds plotted against timepoints of 10, 12, 14, and 16 weeks).

FIG. 16 shows the effect of compound I-8 on animals subjected to the wire suspension test as described herein (i.e., the latency to fall in seconds plotted against timepoints of 10, 12, 14, and 16 weeks).

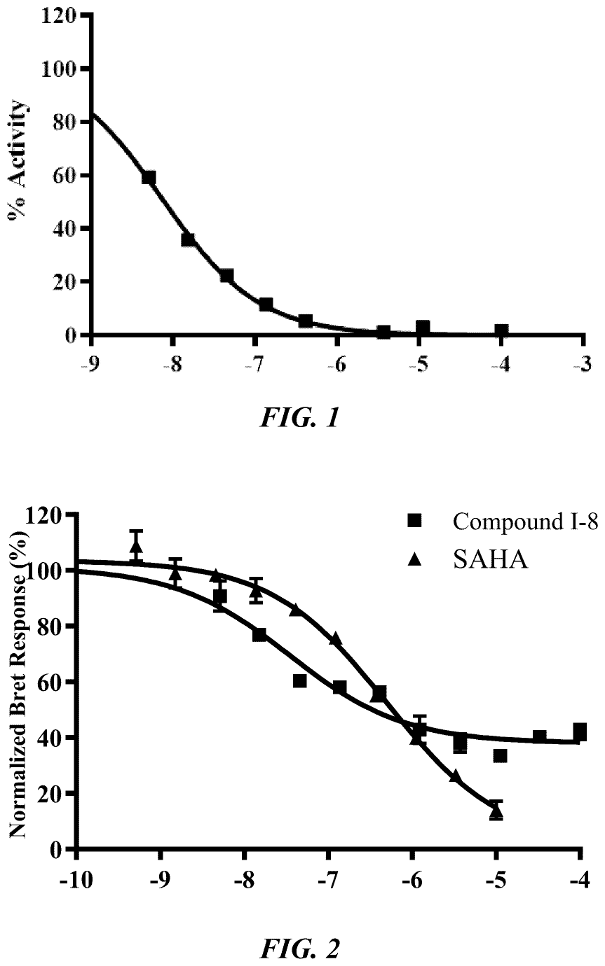

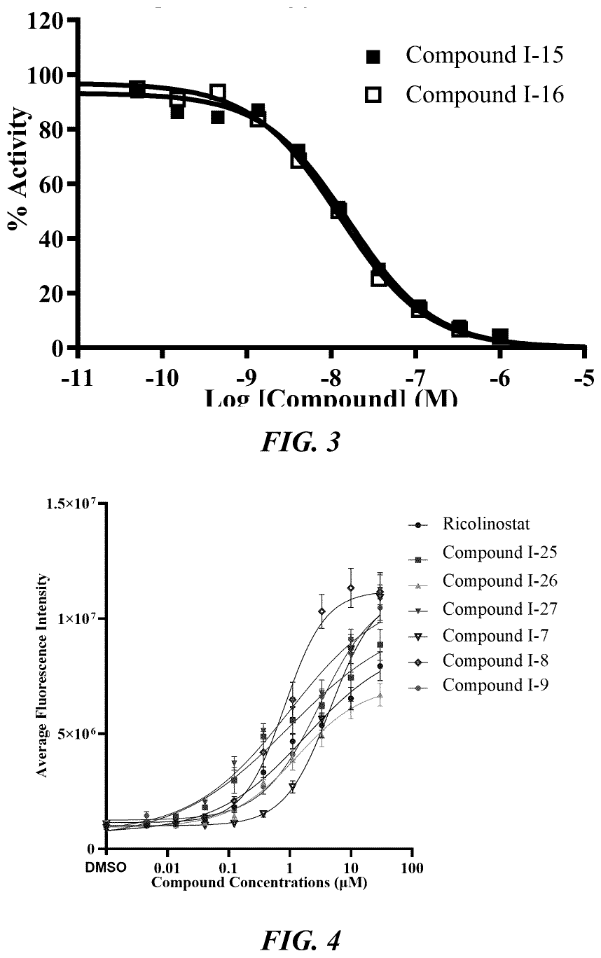

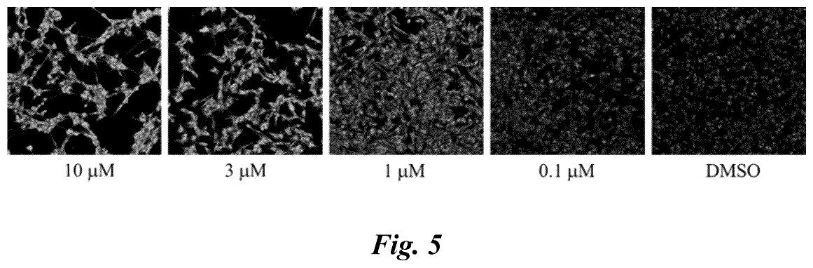

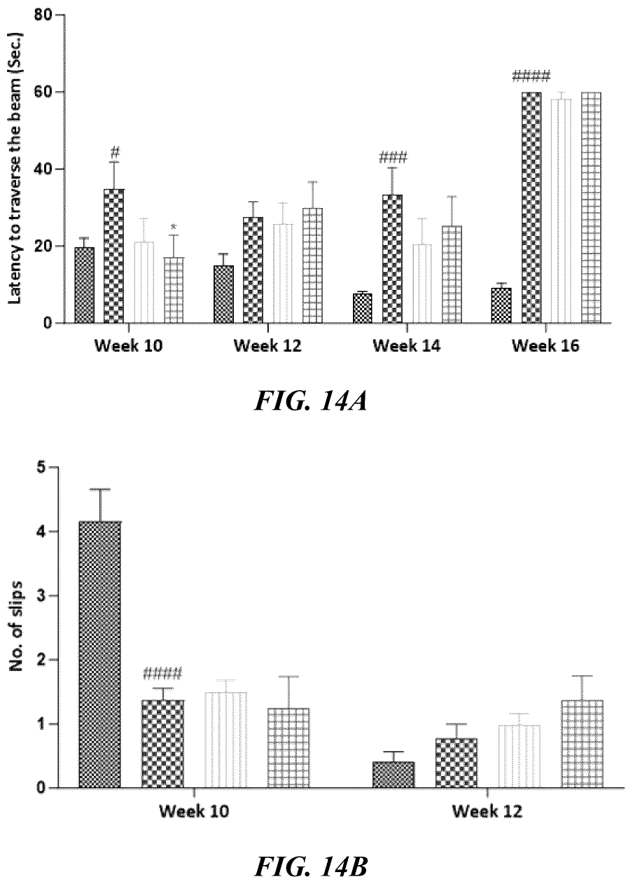

For FIGS. 14A, 14B, 15, and 16 bars show (from left to right) G1, G2, G3, and G4 at each timepoint.

### DETAILED DESCRIPTION

In the following description, certain specific details are set forth to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5% or ±1% of the indicated range, value, or structure, unless otherwise indicated. The terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Cyano” refers to the —CN radical.

“Hydroxy” or “hydroxyl” refers to the —OH radical.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen, and sulfur. Unless stated otherwise specifically in the specification, the heterocyclyl radical is partially or fully saturated and is a monocyclic, bicyclic, tricyclic, or tetracyclic ring system, which may include fused, spirocyclic, and/or bridged ring systems. Nitrogen, carbon, and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“N-heterocyclyl” refers to a heterocyclyl group as defined above that includes at least one ring nitrogen. Unless stated otherwise specifically in the specification, an N-heterocyclyl group is optionally substituted.

The term “substituted” as used herein means any of the above groups (e.g., alkyl, haloalkynyl, alkoxy, aryl, aminoalkyl, hydroxyalkyl, etc.) wherein at least one hydrogen atom (e.g., 1, 2, 3 or all hydrogen atoms) is replaced by a bond to a non-hydrogen substituent. Examples of non-hydrogen substituents include, but are not limited to amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl and/or hydroxyalkyl substituents, each of which may also be optionally substituted with one or more of the above substituents.

The term “effective amount” or “therapeutically effective amount” refers to that amount of a compound described herein that is sufficient to affect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying, or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

The term “inhibitor” refers to a compound having the ability to inhibit a biological function of a target protein, whether by inhibiting the activity or expression of the protein. Accordingly, the term “inhibitor” is defined in the context of the biological role of the target protein. In some embodiments, inhibitors specifically interact with (e.g., bind to) a target. In some embodiments, the biological activity inhibited is the development, growth, or spread of a tumor.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I)). Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of Structure (I).

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some embodiments, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents, or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) are a mixture of enantiomers or diastereomers. In other embodiments, the compounds of Structure (I) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Profesional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

The disclosure provides compounds including pharmaceutically acceptable salts, stereoisomers, and tautomers thereof, which are capable of selectively inhibiting HDAC6.

Accordingly, one embodiment provides a compound having the following Structure (I):

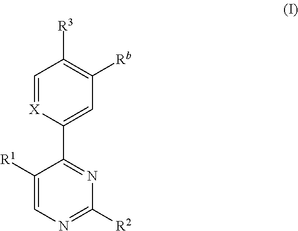

or a pharmaceutically acceptable salt, stereoisomer, or tautomer thereof, wherein:
X is N or CR^{a};
R^{a} is hydrogen or halo;
R^{b} is hydrogen, halo, or C₁-C₆ alkyl;
R¹ is hydrogen or halo;
R² has one of the following structures:

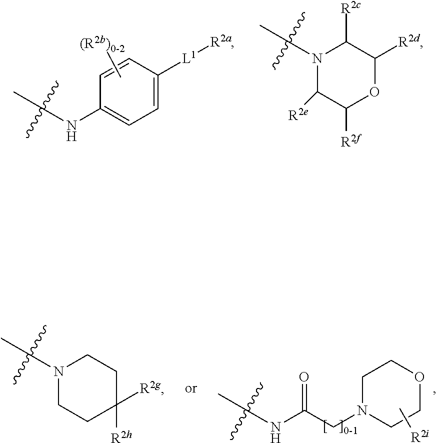

wherein:
L¹ is a direct bond, —CH₂—, —C(CH₃)H— or —C(═O)—;
R^{2a} has one of the following structures:

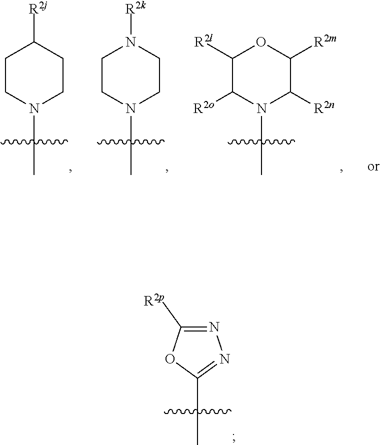

R^{2b} is halo;
R^{2c}, R^{2d}, R^{2e}, and R^{2f} are each independently hydrogen, C₁-C₆ alkyl, or
R^{2c} and R^{2f} join to form a C₁-C₆ alkylene bridge between the carbons to which they are attached, or
R^{2d} and R^{2e} join to form a C₁-C₆ alkylene bridge between the carbons to which they are attached;
R^{2g} and R^{2h} are each independently hydrogen, halo, or
R^{2g} and R^{2h}, together with the carbon to which they are attached join to form a 3-8 membered heterocyclyl;
R²ⁱ is hydrogen or C₁-C₆ alkyl;
R^{2j} is hydrogen, halo, or C₁-C₆ alkyl;
R^{2k} is hydrogen, C₁-C₆ alkyl, or 3 to 8 membered heterocyclyl;
R^{2l}, R^{2m}, R²ⁿn, and R^{2o} are each independently hydrogen, C₁-C₆ alkyl, or
R^{2l} and R²ⁿ join to form a C₁-C₆ alkylene bridge between the carbons to which they are attached, or
R^{2m} and R^{2o} join to form a C₁-C₆ alkylene bridge between the carbons to which they are attached;
R^{2p} is hydrogen, C₁-C₆ alkyl, or C₁-C₆ haloalkyl;
R³ has one of the following structures:

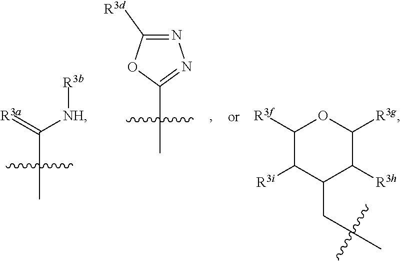

wherein:
R^{3a} is O or S;
R^{3b} is —OH, —NH—OH, —CN, or —NH—NR^{3e};
R^{3c} is hydrogen or —CH₃;
R^{3d} is C₁-C₆ haloalkyl; and
R^{3e} is C₁-C₆ alkyl;
   provided that when L¹ is a direct bond, R^{2b} is halo.

In certain embodiments, X is N. In some embodiments, X is CR^{a}. In certain embodiments, R^{a} is hydrogen. In some embodiments, R^{a} is fluoro.

In certain embodiments, R^{b} is hydrogen. In some embodiments, R^{b} is methyl.

In some embodiments, R¹ is hydrogen. In certain embodiments, R¹ is chloro.

In certain embodiments, R² has the following structure:

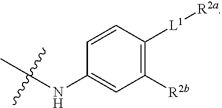

In certain embodiments, R² has the following structure:

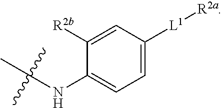

In some embodiments, R² has the following structure:

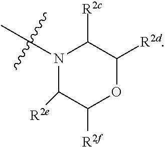

In certain embodiments, R² has the following structure:

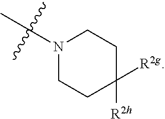

In some embodiments, R² has the following structure:

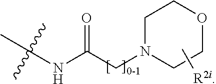

In certain embodiments, R² has one of the following structures:

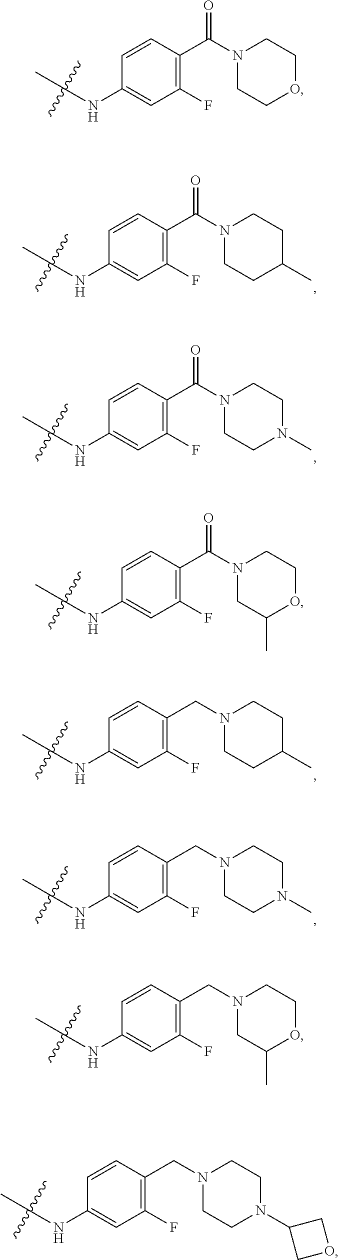

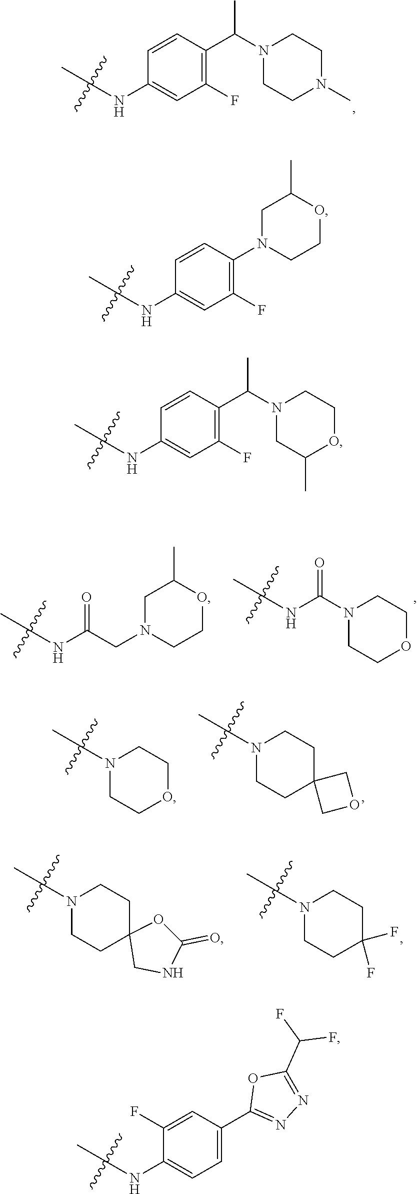

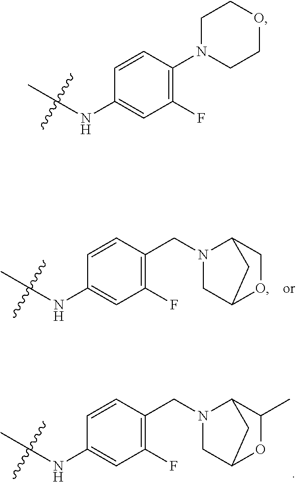 [SMILES: *Nc1ccc(N2CCOCC2)c(F)c1 ; *Nc1ccc(CN2CC3CC2CO3)c(F)c1 ; *Nc1ccc(CN2CC3CC2C(C)O3)c(F)c1]

In some embodiments, R² has one of the following structures:

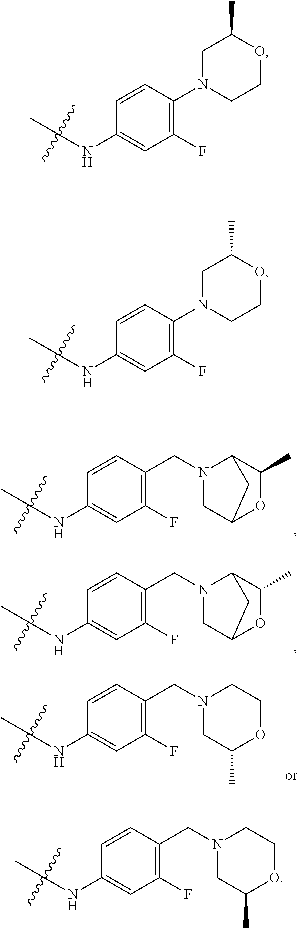

In certain embodiments, R³ has the following structure:

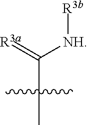

In some embodiments, R³ has the following structure:

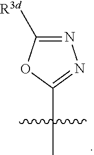

In certain embodiments, R³ has the following structure:

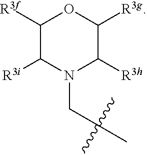

In some embodiments, R³ has one of the following structures:

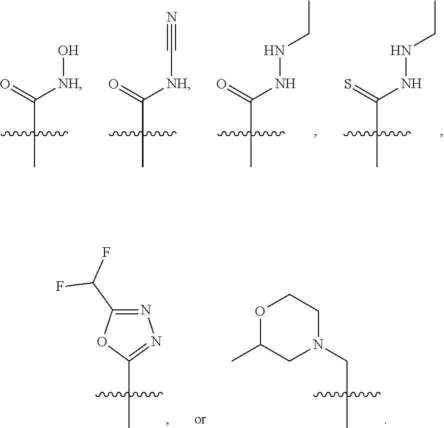

In certain embodiments, R³ has one of the following structures:

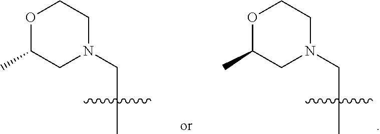

Some embodiments provide a salt form of the compound of Structure (I). In certain embodiments, the salt form is a formic acid salt, a hydrochloric acid salt, or a trifluoroacetic acid salt.

One embodiment provides a pharmaceutical composition comprising the compound of Structure (I), or a pharmaceutically acceptable salt, stereoisomer, or tautomer thereof, and a pharmaceutically acceptable excipient.

In various embodiments, the compound has one of the structures set forth in Table 1 below, or a pharmaceutically acceptable salt, stereoisomer, tautomer, solvate, polymorph, isotopologue, hydrate, or prodrug thereof. Compounds in Table 1 were prepared as described in the Examples or methods known in the art and analyzed by mass spectrometry and/or ¹H NMR.

TABLE 1
Representative compounds of Structure (I)

| No. | Structure | Name |
|---|---|---|
| I-1 | 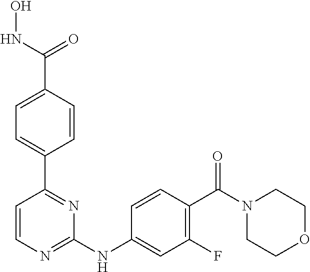 [SMILES: O=C(NO)c1ccc(-c2ccnc(Nc3ccc(C(=O)N4CCOCC4)c(F)c3)n2)cc1] | 4-(2-((3-fluoro-4-(morpholine-4- carbonyl)phenyl)amino)pyrimidin- 4-yl)-N-hydroxybenzamide |
| I-2 | 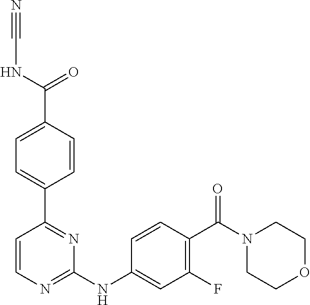 [SMILES: N#CNC(=O)c1ccc(-c2ccnc(Nc3ccc(C(=O)N4CCOCC4)c(F)c3)n2)cc1] | N-cyano-4-(2-((3-fluoro-4- (morpholine-4- carbonyl)phenyl)amino)pyrimidin- 4-yl)benzamide |
| I-3 | 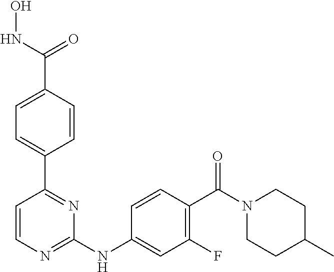 [SMILES: CC1CCN(C(=O)c2ccc(Nc3nccc(-c4ccc(C(=O)NO)cc4)n3)cc2F)CC1] | 4-(2-((3-fluoro-4-(4- methylpiperidine-1- carbonyl)phenyl)amino)pyrimidin- 4-yl)-N-hydroxybenzamide |
| I-4^{†} | 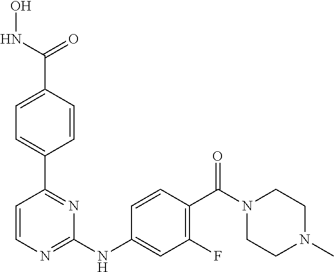 [SMILES: CN1CCN(C(=O)c2ccc(Nc3nccc(-c4ccc(C(=O)NO)cc4)n3)cc2F)CC1] | 4-(2-((3-fluoro-4-(4- methylpiperazine-1- carbonyl)phenyl)amino)pyrimidin- 4-yl)-N-hydroxybenzamide |
| I-5 | 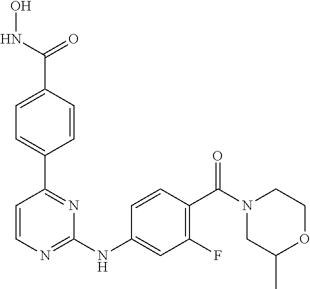 [SMILES: CC1CN(C(=O)c2ccc(Nc3nccc(-c4ccc(C(=O)NO)cc4)n3)cc2F)CCO1] | 4-(2-((3-fluoro-4-(2- methylmorpholine-4- carbonyl)phenyl)amino)pyrimidin- 4-yl)-N-hydroxybenzamide |
| I-6 | 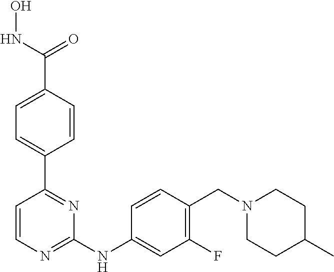 [SMILES: CC1CCN(Cc2ccc(Nc3nccc(-c4ccc(C(=O)NO)cc4)n3)cc2F)CC1] | 4-(2-((3-fluoro-4-((4- methylpiperidin-1- yl)methyl)phenyl)amino)pyrimidin- 4-yl)-N-hydroxybenzamide |
| I-7 | 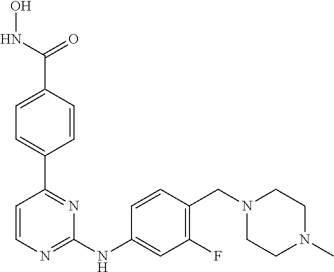 [SMILES: CN1CCN(Cc2ccc(Nc3nccc(-c4ccc(C(=O)NO)cc4)n3)cc2F)CC1] | 4-(2-((3-fluoro-4-((4- methylpiperazin-1- yl)methyl)phenyl)amino)pyrimidin- 4-yl)-N-hydroxybenzamide |
| I-8 | 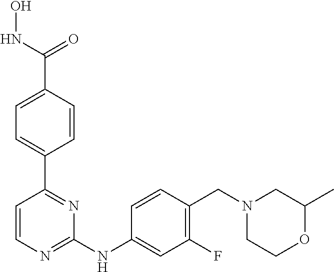 [SMILES: CC1CN(Cc2ccc(Nc3nccc(-c4ccc(C(=O)NO)cc4)n3)cc2F)CCO1] | 4-(2-((3-fluoro-4-((2- methylmorpholino)methyl)phenyl) amino)pyrimidin-4-yl)-N- hydroxybenzamide |
| I-9 | 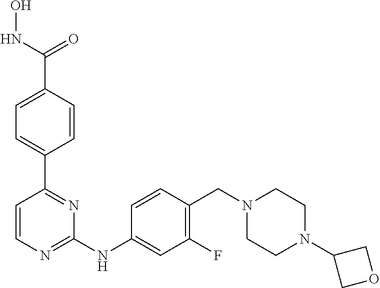 [SMILES: O=C(NO)c1ccc(-c2ccnc(Nc3ccc(CN4CCN(C5COC5)CC4)c(F)c3)n2)cc1] | 4-(2-((3-fluoro-4-((4-(oxetan-3- yl)piperazin-1- yl)methyl)phenyl)amino)pyrimidin- 4-yl)-N-hydroxybenzamide |
| I-10 | 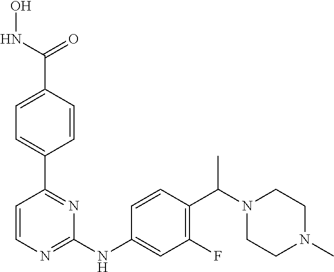 [SMILES: CC(c1ccc(Nc2nccc(-c3ccc(C(=O)NO)cc3)n2)cc1F)N1CCN(C)CC1] | 4-(2-((3-fluoro-4-(1-(4- methylpiperazin-1- yl)ethyl)phenyl)amino)pyrimidin- 4-yl)-N-hydroxybenzamide |
| I-11 | 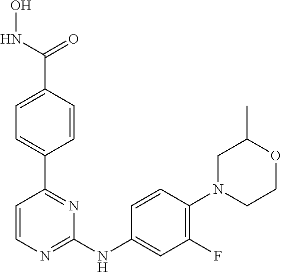 [SMILES: CC1CN(c2ccc(Nc3nccc(-c4ccc(C(=O)NO)cc4)n3)cc2F)CCO1] | 4-(2-((3-fluoro-4-(2- methylmorpholino)phenyl)amino) pyrimidin-4-yl)-N- hydroxybenzamide |
| I-12 | 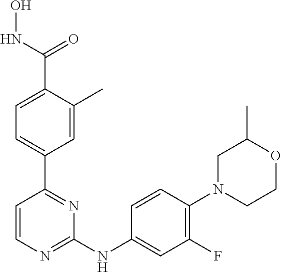 [SMILES: Cc1cc(-c2ccnc(Nc3ccc(N4CCOC(C)C4)c(F)c3)n2)ccc1C(=O)NO] | 4-(2-((3-fluoro-4-(2- methylmorpholino)phenyl)amino) pyrimidin-4-yl)-N-hydroxy-2- methylbenzamide |
| I-13 | 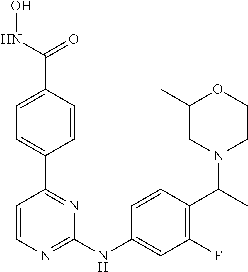 [SMILES: CC1CN(C(C)c2ccc(Nc3nccc(-c4ccc(C(=O)NO)cc4)n3)cc2F)CCO1] | 4-(2-((3-fluoro-4-(1-(2- methylmorpholino)ethyl)phenyl) amino)pyrimidin-4-yl)-N- hydroxybenzamide |
| I-14 | 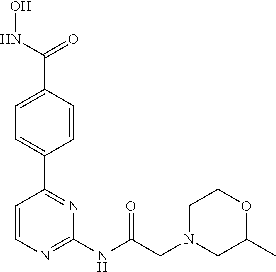 [SMILES: CC1CN(CC(=O)Nc2nccc(-c3ccc(C(=O)NO)cc3)n2)CCO1] | N-hydroxy-4-(2-(2-(2- methylmorpholino)acetamido) pyrimidin-4-yl)benzamide |
| I-15^{†} | 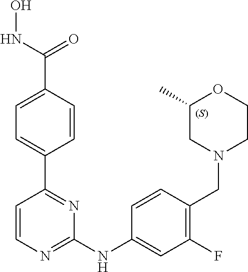 | (S)-4-(2-((3-fluoro-4-((2- methylmorpholino)methyl)phenyl) amino)pyrimidin-4-yl)-N- hydroxybenzamide |
| I-16^{Δ} | 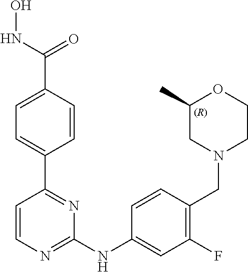 | (R)-4-(2-((3-fluoro-4-((2- methylmorpholino)methyl)phenyl) amino)pyrimidin-4-yl)-N- hydroxybenzamide |
| I-17 | 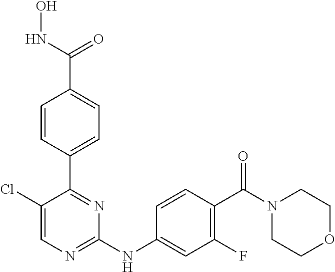 [SMILES: O=C(NO)c1ccc(-c2nc(Nc3ccc(C(=O)N4CCOCC4)c(F)c3)ncc2Cl)cc1] | 4-(5-chloro-2-((3-fluoro-4- (morpholine-4- carbonyl)phenyl)amino)pyrimidin- 4-yl)-N-hydroxybenzamide |
| I-18 | 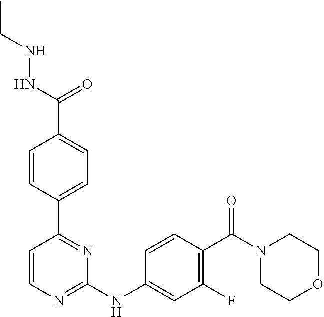 [SMILES: CCNNC(=O)c1ccc(-c2ccnc(Nc3ccc(C(=O)N4CCOCC4)c(F)c3)n2)cc1] | N′-ethyl-4-(2-((3-fluoro-4- (morpholine-4- carbonyl)phenyl)amino)pyrimidin- 4-yl)benzohydrazide |
| I-19 | 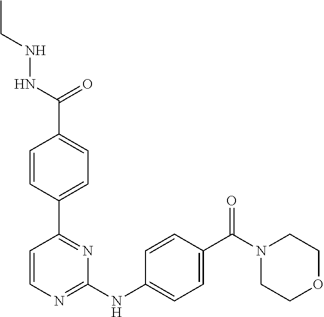 [SMILES: CCNNC(=O)c1ccc(-c2ccnc(Nc3ccc(C(=O)N4CCOCC4)cc3)n2)cc1] | N′-ethyl-4-(2-((4-(morpholine-4- carbonyl)phenyl)amino)pyrimidin- 4-yl)benzohydrazide |
| I-20 | 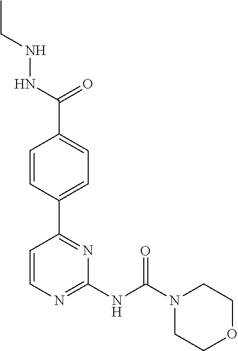 [SMILES: CCNNC(=O)c1ccc(-c2ccnc(NC(=O)N3CCOCC3)n2)cc1] | N-(4-(4-(2-ethylhydrazine-1- carbonyl)phenyl)pyrimidin-2- yl)morpholine-4-carboxamide |
| I-21 | 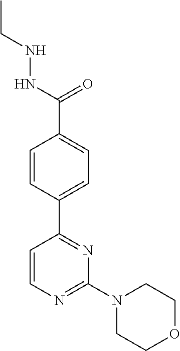 [SMILES: CCNNC(=O)c1ccc(-c2ccnc(N3CCOCC3)n2)cc1] | N′-ethyl-4-(2- morpholinopyrimidin-4- yl)benzohydrazide |
| I-22 | 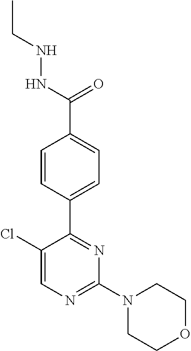 [SMILES: CCNNC(=O)c1ccc(-c2nc(N3CCOCC3)ncc2Cl)cc1] | 4-(5-chloro-2- morpholinopyrimidin-4-yl)-N′- ethylbenzohydrazide |
| I-23 | 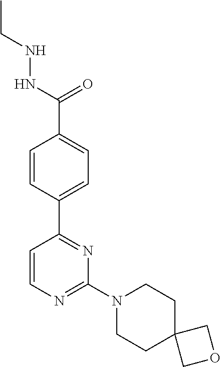 [SMILES: CCNNC(=O)c1ccc(-c2ccnc(N3CCC4(CC3)COC4)n2)cc1] | 4-(2-(2-oxa-7- azaspiro[3.5]nonan-7- yl)pyrimidin-4-yl)-N′- ethylbenzohydrazide |
| I-24 | 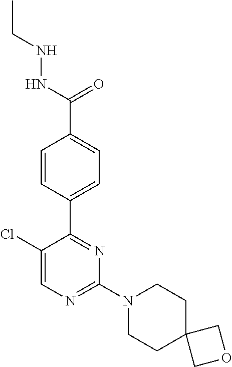 [SMILES: CCNNC(=O)c1ccc(-c2nc(N3CCC4(CC3)COC4)ncc2Cl)cc1] | 4-(5-chloro-2-(2-oxa-7- azaspiro[3.5]nonan-7- yl)pyrimidin-4-yl)-N′- ethylbenzohydrazide |
| I-25 | 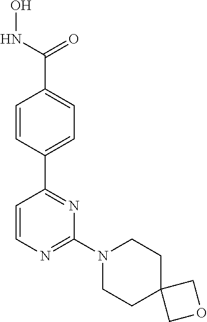 [SMILES: O=C(NO)c1ccc(-c2ccnc(N3CCC4(CC3)COC4)n2)cc1] | 4-(2-(2-oxa-7- azaspiro[3.5]nonan-7- yl)pyrimidin-4-yl)-N- hydroxybenzamide |
| I-26 | 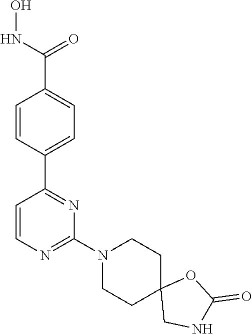 [SMILES: O=C1NCC2(CCN(c3nccc(-c4ccc(C(=O)NO)cc4)n3)CC2)O1] | N-hydroxy-4-(2-(2-oxo-1-oxa- 3,8-diazaspiro[4.5]decan-8- yl)pyrimidin-4-yl)benzamide |
| I-27 | 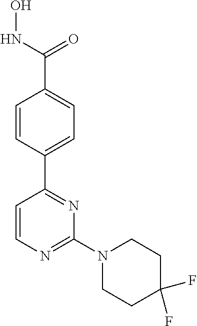 [SMILES: O=C(NO)c1ccc(-c2ccnc(N3CCC(F)(F)CC3)n2)cc1] | 4-(2-(4,4-difluoropiperidin-1- yl)pyrimidin-4-yl)-N- hydroxybenzamide |
| I-28 | 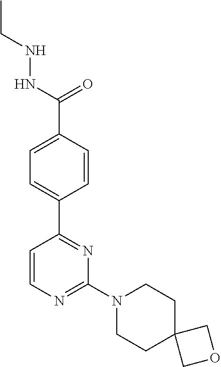 [SMILES: CCNNC(=O)c1ccc(-c2ccnc(N3CCC4(CC3)COC4)n2)cc1] | 4-(2-(2-oxa-7- azaspiro[3.5]nonan-7- yl)pyrimidin-4-yl)-N′- ethylbenzothiohydrazide |
| I-29 | 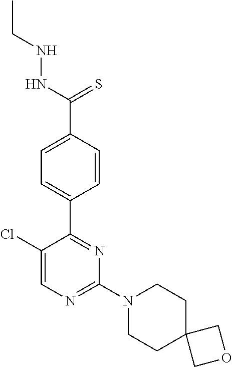 [SMILES: CCNNC(=S)c1ccc(-c2nc(N3CCC4(CC3)COC4)ncc2Cl)cc1] | 4-(5-chloro-2-(2-oxa-7- azaspiro[3.5]nonan-7- yl)pyrimidin-4-yl)-N′- ethylbenzothiohydrazide |
| I-30 | 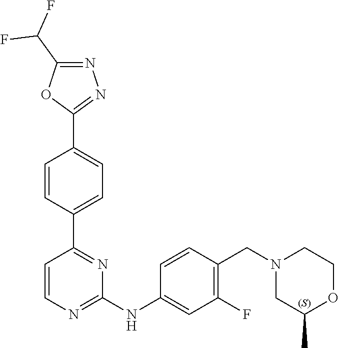 | (S)-4-(4-(5-(difluoromethyl)- 1,3,4-oxadiazol-2-yl)phenyl)-N- (3-fluoro-4-((2- methylmorpholino)methyl)phenyl) pyrimidin-2-amine |
| I-31 | 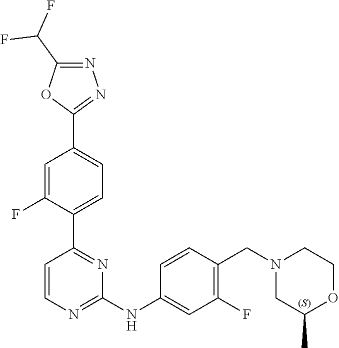 | (S)-4-(4-(5-(difluoromethyl)- 1,3,4-oxadiazol-2-yl)-2- fluorophenyl)-N-(3-fluoro-4-((2- methylmorpholino)methyl)phenyl) pyrimidin-2-amine |
| I-32 | 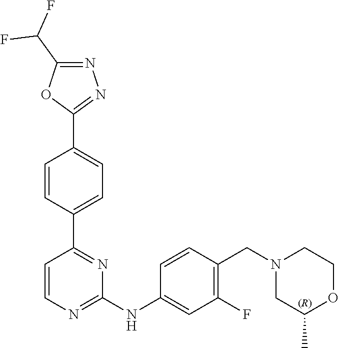 | (R)-4-(4-(5-(difluoromethyl)- 1,3,4-oxadiazol-2-yl)phenyl)-N- (3-fluoro-4-((2- methylmorpholino)methyl)phenyl) pyrimidin-2-amine TFA salt |
| I-33 | 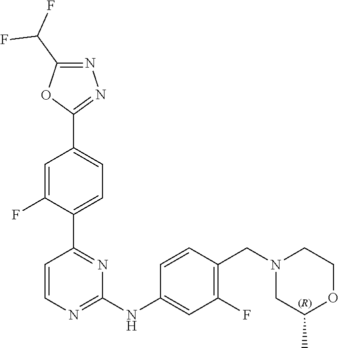 | (R)-4-(4-(5-(difluoromethyl)- 1,3,4-oxadiazol-2-yl)-2- fluorophenyl)-N-(3-fluoro-4-((2- methylmorpholino)methyl)phenyl) pyrimidin-2-amine |
| I-34 | 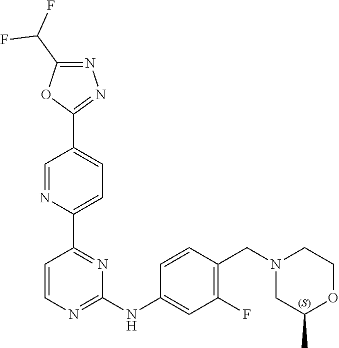 | (S)-4-(5-(5-(difluoromethyl)- 1,3,4-oxadiazol-2-yl)pyridin-2- yl)-N-(3-fluoro-4-((2- methylmorpholino)methyl)phenyl) pyrimidin-2-amine |
| I-35 | 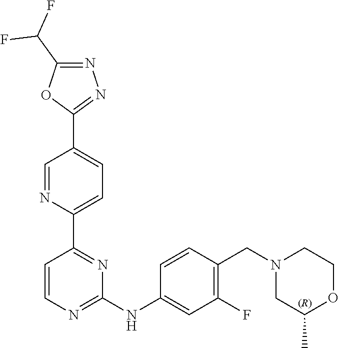 | (R)-4-(5-(5-(difluoromethyl)- 1,3,4-oxadiazol-2-yl)pyridin-2- yl)-N-(3-fluoro-4-((2- methylmorpholino)methyl)phenyl) pyrimidin-2-amine |
| I-36 | 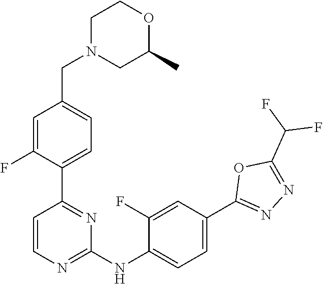 [SMILES: C[C@H]1CN(Cc2ccc(-c3ccnc(Nc4ccc(-c5nnc(C(F)F)o5)cc4F)n3)c(F)c2)CCO1] | (S)-N-(4-(5-(difluoromethyl)- 1,3,4-oxadiazol-2-yl)-2- fluorophenyl)-4-(2-fluoro-4-((2- methylmorpholino)methyl)phenyl) pyrimidin-2-amine |
| I-37 | 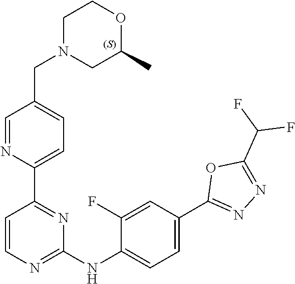 | (S)-N-(4-(5-(difluoromethyl)- 1,3,4-oxadiazol-2-yl)-2- fluorophenyl)-4-(5-((2- methylmorpholino)methyl)pyridin- 2-yl)pyrimidin-2-amine |
| I-38 | 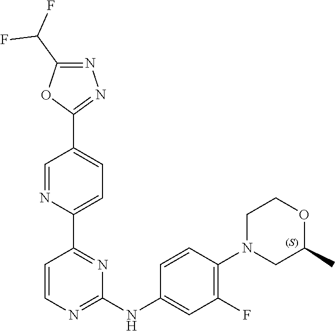 | (S)-4-(5-(5-(difluoromethyl)- 1,3,4-oxadiazol-2-yl)pyridin-2- yl)-N-(3-fluoro-4-(2- methylmorpholino)phenyl)pyrimidin- 2-amine |
| I-39 | 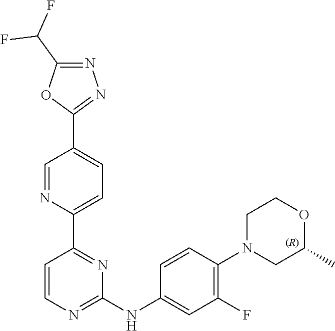 | (R)-4-(5-(5-(difluoromethyl)- 1,3,4-oxadiazol-2-yl)pyridin-2- yl)-N-(3-fluoro-4-(2- methylmorpholino)phenyl)pyrimidin- 2-amine |
| I-40 | 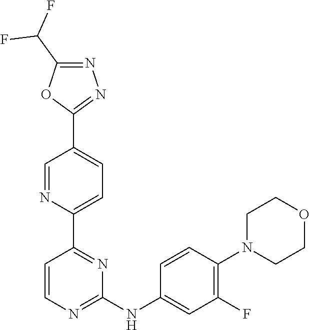 [SMILES: Fc1cc(Nc2nccc(-c3ccc(-c4nnc(C(F)F)o4)cn3)n2)ccc1N1CCOCC1] | 4-(5-(5-(difluoromethyl)-1,3,4- oxadiazol-2-yl)pyridin-2-yl)-N- (3-fluoro-4- morpholinophenyl)pyrimidin-2- amine |
| I-41 | 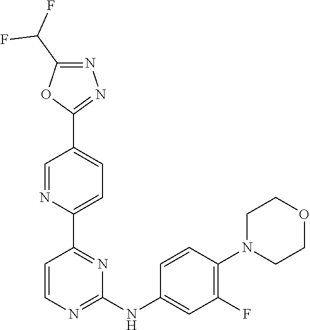 [SMILES: Fc1cc(Nc2nccc(-c3ccc(-c4nnc(C(F)F)o4)cn3)n2)ccc1N1CCOCC1] | 4-(5-(5-(difluoromethyl)-1,3,4- oxadiazol-2-yl)pyridin-2-yl)-N- (3-fluoro-4- morpholinophenyl)pyrimidin-2- amine |
| I-42 | 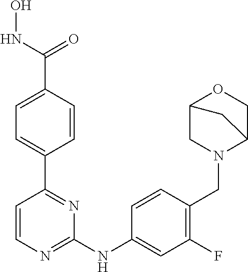 [SMILES: O=C(NO)c1ccc(-c2ccnc(Nc3ccc(CN4CC5CC4CO5)c(F)c3)n2)cc1] | 4-(2-((4-((2-oxa-5- azabicyclo[2.2.1]heptan-5- yl)methyl)-3- fluorophenyl)amino)pyrimidin-4- yl)-N-hydroxybenzamide |
| I-43 | 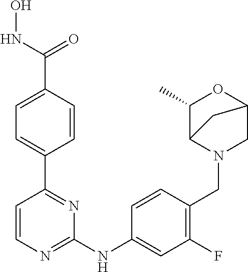 [SMILES: C[C@@H]1OC2CC1N(Cc1ccc(Nc3nccc(-c4ccc(C(=O)NO)cc4)n3)cc1F)C2] | 4-(2-((3-fluoro-4-(((3S)-3- methyl-2-oxa-5- azabicyclo[2.2.1]heptan-5- yl)methyl)phenyl)amino)pyrimidin- 4-yl)-N-hydroxybenzamide |

^{†}also obtained as a formic acid (HCOOH) salt
^{Δ}also obtained as a trifluoroacetic acid (TFA) salt

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” Prodrugs of compounds of this disclosure are included within the scope of embodiments of the disclosure.

### Pharmaceutical Compositions

Other embodiments are directed to pharmaceutical compositions. The pharmaceutical composition comprises anyone (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of at least one compound of Structure (I) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Provided herein are pharmaceutical compositions comprising one or more compounds of Structure (I), and a pharmaceutically acceptable carrier.

Provided herein are pharmaceutical compositions comprising one or more compounds selected from compounds of Structure (I) and pharmaceutically acceptable diluent(s), excipient(s), and carrier(s). In certain embodiments, the compounds described are administered as pharmaceutical compositions in which one or more compounds selected from compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

In a certain embodiment, pharmaceutical compositions of the compounds of Structure (I) are inhibitors of HDAC6.

A pharmaceutical composition, as used herein, refers to a mixture of one or more compounds selected from compounds of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) provided herein are administered in a pharmaceutical composition to a mammal having a disease, disorder, or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions, or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent, or excipient, and one or more compounds selected from compounds of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) illustratively takes the form of a liquid where the agents are present in solution, in suspension or both. Typically, when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite, and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds selected from compounds of Structure (I) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded, or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein.

For example, the pack may (1) contain metal or plastic foil (e.g., a blister pack), (2) be accompanied by instructions for administration, (3) be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound of Structure (I) formulated in a compatible pharmaceutical carrier are prepared, placed in an appropriate container, and labeled for treatment of an indicated condition.

### Methods

Embodiments of the present disclosure provide compounds that are useful as HDAC6 inhibitors in a host species. Therefore, the compounds of Structure (I) are also useful in the treatment of conditions mediated by HDAC6.

The host or patient can belong to any mammalian species, for example a primate species, particularly humans; rodents, including mice, rats, hamsters, rabbits, horses, cows, dogs, cats, etc. Animal models are of interest for experimental investigations, providing a model for treatment of human disease.

In one embodiment, the present disclosure is useful as an inhibitor of HDAC6. Therefore, the compounds of Structure (I) are also useful in the treatment of conditions resulting from overexpression of HDAC6 or HDAC6 activity.

Embodiments also relate to the use of compounds according to Structure (I) and/or physiologically acceptable salts thereof for the prophylactic or therapeutic treatment and/or monitoring of diseases that are caused, mediated, and/or modulated by HDAC6. Furthermore, embodiments relate to the use of compounds according to Structure (I) and/or physiologically acceptable salts thereof to produce a medicament for the prophylactic or therapeutic treatment and/or monitoring of diseases that are caused, mediated, and/or modulated by HDAC6. In certain embodiments, the disclosure provides the use of a compound according to Structure (I) or physiologically acceptable salts thereof, to produce a medicament for the prophylactic or therapeutic treatment of a HDAC6-mediated disorder.

In another embodiment, the present disclosure relates to a method of treating diseases or conditions mediated by HDAC6 by administering to a patient in need thereof a therapeutically effective amount of the compound of Structure (I).

One embodiment provides a method of treating a HDAC6 mediated disease, the method comprising administering the compound of Structure (I) or a pharmaceutically acceptable salt, stereoisomer, or tautomer thereof or the pharmaceutical composition comprising a compound of Structure (I) to a subject in need thereof.

In some embodiments, the HDAC6 mediated disease is cancer, a neurodegenerative disease, a neurological disorder, or an autoimmune disease.

In certain embodiments, the HDAC6 mediated disease is Alzheimer's disease, amyotrophic lateral sclerosis (ALS), Rett syndrome, idiopathic pulmonary fibrosis, and combinations thereof.

In certain embodiments, the HDAC6 mediated disease is non-small cell lung cancer, chronic lymphocytic leukemia, multiple myeloma, and combinations thereof.

In certain embodiments, the compounds of Structure (I) inhibit HDAC6 selectively over other HDAC family members (e.g., HDAC1, HDAC2, HDAC3, HDAC4, HDAC5, HDAC7, HDAC8, HDAC9, HDAC10, HDAC11, and combinations thereof. In some embodiments, the compounds of Structure (I) inhibit HDAC6 selectively as part of the treatment of amyotrophic lateral sclerosis (ALS).

Also included herein are methods of treatment in which at least one compound of Structure (I) is administered in combination with an anti-inflammatory or a therapeutic agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine.

Still other embodiments of the disclosure pertain to combinations in which at least one active agent is an immunosuppressant compound such as an immunosuppressant compound chosen from methotrexate, leflunomide, cyclosporine, tacrolimus, azathioprine, and mycophenolate mofetil.

The disclosed compounds of Structure (I) can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-3024, VAL-0834;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, or laromustine; Topoisomerase Inhibitors: such as etoposide, irinotecan, razoxane, sobuzoxane, teniposide, topotecan; amonafide, belotecan, elliptinium acetate, or voreloxin;
Microtubule modifiers: such as cabazitaxel, docetaxel, eribulin, ixabepilone, paclitaxel, vinblastine, vincristine, vinorelbine, vindesine, vinflunine; fosbretabulin, or tesetaxel;
Antimetabolites: such as asparaginase3, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, or trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, or pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, or enzalutamide;
Aromatase inhibitors: such as aminoglutethimide, anastrozole, exemestane, fadrozole, letrozole, testolactone, or formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, or cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein, or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the compounds of Structure (I) are administered as a monotherapy.

In some embodiments, the methods of the disclosure can be performed either in vitro, in vivo, or as a combination thereof. The susceptibility of a particular cell to treatment with the compounds of Structure (I) can be particularly determined by in vitro tests, whether during research or clinical application. Typically, a culture of the cell is combined with a compound at various concentrations for a period which is sufficient to allow the active agents to inhibit HDAC6 activity, usually between about one hour and one week. In vitro treatment can be carried out using cultivated cells from a biopsy sample or cell line.

In some embodiments, the IC₅₀ of the compounds of Structure (I) to inhibit HDAC6 was determined by the concentration of the compound required to inhibit 50% of the activity of HDAC6.

It is understood that one skilled in the art may be able to make these compounds by similar methods or by combining other methods known to one skilled in the art. It is also understood that one skilled in the art would be able to make, in a similar manner as described below, other compounds of Structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this disclosure.

The following examples are provided for purpose of illustration and not limitation.

### Abbreviations

° C. (degree Celsius); ¹H NMR (proton Nuclear Magnetic Resonance); DCM (dichloromethane); DIPEA (diisopropylethylamine); DMSO (dimethylsulfoxide); DMF (dimethylformamide); EDC-HCl (1-ethyl-3-(3-dimethylaminopropyl)carbodiimide); eq (equivalent); EtOAc (ethyl acetate); Et₃N or TEA (triethylamine); g (gram); h (hour); MeOH (methanol); mg (milligram); min (minute); mL (milliliter); mmol (millimole); TFA (trifluoroacetic acid); THF (tetrahydrofuran); TLC (Thin Layer Chromatography); LDA (lithium diisopropylamide); AcOH (acetic acid); mCPBA (3-chloroperbenzoic acid), p-TSA (para toluene sulfonic acid); HOBt (hydroxybenzotrazole).

### Additional Abbreviations

| Abbreviation | Expansion |
|---|---|
| ALS | Amyotrophic Lateral Sclerosis |
| ad libitum | meaning “without restriction” |
| ANOVA | Analysis of Variance |
| g | Gram |
| h | Hour or hours |
| s | Second or seconds |
| kg | Kilogram |
| mg | Milligram |
| mL | Milliliter |
| QD | quaque die (Once daily) |
| P value | Calculated Probability |
| p.o. | “Per os” (Latin)[English Meaning- Mouth/orally] or per |
| | oral |
| RO | Reverse Osmosis |
| RPM | Revolutions per minute |
| SEM | Standard Error of Mean |
| UV | Ultraviolet |
| ° C. | Degree Celsius |
| % | Percentage |
| CSF | Cerebrospinal fluid |

### Synthetic Example 1

### Synthesis of 4-(2-((3-Fluoro-4-(Morpholine-4-Carbonyl) Phenyl)Amino)Pyrimidin-4-Yl)-N-Hydroxybenzamide

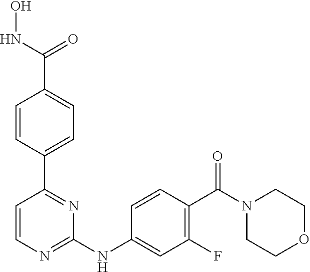 [SMILES: O=C(NO)c1ccc(-c2ccnc(Nc3ccc(C(=O)N4CCOCC4)c(F)c3)n2)cc1]

### Step 1: Synthesis of (2-fluoro-4-nitrophenyl)(morpholino)methanone

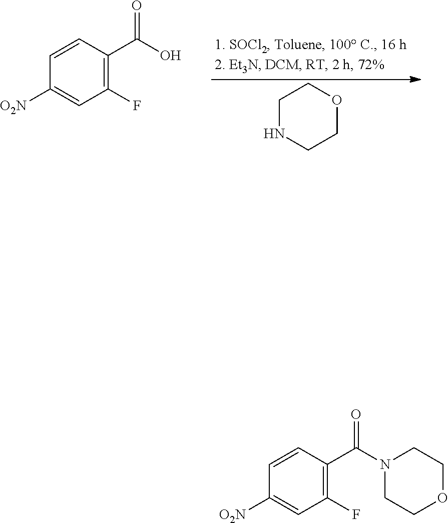 [SMILES: O=C(O)c1ccc([N+](=O)[O-])cc1F ; C1COCCN1 ; O=C(c1ccc([N+](=O)[O-])cc1F)N1CCOCC1]

To a stirred solution of 2-fluoro-4-nitrobenzoic acid (5 g, 27.0 mmol, 1.0 eq) in toluene (50 mL), at 0° C., was added SOCl₂ (9.64 g, 81.1 mmol, 3 eq) and stirred at 100° C. for 16 h. After completion of reaction by TLC, volatiles removed under vacuum, dissolved in dichloromethane (53 mL) at room temperature and added Et₃N (7.91 g, 78.3 mmol, 2.9 eq.), morpholine (2.72 g, 31.3 mmol, 1.15 eq) and stirred for 2 h. After completion of reaction by TLC, diluted with water (50 mL) and extracted with dichloromethane (2×100 mL). The organic layer was dried over Na₂SO₄, filtered, and concentrated under reduced pressure to afford (2-fluoro-4-nitrophenyl) (morpholino)methanone as yellow solid (5.0 g, yield: 72%).

TLC system: EtOAc/Hexane (1:0)

R_{f} value: ˜0.5

LCMS(m/z): 255.0 (M+H)⁺

¹H NMR (400 MHz, CDCl₃) δ: 8.15-8.12 (m, 1H), 8.01 (dd, J=8.8, 2.0 Hz, 1H), 7.61 (d, J=8.8, 6.8 Hz, 1H), 3.85-3.78 (m, 4H), 3.68-3.65 (m, 2H), 3.34-3.31 (m, 2H)

### Step 2: Synthesis of (4-amino-2-fluorophenyl)(morpholino)methanone

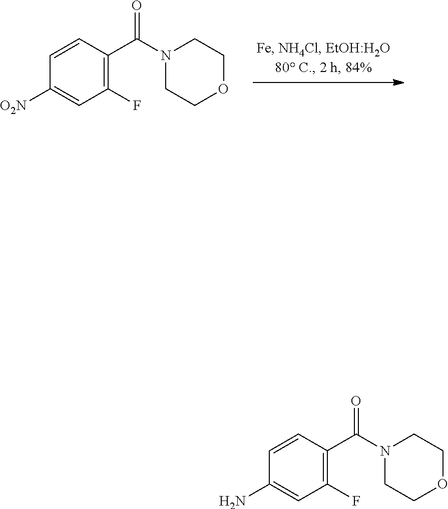 [SMILES: O=C(c1ccc([N+](=O)[O-])cc1F)N1CCOCC1 ; Nc1ccc(C(=O)N2CCOCC2)c(F)c1]

To a stirred solution of (2-fluoro-4-nitrophenyl)(morpholino)methanone (3 g, 11.7 mmol, 1 eq) in EtOH:H₂O (2:1; 30 mL) at room temperature was added Fe (3.3 g, 58.8 mmol, 5 eq) and NH₄Cl (3.12 g, 58.8 mmol, 5 eq) and stirred at 80° C. for 2 h. After completion of reaction by TLC, reaction mixture was filtered through a pad of diatomaceous earth (e.g., Celite®) and washed with EtOAc (50 mL). Filtrate evaporated, diluted with water (30 mL) and extracted with EtOAc (3×30 mL). The organic layer was dried over Na₂SO₄, filtered, and concentrated under reduced pressure to afford (4-amino-2-fluorophenyl) (morpholino)methanone as yellow gummy mass (2.2 g, yield: 84%).

TLC system: EtOAc/Hexane (1:0)

R_{f} value: ˜0.4

LCMS(m/z): 225.0 (M+H)⁺.

### Step 3: Synthesis of methyl 4-(2-chloropyrimidin-4-yl)benzoate

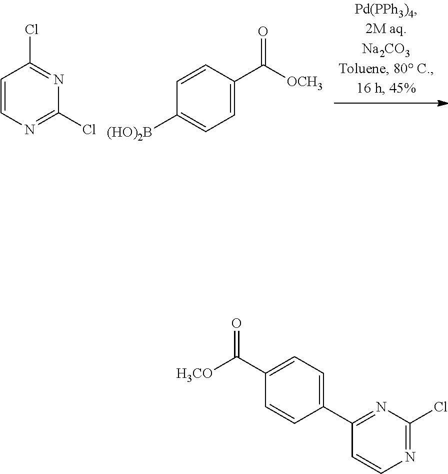 [SMILES: Clc1ccnc(Cl)n1 ; COC(=O)c1ccc(B(O)O)cc1 ; COC(=O)c1ccc(-c2ccnc(Cl)n2)cc1]

In a sealed tube, to a solution of 2,4-dichloropyrimidine (4 g, 26.8 mmol, 1 eq) and (4-(methoxycarbonyl)phenyl)boronic acid (7.25 g, 40.2 mmol, 1.5 eq) in toluene (56 mL) was added 2M aq. Na₂CO₃ (13.2 mL, 3.3 Vol) and degassed for 10 min, Later, added Pd(PPh₃)₄ (0.62 g, 0.54 mmol, 0.02 eq) and heated to 80° C., stirred for 16 h. After completion of reaction by TLC, reaction mixture was diluted with water (100 mL) and extracted with EtOAc (3×75 mL). The organic layer was dried over Na₂SO₄, filtered, and concentrated under reduced pressure to afford crude product. The crude product was triturated with MeOH (20 mL), filtered and collected solid was dried under vacuum to afford methyl 4-(2-chloropyrimidin-4-yl)benzoate as pale yellow solid (3 g, yield: 45%).

TLC system: EtOAc/Hexane (50:50)

R_{f} value: ˜0.4

LCMS(m/z): 249.0 (M+H)⁺

¹H NMR (400 MHz, CDCl₃) δ: 8.71 (d, J=5.2 Hz, 1H), 8.20-8.15 (m, 4H), 7.71 (d, J=5.2 Hz, 1H), 3.97 (s, 3H)

### Step 4: Synthesis of methyl 4-(2-((3-fluoro-4-(morpholine-4-carbonyl) phenyl)amino) pyrimidin-4-yl)benzoate

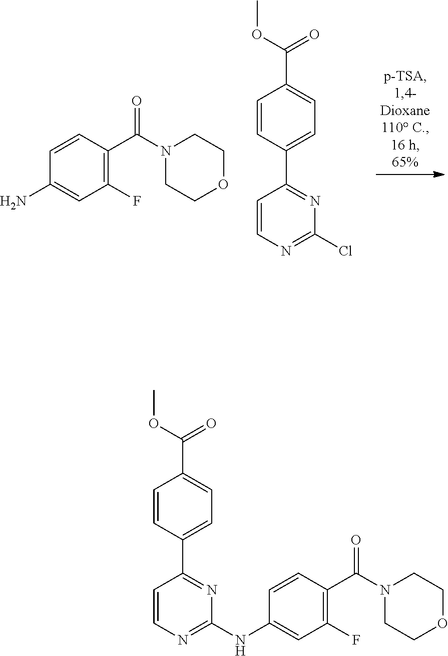

To a stirred solution of methyl 4-(2-chloropyrimidin-4-yl)benzoate (2 g, 8.03 mmol, 1 eq) in 1,4-dioxane (50 mL) at room temperature, was added p-TSA (1.37 g, 7.23 mmol, 0.9 eq), (4-amino-2-fluorophenyl)(morpholino)methanone (1.8 g, 8.03 mmol, 1 eq.) and stirred at 110° C. for 16 h. After completion of reaction by TLC, reaction mixture was diluted with water (80 mL) and extracted with EtOAc (2×80 mL). The combined organic layer was washed with Aq. NaHCO₃ solution, dried over anhydrous Na₂SO₄, filtered, and concentrated under reduced pressure to afford crude product. The crude product was purified by silica gel (100-200 mesh) column chromatography [elution with 70% EtOAc in Hexane] to afford methyl 4-(2-((3-fluoro-4-(morpholine-4-carbonyl) phenyl) amino)pyrimidin-4-yl)benzoate as pale yellow solid (2.3 g, yield: 65%).

TLC system: EtOAc (100%)

R_{f} value: ˜0.3

¹H NMR (400 MHz, CDCl₃) δ: 8.58 (d, J=5.2 Hz, 1H), 8.19 (d, J=8.8 Hz, 2H), 8.14 (d, J=8.8 Hz, 2H), 7.95 (dd, J=12, 2.0 Hz, 1H), 7.43-7.39 (m, 2H), 7.30 (d, J=5.2 Hz, 1H), 3.97 (s, 3H), 3.82-3.79 (m, 4H), 3.69-3.67 (m, 2H), 3.46-3.43 (m, 2H)

### Step 5: Synthesis of 4-(2-((3-fluoro-4-(morpholine-4-carbonyl) phenyl) amino)pyrimidin-4-yl)benzoic acid

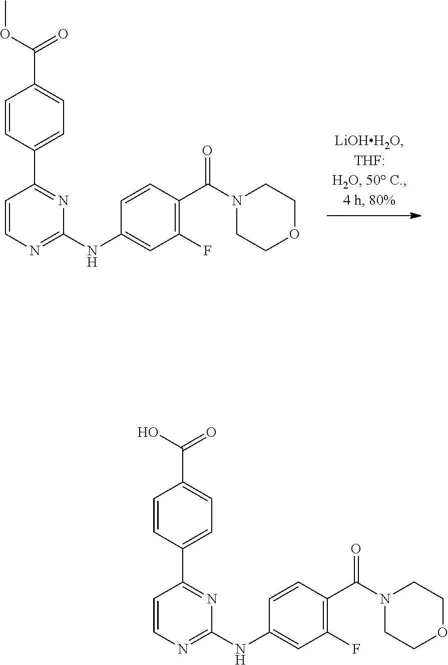 [SMILES: COC(=O)c1ccc(-c2ccnc(Nc3ccc(C(=O)N4CCOCC4)c(F)c3)n2)cc1 ; O=C(O)c1ccc(-c2ccnc(Nc3ccc(C(=O)N4CCOCC4)c(F)c3)n2)cc1]

To a stirred solution of methyl 4-(2-((3-fluoro-4-(morpholine-4-carbonyl)phenyl)amino)pyrimidin-4-yl)benzoate (2.3 g, 5.27 mmol, 1 eq) in THF:H₂O (2:1) (25 mL) at 0° C., added LiOH·H₂O (443 mg, 10.5 mmol, 2 eq) and stirred at 50° C. for 4 h. After completion of reaction by TLC, reaction mixture was washed with diethyl ether (30 mL). The aqueous layer was acidified with 2N HCl solution (pH-2), precipitated solid was filtered dried under vacuum to afford 4-(2-((3-fluoro-4-(morpholine-4-carbonyl)phenyl)amino)pyrimidin-4-yl)benzoic acid as a yellow solid (1.75 g, yield: 80%).

TLC system: MeOH/dichloromethane (10:90)

R_{f} value: ˜0.2

LCMS(m/z): 423.2 (M+H)⁺

¹H NMR (400 MHz, DMSO-d₆) δ: 13.2 (brs, 1H), 10.24 (s, 1H), 8.70 (d, J=5.2 Hz, 1H), 8.29 (d, J=8.4 Hz, 2H), 8.12 (d, J=8.4 Hz, 2H), 7.95 (dd, J=12.8, 1.6 Hz, 1H), 7.67 (dd, J=8.4, 2.0 Hz, 1H), 7.59 (d, J=5.2 Hz, 1H), 7.37 (t, J=8.0 Hz, 1H), 3.64 (brs, 4H), 3.55 (br, 2H), 3.30 (br, 2H)

### Step 6: Synthesis of 4-(2-((3-fluoro-4-(morpholine-4-carbonyl)phenyl)amino)pyrimidin-4-yl)-N-((tetrahydro-2H-pyran-2-yl)oxy)benzamide

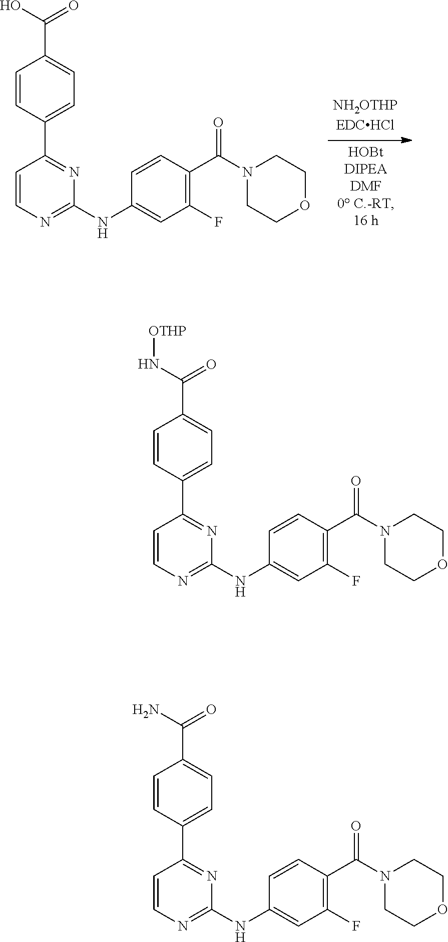

To a stirred solution of 4-(2-((3-fluoro-4-(morpholine-4-carbonyl)phenyl)amino)pyrimidin-4-yl)benzoic acid (400 mg, 0.95 mmol, 1 eq) in DMF (4 mL) at 0° C., was added DIPEA (330 mg, 2.56 mmol, 2.7 eq), HOBt (128 mg, 0.95 mmol, 1 eq), EDC-HCl (383 mg, 1.99 mmol, 2.1 eq) and stirred for 10-15 min. Later, added NH₂OTHP (133 mg, 1.13 mmol, 1.2 eq) and stirred at room temperature for 16 h. After completion of reaction by TLC, diluted with ice cold water (15 mL), and extracted with 10% MeOH in dichloromethane (2×20 mL). The combined organic layer was dried over Na₂SO₄ and evaporated under reduced pressure to afford 4-(2-((3-fluoro-4-(morpholine-4-carbonyl)phenyl)amino)pyrimidin-4-yl)-N-((tetrahydro-2H-pyran-2-yl)oxy)benzamide as yellow gummy mass (300 mg crude). Crude compound was taken forward to next step without purification.

TLC system: MeOH/dichloromethane (10:90)

R_{f} value: ˜0.5; LCMS(m/z): 522.3 (M+H)⁺; 48% desired produce and 39% byproduct as shown

### Step 7: Synthesis of 4-(2-((3-fluoro-4-(morpholine-4-carbonyl)phenyl)amino)pyrimidin-4-yl)-N-hydroxybenzamide

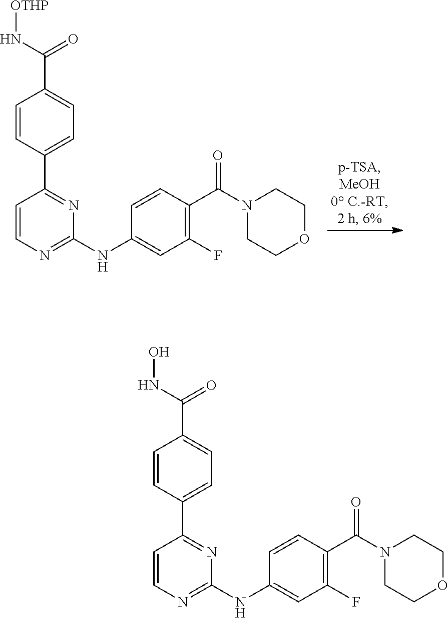

To a stirred solution of 4-(2-((3-fluoro-4-(morpholine-4-carbonyl)phenyl)amino)pyrimidin-4-yl)-N-((tetrahydro-2H-pyran-2-yl)oxy)benzamide (300 mg, 0.57 mmol, 1 eq (LCMS: 49%)) in MeOH (3 mL) at room temperature, was added p-TSA (77 mg, 0.4 mmol, 0.7 eq) and stirred for 2 h. After completion of reaction by TLC, diluted with water (20 mL) and extracted with 10% MeOH in dichloromethane (2×20 mL). Organic layer was dried over anhydrous Na₂SO₄, filtered, and concentrated under reduced pressure to afford crude product. The crude product was purified by reverse phase column chromatography (Grace) [elution with 20% acetonitrile in 0.1% formic acid in H₂O] to afford 4-(2-((3-fluoro-4-(morpholine-4-carbonyl)phenyl)amino)pyrimidin-4-yl)-N-hydroxybenzamide as white solid (15 mg, yield: 6%).

TLC system: MeOH/dichloromethane (10:90)

R_{f} value: ˜0.3

LCMS (m/z): 436.3 (M−H)⁺

¹H NMR (400 MHz, DMSO-d₆) δ: 11.39 (br, 1H), 10.21 (s, 1H), 9.18 (br, 1H), 8.67 (d, J=5.2 Hz, 1H), 8.25 (d, J=8.4 Hz, 2H), 7.99-7.92 (m, 3H), 7.64 (dd, J=8.4, 2.0 Hz, 1H), 7.58 (d, J=5.2 Hz, 1H), 7.36 (t, J=8.0 Hz, 1H), 3.64 (brs, 4H), 3.55 (brs, 2H), 3.32 (brs, 2H)

### Synthetic Example 2

### Synthesis of N-Cyano-4-(2-((3-Fluoro-4-(Morpholine-4-Carbonyl)Phenyl)Amino)Pyrimidin-4-Yl)Benzamide

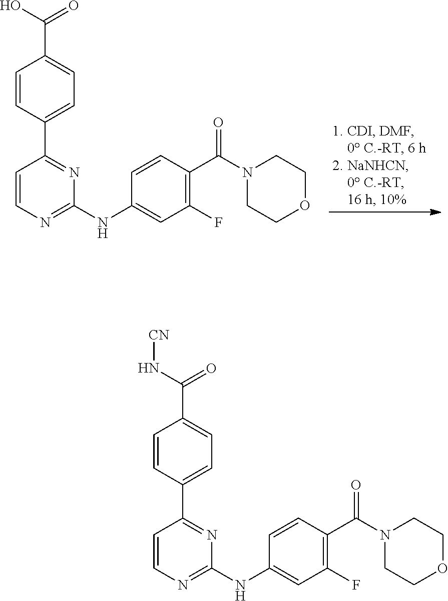 [SMILES: O=C(O)c1ccc(-c2ccnc(Nc3ccc(C(=O)N4CCOCC4)c(F)c3)n2)cc1 ; N#CNC(=O)c1ccc(-c2ccnc(Nc3ccc(C(=O)N4CCOCC4)c(F)c3)n2)cc1]

To a stirred solution of 4-(2-((3-fluoro-4-(morpholine-4-carbonyl)phenyl)amino)pyrimidin-4-yl)benzoic acid (350 mg, 0.83 mmol, 1.0 eq) in DMF (3.5 mL) at 0° C., was added CDI (269 mg, 1.66 mmol, 2.0 eq) and stirred at room temperature for 6 h. Later at 0° C., added NaNHCN (213 mg, 3.32 mmol, 4 eq) and stirred at room temperature for 16 h. After completion of reaction by TLC, reaction mixture was concentrated under reduced pressure, obtained residue was triturated with EtOAc (10 mL), filtered and solid was dried under vacuum to afford semi-pure product. Purified by prep-HPLC and after lyophilization found that it contains NH₄Cl salts which was re-purified by reverse phase column chromatography (Grace) [elution with 40% acetonitrile in 0.1% formic acid in H₂O] to afford N-cyano-4-(2-((3-fluoro-4-(morpholine-4-carbonyl)phenyl)amino)pyrimidin-4-yl)benzamide as an off white solid (35 mg, yield: 10%).

TLC system: MeOH/dichloromethane (20:80)

R_{f} value: ˜0.3

LCMS (m/z): 447.0 (M+H)⁺

¹H NMR (400 MHz, DMSO-d₆) δ: 10.22 (s, 1H), 8.70 (d, J=5.2 Hz, 1H), 8.30 (d, J=8.4 Hz, 2H), 8.08 (d, J=8.4 Hz, 2H), 7.96 (d, J=13.2, 2.0 Hz, 1H), 7.64 (d, J=8.4, 2.0 Hz, 1H), 7.61 (d, J=5.2 Hz, 1H), 7.36 (t, J=8.4 Hz, 1H), 3.64 (brs, 4H), 3.55 (br, 2H), 3.31 (br, 2H)

### Synthetic Example 3

### Synthesis of 4-(2-((3-Fluoro-4-(4-Methylpiperidine-1-Carbonyl)Phenyl)Amino)Pyrimidin-4-Yl)-N-Hydroxybenzamide

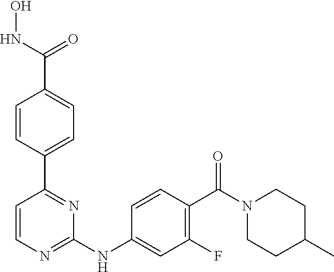 [SMILES: CC1CCN(C(=O)c2ccc(Nc3nccc(-c4ccc(C(=O)NO)cc4)n3)cc2F)CC1]

### Step 1: Synthesis of (2-fluoro-4-nitrophenyl)(4-methylpiperidin-1-yl)methanone

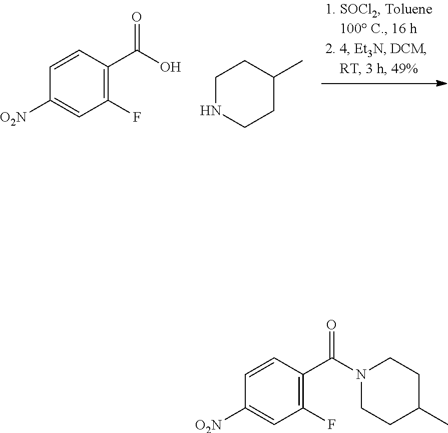

To a stirred solution of 2-fluoro-4-nitrobenzoic acid (5 g, 27.0 mmol, 1.0 eq) in toluene (50 mL), at room temperature, was added SOCl₂ (9.24 g, 81.0 mmol, 3.0 eq) and stirred at 100° C. for 16 h. After completion of reaction by TLC, volatiles removed under vacuum, dissolved in dichloromethane (50 mL), added Et₃N (4.47 g, 44.3 mmol, 1.6 eq) and 1-methylpiperazine (3.21 g, 32.4 mmol, 1.2 eq) at room temperature and stirred for 3 h. After completion of reaction by TLC, diluted with water (100 mL) and extracted with EtOAc (3×50 mL). Organic layer was dried over Na₂SO₄, filtered and concentrated under reduced pressure to afford (2-fluoro-4-nitrophenyl)(4-methylpiperidin-1-yl)methanone as yellow gum (3.5 g, yield: 49%).

TLC system: EtOAc/Hexane (100:00)

R_{f} value: ˜0.5

LCMS(m/z): 267.1 (M+H)⁺

¹H NMR (400 MHz, CDCl₃) δ: 8.11-8.08 (m, 1H), 8.00-7.97 (m, 1H), 7.58-7.54 (m, 1H), 4.73-4.68 (m, 1H), 3.43-3.38 (m, 1H), 3.08-3.05 (m, 1H), 2.81 (t, J=11.6 Hz, 1H), 1.82-1.77 (m, 1H), 1.69-1.62 (m, 2H), 1.33 (t, J=7.2 Hz, 1H), 1.26-1.23 (m, 1H), 0.99 (d, J=6.4 Hz, 3H)

### Step 2: Synthesis of (4-amino-2-fluorophenyl)(4-methylpiperidin-1-yl)methanone

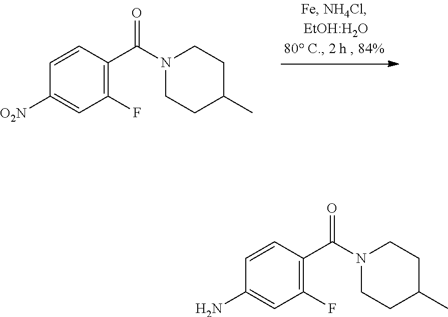 [SMILES: CC1CCN(C(=O)c2ccc([N+](=O)[O-])cc2F)CC1 ; CC1CCN(C(=O)c2ccc(N)cc2F)CC1]

To a stirred solution of (2-fluoro-4-nitrophenyl)(4-methylpiperidin-1-yl)methanone (3.5 g, 13.1 mmol, 1 eq) in EtOH:H₂O (50 mL) at room temperature was added iron (3.67 g, 65.5 mmol, 5 eq) and NH₄Cl (3.47 g, 65.5 mmol, 5 eq) and heated to 80° C., stirred for 2 h. After completion of reaction by TLC, reaction mixture was filtered through a pad of diatomaceous earth (e.g., Celite®) and washed with EtOAc (30 mL). The filtrate was concentrated, diluted with water (50 mL) and extracted with EtOAc (2×40 mL). The organic layer was dried over Na₂SO₄, filtered and concentrated under reduced pressure to afford (4-amino-2-fluorophenyl)(4-methylpiperidin-1-yl)methanone as yellow gum (2.6 g, yield: 84%).

TLC system: EtOAc/Hexane (100:00)

R_{f} value: ˜0.3

LCMS(m/z): 237.1 (M+H)⁺

¹H NMR (400 MHz, CDCl₃) δ: 7.17 (t, J=8.0 Hz, 1H), 6.45 (dd, J=2.4 Hz, 8.4 Hz, 1H), 6.33 (dd, J=2.4 Hz, 11.6 Hz, 1H), 4.68-4.65 (m, 1H), 3.92 (brs, 2H), 3.63-3.60 (m, 1H), 3.00 (t, 1H), 2.74 (t, J=12 Hz, 1H), 1.75-1.71 (m, 1H), 1.63-1.58 (m, 2H), 1.18-1.11 (m, 2H), 0.96 (d, J=6.4 Hz, 3H)

### Step 3: Synthesis of N-(benzyloxy)-4-bromobenzamide

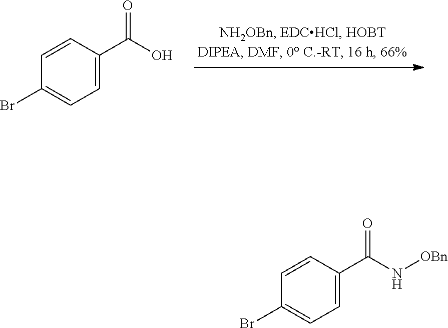 [SMILES: O=C(O)c1ccc(Br)cc1 ; O=C(NOCc1ccccc1)c1ccc(Br)cc1]

To a stirred solution of 4-bromobenzoic acid (3 g, 14.9 mmol, 1 eq) in DMF (2.5 mL) at 0° C., was added DIPEA (5.76 g, 44.8 mmol, 3.0 eq), HOBt (2.0 g, 14.9 mmol, 1.0 eq) followed by EDC-HCl (2.86 g, 14.9 mmol, 1.0 eq) and stirred for 10 min. Later added NH₂OBn (2.0 g, 17.9 mmol, 1.2 eq) and stirred at room temperature for 16 h. After completion of reaction by TLC, reaction mixture was quenched with ice cold water (80 mL) and extracted with 10% MeOH in dichloromethane (3×30 mL). Combined organic layer was dried over Na₂SO₄ and evaporated under reduced pressure to afford N-(benzyloxy)-4-bromobenzamide as yellow solid (3 g, Yield: 66%).

TLC system: EtOAc/Hexane (50:50)

R_{f} value: ˜0.4

LCMS(m/z): 305.9 (M+H)⁺

¹H NMR (400 MHz, CDCl₃) δ: 8.50 (bs, 1H), 7.56 (brs, 4H), 7.47-7.40 (m, 5H), 5.05 (s, 2H)

### Step 4: Synthesis of N-(benzyloxy)-4-(4,4,5,5-tetramethyl-1,3,2-dioxaborolan-2-yl)benzamide

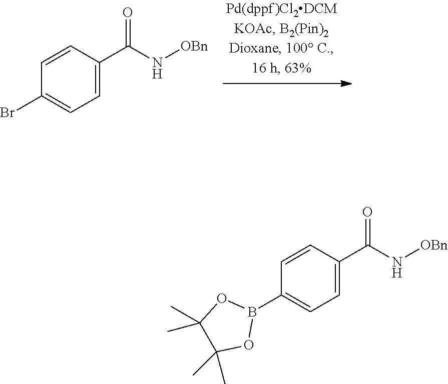 [SMILES: O=C(NOCc1ccccc1)c1ccc(Br)cc1 ; CC1(C)OB(c2ccc(C(=O)NOCc3ccccc3)cc2)OC1(C)C]

To a degassed stirred solution of N-(benzyloxy)-4-bromobenzamide (3 g, 9.83 mmol, 1 eq) and B₂(Pin)₂ (2.9 g, 11.8 mmol, 1.2 eq) in 1,4-dioxane (60 mL) was added KOAc (2.89 g, 29.5 mmol, 3.0 eq) followed by Pd(dppf)Cl₂. dichloromethane (0.4 g, 0.49 mmol, 0.05 eq) and heated to 100° C., stirred for 16 h in a sealed tube. After completion of reaction by TLC, reaction mixture was diluted with water (40 mL) and extracted with (2×40 mL). Organic layer was dried over Na₂SO₄, filtered, and concentrated under reduced pressure to afford crude product. The crude product was purified by silica gel (100-200 mesh) column chromatography [elution with 30% EtOAc in Hexane] to afford N-(benzyloxy)-4-(4,4,5,5-tetramethyl-1,3,2-dioxaborolan-2-yl)benzamide as yellow solid (2.2 g, yield: 63%).

TLC system: EtOAc/Hexane (50:50)

R_{f} value: ˜0.5

¹H NMR (400 MHz, CDCl₃) δ: 8.51 (bs, 1H), 7.83 (d, J=8.4 Hz, 2H), 7.64 (d, J=8.0 Hz, 2H), 7.46-7.41 (m, 2H), 7.40-7.37 (m, 3H), 5.04 (s, 2H), 1.34 (s, 12H)

### Step 5: Synthesis of N-(benzyloxy)-4-(2-chloropyrimidin-4-yl)benzamide

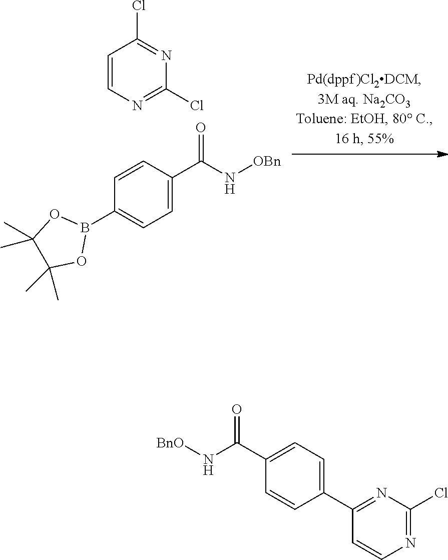

To degassed stirred solution of 2,4-dichloropyrimidine (500 mg, 3.37 mmol, 1 eq) and N-(benzyloxy)-4-(4,4,5,5-tetramethyl-1,3,2-dioxaborolan-2-yl)benzamide (1.4 g, 4.04 mmol, 1.2 eq) in toluene:EtOH (2:1, 20 mL) was added 2M aq. Na₂CO₃ (1.65 mL, 3.3 Vol) followed by Pd(dppf)Cl₂ in dichloromethane (137 mg, 0.17 mmol, 0.05 eq). The reaction mixture was heated to 80° C. and stirred for 16 h in sealed tube. After completion of reaction by TLC, diluted with water (40 mL) and extracted with (2×40 mL). Organic layer was dried over Na₂SO₄, filtered, and concentrated under reduced pressure. The crude product was purified by silica gel (100-200 mesh) column chromatography [elution with 30% EtOAc in Hexane] to afford N-(benzyloxy)-4-(2-chloropyrimidin-4-yl)benzamide as pale yellow solid (500 mg, yield: 55%).

TLC system: EtOAc/Hexane (50:50)

R_{f} value: ˜0.4

LCMS(m/z): 340.1 (M+H)⁺

¹H NMR (400 MHz, CDCl₃) δ: 8.69 (d, J=5.6 Hz, 1H), 8.60 (s, 1H), 8.15-8.13 (m, 2H), 7.81 (d, J=8.4 Hz, 2H), 7.67 (d, J=5.2 Hz, 1H), 7.48-7.39 (m, 5H), 5.07 (s, 2H)

### Step 6: Synthesis of 4-(2-((3-fluoro-4-(morpholine-4-carbonyl)phenyl)amino)pyrimidin-4-yl)benzoic acid

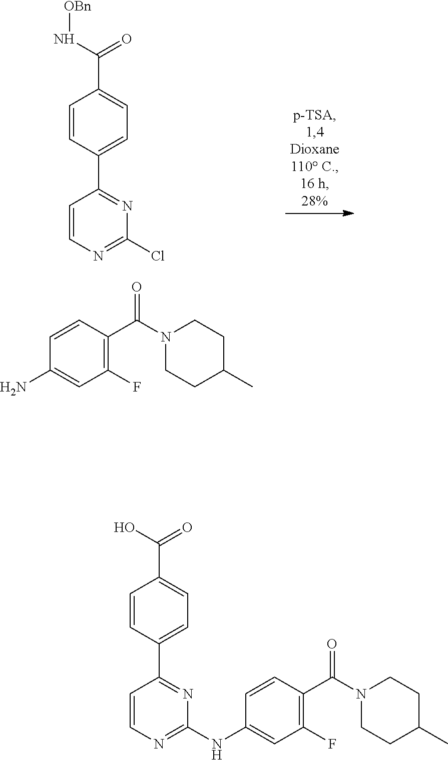

To a stirred solution of N-(benzyloxy)-4-(2-chloropyrimidin-4-yl)benzamide (500 mg, 1.48 mmol, 1 eq) in 1,4-dioxane (10 mL) at room temperature, was added p-TSA (0.23 g, 1.33 mmol, 0.9 eq) followed by (4-amino-2-fluorophenyl)(4-methylpiperidin-1-yl)methanone (348 mg, 1.48 mmol, 1.0 eq) and stirred at 110° C. for 16 h. After completion of reaction by TLC, diluted with water (30 mL) and extracted with EtOAc (2×30 mL). Combined organic layer was dried over anhydrous Na₂SO₄, filtered, and concentrated under reduced pressure to afford crude product. The crude product was purified by silica gel (100-200 mesh) column chromatography [elution with 80% EtOAc in Hexane] to afford 4-(2-((3-fluoro-4-(morpholine-4-carbonyl)phenyl)amino)pyrimidin-4-yl)benzoic acid as pale yellow solid (180 mg, yield: 28%).

TLC system: EtOAc/Hexane (100:00)

R_{f} value: ˜0.2

LCMS(m/z): 435.2 (M+H)⁺

### Step 7: Synthesis of 4-(2-((3-fluoro-4-(4-methylpiperidine-1-carbonyl)phenyl)amino)pyrimidin-4-yl)-N-((tetrahydro-2H-pyran-2-yl)oxy)benzamide

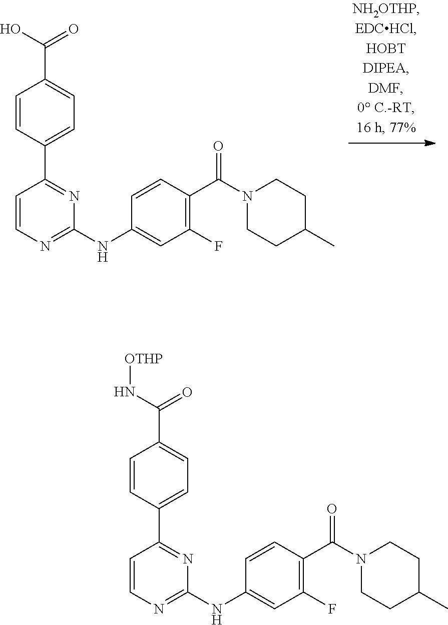

To a stirred solution of 4-(2-((3-floro-4-(morpholine-4-carbonyl)phenyl)amino)pyrimidin-4-yl)benzoic acid (180 mg, 0.41 mmol, 1 eq) in DMF (2.0 mL) at 0° C., was added DIPEA (158 mg, 1.24 mmol, 3.0 eq), HOBt (55 mg, 0.41 mmol, 1.0 eq) followed by EDC-HCl (79 mg, 0.41 mmol, 1.0 eq) and stirred for 10 min. After that NH₂OTHP (58 mg, 0.49 mmol, 1.2 eq) was added to the reaction mixture and stirred at room temperature for 16 h. After completion of reaction by TLC, reaction mixture was quenched with ice cold water (10 mL) and extracted with 10% MeOH in dichloromethane (2×10 mL). The combined organic layer was dried under reduced pressure to afford 4-(2-((3-fluoro-4-(4-methylpiperidine-1-carbonyl) phenyl)amino)pyrimidin-4-yl)-N-((tetrahydro-2H-pyran-2-yl)oxy)benzamide as yellow gum (170 mg, Yield: 77%).

TLC system: MeOH/dichloromethane (10:90)

R_{f} value: ˜0.5

LCMS(m/z): 534.2 (M+H)⁺

### Step 8: Synthesis of 4-(2-((3-fluoro-4-(4-methylpiperidine-1-carbonyl)phenyl)amino)pyrimidin-4-yl)-N-hydroxybenzamide

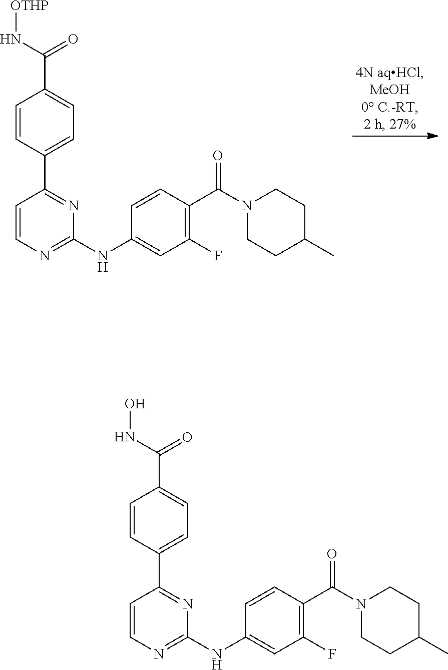

To a stirred solution of 4-(2-((3-fluoro-4-(4-methylpiperidine-1-carbonyl)phenyl)amino)pyrimidin-4-yl)-N-((tetrahydro-2H-pyran-2-yl)oxy)benzamide (160 mg, 0.46 mmol, 1.0 eq (LCMS: 73%)) in MeOH (2.0 mL) at 0° C., was added 4N aq. HCl (3.0 mL, 20 Vol) and stirred at room temperature for 2 h. After completion of reaction by TLC, volatiles removed under vacuum and purified by reverse phase column chromatography (Grace) [elution with 20% acetonitrile in 0.1% formic acid in H₂O] to afford 4-(2-((3-fluoro-4-(4-methylpiperidine-1-carbonyl)phenyl)amino)pyrimidin-4-yl)-N-hydroxybenzamide as yellow solid (35 mg, yield: 27%).

TLC system: MeOH/dichloromethane (10:90)

R_{f} value: ˜0.1

LCMS (m/z): 450.2 (M+H)⁺

¹H NMR (400 MHz, DMSO-d₆) δ: 11.36 (s, 1H), 10.16 (s, 1H), 9.15 (s, 1H), 8.67 (d, J=5.2 Hz, 1H), 8.25 (d, J=8.8 Hz, 2H), 7.96-7.92 (m, 3H), 7.62 (dd, J=1.6 Hz, 8.4 Hz, 1H), 7.57 (d, J=5.2 Hz, 1H), 7.31 (t, J=8.4 Hz, 1H), 4.48-4.45 (m, 1H), 3.49-3.46 (m, 1H), 3.12 (t, 1H), 2.75 (t, J=12 Hz, 1H), 1.72-1.69 (m, 1H), 1.61-1.57 (m, 2H), 1.07-1.01 (m, 2H), 0.92 (d, J=6.4 Hz, 3H)

### Synthetic Example 4

### Synthesis of 4-(2-((3-Fluoro-4-(4-Methylpiperazine-1-Carbonyl)Phenyl)Amino)Pyrimidin-4-Yl)-N-Hydroxybenzamide Formic Acid Salt

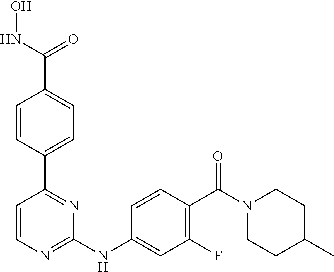 [SMILES: CC1CCN(C(=O)c2ccc(Nc3nccc(-c4ccc(C(=O)NO)cc4)n3)cc2F)CC1]

### Step 1: Synthesis of (2-fluoro-4-nitrophenyl)(4-methylpiperazin-1-yl)methanone

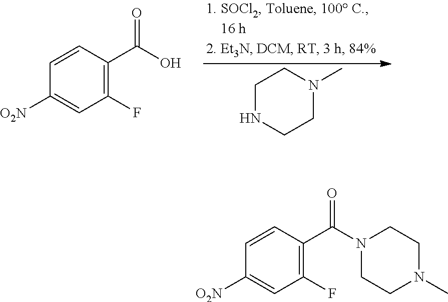

To a stirred solution of 2-fluoro-4-nitrobenzoic acid (3 g, 16.2 mmol, 1.0 eq) in Toluene (30 mL), at 0° C., was added SOCl₂ (5.54 g, 48.6 mmol, 3.0 eq) and stirred at 100° C. for 16 h. After completion of reaction by TLC, the reaction mixture was concentrated under reduced pressure to afford crude acid-chloride. The crude product was dissolved in dichloromethane (25 mL) at room temperature, added Et₃N (0.67 mL, 48.6 mmol, 3.0 eq) and 1-methylpiperazine (1.94 g, 19.4 mmol, 1.2 eq) and stirred for additional 3 h. After completion of reaction by TLC, diluted with water (30 mL) and extracted with EtOAc (3×30 mL). Combined organic layer was dried over Na₂SO₄, filtered and concentrated under reduced pressure to afford (2-fluoro-4-nitrophenyl)(4-methylpiperazin-1-yl)methanone as a yellow gummy mass (3.6 g, yield: 84%).

TLC system: EtOAc/Hexane (50:50)

R_{f} value: ˜0.5

LCMS(m/z): 268.1 (M+H)⁺

¹H NMR (400 MHz, CDCl₃) δ: 8.13-8.10 (m, 1H), 8.01-7.98 (m, 1H), 7.61-7.57 (m, 1H), 3.84 (brs, 2H), 3.32 (brs, 2H), 2.51 (t, J=5.2 Hz, 2H), 2.2-2.38 (m, 2H), 2.34 (s, 3H)

### Step 2: Synthesis of (4-amino-2-fluorophenyl)(4-methylpiperazin-1-yl)methanone

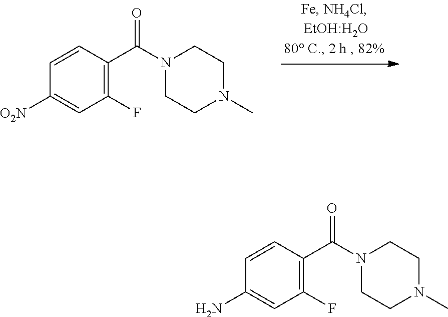 [SMILES: CN1CCN(C(=O)c2ccc([N+](=O)[O-])cc2F)CC1 ; CN1CCN(C(=O)c2ccc(N)cc2F)CC1]

To a stirred solution of (2-fluoro-4-nitrophenyl) (4-methylpiperazin-1-yl) methanone (3.6 g, 13.4 mmol, 1 eq) in EtOH:H₂O (40 mL) at room temperature, was added Fe (3.76 g, 67.2 mmol, 5 eq) and NH₄Cl (3.56 g, 67.2 mmol, 5 eq) and reaction mixture was heated to 80° C., stirred for 2 h. After completion of reaction by TLC, reaction mixture was filtered through a pad of diatomaceous earth (e.g., Celite®) and washed with EtOAc (30 mL). Filtrate was concentrated, diluted with water (80 mL) and extracted with EtOAc (3×50 mL). The combined organic layer was dried over Na₂SO₄, filtered and concentrated under reduced pressure to afford (4-amino-2-fluorophenyl) (4-methylpiperazin-1-yl)methanone as yellow gummy mass (2.6 g, yield: 82%).

TLC system: EtOAc/Hexane (100:00)

R_{f} value: ˜0.4

LCMS(m/z): 238.1 (M+H)⁺

¹H NMR (400 MHz, DMSO-d₆) δ: 7.00 (t, J=8.4 Hz, 1H), 6.39 (dd, J=2.0 Hz, 8.4 Hz, 1H), 6.29 (dd, J=2.0 Hz, 12.8 Hz, 1H), 5.74 (s, 2H), 3.43-3.27 (m, 4H), 2.29-2.27 (brs, 4H), 2.21 (s, 3H)

### Step 3: Synthesis of methyl 4-(2-chloropyrimidin-4-yl)benzoate

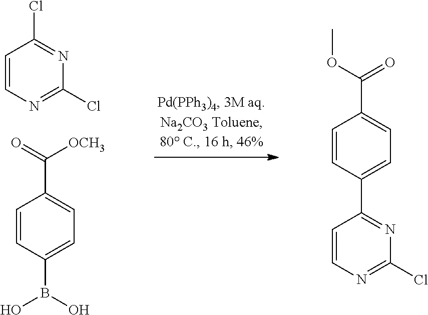

In a degassed stirred solution of 2,4-dichloropyrimidine (3 g, 20.3 mmol, 1 eq) and (4-(methoxycarbonyl)phenyl)boronic acid (5.47 g, 30.4 mmol, 1.5 eq) in Toluene (45 mL) was added 3M aq. Na₂CO₃ (9.9 mL, 3.3 Vol) followed by Pd(PPh₃)₄ (0.46 g, 0.41 mmol, 0.02 eq). The reaction mixture was heated to 80° C. and stirred for 16 h in sealed tube. After completion of reaction by TLC, the reaction mixture was diluted with water (40 mL) and extracted with EtOAc (3×40 mL). Combined organic layer was dried over Na₂SO₄, filtered, and concentrated under reduced pressure. Obtained crude was diluted with MeOH (20 mL), stirred for 30 min, solid was filtered and dried under reduced vacuum to afford methyl 4-(2-chloropyrimidin-4-yl) benzoate as brown solid (2.3 g, yield: 46%).

TLC system: EtOAc/Hexane (50:50)

R_{f} value: ˜0.4

¹H NMR (400 MHz, CDCl₃) δ: 8.70 (d, J=5.2 Hz, 1H), 8.19-8.17 (m, 4H), 7.71 (d, J=5.2 Hz, 1H), 3.97 (s, 3H)

### Step 4: Synthesis of methyl 4-(2-((3-fluoro-4-(4-methylpiperazine-1-carbonyl)phenyl) amino)pyrimidin-4-yl)benzoate

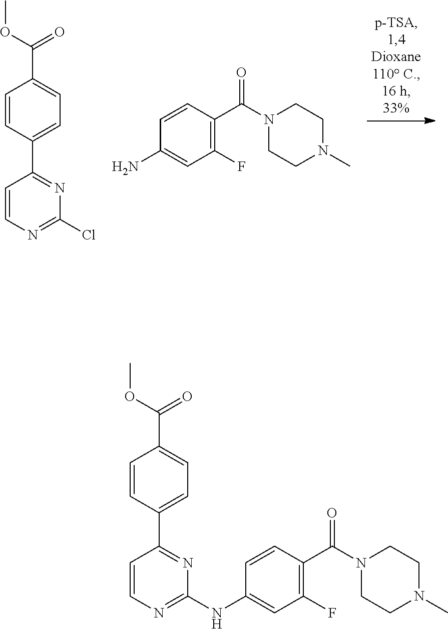 [SMILES: COC(=O)c1ccc(-c2ccnc(Cl)n2)cc1 ; CN1CCN(C(=O)c2ccc(N)cc2F)CC1 ; COC(=O)c1ccc(-c2ccnc(Nc3ccc(C(=O)N4CCN(C)CC4)c(F)c3)n2)cc1]

To a stirred solution of methyl 4-(2-chloropyrimidin-4-yl)benzoate (500 mg, 2.01 mmol, 1 eq) in 1,4-dioxane (15 mL) at room temperature, was added p-TSA (0.31 g, 1.81 mmol, 0.9 eq) followed by (4-amino-2-fluorophenyl)(4-methylpiperazin-1-yl)methanone (0.48 g, 2.01 mmol, 1.0 eq) and stirred at 110° C. for 16 h. After completion of reaction by TLC, diluted with water (20 mL) and extracted with EtOAc (2×20 mL). Combined organic layer was dried over anhydrous Na₂SO₄, filtered, and concentrated under reduced pressure. The crude product was purified by reverse phase column chromatography [elution with 35% acetonitrile in 0.1% formic acid in H₂O] to afford methyl 4-(2-((3-fluoro-4-(4-methylpiperazine-1-carbonyl) phenyl) amino)pyrimidin-4-yl)benzoate as brown solid (300 mg, yield: 33%).

TLC system: MeOH/dichloromethane (10:90)

R_{f} value: ˜0.4

LCMS(m/z): 450.2 (M+H)⁺

### Step 5: Synthesis of 4-(2-((3-fluoro-4-(4-methylpiperazine-1-carbonyl)phenyl)amino)pyrimidin-4-yl)benzoic acid

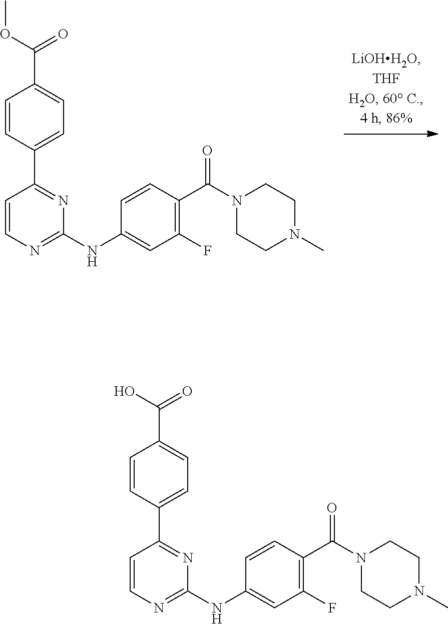 [SMILES: COC(=O)c1ccc(-c2ccnc(Nc3ccc(C(=O)N4CCN(C)CC4)c(F)c3)n2)cc1 ; CN1CCN(C(=O)c2ccc(Nc3nccc(-c4ccc(C(=O)O)cc4)n3)cc2F)CC1]

To a stirred solution of methyl 4-(2-((3-fluoro-4-(4-methylpiperazine-1-carbonyl)phenyl)amino)pyrimidin-4-yl)benzoate (300 mg, 0.66 mmol, 1.0 eq) in THF:H₂O (2:1) (3 mL) at 0° C., was added LiOH·H₂O (56 mg, 1.33 mmol, 2 eq) and stirred at 60° C. for 4 h. After completion of reaction by TLC, reaction mixture was washed with diethyl ether (10 mL). Aqueous layer was acidified with 2N HCl solution (5 mL), precipitated solid was filtered and dried under reduced pressure to afford 4-(2-((3-fluoro-4-(4-methylpiperazine-1-carbonyl)phenyl)amino)pyrimidin-4-yl)benzoic acid as yellow solid (250 mg, yield: 86%).

TLC system: MeOH/dichloromethane (10:90)

R_{f} value: ˜0.2

LCMS(m/z): 436.2 (M+H)⁺

¹H NMR (400 MHz, DMSO-d₆) δ: 11.09 (s, 1H), 10.28 (s, 1H), 8.70 (d, J=5.2 Hz, 1H), 8.29 (d, J=8.4 Hz, 2H), 8.12 (d, J=8.4 Hz, 2H), 7.98 (dd, J=13.6 & 1.6 Hz, 1H), 7.69 (dd, J=8.4 & 1.6 Hz 1H), 7.60 (d, J=5.2 Hz, 1H), 7.42 (t, J=8.4 Hz, 1H), 4.19-4.05 (br, 2H), 3.62 (bs, 2H), 3.17 (brs, 4H), 2.78 (s, 3H)

### Step 6: Synthesis of 4-(2-((3-fluoro-4-(4-methylpiperazine-1-carbonyl)phenyl)amino)pyrimidin-4-yl)-N-((tetrahydro-2H-pyran-2-yl)oxy)benzamide

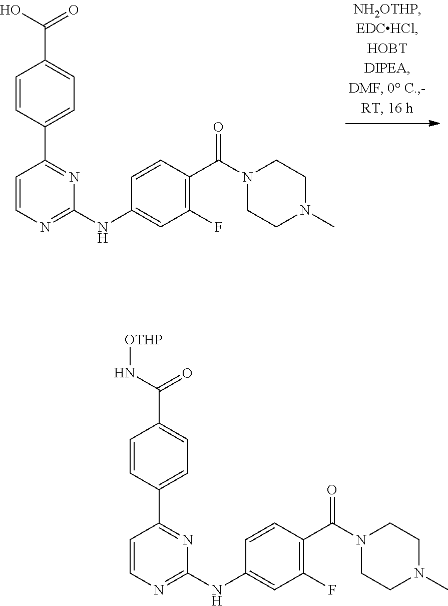

To a stirred solution of 4-(2-((3-fluoro-4-(4-methylpiperazine-1-carbonyl)phenyl)amino)pyrimidin-4-yl)benzoic acid (250 mg, 0.57 mmol, 1 eq) in DMF (2.5 mL) at 0° C., was added DIPEA (0.3 mL, 1.54 mmol, 3.0 eq), HOBt (77 mg, 0.57 mmol, 1.0 eq) followed by EDC-HCl (109 mg, 0.57 mmol, 1.0 eq) and stirred for 10 min. Later, added NH₂OTHP (81 mg, 0.68 mmol, 1.2 eq) and stirred at room temperature for 16 h. After completion of reaction by TLC, the reaction mixture was quenched with ice cold water (10 mL) and extracted with 10% MeOH in dichloromethane (2×10 mL). The combined organic layer was dried under reduced pressure to afford 4-(2-((3-fluoro-4-(4-methylpiperazine-1-carbonyl)phenyl) amino)pyrimidin-4-yl)-N-((tetrahydro-2H-pyran-2-yl)oxy)benzamide as a yellow gummy mass (250 mg, Yield: Crude).

TLC system: MeOH/dichloromethane (10:90)

R_{f} value: ˜0.4

LCMS(m/z): 535.3 (M+H)+

### Step 7: Synthesis of 4-(2-((3-fluoro-4-(4-methylpiperazine-1-carbonyl)phenyl)amino)pyrimidin-4-yl)-N-hydroxybenzamide formic acid salt

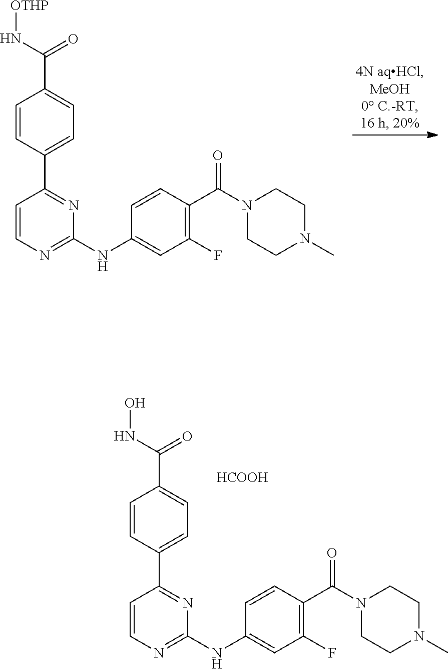

To a stirred solution of 4-(2-((3-fluoro-4-(4-methylpiperazine-1-carbonyl)phenyl)amino)pyrimidin-4-yl)-N-((tetrahydro-2H-pyran-2-yl)oxy)benzamide (250 mg, 0.46 mmol, 1 eq (LCMS: 62%)) in MeOH (2.5 mL) at 0° C., was added 4N aq. HCl (5.0 mL, 20 Vol) and stirred at room temperature for 16 h. After completion of reaction by TLC, the reaction mixture was concentrated under reduced pressure and purified by reverse phase column chromatography [elution with 20% acetonitrile in 0.1% formic acid in H₂O] to afford 4-(2-((3-fluoro-4-(4-methylpiperazine-1-carbonyl)phenyl)amino)pyrimidin-4-yl)-N-hydroxybenzamide as formic acid salt as yellow solid (40 mg, yield: 20%).

TLC system: MeOH/dichloromethane (10:90)

R_{f} value: ˜0.2

LCMS (m/z): 451.2 (M+H)⁺

¹H NMR (400 MHz, DMSO-d₆) δ: 11.37 (s, 1H), 10.18 (s, 1H), 9.15 (br, 1H), 8.67 (d, J=5.2 Hz, 1H), 8.25 (d, J=8.4 Hz, 2H), 8.14 (s, 1H; formic acid), 7.98-7.92 (m, 3H), 7.63 (dd, J=2.0 Hz, 8.4 Hz, 1H), 7.58 (d, J=5.2 Hz, 1H), 7.33 (t, J=8.4 Hz, 1H), 3.64 (brs, 2H), 3.30 (brs, 2H), 2.39 (brs, 2H), 2.33-2.31 (m, 2H), 2.22 (s, 3H)

### Synthetic Example 5

### Synthesis of 4-(2-((3-Fluoro-4-(2-Methylmorpholine-4-Carbonyl)Phenyl)Amino)Pyrimidin-4-Yl)-N-Hydroxybenzamide

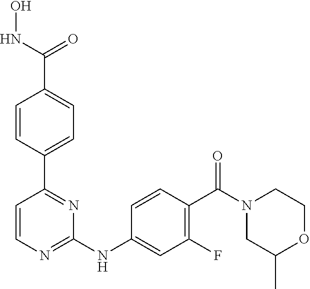 [SMILES: CC1CN(C(=O)c2ccc(Nc3nccc(-c4ccc(C(=O)NO)cc4)n3)cc2F)CCO1]

### Step 1: Synthesis of (2-fluoro-4-nitrophenyl)(2-methylmorpholino)methanone

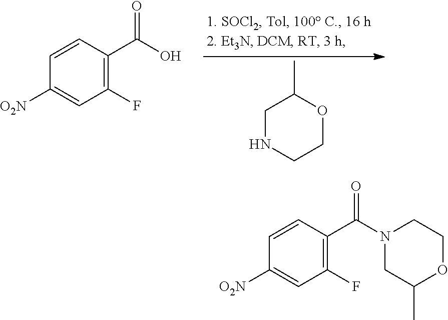

To a stirred solution of 2-fluoro-4-nitrobenzoic acid (2 g, 10.8 mmol, 1.0 eq) in Toluene (20 mL), at 0° C., was added SOCl₂ (3.27 g, 27.4 mmol, 3.0 eq) and stirred at 100° C. for 16 h. After completion of reaction by TLC, the reaction mixture was concentrated under reduced pressure to afford crude product. The crude product was dissolved in dichloromethane (25 mL) at room temperature, added Et₃N (2.98 g, 29.6 mmol, 3.0 eq), 2-methylmorpholine (3.85 g, 11.8 mmol, 1.2 eq) and stirred for 2 h. After completion of reaction by TLC, the reaction mixture was diluted with water (30 mL) and extracted with EtOAc (2×30 mL). The organic layer was dried over Na₂SO₄, filtered and concentrated under reduced pressure to afford (2-fluoro-4-nitrophenyl)(2-methylmorpholino)methanone as a yellow gummy mass (2.6 g, crude).

TLC system: EtOAc/Hexane (100:00)

R_{f} value: ˜0.4

LCMS(m/z): 269.1 (M+H)⁺

### Step 2: Synthesis of (4-amino-2-fluorophenyl)(2-methylmorpholino)methanone

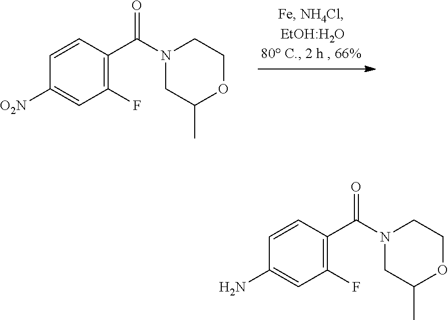 [SMILES: CC1CN(C(=O)c2ccc([N+](=O)[O-])cc2F)CCO1 ; CC1CN(C(=O)c2ccc(N)cc2F)CCO1]

To a stirred solution of (2-fluoro-4-nitrophenyl)(2-methylmorpholino)methanone (2.6 g, 9.70 mmol, 1 eq) in EtOH:H₂O (30 mL) at room temperature, was added Fe (2.7 g, 48.5 mmol, 5 eq) and NH₄Cl (2.57 g, 48.5 mmol, 5 eq) and heated at 80° C. for 2 h. After completion of reaction by TLC, the reaction mixture was filtered through a pad of diatomaceous earth (e.g., Celite®) and washed with EtOAc (50 mL). Filtrate was concentrated, diluted with water (30 mL) and extracted with EtOAc (2×50 mL). Combined organic layer was dried over Na₂SO₄, filtered and concentrated under reduced pressure to afford (4-amino-2-fluorophenyl)(2-methylmorpholino)methanone as a yellow gummy mass (1.7 g, yield: ˜66%).

TLC system: EtOAc/Hexane (100:00)

R_{f} value: ˜0.2

LCMS(m/z): 239.1 (M+H)⁺

### Step 3: Synthesis of methyl 4-(2-chloropyrimidin-4-yl)benzoate

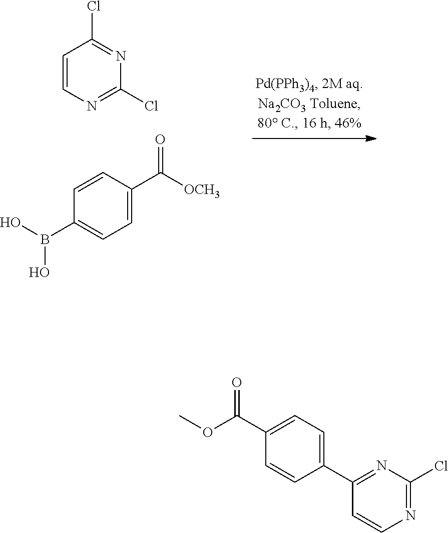

To a degassed solution of 2,4-dichloropyrimidine (3 g, 20.3 mmol, 1.0 eq) and (4-(methoxycarbonyl)phenyl)boronic acid (5.47 g, 30.4 mmol, 1.5 eq) in Toluene (45 mL) was added 2M aq. Na₂CO₃ (9.9 mL, 3.3 Vol) followed by Pd(PPh₃)₄ (0.46 g, 0.40 mmol, 0.02 eq). The reaction mixture was heated to 80° C. and stirred for 16 h in sealed tube. After completion of reaction by TLC, the reaction mixture was diluted with water (40 mL) and extracted with EtOAc (2×40 mL). The organic layer was dried over Na₂SO₄, filtered, and concentrated under reduced pressure to afford crude product. The crude product was triturated with MeOH (20 mL), filtered and collected solid was dried under vacuum to afford methyl 4-(2-chloropyrimidin-4-yl)benzoate as brown solid (2.3 g, yield: 46%).

TLC system: EtOAc/Hexane (70:30)

R_{f} value: ˜0.4

¹H NMR (400 MHz, CDCl₃) δ: 8.70 (d, J=5.2 Hz, 1H), 8.18-8.16 (m, 4H), 7.71 (d, J=5.2 Hz, 1H), 3.97 (s, 3H)

### Step 4: Synthesis of methyl 4-(2-((3-fluoro-4-(2-methylmorpholine-4-carbonyl)phenyl)amino)pyrimidin-4-yl)benzoate

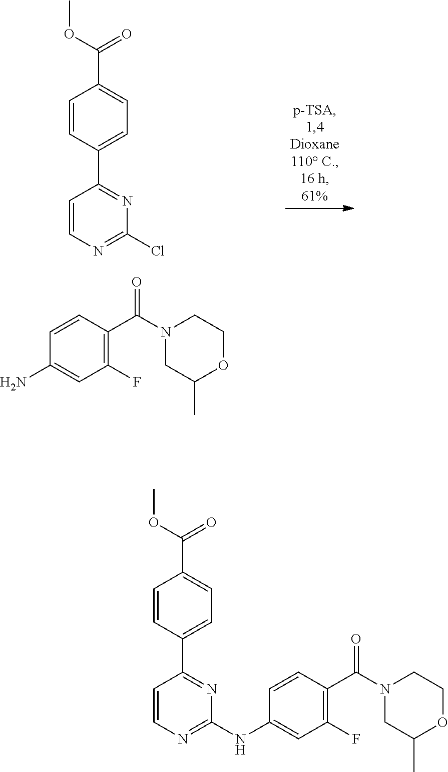

To a stirred solution of methyl 4-(2-chloropyrimidin-4-yl)benzoate (500 mg, 2.01 mmol, 1 eq) in 1,4-dioxane (5 mL) at room temperature, was added p-TSA (0.31 g, 1.81 mmol, 0.9 eq) followed by (4-amino-2-fluorophenyl)(2-methylmorpholino)methanone (0.48 g, 2.01 mmol, 1.0 eq) and stirred at 110° C. for 16 h. After completion of reaction by TLC, the reaction mixture was diluted with water (10 mL) and extracted with EtOAc (2×10 mL). The combined organic layer was dried over anhydrous Na₂SO₄, filtered, and concentrated under reduced pressure to afford crude product. The crude product was purified by silica gel (100-200 mesh) column chromatography [elution with 60% EtOAc in Hexane] to afford methyl 4-(2-((3-fluoro-4-(2-methylmorpholine-4-carbonyl)phenyl)amino)pyrimidin-4-yl)benzoate as a brown solid (550 mg g, yield: 61%).

TLC system: EtOAc/Hexane (100:00)

R_{f} value: ˜0.3

LCMS(m/z): 451.2 (M+H)⁺

¹H NMR (400 MHz, CDCl₃) δ: 8.57 (d, J=5.2 Hz, 1H), 8.19 (d, J=6.8 Hz, 2H), 8.15 (d, J=6.8 Hz, 2H), 7.95-7.92 (m, 1H), 7.55-7.52 (m, 1H), 7.40 (t, J=8.0 Hz, 1H), 7.30-7.27 (m, 1H), 4.57-4.54 (m, 1H), 4.01-3.83 (m, 4H), 3.67-3.56 (m, 2H), 3.49-3.46 (m, 1H), 3.32-3.29 & 2.69-2.63 (m, 1H), 3.03-2.94 (m, 1H), 2.16-1.12 (2d, 3H)

### Step 5: Synthesis of 4-(2-((3-fluoro-4-(2-methylmorpholine-4-carbonyl)phenyl)amino)pyrimidin-4-yl)benzoic acid

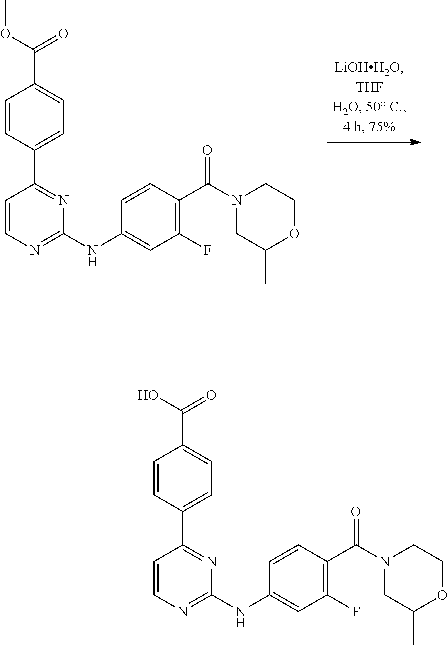 [SMILES: COC(=O)c1ccc(-c2ccnc(Nc3ccc(C(=O)N4CCOC(C)C4)c(F)c3)n2)cc1 ; CC1CN(C(=O)c2ccc(Nc3nccc(-c4ccc(C(=O)O)cc4)n3)cc2F)CCO1]

To a stirred solution of methyl 4-(2-((3-fluoro-4-(2-methylmorpholine-4-carbonyl)phenyl)amino)pyrimidin-4-yl)benzoate (550 mg, 1.22 mmol, 1.0 eq) in THF:H₂O (2:1) (5 mL) at 0° C., was added LiOH·H₂O (103 mg, 2.44 mmol, 2 eq) and stirred at 50° C. for 4 h. After completion of reaction by TLC, the reaction mixture was washed with diethylether (10 mL). Aqueous layer was acidified with 2N HCl solution (5 mL), precipitated solid was filtered, dried under vacuum to afford 4-(2-((3-fluoro-4-(2-methylmorpholine-4-carbonyl)phenyl)amino)pyrimidin-4-yl)benzoic acid as a yellow solid (400 mg, yield: 75%).

TLC system: MeOH/dichloromethane (10:90)

R_{f} value: ˜0.4

LCMS(m/z): 437.1 (M+H)⁺

¹H NMR (400 MHz, DMSO-d₆) δ: 13.1 (br, 1H), 10.23 (s, 1H), 8.69 (d, J=5.2 Hz, 1H), 8.29 (d, J=8.8 Hz, 2H), 8.12 (d, J=8.4 Hz, 2H), 7.95 (dd, J=1.6 Hz, 13.2 Hz, 1H), 7.66 (dd, J=2.0 Hz, 8.4 Hz, 1H), 7.59 (d, J=5.2 Hz, 1H), 7.36 (t, J=8.4 Hz, 1H), 4.37-4.29 (m, 1H), 3.92-3.75 (m, 1H), 3.48-3.44 (m, 2H), 2.89 (brs, 1H), 2.55-2.50 (m, 2H), 1.18-1.01 (m, 3H)

### Step 6: Synthesis of 4-(2-((3-fluoro-4-(2-methylmorpholine-4-carbonyl)phenyl)amino)pyrimidin-4-yl)-N-((tetrahydro-2H-pyran-2-yl)oxy)benzamide

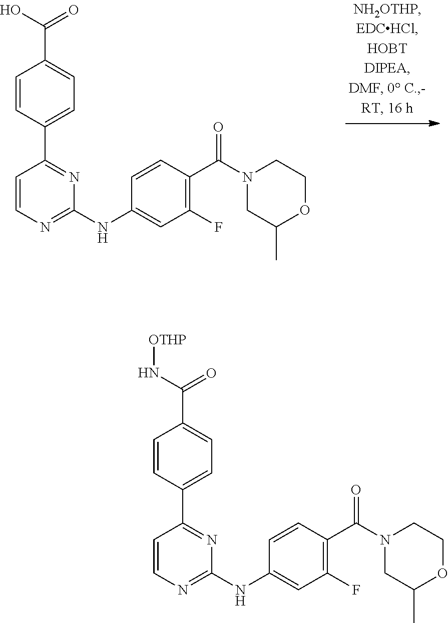

To a stirred solution of 4-(2-((3-fluoro-4-(2-methylmorpholine-4-carbonyl)phenyl)amino)pyrimidin-4-yl)benzoic acid (250 mg, 0.57 mmol, 1 eq) in DMF (2.5 mL) at 0° C., was added DIPEA (0.3 mL, 1.54 mmol, 3.0 eq), HOBt (77 mg, 0.57 mmol, 1.0 eq) followed by EDC-HCl (109 mg, 0.57 mmol, 1.0 eq) and stirred for 10 min. After that NH₂OTHP (80 mg, 0.68 mmol, 1.2 eq) was added to the reaction mixture and stirred at room temperature for 16 h. After completion of reaction by TLC, the reaction mixture was quenched with ice cold water (10 mL) and extracted with 10% MeOH in dichloromethane (2×10 mL). The combined organic layer was dried over Na₂SO₄ and evaporated under reduced pressure to afford 4-(2-((3-fluoro-4-(2-methylmorpholine-4-carbonyl)phenyl)amino)pyrimidin-4-yl)-N-((tetrahydro-2H-pyran-2-yl)oxy)benzamide as yellow solid (230 mg, Yield: 76%).

TLC system: MeOH/dichloromethane (10:90)

R_{f} value: ˜0.5

LC/MS(m/z): 536.2 (M+H)⁺

### Step 7: Synthesis of 4-(2-((3-fluoro-4-(2-methylmorpholine-4-carbonyl)phenyl)amino)pyrimidin-4-yl)-N-hydroxybenzamide

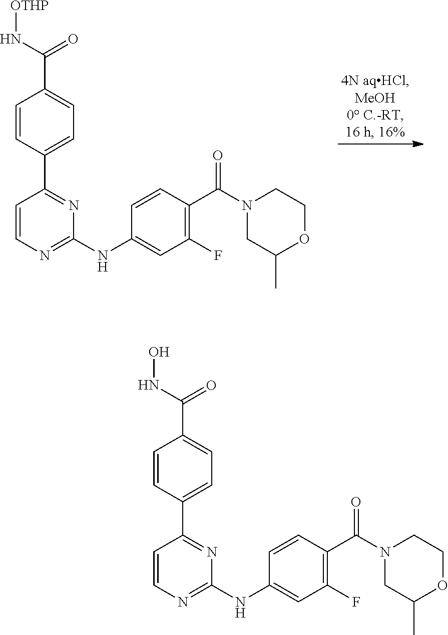

To a stirred solution of 4-(2-((3-fluoro-4-(2-methylmorpholine-4-carbonyl)phenyl)amino)pyrimidin-4-yl)-N-((tetrahydro-2H-pyran-2-yl)oxy)benzamide (230 mg, 0.43 mmol, 1 eq (LCMS: 62%)) in MeOH (2.5 mL) at 0° C., was added 4N aq. HCl (4.5 mL, 20 Vol) and stirred at room temperature for 16 h. After completion of reaction by TLC, reaction mixture was concentrated under reduced pressure and purified by reverse phase column chromatography (Grace) [elution with 20% acetonitrile in 0.1% formic acid in H₂O] to afford 4-(2-((3-fluoro-4-(2-methylmorpholine-4-carbonyl)phenyl)amino)pyrimidin-4-yl)-N-hydroxybenzamide a white solid (32 mg, yield: 16%).

TLC system: MeOH/dichloromethane (10:90)

R_{f} value: ˜0.3

LCMS (m/z): 452.2 (M+H)⁺

¹H NMR (400 MHz, DMSO-d₆) δ: 11.4 (br, 1H), 10.19 (s, 1H), 9.15 (br, 1H), 8.67 (d, J=5.2 Hz, 1H), 8.23 (d, J=8.4 Hz, 2H), 7.98-7.92 (m, 3H), 7.64 (dd, J=8.4 Hz, 1.6 Hz, 1H), 7.57 (d, J=5.2 Hz, 1H), 7.36 (t, J=8.4 Hz, 1H), 4.38-4.30 (m, 1H), 3.92-3.89 (m, 1H), 3.77-3.75 (m, 1H), 3.46-3.35 (m, 2H), 3.21-3.17 (m, 1H), 2.91-2.87 (m, 1H), 1.16-1.02 (m, 3H)

### Synthetic Example 6

### Synthesis of 4-(2-((3-Fluoro-4-((4-Methylpiperidin-1-Yl)Methyl)Phenyl)Amino)Pyrimidin-4-Yl)-N-Hydroxybenzamide

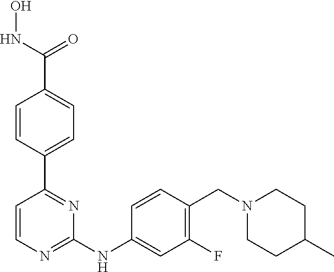 [SMILES: CC1CCN(Cc2ccc(Nc3nccc(-c4ccc(C(=O)NO)cc4)n3)cc2F)CC1]

### Step 1: Synthesis of 3-fluoro-4-((4-methylpiperidin-1-yl)methyl)aniline

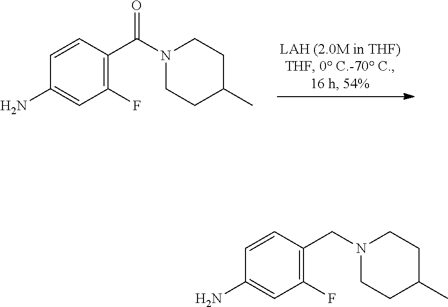 [SMILES: CC1CCN(C(=O)c2ccc(N)cc2F)CC1 ; CC1CCN(Cc2ccc(N)cc2F)CC1]

To a stirred solution of (4-amino-2-fluorophenyl)(4-methylpiperidin-1-yl)methanone (1.5 g, 6.35 mmol, 1.0 eq) in THF (15 mL) at 0° C., was added LAH (2.0 M in THF) (12.5 mL, 25.4 mmol, 4.0 eq) and stirred at 70° C. for 16 h. After completion of reaction by TLC, the reaction mixture was quenched with NH₄Cl solution (15 mL) and extracted with 10% MeOH in dichloromethane (2×30 mL). Combined organic layer was dried over Na₂SO₄, filtered, and concentrated under reduced pressure. Crude product was purified by reverse phase column chromatography [elution with 30% acetonitrile in 0.1% formic acid in H₂O] to afford 3-fluoro-4-((4-methylpiperidin-1-yl)methyl)aniline as off white solid (750 mg, yield: 54%).

TLC system: EtOAc/Hexane (100:00)

R_{f} value: ˜0.2

LCMS(m/z): 223.1 (M+H)⁺

¹H NMR (400 MHz, CDCl₃) δ: 7.29-7.25 (m, 1H), 6.45 (dd, J=2.0 Hz, 8.4 Hz, 1H), 6.36 (dd, J=2.0 Hz, 11.6 Hz, 1H), 3.99 (s, 2H), 3.34-3.31 (m, 2H), 2.53-2.47 (m, 2H), 1.75-1.72 (m, 2H), 1.65-1.55 (m, 2H), 1.51-1.48 (m, 1H), 0.96 (d, J=6.0 Hz, 3H)

### Step 2: Synthesis of methyl 4-(2-((3-fluoro-4-((4-methylpiperidin-1-yl)methyl)phenyl)amino)pyrimidin-4-yl)benzoate

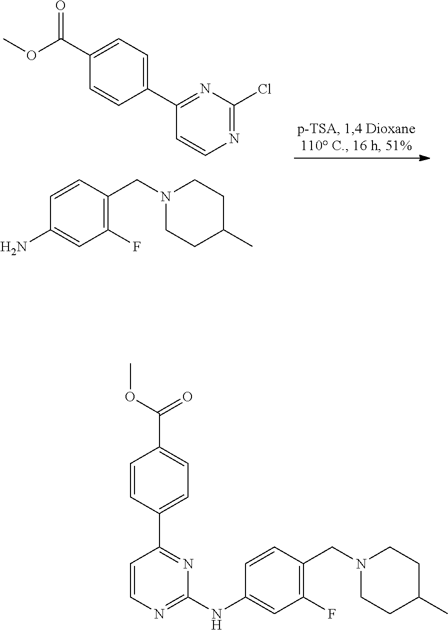

To a stirred solution of methyl 4-(2-chloropyrimidin-4-yl)benzoate (804 mg, 3.24 mmol, 1.2 eq) in 1,4-dioxane (12 mL) at room temperature, was added p-TSA (420 mg, 2.43 mmol, 0.9 eq) followed by 3-fluoro-4-((4-methylpiperidin-1-yl)methyl)aniline (600 mg, 2.70 mmol, 1.0 eq) and stirred at 110° C. for 16 h. After completion of reaction by TLC, reaction mixture was diluted with water (50 mL) and extracted with 10% MeOH in dichloromethane (3×20 mL). The combined organic layer was dried over anhydrous Na₂SO₄, filtered, and concentrated under reduced pressure to afford crude product. The Crude product was purified by reverse phase column chromatography (Grace) [elution with 40% acetonitrile in 0.1% formic acid in H₂O] to afford methyl 4-(2-((3-fluoro-4-((4-methylpiperidin-1-yl)methyl)phenyl)amino)pyrimidin-4-yl)benzoate as orange solid (600 mg, yield: 51%).

TLC system: MeOH/dichloromethane (10:90)

R_{f} value: ˜0.2

LCMS(m/z): 435.9 (M+H)⁺

### Step 3: Synthesis of 4-(2-((3-fluoro-4-((4-methylpiperidin-1-yl)methyl)phenyl)amino)pyrimidin-4-yl)benzoic acid

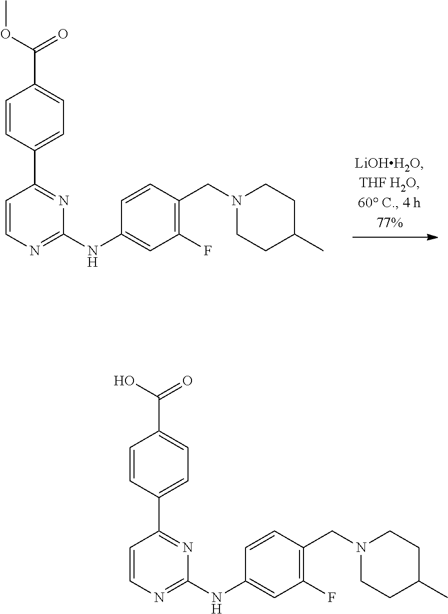 [SMILES: COC(=O)c1ccc(-c2ccnc(Nc3ccc(CN4CCC(C)CC4)c(F)c3)n2)cc1 ; CC1CCN(Cc2ccc(Nc3nccc(-c4ccc(C(=O)O)cc4)n3)cc2F)CC1]

To a stirred solution of methyl 4-(2-((3-fluoro-4-((4-methylpiperidin-1-yl)methyl)phenyl)amino)pyrimidin-4-yl)benzoate (600 mg, 1.38 mmol, 1.0 eq) in THF:H₂O (2:1) (6 mL) at 0° C., was added LiOH·H₂O (117 mg, 2.76 mmol, 2.0 eq) and stirred at 60° C. for 4 h. After completion of reaction by TLC, reaction mixture was washed with diethylether (20 mL). Aqueous layer was acidified with 2N HCl solution (10 mL), precipitated solid was filtered and dried under reduced pressure to afford 4-(2-((3-fluoro-4-((4-methylpiperidin-1-yl)methyl)phenyl)amino)pyrimidin-4-yl)benzoic acid as orange solid (450 mg, yield: 77%).

TLC system: MeOH/dichloromethane (10:90)

R_{f} value: ˜0.1

LCMS(m/z): 421.9 (M+H)⁺

¹H NMR (400 MHz, DMSO-d₆) δ: 13.18 (br, 1H), 10.26 (s, 1H), 8.71 (d, J=5.2 Hz, 1H), 8.31 (d, J=8.4 Hz, 2H), 8.13 (d, J=8.4 Hz, 2H), 8.01 (dd, J=1.6 Hz, 13.2 Hz, 1H), 7.69-7.67 (m, 1H), 7.50 (d, J=8.0 Hz, 1H), 7.13 (d, J=8.0 Hz, 1H), 4.26 (s, 2H), 2.97-2.94 (m, 2H), 2.30 (s, 2H), 1.82-1.78 (m, 2H), 1.62-1.60 (m, 1H), 1.44-1.37 (m, 2H), 0.92 (d, J=4.4 Hz, 3H)

### Step 4: Synthesis of 4-(2-((3-fluoro-4-((4-methylpiperidin-1-yl)methyl)phenyl)amino)pyrimidin-4-yl)-N-((tetrahydro-2H-pyran-2-yl)oxy)benzamide

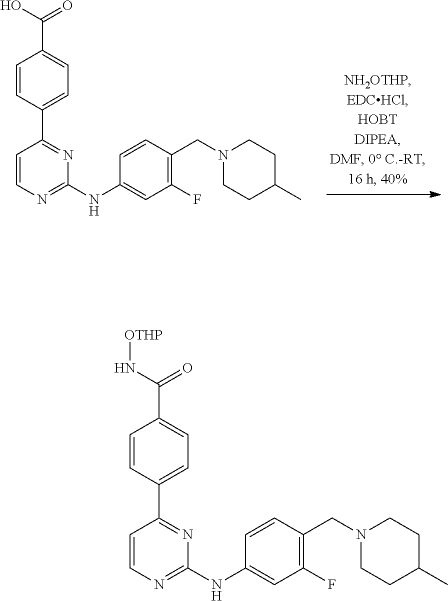

To a stirred solution of 4-(2-((3-fluoro-4-((4-methylpiperidin-1-yl)methyl)phenyl)amino)pyrimidin-4-yl)benzoic acid (400 mg, 0.95 mmol, 1 eq) in DMF (4 mL) at 0° C., was added DIPEA (0.5 mL, 2.85 mmol, 3.0 eq), HOBt (167 mg, 1.24 mmol, 1.3 eq) followed by EDC-HCl (236 mg, 1.24 mmol, 1.3 eq) and stirred for 30 min. After that NH₂OTHP (168 mg, 1.43 mmol, 1.5 eq) was added and stirred at room temperature for 16 h. After completion of reaction by TLC, diluted with ice cold water (50 mL) and extracted with 10% MeOH in dichloromethane (2×20 mL). The combined organic layer was dried under reduced pressure crude product. The Crude product was purified by reverse phase column chromatography (Grace) [elution with 30% acetonitrile in 0.1% formic acid in H₂O] to afford 4-(2-((3-fluoro-4-((4-methylpiperidin-1-yl)methyl)phenyl)amino)pyrimidin-4-yl)-N-((tetrahydro-2H-pyran-2-yl)oxy)benzamide as orange solid (200 mg, yield: 40%).

TLC system: MeOH/dichloromethane (10:90)

R_{f} value: ˜0.5

LCMS(m/z): 520.6 (M+H)+

### Step 5: Synthesis of 4-(2-((3-fluoro-4-((4-methylpiperidin-1-yl)methyl)phenyl)amino)pyrimidin-4-yl)-N-hydroxybenzamide

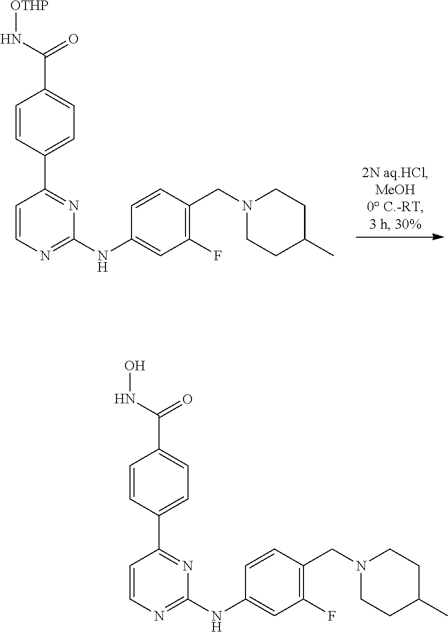

To a stirred solution of 4-(2-((3-fluoro-4-((4-methylpiperidin-1-yl)methyl)phenyl)amino)pyrimidin-4-yl)-N-((tetrahydro-2H-pyran-2-yl)oxy)benzamide (200 mg, 0.38 mmol, 1.0 eq) in MeOH (2.0 mL) at 0° C., was added 2N aq. HCl (0.96 mL, 5 Vol) and stirred at room temperature for 3 h. After completion of reaction by TLC, the reaction mixture was concentrated, quenched with NaHCO₃ solution (10 mL) and extracted with 10% MeOH in dichloromethane (2×20 mL). The combined organic layer was dried over anhydrous Na₂SO₄, filtered, and concentrated under reduced pressure. The crude product was purified by reverse phase column chromatography (Grace) [elution with 35% acetonitrile in 0.1% formic acid in H₂O] to afford 4-(2-((3-fluoro-4-((4-methylpiperidin-1-yl)methyl)phenyl)amino)pyrimidin-4-yl)-N-hydroxybenzamide as off white solid (50 mg, yield: 30%).

TLC system: MeOH/dichloromethane (10:90)

R_{f} value: ˜0.1

LCMS (m/z): 436.2 (M+H)⁺

¹H NMR (400 MHz, DMSO-d₆) δ: 11.36 (s, 1H), 10.01 (s, 1H), 9.11 (br, 1H), 8.64 (d, J=5.2 Hz, 1H), 8.30 (d, J=8.4 Hz, 2H), 7.93 (d, J=8.4 Hz, 2H), 7.87 (d, J=1.6 Hz, 13.2 Hz, 1H), 7.57-7.53 (m, 2H), 7.34 (t, J=8.4 Hz, 1H), 3.63 (bs, 2H), 2.93-2.91 (m, 2H), 2.19-2.17 (m, 2H), 1.63-1.60 (m, 2H), 1.38-1.35 (m, 1H), 1.22-1.13 (m, 2H), 0.88 (d, J=6.4 Hz, 3H)

### Synthetic Example 7

### Synthesis of 4-(2-((3-Fluoro-4-((4-Methylpiperazin-1-Yl)Methyl)Phenyl)Amino)Pyrimidin-4-Yl)-N-Hydroxybenzamide

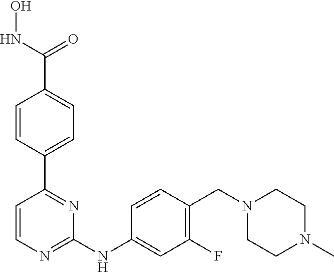 [SMILES: CN1CCN(Cc2ccc(Nc3nccc(-c4ccc(C(=O)NO)cc4)n3)cc2F)CC1]

### Step 1: Synthesis of 3-fluoro-4-((4-methylpiperazin-1-yl)methyl)aniline

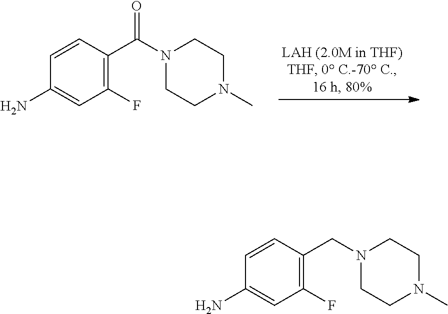 [SMILES: CN1CCN(C(=O)c2ccc(N)cc2F)CC1 ; CN1CCN(Cc2ccc(N)cc2F)CC1]

To a stirred solution of (4-amino-2-fluorophenyl)(4-methylpiperazin-1-yl)methanone (2.4 g, 10.2 mmol, 1.0 eq) in THF (24 mL) at 0° C., was added LAH (2.0 M in THF) (20.3 mL, 40.7 mmol, 4.0 eq) and stirred at 70° C. for 16 h. After completion of reaction by TLC, the reaction mixture was quenched with NH₄Cl solution (50 mL) and extracted with 10% MeOH in dichloromethane (2×30 mL). The organic layer was dried over Na₂SO₄, filtered, and concentrated under reduced pressure. The Crude product was purified by reverse phase column chromatography (Grace) [elution with 30% acetonitrile in 0.1% formic acid in H₂O] to afford 3-fluoro-4-((4-methylpiperazin-1-yl)methyl)aniline as pale brown solid (1.8 g, yield: 80%).

TLC system: EtOAc/Hexane (100:00)

R_{f} value: ˜0.1

¹H NMR (400 MHz, CDCl₃) δ: 8.50 (s, 1H; formic acid), 7.10 (t, J=8.0 Hz, 1H), 6.46-6.37 (m, 2H), 3.61 (s, 2H), 2.91-2.71 (m, 8H), 2.52 (s, 3H)

### Step 2: Synthesis of methyl 4-(2-((3-fluoro-4-((4-methylpiperazin-1-yl)methyl)phenyl)amino)pyrimidin-4-yl)benzoate

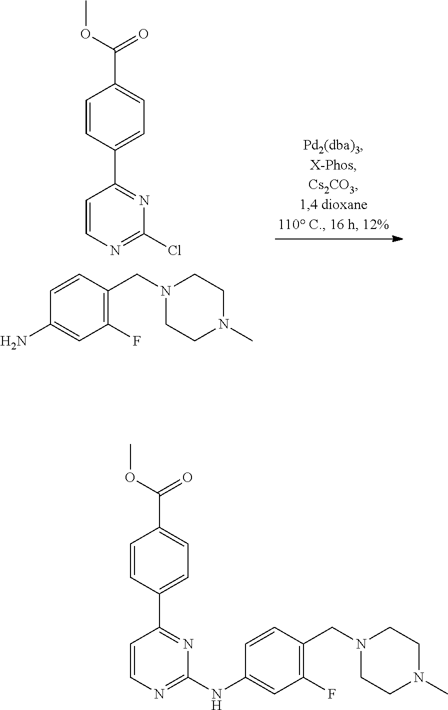

To a degassed stirred solution of methyl 4-(2-chloropyrimidin-4-yl)benzoate (700 mg, 2.82 mmol, 1.0 eq) and 3-fluoro-4-((4-methylpiperazin-1-yl)methyl)aniline (627 mg, 2.82 mmol, 1.0 eq) in 1,4-dioxane (14 mL) at room temperature, was added Cs₂CO₃ (1.84 g, 5.64 mmol, 2.0 eq), X-Phos (135 mg, 0.28 mmol, 0.1 eq) and Pd₂(dba)₃ (130 mg, 0.14 mmol, 0.05 eq). The reaction mixture was stirred at 110° C. for 16 h. After completion of reaction by TLC, reaction mixture was filtered through a pad of diatomaceous earth (e.g., Celite®), washed with 10% MeOH in dichloromethane (30 mL), collected filtrate was concentrated under reduced pressure and purified by reverse phase column chromatography (Grace) [elution with 35% acetonitrile in 0.1% formic acid in H₂O] to afford methyl 4-(2-((3-fluoro-4-((4-methylpiperazin-1-yl)methyl)phenyl)amino)pyrimidin-4-yl)benzoate as orange solid (150 mg, yield: 12%).

TLC system: MeOH/dichloromethane (10:90)

R_{f} value: ˜0.2

LCMS(m/z): 436.2 (M+H)⁺

### Step 3: Synthesis of 4-(2-((3-fluoro-4-((4-methylpiperazin-1-yl)methyl)phenyl)amino)pyrimidin-4-yl)benzoic acid

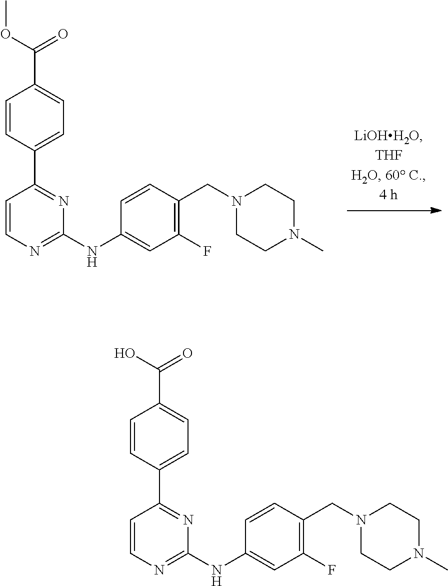 [SMILES: COC(=O)c1ccc(-c2ccnc(Nc3ccc(CN4CCN(C)CC4)c(F)c3)n2)cc1 ; CN1CCN(Cc2ccc(Nc3nccc(-c4ccc(C(=O)O)cc4)n3)cc2F)CC1]

To a stirred solution of methyl 4-(2-((3-fluoro-4-((4-methylpiperazin-1-yl)methyl)phenyl)amino)pyrimidin-4-yl)benzoate (160 mg, 0.36 mmol, 1.0 eq) in THF:H₂O (2:1) (1.6 mL) at 0° C., was added LiOH·H₂O (30 mg, 0.73 mmol, 2 eq) and reaction mixture was stirred at 60° C. for 4 h. After completion of reaction by TLC, the reaction mixture was washed with diethyl ether (20 mL). Aqueous layer was acidified with 2N HCl solution (5 mL), precipitated solid was filtered and dried under reduced pressure to afford 4-(2-((3-fluoro-4-((4-methylpiperazin-1-yl)methyl)phenyl)amino)pyrimidin-4-yl)benzoic acid as yellow solid (150 mg, yield: Crude).

TLC system: MeOH/dichloromethane (10:90)

R_{f} value: ˜0.1

LCMS(m/z): 422.49 (M+H)⁺

### Step 4: Synthesis of 4-(2-((3-fluoro-4-((4-methylpiperazin-1-yl)methyl)phenyl)amino)pyrimidin-4-yl)-N-((tetrahydro-2H-pyran-2-yl)oxy)benzamide

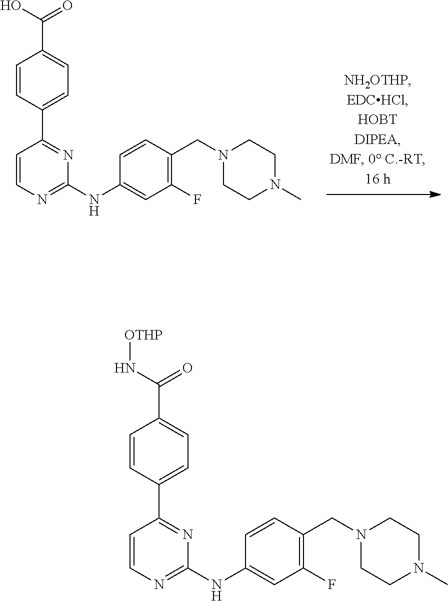

To a stirred solution of 4-(2-((3-fluoro-4-((4-methylpiperazin-1-yl)methyl)phenyl)amino)pyrimidin-4-yl)benzoic acid (150 mg, 0.35 mmol, 1 eq) in DMF (2.5 mL) at 0° C., was added DIPEA (130 mg, 1.05 mmol, 3.0 eq), HOBt (47 mg, 0.35 mmol, 1.0 eq), EDC-HCl (67 mg, 0.35 mmol, 1.0 eq) and stirred for 30 min. After that NH₂OTHP (50 mg, 0.43 mmol, 1.2 eq) was added and stirred at room temperature for 16 h. After completion of reaction by TLC, the reaction mixture was quenched with ice cold water (30 mL) and extracted with 10% MeOH in dichloromethane (2×15 mL). The combined organic layer was washed with brine solution (15 mL), dried over Na₂SO₄, filtered and concentrated under reduced pressure to afford 4-(2-((3-fluoro-4-((4-methylpiperazin-1-yl)methyl)phenyl)amino)pyrimidin-4-yl)-N-((tetrahydro-2H-pyran-2-yl)oxy)benzamide as yellow gum (150 mg, Crude).

TLC system: MeOH/dichloromethane (10:90)

R_{f} value: ˜0.4

LCMS(m/z): 521.47 (M+H)⁺

### Step 5: Synthesis of 4-(2-((3-fluoro-4-((4-methylpiperazin-1-yl)methyl)phenyl)amino)pyrimidin-4-yl)-N-hydroxybenzamide

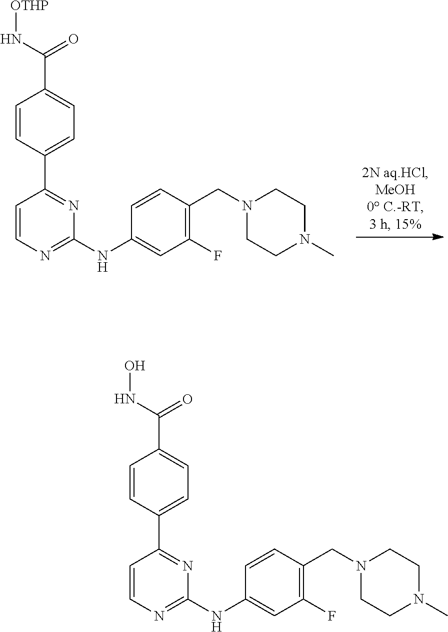

To a stirred solution of 4-(2-((3-fluoro-4-((4-methylpiperazin-1-yl)methyl)phenyl)amino)pyrimidin-4-yl)-N-((tetrahydro-2H-pyran-2-yl)oxy)benzamide (140 mg, 0.26 mmol, 1 eq (LCMS: 62%)) in MeOH (1.4 mL) at 0° C., was added 2N aq. HCl (1.4 mL, 10 Vol) and stirred at room temperature for 3 h. After completion of reaction by TLC, the reaction mixture was concentrated under reduced pressure and purified by Prep-HPLC. The collected fractions were frozen and lyophilized to afford 4-(2-((3-fluoro-4-((4-methylpiperazin-1-yl)methyl)phenyl)amino)pyrimidin-4-yl)-N-hydroxybenzamide as yellow solid (24 mg, yield: 15% in 3 steps).

TLC system: MeOH/dichloromethane (20:10)

R_{f} value: ˜0.1

LCMS (m/z): 437.3 (M+H)⁺

¹H NMR (400 MHz, DMSO-d₆) δ: 11.39 (s, 1H), 10.06 (s, 1H), 9.29 (br, 1H), 8.67 (d, J=5.2 Hz, 1H), 8.26 (d, J=8.4 Hz, 1H), 7.95 (d, J=8.4 Hz, 2H), 7.93-7.90 (m, 1H), 7.61-7.59 (m, 1H), 7.57 (d, J=5.2 Hz, 1H), 7.36 (t, J=8.4 Hz, 1H), 3.55 (br, 2H), 3.45-3.38 (br, 4H), 3.09-3.01 (br, 4H), 2.79 (s, 3H)

### Synthetic Example 8

### Synthesis of 4-(2-((3-Fluoro-4-((2-Methylmorpholino)Methyl)Phenyl)Amino)Pyrimidin-4-Yl)-N-Hydroxybenzamide

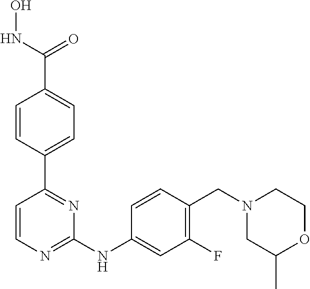 [SMILES: CC1CN(Cc2ccc(Nc3nccc(-c4ccc(C(=O)NO)cc4)n3)cc2F)CCO1]

### Step 1: Synthesis of 3-fluoro-4-((2-methylmorpholino)methyl)aniline

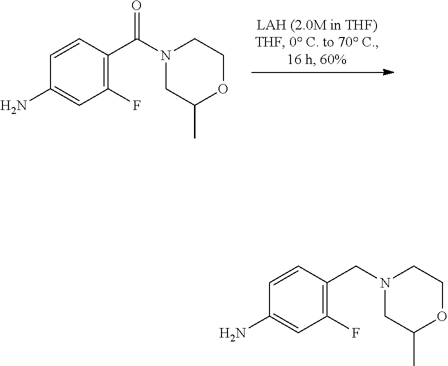 [SMILES: CC1CN(C(=O)c2ccc(N)cc2F)CCO1 ; CC1CN(Cc2ccc(N)cc2F)CCO1]

To a stirred solution of (4-amino-2-fluorophenyl)(2-methylmorpholino)methanone (2.2 g, 9.24 mmol, 1.0 eq) in THF (22 mL) at 0° C., was added LAH (2.0 M in THF) (14.0 mL, 27.7 mmol, 3.0 eq) and stirred at 70° C. for 16 h. After completion of reaction by TLC, the reaction mixture was quenched with NH₄Cl solution (25 mL) and extracted with 10% MeOH in dichloromethane (2×30 mL). The combined organic layer was dried over Na₂SO₄, filtered, concentrated under reduced pressure and purified by reverse phase column chromatography (Grace) [elution with 10% acetonitrile in 0.1% formic acid in H₂O] to afford 3-fluoro-4-((2-methylmorpholino)methyl)aniline as pale yellow gummy (1.2 g, yield: 60%).

TLC system: EtOAc/Hexane (100:00)

R_{f} value: ˜0.4

¹H NMR (400 MHz, CDCl₃) δ: 7.35-7.30 (m, 1H), 6.50 (dd, J=2.0 Hz, 8.0 Hz, 1H), 6.41 (dd, J=2.0 Hz, 12 Hz, 1H), 5.43 (bs, 2H), 4.05-3.91 (m, 5H), 3.18-3.15 (m, 2H), 2.65-2.62 (m, 1H), 2.30 (t, J=11.2 Hz, 1H), 1.19 (d, J=6.0 Hz, 3H)

### Step 2: Synthesis of methyl 4-(2-((3-fluoro-4-((2-methylmorpholino)methyl)phenyl)amino)pyrimidin-4-yl)benzoate

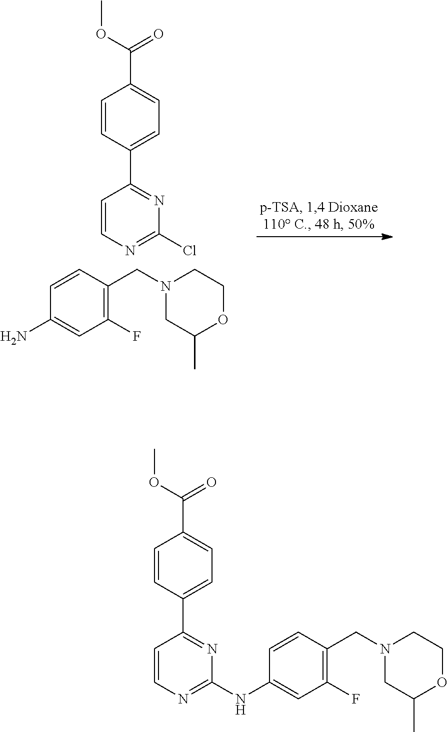

To a degassed stirred solution of methyl 4-(2-chloropyrimidin-4-yl)benzoate (600 mg, 2.41 mmol, 1 eq) and 3-fluoro-4-((2-methylmorpholino)methyl)aniline (540 mg, 2.41 mmol, 1.0 eq) in 1,4-dioxane (12 mL) at room temperature, was added p-TSA (373 mg, 2.17 mmol, 0.9 eq) and stirred at 110° C. for 48 h. After completion of reaction by TLC, reaction mixture was diluted with water (80 mL) and extracted with EtOAc (2×50 mL). The combined organic layer was dried over Na₂SO₄, filtered, concentrated and purified by reverse phase column chromatography (Grace) [elution with 30% acetonitrile in 0.1% formic acid in H₂O] to afford methyl 4-(2-((3-fluoro-4-((2-methylmorpholino)methyl)phenyl)amino)pyrimidin-4-yl)benzoate as pale brown solid (500 mg, yield: 50%).

TLC system: MeOH/dichloromethane (10:90)

R_{f} value: ˜0.3

LCMS(m/z): 437.5 (M+H)⁺

### Step 3: Synthesis of 4-(2-((3-fluoro-4-((2-methylmorpholino)methyl)phenyl)amino)pyrimidin-4-yl)benzoic acid

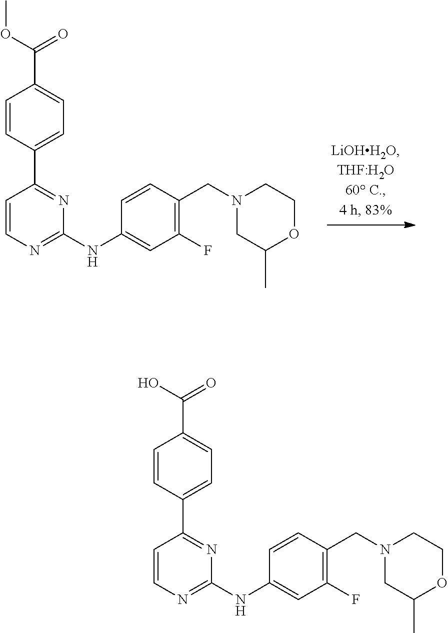 [SMILES: COC(=O)c1ccc(-c2ccnc(Nc3ccc(CN4CCOC(C)C4)c(F)c3)n2)cc1 ; CC1CN(Cc2ccc(Nc3nccc(-c4ccc(C(=O)O)cc4)n3)cc2F)CCO1]

To a stirred solution of methyl 4-(2-((3-fluoro-4-((2-methylmorpholino)methyl)phenyl)amino)pyrimidin-4-yl)benzoate (500 mg, 1.15 mmol, 1.0 eq) in THF:H₂O (2:1) (5.0 mL) at 0° C., was added LiOH·H₂O (96 mg, 2.29 mmol, 2 eq) and stirred at 60° C. for 4 h. After completion of reaction by TLC, reaction mixture washed with diethyl ether (15 mL). Aqueous layer was acidified with 2N HCl solution (10 mL), solidification observed, filtered and dried under vacuum to afford 4-(2-((3-fluoro-4-((2-methylmorpholino)methyl)phenyl)amino)pyrimidin-4-yl)benzoic acid as off white solid (400 mg, yield: 83%).

TLC system: MeOH/dichloromethane (10:90)

R_{f} value: ˜0.2

LCMS(m/z): 423.2 (M+H)⁺

### Step 4: Synthesis of 4-(2-((3-fluoro-4-((2-methylmorpholino)methyl)phenyl)amino)pyrimidin-4-yl)-N-((tetrahydro-2H-pyran-2-yl)oxy)benzamide

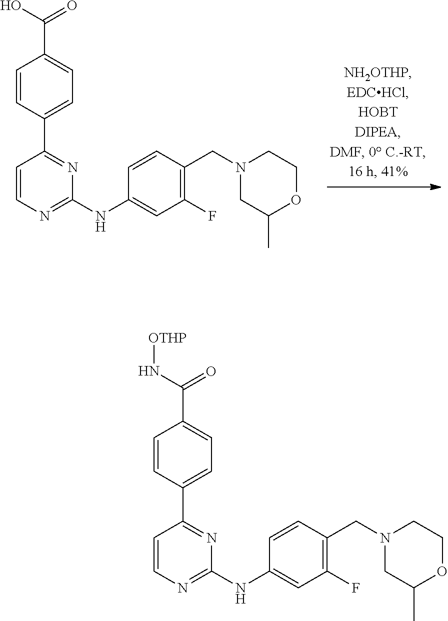

To a stirred solution of 4-(2-((3-fluoro-4-((2-methylmorpholino)methyl)phenyl)amino)pyrimidin-4-yl)benzoic acid (400 mg, 0.95 mmol, 1 eq) in DMF (4.0 mL) at 0° C., was added DIPEA (368 mg, 2.85 mmol, 3.0 eq), HOBt (128 mg, 0.95 mmol, 1.0 eq) followed by EDC-HCl (182 mg, 0.95 mmol, 1.0 eq) and stirred for 30 min. Later, NH₂OTHP (133 mg, 1.14 mmol, 1.2 eq) was added and stirred at room temperature for 16 h. After completion of reaction by TLC, reaction mixture was quenched with ice cold water (20 mL) and extracted with 10% MeOH in dichloromethane (2×25 mL). The combined organic layer was washed with brine solution (20 mL), dried over Na₂SO₄, filtered and concentrated under reduced pressure to afford 4-(2-((3-fluoro-4-((2-methylmorpholino)methyl)phenyl)amino)pyrimidin-4-yl)-N-((tetrahydro-2H-pyran-2-yl)oxy)benzamide as pale yellow solid (210 mg, Yield: 41%).

TLC system: MeOH/dichloromethane (10:90)

R_{f} value: ˜0.4

LCMS(m/z): 522.8 (M+H)⁺

### Step 5: Synthesis of 4-(2-((3-fluoro-4-((2-methylmorpholino)methyl)phenyl)amino)pyrimidin-4-yl)-N-hydroxybenzamide

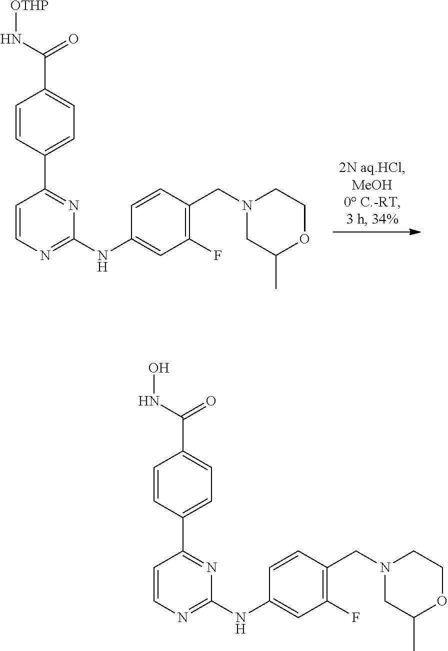

To a stirred solution of 4-(2-((3-fluoro-4-((2-methylmorpholino)methyl)phenyl)amino)pyrimidin-4-yl)-N-((tetrahydro-2H-pyran-2-yl)oxy)benzamide (200 mg, 0.38 mmol, 1.0 eq) in MeOH (2.0 mL) at 0° C., was added 2N aq. HCl (2.0 mL, 10 Vol) and stirred at room temperature for 3 h. After completion of reaction by TLC, the reaction mixture was concentrated and purified by reverse phase column chromatography (Grace) [elution with 25% acetonitrile in 0.1% formic acid in H₂O] to afford 4-(2-((3-fluoro-4-((2-methylmorpholino)methyl)phenyl)amino)pyrimidin-4-yl)-N-hydroxybenzamide as off white solid (54 mg, yield: 34%).

TLC system: MeOH/dichloromethane (10:10)

R_{f} value: ˜0.1

LCMS (m/z): 438.2 (M+H)⁺

¹H NMR (400 MHz, DMSO-d₆) δ: 11.36 (s, 1H), 9.96 (s, 1H), 9.14 (brs, 1H), 8.63 (d, J=5.2 Hz, 1H), 8.24 (d, J=8.4 Hz, 2H), 7.93 (d, J=8.8 Hz, 2H), 7.85 (dd, J=2.0 Hz, 13.2 Hz, 1H), 7.56-7.51 (m, 2H), 7.30 (t, J=8.8 Hz, 1H), 3.74-3.71 (m, 1H), 3.50-3.44 (m, 4H), 2.71-2.62 (m, 2H), 2.08-2.05 (m, 1H), 1.78-1.72 (m, 1H), 1.02 (d, J=6.4 Hz, 3H)

### Synthetic Example 9

### Synthesis of 4-(2-((3-Fluoro-4-((4-(Oxetan-3-Yl)Piperazin-1-Yl)Methyl)Phenyl)Amino)Pyrimidin-4-Yl)-N-Hydroxybenzamide

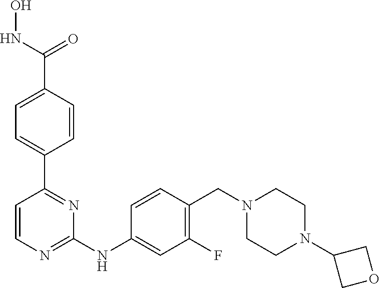 [SMILES: O=C(NO)c1ccc(-c2ccnc(Nc3ccc(CN4CCN(C5COC5)CC4)c(F)c3)n2)cc1]

### Step 1: Synthesis of (2-fluoro-4-nitrophenyl)methanol

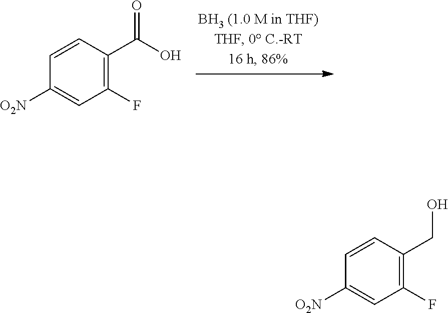 [SMILES: O=C(O)c1ccc([N+](=O)[O-])cc1F ; O=[N+]([O-])c1ccc(CO)c(F)c1]

To a stirred solution of 2-fluoro-4-nitrobenzoic acid (5 g, 27.0 mmol, 1.0 eq) in THF (50 mL) at 0° C., was added BH₃ (1.0 M in THF) (81 mL, 81.1 mmol, 3.0 eq) and stirred at room temperature for 16 h. After completion of reaction by TLC, the reaction mixture was quenched with MeOH (10 mL), concentrated, diluted with water (50 mL) and extracted with EtOAc (2×50 mL). The combined organic layer was dried over Na₂SO₄, filtered and concentrated under reduced pressure to afford (2-fluoro-4-nitrophenyl)methanol as off white solid (4 g, yield: 86%).

TLC system: EtOAc/Hexane (50:50)

R_{f} value: ˜0.5

¹H NMR (400 MHz, CDCl₃) δ: 8.06 (dd, J=8.4 & 2.0 Hz, 1H), 7.95-7.90 (m, 1H), 7.71 (t, J=7.6 Hz, 1H), 4.88 (s, 2H).

### Step 2: Synthesis of 2-fluoro-4-nitrobenzaldehyde

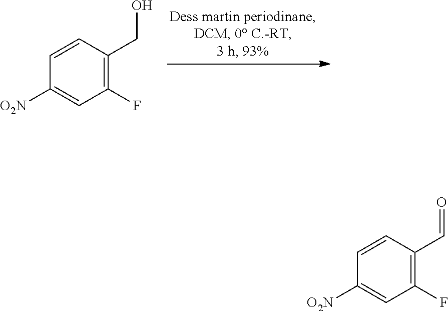 [SMILES: O=[N+]([O-])c1ccc(CO)c(F)c1 ; O=Cc1ccc([N+](=O)[O-])cc1F]

To a stirred solution of (2-fluoro-4-nitrophenyl)methanol (3 g, 17.5 mmol, 1.0 eq) in dichloromethane (30 mL) at 0° C., was added Dess martin periodinane (14.8 g, 35.1 mmol, 2.0 eq) and stirred at room temperature for 3 h. After completion of reaction by TLC, reaction mixture was diluted with water (100 mL) and extracted with dichloromethane (2×50 mL). Combined organic layer was dried over Na₂SO₄, filtered, concentrated under reduced pressure a purified by silica gel column (100-200 mesh) [elution with 20% EtOAc in pet ether] to afford 2-fluoro-4-nitrobenzaldehyde as off white solid (2.5 g, yield: 93%).

TLC system: EtOAc/Hexane (40:60)

R_{f} value: ˜0.4

¹H NMR (400 MHz, CDCl₃) δ: 10.48 (s, 1H), 8.18-8.11 (m, 3H)

### Step 3: Synthesis of 1-(2-fluoro-4-nitrobenzyl)-4-(oxetan-3-yl)piperazine

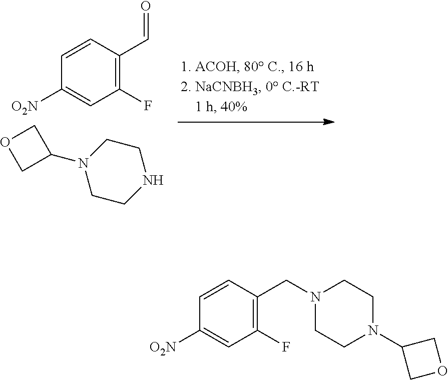

To a stirred solution of 2-fluoro-4-nitrobenzaldehyde (2.4 g, 14.2 mmol, 1.0 eq) in MeOH (25 mL) at 0° C., was added 1-(oxetan-3-yl)piperazine (2.42 g, 17.04 mmol, 1.2 eq), acetic acid (1.79 g, 29.8 mmol, 2.1 eq) and stirred at 80° C. for 16 h. Later cooled to 0° C., added N acetonitrile BH₃ (1.76 g, 28.4 mmol, 2.0 eq) and stirred at room temperature for 1 h. After completion of reaction by TLC, reaction mixture was diluted with water (80 mL) and extracted with 10% MeOH in dichloromethane (3×30 mL). Combined organic layer was dried over Na₂SO₄, filtered, concentrated under reduced pressure and purified by reverse phase column chromatography [elution with 35% acetonitrile in 0.1% formic acid in H₂O] to afford 1-(2-fluoro-4-nitrobenzyl)-4-(oxetan-3-yl)piperazine as yellow gum (1.7 g, yield: 40%).

TLC system: MeOH/dichloromethane (10:90)

R_{f} value: ˜0.3

LCMS(m/z): 296.2 (M+H)⁺

¹H NMR (400 MHz, CDCl₃) δ: 8.02 (dd, J=8.4 & 2.0 Hz, 1H), 7.92 (dd, J=9.6 & 2.4 Hz, 1H), 7.65 (t, J=8.0 Hz, 1H), 4.69-4.61 (m, 4H), 3.72 (s, 2H), 3.59-3.52 (m, 1H), 2.64 (brs, 4H), 2.45 (brs, 4H)

### Step 4: Synthesis of 3-fluoro-4-((4-(oxetan-3-yl)piperazin-1-yl)methyl)aniline

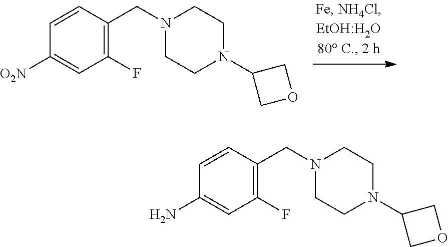 [SMILES: O=[N+]([O-])c1ccc(CN2CCN(C3COC3)CC2)c(F)c1 ; Nc1ccc(CN2CCN(C3COC3)CC2)c(F)c1]

To a stirred solution of 1-(2-fluoro-4-nitrobenzyl)-4-(oxetan-3-yl)piperazine (1.5 g, 5.06 mmol, 1 eq) in EtOH:H₂O (2:1, 15 mL) at room temperature, was added Fe (1.42 g, 25.3 mmol, 5 eq) and NH₄Cl (1.34 g, 25.3 mmol, 5 eq) and heated to 80° C., stirred for 2 h. After completion of reaction by TLC, reaction mixture was filtered through a pad of diatomaceous earth (e.g., Celite®) and washed with EtOAc (20 mL). The collected filtrate was concentrated, diluted with water (80 mL) and extracted with EtOAc 3×30 mL). Organic layer was dried over Na₂SO₄, filtered and concentrated under reduced pressure to afford 3-fluoro-4-((4-(oxetan-3-yl)piperazin-1-yl)methyl)aniline as pale brown gum (1.3 g, yield: Crude).

TLC system: MeOH/dichloromethane (10:80)

R_{f} value: ˜0.4

LCMS(m/z): 266.2 (M+H)⁺

### Step 5: Synthesis of methyl 4-(2-((3-fluoro-4-((4-(oxetan-3-yl)piperazin-1-yl)methyl)phenyl)amino)pyrimidin-4-yl)benzoate

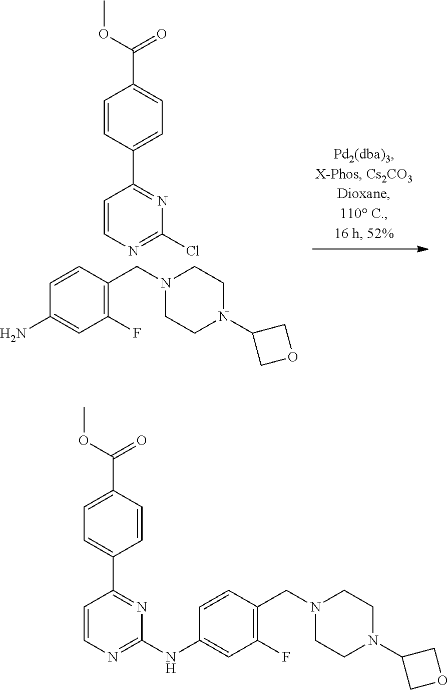

To a degassed stirred solution of methyl 4-(2-chloropyrimidin-4-yl)benzoate (500 mg, 2.02 mmol, 1 eq) and 3-fluoro-4-((4-(oxetan-3-yl)piperazin-1-yl)methyl)aniline (540 mg, 2.02 mmol, 1.0 eq) in 1,4-dioxane (5 mL) at room temperature, was added Cs₂CO₃ (1.32 g, 4.04 mmol, 2.0 eq), X-Phos (96 mg, 0.2 mmol, 0.1 eq) followed by Pd₂(dba)₃ (92 mg, 0.1 mmol, 0.05 eq). The reaction mixture was stirred at 110° C. for 16 h. After completion of reaction by TLC, reaction mixture diluted with water (30 mL) and extracted with 10% MeOH in dichloromethane (3×15 mL). Organic layer was dried over Na₂SO₄, filtered, concentrated under reduced pressure and purified by reverse phase column chromatography (Grace) [elution with 30% acetonitrile in 0.1% formic acid in H₂O] to afford methyl 4-(2-((3-fluoro-4-((4-(oxetan-3-yl)piperazin-1-yl)methyl)phenyl)amino)pyrimidin-4-yl)benzoate as pale brown solid (500 mg, yield: 52%).

TLC system: MeOH/dichloromethane (10:90)

R_{f} value: ˜0.4

LCMS(m/z): 478.3 (M+H)⁺

### Step 6: Synthesis of 4-(2-((3-fluoro-4-((4-(oxetan-3-yl)piperazin-1-yl)methyl)phenyl)amino)pyrimidin-4-yl)benzoic acid

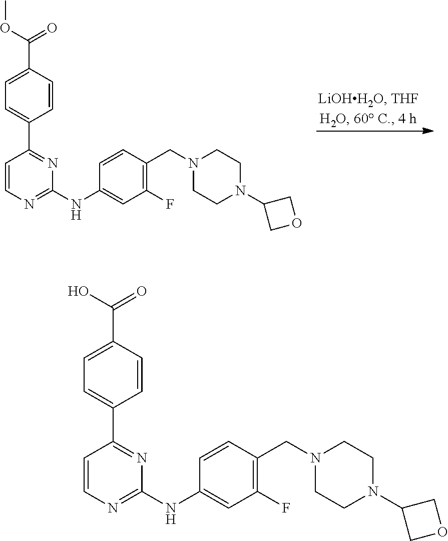 [SMILES: COC(=O)c1ccc(-c2ccnc(Nc3ccc(CN4CCN(C5COC5)CC4)c(F)c3)n2)cc1 ; "LiOH·H2O, THFH2O, 60° C., 4 h" ; O=C(O)c1ccc(-c2ccnc(Nc3ccc(CN4CCN(C5COC5)CC4)c(F)c3)n2)cc1]

To a stirred solution of methyl 4-(2-((3-fluoro-4-((4-(oxetan-3-yl)piperazin-1-yl)methyl)phenyl)amino)pyrimidin-4-yl)benzoate (500 mg, 1.05 mmol, 1.0 eq) in THF:H₂O (2:1) (5 mL) at 0° C., was added LiOH·H₂O (88 mg, 2.09 mmol, 2 eq) and stirred at 60° C. for 4 h. After completion of reaction by TLC, reaction mixture was washed with diethyl ether (10 mL). Aqueous layer was acidified with 2N HCl solution (5 mL), solidification observed, filtered and solid was dried under vacuum to afford 4-(2-((3-fluoro-4-((4-(oxetan-3-yl)piperazin-1-yl)methyl)phenyl)amino)pyrimidin-4-yl)benzoic acid as pale yellow solid (450 mg, yield: Crude).

TLC system: MeOH/dichloromethane (10:90)

R_{f} value: ˜0.2

LCMS(m/z): 464.3 (M+H)⁺

¹H NMR (400 MHz, CDCl₃) δ: 11.52 (s, 1H), 10.25 (s, 1H), 8.70 (d, J=5.2 Hz, 1H), 8.29 (d, J=8.4 Hz, 2H), 8.11 (d, J=8.8 Hz, 2H), 8.02-7.98 (m, 1H), 7.67-7.59 (m, 3H), 4.68-4.61 (m, 4H), 4.32 (brs, 2H), 4.13-3.37 (br, 5H), 3.51 (brs, 2H), 2.97 (brs, 2H)

### Step 7: Synthesis of 4-(2-((3-fluoro-4-((4-(oxetan-3-yl)piperazin-1-yl)methyl)phenyl)amino)pyrimidin-4-yl)-N-((tetrahydro-2H-pyran-2-yl)oxy)benzamide

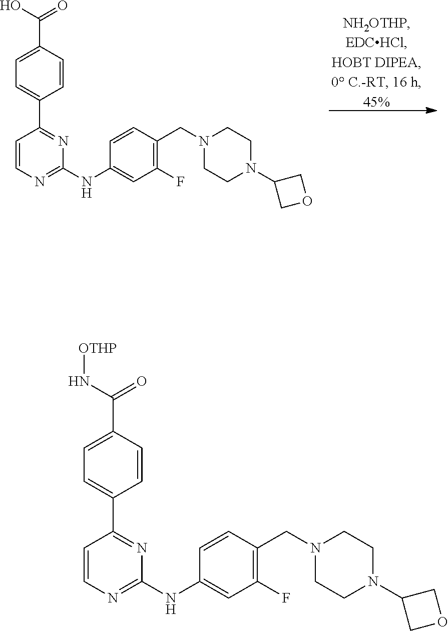

To a stirred solution of 4-(2-((3-fluoro-4-((4-(oxetan-3-yl)piperazin-1-yl)methyl)phenyl)amino)pyrimidin-4-yl)benzoic acid (450 mg, 0.97 mmol, 1 eq) in DMF (4.5 mL) at 0° C., was added DIPEA (375 mg, 2.91 mmol, 3.0 eq), HOBt (131 mg, 0.97 mmol, 1.0 eq) followed by EDC-HCl (186 mg, 0.97 mmol, 1.0 eq) and stirred for 30 min. Later, added NH₂OTHP (136 mg, 1.16 mmol, 1.2 eq) and stirred at room temperature for 16 h. After completion of reaction by TLC, quenched with ice cold water (40 mL), and extracted with 10% MeOH in dichloromethane (3×20 mL). The combined organic layer was washed with brine solution (20 mL), dried over Na₂SO₄, filtered, concentrated under reduced pressure and purified by reverse phase column chromatography (Grace) [elution with 30% acetonitrile in 0.1% formic acid in H₂O] to afford 4-(2-((3-fluoro-4-((4-(oxetan-3-yl)piperazin-1-yl)methyl)phenyl)amino)pyrimidin-4-yl)-N-((tetrahydro-2H-pyran-2-yl)oxy)benzamide as pale yellow solid (250 mg, Yield: 45%).

TLC system: MeOH/dichloromethane (10:90)

R_{f} value: ˜0.4

LCMS(m/z): 563.8 (M+H)⁺

### Step 8: Synthesis of 4-(2-((3-fluoro-4-((4-(oxetan-3-yl)piperazin-1-yl)methyl)phenyl)amino)pyrimidin-4-yl)-N-hydroxybenzamide

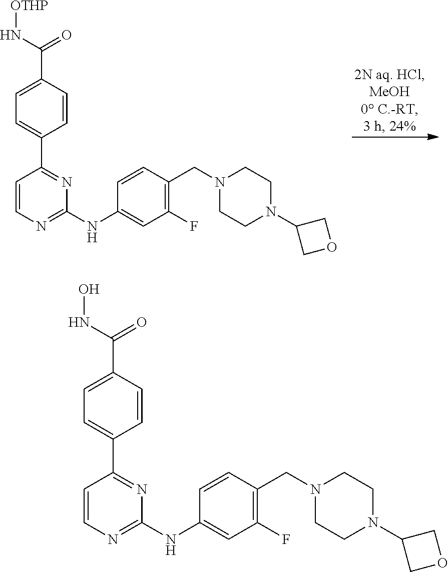

To a stirred solution of 4-(2-((3-fluoro-4-((4-(oxetan-3-yl)piperazin-1-yl)methyl)phenyl)amino)pyrimidin-4-yl)-N-((tetrahydro-2H-pyran-2-yl)oxy)benzamide (240 mg, 0.45 mmol, 1 eq) in MeOH (2.4 mL) at 0° C., was added 2N aq. HCl (2.4 mL, 10 Vol) and stirred at room temperature for 3 h. After completion of reaction by TLC, concentrated under reduced pressure and purified by reverse phase column chromatography [elution with 20% acetonitrile in 0.1% formic acid in H₂O] to afford 4-(2-((3-fluoro-4-((4-(oxetan-3-yl)piperazin-1-yl)methyl)phenyl)amino)pyrimidin-4-yl)-N-hydroxybenzamide as off white solid (55 mg, yield: 24%).

TLC system: MeOH/dichloromethane (20:10)

R_{f} value: ˜0.1

LCMS (m/z): 479.3 (M+H)⁺

¹H NMR (400 MHz, DMSO-d₆) δ: 11.37 (s, 1H), 9.99 (s, 1H), 9.18 (s, 1H), 8.64 (d, J=5.2 Hz, 1H), 8.24 (d, J=8.4 Hz, 2H), 7.93 (d, J=8.4 Hz, 2H), 7.86 (d, J=12 Hz, 1H), 7.57-7.52 (m, 2H), 7.32 (t, J=8.4 Hz, 1H), 4.52 (t, J=6.4 Hz, 2H), 4.40 (t, J=6.4 Hz, 2H), 3.59 (brs, 2H), 3.47-3.43 (br, 1H), 2.58-2.51 (br, 4H), 2.31-2.25 (br, 4H)

### Synthetic Example 10

### Synthesis of 4-(2-((3-Fluoro-4-(1-(4-Methylpiperazin-1-Yl)Ethyl)Phenyl)Amino)Pyrimidin-4-Yl)-N-Hydroxybenzamide.Formic Acid Salt

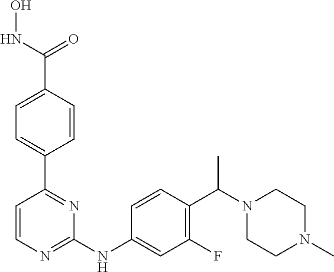 [SMILES: CC(c1ccc(Nc2nccc(-c3ccc(C(=O)NO)cc3)n2)cc1F)N1CCN(C)CC1]

### Steps 1 and 2: Synthesis of 1-(2-fluoro-4-nitrophenyl)ethan-1-one

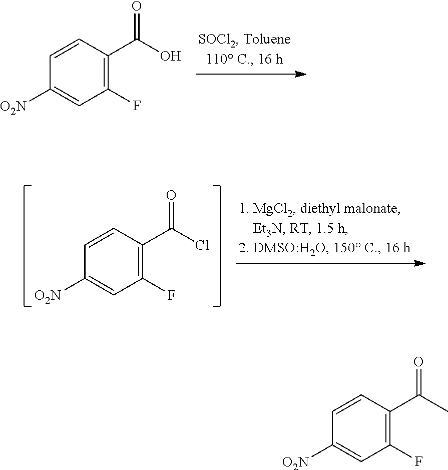

To a stirred solution of 2-fluoro-4-nitrobenzoic acid (5 g, 27.02 mmol, 1.0 eq) in Toluene (50 mL), at 0° C., was added SOCl₂ (9.5 g, 81.08 mmol, 3.0 eq) and stirred at 100° C. for 16 h. After completion of reaction by TLC, e reaction mixture was concentrated under reduced pressure to afford 2-fluoro-4-nitrobenzoyl chloride as an orange gummy liquid (5.0 g, Yield: crude).
TLC system: EtOAc/Hexane (10:90)
R_{f} value: ˜0.4

Crude directly used for next step.

To a stirred solution of MgCl₂ (1.8 g, 18.9 mmol, 0.8 eq) in Toluene (50 mL) at room temperature, was added Et₃N (6.3 g, 62.5 mmol, 2.5 eq) followed by dimethyl malonate (4.3 g, 32.5 mmol, 1.3 eq) and stirred at room temperature for 45 min. After that at 0° C., was added 2-fluoro-4-nitrobenzoyl chloride (5 g, 24.6 mmol, 1.0 eq) and stirred for 1 h, later allowed to room temperature after addition of concentrated HCl (1.3 mL), stirred for 1 h. After completion of reaction by TLC, diluted with water (100 mL) and extracted with EtOAc (3×80 mL). The combined organic layer was dried over Na₂SO₄, filtered and concentrated. Obtained crude was dissolved in DMSO (20 mL) and H₂O (5 mL) and stirred at 150° C. for 16 h. The reaction mixture was diluted with water (100 mL) and extracted with EtOAc (2×100 mL). The combined organic layer was dried over Na₂SO₄, filtered, and concentrated under reduced pressure to afford crude product. The crude product was purified by silica gel (100-200) [elution with 5-10% EtOAc in pet ether] to afford 1-(2-fluoro-4-nitrophenyl)ethan-1-one as an off white solid (2.5 g, yield: 51% over two steps).

TLC system: EtOAc/Hexane (10:90)

R_{f} value: ˜0.3

¹H NMR (400 MHz, CDCl₃) δ: 8.11-8.02 (m, 3H), 2.71 (d, 3H)

### Step 3: Synthesis of 1-(1-(2-fluoro-4-nitrophenyl)ethyl)-4-methylpiperazine

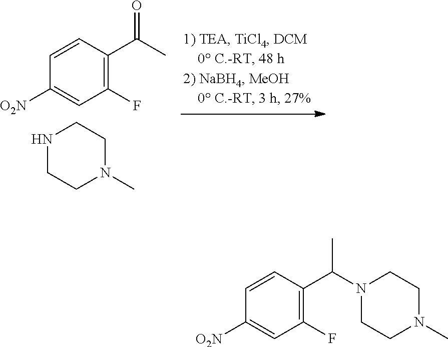

To a stirred solution of 1-(2-fluoro-4-nitrophenyl)ethan-1-one (2.5 g, 13.7 mmol, 1.0 eq) in dichloromethane (25 mL) at 0° C., was added 1-methylpiperazine (2.73 g, 27.3 mmol, 2 eq), Et₃N (3.4 g, 34.2 mmol, 2.5 eq), TiCl₄ (1.3 g, 6.83 mmol, 0.5 eq) and stirred at room temperature for 48 h. Later cooled to 0° C., added NaBH₄ (1.1 g, 27.3 mmol, 2.0 eq) and MeOH (5 mL and stirred at room temperature for 3 h. After completion of reaction by TLC, diluted with water (50 mL) and extracted with 10% MeOH in dichloromethane (2×50 mL). The combined organic layer was dried over Na₂SO₄, filtered, and concentrated under reduced pressure. The crude product was purified by reverse phase column chromatography [elution with 35-40% acetonitrile in 0.1% formic acid in H₂O] to afford 1-(1-(2-fluoro-4-nitrophenyl)ethyl)-4-methylpiperazine as a yellow gummy mass (1 g, yield: 27%).

TLC system: MeOH/dichloromethane (10:90)

R_{f} value: ˜0.2

LCMS(m/z): 268.1 (M+H)⁺

¹H NMR (400 MHz, CDCl₃) δ: 8.38 (1H; formic acid), 8.02 (dd, J=2.0 Hz, 8.4 Hz, 1H), 7.92 (dd, J=2.0 Hz, 9.6 Hz, 1H), 7.66-7.62 (m, 1H), 3.93 (q, J=6.8 Hz, 1H), 2.89-2.71 (m, 6H), 2.65-2.57 (m, 2H), 2.53 (s, 3H), 1.37 (d, J=6.8 Hz, 3H)

### Step 4: Synthesis of 3-fluoro-4-(1-(4-methylpiperazin-1-yl)ethyl)aniline

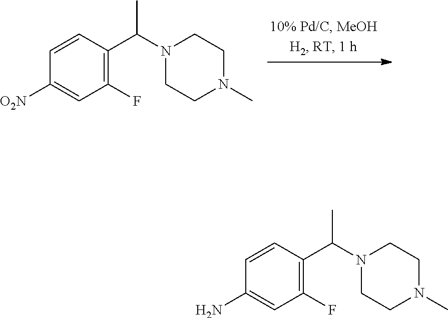 [SMILES: CC(c1ccc([N+](=O)[O-])cc1F)N1CCN(C)CC1 ; CC(c1ccc(N)cc1F)N1CCN(C)CC1]

To a stirred solution of 1-(1-(2-fluoro-4-nitrophenyl)ethyl)-4-methylpiperazine (1 g, 3.74 mmol, 1 eq) in MeOH (10 mL) at room temperature, was added 10% Pd/C (300 mg, 30% wt/wt) and stirred for 1 h under H₂ atmosphere. After completion of reaction by TLC, reaction mixture was filtered through a pad of diatomaceous earth (e.g., Celite®) and washed with 10% MeOH in dichloromethane (20 mL). The collected filtrate was concentrated under reduced pressure to afford 3-fluoro-4-(1-(4-methylpiperazin-1-yl)ethyl)aniline as orange gummy solid (930 mg, yield: Crude).

TLC system: MeOH/dichloromethane (10:80)

R_{f} value: ˜0.2; LCMS (m/z): 238.3 (M+H)⁺

¹H NMR (400 MHz, DMSO-d₆) δ: 6.97 (t, J=8.4 Hz, 1H), 6.34 (dd, J=2.0 Hz, 8.4 Hz, 1H), 6.25 (dd, J=2 Hz, 12.8 Hz, 1H), 5.24 (s, 2H), 3.56 (t, J=7.2 Hz, 1H), 2.35-2.22 (m, 8H), 2.10 (s, 3H), 1.22 (d, J=6.8 Hz, 3H)

### Step 5: Synthesis of methyl 4-(2-((3-fluoro-4-(1-(4-methylpiperazin-1-yl)ethyl)phenyl)amino)pyrimidin-4-yl)benzoate

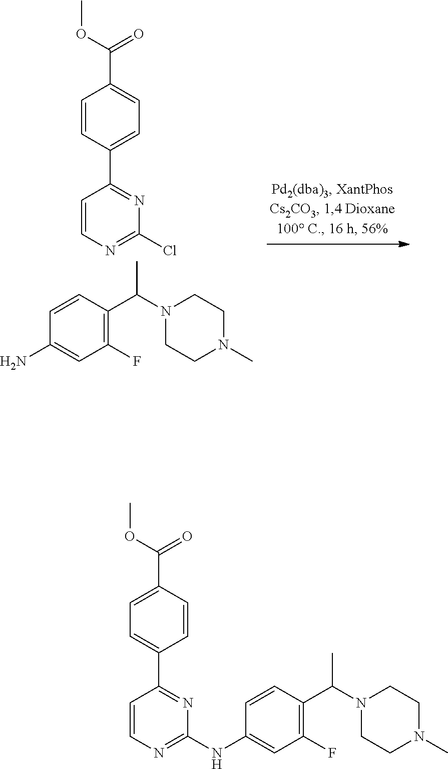

To a degassed stirred solution of methyl 4-(2-chloropyrimidin-4-yl)benzoate (850 mg, 3.42 mmol, 1 eq) and 3-fluoro-4-(1-(4-methylpiperazin-1-yl)ethyl)aniline (850 mg, 3.42 mmol, 1.0 eq) in 1,4-dioxane (8.5 mL) at room temperature, was added Cs₂CO₃ (2.23 g, 6.85 mmol, 2.0 eq), XantPhos (200 mg, 0.34 mmol, 0.1 eq) followed by Pd₂(dba)₃ (158 mg, 0.17 mmol, 0.05 eq). The reaction mixture was stirred at 100° C. for 16 h. After completion of reaction by TLC, diluted with water (50 mL) and extracted with 10% MeOH in dichloromethane (2×50 mL). The combined organic layer was dried over Na₂SO₄, filtered, and concentrated under reduced pressure. The crude product was purified by reverse phase column chromatography (Grace) [elution with 18-20% acetonitrile in 0.1% formic acid in H₂O] to afford methyl 4-(2-((3-fluoro-4-(1-(4-methylpiperazin-1-yl)ethyl)phenyl)amino)pyrimidin-4-yl)benzoate as orange solid (850 mg, yield: 56%).

TLC system: MeOH/dichloromethane (10:90)

R_{f} value: ˜0.2

LCMS(m/z): 450.3 (M+H)⁺

¹H NMR (400 MHz, CDCl₃) δ: 8.53 (d, J=5.2 Hz, 1H), 8.38 (bs, 1H; formic acid), 8.19 (d, J=8.4 Hz, 2H), 8.13 (d, J=8.4 Hz, 2H), 7.78 (dd, J=2.0 Hz, 12.8 Hz, 1H), 7.54 (brs, 1H), 7.34-7.30 (m, 2H), 7.27-7.25 (m, 1H), 3.99-3.94 (m, 4H), 2.98 (br, 4H), 2.75 (br, 4H), 2.59 (s, 3H), 1.43 (d, J=6.8 Hz, 3H)

### Step 6: Synthesis of 4-(2-((3-fluoro-4-(1-(4-methylpiperazin-1-yl)ethyl)phenyl)amino)pyrimidin-4-yl)benzoic acid

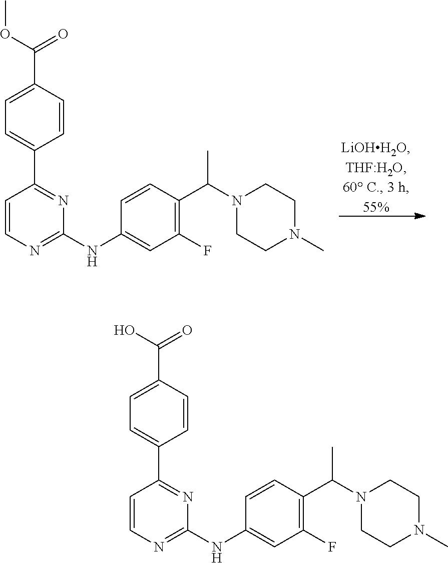 [SMILES: COC(=O)c1ccc(-c2ccnc(Nc3ccc(C(C)N4CCN(C)CC4)c(F)c3)n2)cc1 ; "LiOH·H2O,THF:H2O,60° C., 3 h,55%" ; CC(c1ccc(Nc2nccc(-c3ccc(C(=O)O)cc3)n2)cc1F)N1CCN(C)CC1]

To a stirred solution of methyl 4-(2-((3-fluoro-4-(1-(4-methylpiperazin-1-yl)ethyl)phenyl)amino)pyrimidin-4-yl)benzoate (850 mg, 1.89 mmol, 1.0 eq) in THF:H₂O (2:1) (8.5 mL) at 0° C., was added LiOH·H₂O (160 mg, 3.78 mmol, 2 eq) and stirred at 60° C. for 3 h. After completion of reaction by TLC, reaction mixture was washed with diethyl ether (10 mL), aqueous layer was acidified with 2N HCl solution (5 mL), solid formation observed, filtered and dried under vacuum to afford 4-(2-((3-fluoro-4-(1-(4-methylpiperazin-1-yl)ethyl)phenyl)amino)pyrimidin-4-yl)benzoic acid as pale yellow solid (450 mg, yield: 55%).

TLC system: MeOH/dichloromethane (10:90)

R_{f} value: ˜0.1

LCMS(m/z): 436.3 (M+H)⁺

### Step 7: Synthesis of 4-(2-((3-fluoro-4-(1-(4-methylpiperazin-1-yl)ethyl)phenyl)amino)pyrimidin-4-yl)-N-((tetrahydro-2H-pyran-2-yl)oxy)benzamide

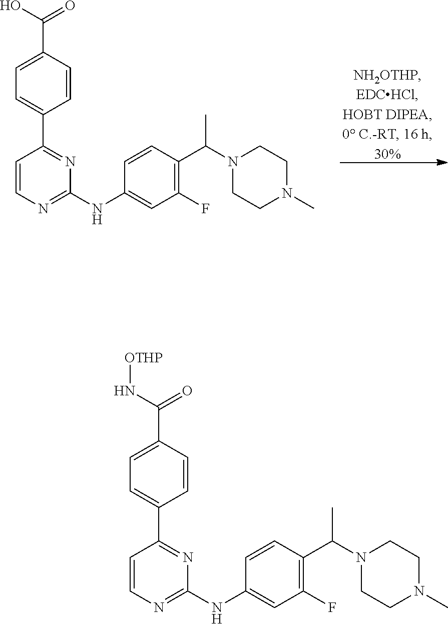

To a stirred solution of 4-(2-((3-fluoro-4-(1-(4-methylpiperazin-1-yl)ethyl)phenyl)amino)pyrimidin-4-yl)benzoic acid (400 mg, 0.92 mmol, 1 eq) in DMF (4.0 mL) at 0° C., was added DIPEA (0.45 mL, 2.76 mmol, 3.0 eq), HOBt (124.2 mg, 0.92 mmol, 1.0 eq), EDC-HCl (177 mg, 0.92 mmol, 1.0 eq) and stirred for 30 min. After that NH₂OTHP (212 mg, 1.10 mmol, 1.2 eq) was added to the reaction mixture and stirred at room temperature for 16 h. After completion of reaction by TLC, quenched with ice cold water (20 mL), and extracted with 10% MeOH in dichloromethane (2×20 mL). The combined organic layer was washed with brine solution (20 mL), dried over Na₂SO₄, filtered, and concentrated under reduced pressure. The crude product was purified by reverse phase column chromatography (Grace) [elution with 20% acetonitrile in 0.1% formic acid in H₂O] to afford 4-(2-((3-fluoro-4-(1-(4-methylpiperazin-1-yl)ethyl)phenyl)amino)pyrimidin-4-yl)-N-((tetrahydro-2H-pyran-2-yl)oxy)benzamide as an orange gummy solid (150 mg, Yield: 30%).

TLC system: MeOH/dichloromethane (10:90)

R_{f} value: ˜0.3

LCMS(m/z): 535.4 (M+H)⁺

### Step 8: Synthesis of 4-(2-((3-fluoro-4-(1-(4-methylpiperazin-1-yl)ethyl)phenyl)amino)pyrimidin-4-yl)-N-hydroxybenzamide formic acid salt

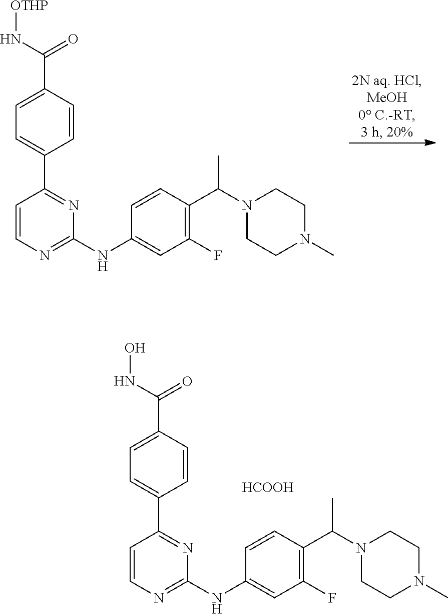

To a stirred solution of 4-(2-((3-fluoro-4-(1-(4-methylpiperazin-1-yl)ethyl)phenyl)amino)pyrimidin-4-yl)-N-((tetrahydro-2H-pyran-2-yl)oxy)benzamide (140 mg, 0.26 mmol, 1 eq) (LCMS: 83%)) in MeOH (1.4 mL) at 0° C., was added 2N aq. HCl (1.4 mL, 10 Vol) and stirred at room temperature for 3 h. After completion of reaction by TLC, reaction mixture was concentrated under reduced pressure and purified by reverse phase column chromatography [elution with 20% acetonitrile in 0.1% formic acid in H₂O] to afford 4-(2-((3-fluoro-4-(1-(4-methylpiperazin-1-yl)ethyl)phenyl)amino)pyrimidin-4-yl)-N-hydroxybenzamide formic acid salt as an off white solid (27 mg, yield: %).

TLC system: MeOH/dichloromethane (10:90)

R_{f} value: ˜0.1

LCMS (m/z): 451.3 (M+H)⁺

¹H NMR (400 MHz, DMSO-d₆) δ: 11.36 (brs, 1H), 9.95 (s, 1H), 9.14 (br, 1H) (D2O exchangeable), 8.62 (d, J=5.6 Hz, 1H), 8.24 (d, J=8.8 Hz, 2H), 7.93 (d, J=8.4 Hz, 2H), 7.82 (dd, J=13.6 & 2 Hz, 1H), 7.55 (dd, J=8.8 & 2 Hz, 1H), 7.52 (d, J=5.2 Hz, 1H), 7.33 (t, J=8.4 Hz, 1H), 3.76 (d, J=6.8 Hz, 1H), 2.58-2.52 (m, 4H), 2.47-2.39 (m, 4H), 2.28 (s, 3H), 1.32 (d, J=6.8 Hz, 3H)

### Synthetic Example 11

### Synthesis of 4-(2-((3-Fluoro-4-(2-Methylmorpholino)Phenyl)Amino)Pyrimidin-4-Yl)-N-Hydroxybenzamide

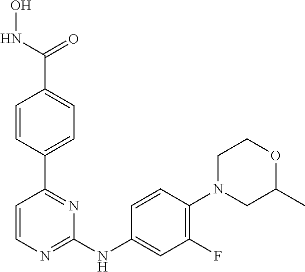 [SMILES: CC1CN(c2ccc(Nc3nccc(-c4ccc(C(=O)NO)cc4)n3)cc2F)CCO1]

### Step 1: Synthesis of 4-(2-fluoro-4-nitrophenyl)-2-methylmorpholine

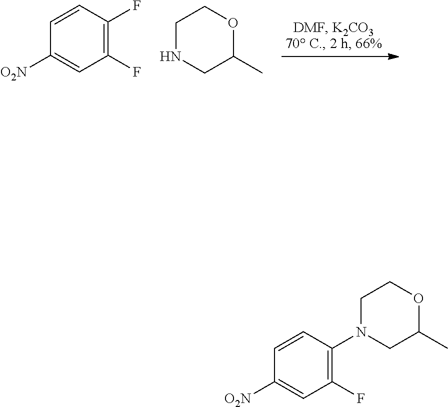

To a stirred solution of 1,2-difluoro-4-nitrobenzene (1.5 g, 9.43 mmol, 1.0 eq) in DMF (15 mL) at room temperature, was added 2-methylmorpholine (1.2 mL, 11.3 mmol, 1.2 eq) and K₂CO₃ (3.9 g, 28.3 mmol, 3.0 eq) and stirred at 70° C. for 2 h. After completion of reaction by TLC, reaction mixture was diluted with water (40 mL) and extracted with EtOAc (2×40 mL). The combined organic layer was dried over Na₂SO₄, filtered, and concentrated under reduced pressure to crude product. The crude product was purified by silica gel (60-120) column chromatography [elution with 10-15% EtOAc in Hexane] to afford 4-(2-fluoro-4-nitrophenyl)-2-methylmorpholine as yellow solid (1.5 g, yield: 66%).

TLC system: EtOAc/Hexane (20:80)

R_{f} value: ˜0.5

¹H NMR (400 MHz, CDCl₃) δ: 7.99 (dd, J=9.2 & 2.4 Hz, 1H), 7.91 (dd, J=13.2 & 2.4 Hz, 1H), 6.91 (t, J=8.8 Hz, 1H), 3.98-3.97 (m, 1H), 3.87-3.78 (m, 2H), 3.51-3.45 (m, 2H), 3.05-2.99 (m, 1H), 2.71-2.66 (m, 1H), 1.24 (d, J=6.4 Hz, 3H)

### Step 2: Synthesis of 3-fluoro-4-(2-methylmorpholino)aniline

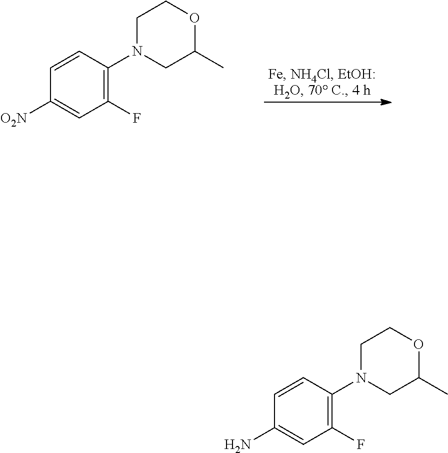 [SMILES: CC1CN(c2ccc([N+](=O)[O-])cc2F)CCO1 ; "Fe, NH4Cl, EtOH:H2O, 70° C., 4 h" ; CC1CN(c2ccc(N)cc2F)CCO1]

To a stirred solution of 4-(2-fluoro-4-nitrophenyl)-2-methylmorpholine (1.1 g, 4.58 mmol, 1 eq) in EtOH:H₂O (11 mL) at room temperature, was added Fe (1 g, 18.3 mmol, 4 eq) and NH₄Cl (0.98 g, 18.3 mmol, 4 eq) and stirred at 70° C. for 4 h. After completion of reaction by TLC, reaction mixture was filtered through a pad of diatomaceous earth (e.g., Celite®), washed with EtOAc (50 mL). Collected filtrate was concentrated, diluted with water (35 mL) and extracted with EtOAc (2×35 mL). The combined organic layer was dried over Na₂SO₄, filtered and concentrated under reduced pressure to afford 3-fluoro-4-(2-methylmorpholino)aniline as brown gummy liquid (850 mg, Crude).

TLC system: EtOAc/Hexane (30:70)

R_{f} value: ˜0.4

LCMS(m/z): 211.42 (M+H)⁺

### Step 3: Synthesis of methyl 4-(2-((3-fluoro-4-(2-methylmorpholino)phenyl)amino)pyrimidin-4-yl)benzoate

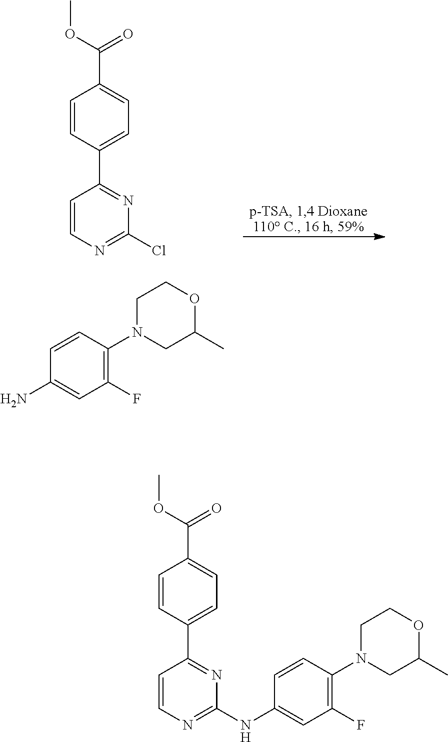

To a stirred solution of methyl 4-(2-chloropyrimidin-4-yl)benzoate (1 g, 4.03 mmol, 1.0 eq) in 1,4-dioxane (20 mL) at room temperature, was added p-TSA (624 mg, 3.63 mmol, 0.9 eq) followed by 3-fluoro-4-(2-methylmorpholino)aniline (0.9 g, 4.03 mmol, 1.0 eq) and stirred at 110° C. for 16 h. After completion of reaction by TLC, reaction mixture was diluted with water (30 mL) and extracted with EtOAc (3×30 mL). The combined organic layer was dried over anhydrous Na₂SO₄, filtered, and concentrated under reduced pressure. Obtained crude was purified by silica gel (100-200 mesh) column chromatography [elution with 20% EtOAc in Hexane] to afford methyl 4-(2-((3-fluoro-4-(2-methylmorpholino)phenyl)amino)pyrimidin-4-yl)benzoate as brown gummy liquid (1.0 g, yield: 59%).

TLC system: EtOAc/Hexane (30:70)

R_{f} value: ˜0.4

LCMS(m/z): 423.77 (M+H)⁺

¹H NMR (400 MHz, CDCl₃) δ: 8.54 (d, J=5.2 Hz, 1H), 8.22-8.15 (m, 4H), 7.73 (dd, J=14.4 & 2.4 Hz, 1H), 7.28-7.23 (m, 2H), 6.98 (t, J=9.2 Hz, 1H), 4.03-3.95 (m, 4H), 3.93-3.87 (m, 2H), 3.30-3.22 (m, 2H), 2.93-2.86 (m, 1H), 2.59-2.54 (m, 1H), 1.27 (d, J=6.4 Hz, 3H)

### Step 4: Synthesis of 4-(2-((3-fluoro-4-(2-methylmorpholino)phenyl)amino)pyrimidin-4-yl)benzoic acid

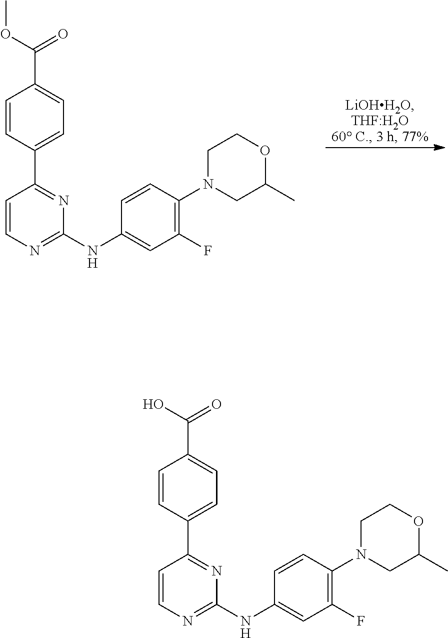 [SMILES: COC(=O)c1ccc(-c2ccnc(Nc3ccc(N4CCOC(C)C4)c(F)c3)n2)cc1 ; "LiOH·H2O,THF:H2O60° C., 3 h, 77%" ; CC1CN(c2ccc(Nc3nccc(-c4ccc(C(=O)O)cc4)n3)cc2F)CCO1]

To a stirred solution of methyl 4-(2-((3-fluoro-4-(2-methylmorpholino)phenyl)amino)pyrimidin-4-yl)benzoate (1 g, 2.37 mmol, 1.0 eq) in THF:H₂O (2:1) (10 mL) at 0° C., was added LiOH·H₂O (200 mg, 4.74 mmol, 2.0 eq) and stirred at 60° C. for 3 h. After completion of reaction by TLC, reaction mixture was washed with EtOAc (20 mL). Aqueous layer was acidified with 2N HCl solution (5 mL), precipitated solid was filtered and dried under vacuum to afford 4-(2-((3-fluoro-4-(2-methylmorpholino)phenyl)amino)pyrimidin-4-yl)benzoic acid as yellow solid (750 mg, yield: 77%).

TLC system: MeOH/dichloromethane (10:90)

R_{f} value: ˜0.3; LCMS(m/z): 409.2 (M+H)⁺

¹H NMR (400 MHz, DMSO-d₆) δ: 13.0 (br, 1H), 9.82 (s, 1H), 8.63 (d, J=5.2 Hz, 1H), 8.28 (d, J=8.4 Hz, 2H), 8.12 (d, J=8.4 Hz, 2H), 7.78 (dd, J=15.6 & 2.4 Hz, 1H), 7.56-7.54 (m, 1H), 7.49 (d, J=5.2 Hz, 1H), 7.04 (t, J=9.6 Hz, 1H), 3.90-3.87 (m, 1H), 3.74-3.67 (m, 2H), 3.21-3.12 (m, 2H), 2.77-2.71 (m, 1H), 2.47-2.41 (m, 1H), 1.14 (d, J=6.4 Hz, 3H)

### Step 5: Synthesis of 4-(2-((3-fluoro-4-(2-methylmorpholino)phenyl)amino)pyrimidin-4-yl)-N-((tetrahydro-2H-pyran-2-yl)oxy)benzamide

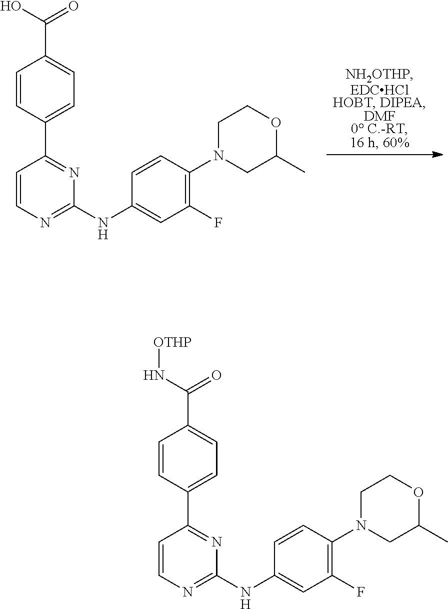

To a stirred solution of 4-(2-((3-fluoro-4-(2-methylmorpholino)phenyl)amino)pyrimidin-4-yl)benzoic acid (400 mg, 0.98 mmol, 1 eq) in DMF (4 mL) at 0° C., was added DIPEA (0.5 mL, 2.94 mmol, 3.0 eq), HOBt (132 mg, 0.98 mmol, 1.0 eq) followed by EDC-HCl (188 mg, 0.98 mmol, 1.0 eq) and stirred for 30 min. After that NH₂OTHP (137 mg, 1.18 mmol, 1.2 eq) was added to the reaction mixture and stirred at room temperature for 16 h. After completion of reaction by TLC, the reaction mixture was quenched with ice cold water (20 mL) and extracted with 10% MeOH in dichloromethane (3×20 mL) The combined organic layer was dried over Na₂SO₄, filtered, and concentrated under reduced pressure. The crude product was purified by silica gel (60-120 mesh) column chromatography [elution with 10% MeOH in dichloromethane] to afford 4-(2-((3-fluoro-4-(2-methylmorpholino)phenyl)amino)pyrimidin-4-yl)-N-((tetrahydro-2H-pyran-2-yl)oxy)benzamide as yellow gummy liquid (300 mg, yield: 60%).

TLC system: MeOH/dichloromethane (10:90)

R_{f} value: ˜0.5

LCMS(m/z): 508.79 (M+H)⁺

### Step 6: Synthesis of 4-(2-((3-fluoro-4-(2-methylmorpholino)phenyl)amino)pyrimidin-4-yl)-N-hydroxybenzamide

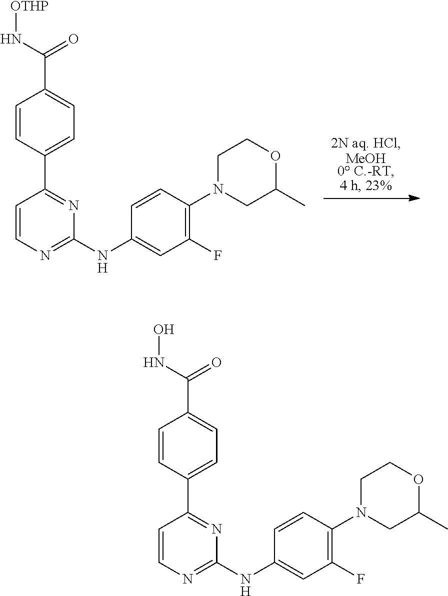

To a stirred solution of 4-(2-((3-fluoro-4-(2-methylmorpholino)phenyl)amino)pyrimidin-4-yl)-N-((tetrahydro-2H-pyran-2-yl)oxy)benzamide (300 mg, 0.59 mmol, 1 eq) in MeOH (3 mL) at 0° C., was added 2N aq. HCl (1.5 mL, 5 Vol) and stirred at room temperature for 4 h. After completion of reaction by TLC, reaction mixture was concentrated under reduced pressure and purified by reverse phase column chromatography (Grace) [elution with 50-55% acetonitrile in 0.1% formic acid in H₂O] to afford 4-(2-((3-fluoro-4-(2-methylmorpholino)phenyl)amino)pyrimidin-4-yl)-N-hydroxybenzamide as pale brown solid (59 mg, yield: 23%).

TLC system: MeOH/dichloromethane (10:90)

R_{f} value: ˜0.2

LCMS (m/z): 424.3 (M+H)⁺

¹H NMR (400 MHz, DMSO-d₆) δ: 11.4 (br, 1H), 9.80 (s, 1H), 8.59 (d, J=5.2 Hz, 1H), 8.23 (d, J=8.4 Hz, 2H), 7.92 (d, J=8.4 Hz, 2H), 7.78 (dd, J=2.4 Hz, 15.2 Hz, 1H), 7.53-7.50 (m, 1H), 7.47 (d, J=5.2 Hz, 1H), 7.04 (t, J=9.6 Hz, 1H), 3.88-3.85 (m, 1H), 3.75-3.66 (m, 2H), 3.20-3.17 (m, 1H), 3.14-3.11 (m, 1H), 2.77-2.71 (m, 1H), 2.50-2.41 (m, 1H), 1.13 (d, J=6.0 Hz, 3H)

### Synthetic Example 12

### Synthesis of 4-(2-((3-Fluoro-4-(2-Methylmorpholino)Phenyl)Amino)Pyrimidin-4-Yl)-N-Hydroxy-2-Methylbenzamide

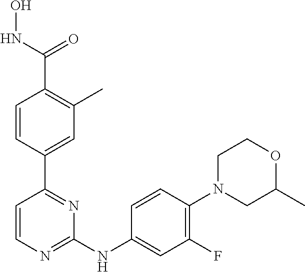 [SMILES: Cc1cc(-c2ccnc(Nc3ccc(N4CCOC(C)C4)c(F)c3)n2)ccc1C(=O)NO]

### Step 1: Synthesis of methyl 4-(2-chloropyrimidin-4-yl)-2-methylbenzoate

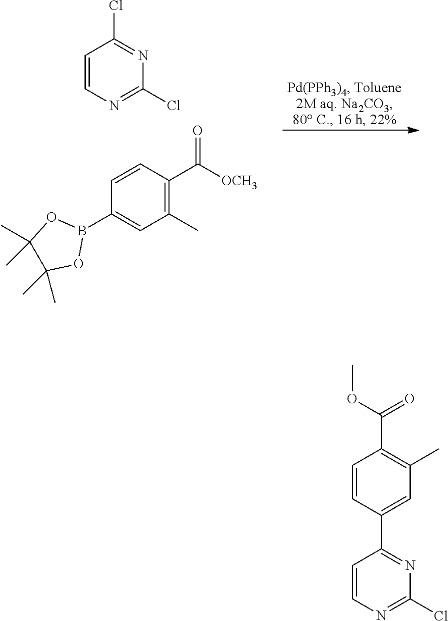

To a degassed solution of methyl 2-methyl-4-(4,4,5,5-tetramethyl-1,3,2-dioxaborolan-2-yl)benzoate (1.6 g, 6.12 mmol, 0.6 eq) and 2,4-dichloropyrimidine (1.5 g, 10.2 mmol, 1.0 eq) in toluene (15 mL) at room temperature was added 2M aq. Na₂CO₃ (5 mL, 3.3 vol) followed by Pd(PPh₃)₄ (581 mg, 0.40 mmol, 0.05 eq). The reaction mixture was heated to 100° C. and stirred for 16 h in a sealed tube. After completion of reaction by TLC, reaction mixture was diluted with water (50 mL) and extracted with EtOAc (2×200 mL). The organic layer was dried over Na₂SO₄, filtered, and concentrated under reduced pressure. The crude product was triturated with MeOH (100 mL), filtered, and collected solid was dried under vacuum to afford methyl 4-(2-chloropyrimidin-4-yl)-2-methylbenzoate as off white solid (580 mg, yield: 22%).

TLC system: EtOAc/Hexane (85:15)

R_{f} value: ˜0.4

LCMS (m/z): 263.2 (M+H)⁺

¹H NMR (400 MHz, CDCl₃) δ: 8.68 (d, J=5.2 Hz, 1H), 8.02 (d, J=8.0 Hz, 1H), 7.98 (s, 1H), 7.92 (d, J=8.0 Hz, 1H), 7.68 (d, J=5.2 Hz, 1H), 3.93 (s, 3H), 2.69 (s, 3H)

### Step 2: Synthesis of methyl 4-(2-((3-fluoro-4-(2-methylmorpholino)phenyl)amino)pyrimidin-4-yl)-2-methylbenzoate

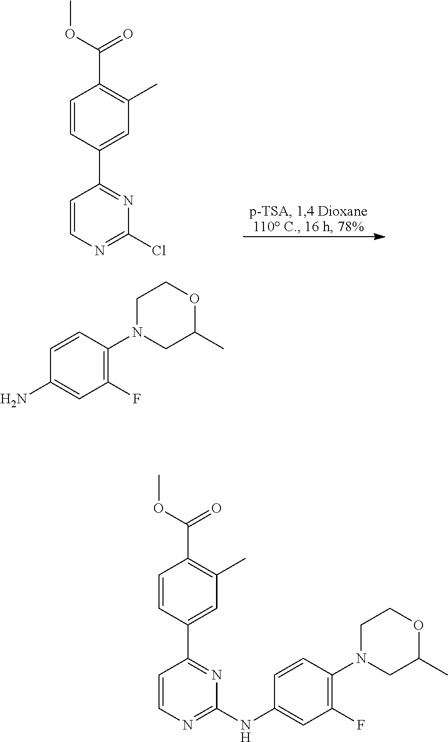

To a stirred solution of methyl 4-(2-chloropyrimidin-4-yl)-2-methylbenzoate (540 mg, 2.06 mmol, 1 eq) in 1,4-dioxane (11 mL) at room temperature, was added p-TSA (319 mg, 1.85 mmol, 0.9 eq) followed by 3-fluoro-4-(2-methylmorpholino)aniline (432 mg, 2.06 mmol, 1.0 eq) and stirred at 110° C. for 16 h. After completion of reaction by TLC, reaction mixture was diluted with water (30 mL) and extracted with EtOAc (2×50 mL). The combined organic layer was dried over anhydrous Na₂SO₄, filtered, and concentrated under reduced pressure. The crude product was purified by silica gel (100-200 mesh) column chromatography [elution with 50% EtOAc in Hexane] to afford methyl 4-(2-((3-fluoro-4-(2-methylmorpholino)phenyl)amino)pyrimidin-4-yl)-2-methylbenzoate as yellow solid (700 mg, yield: 78%).

TLC system: EtOAc/Hexane (50:50)

R_{f} value: ˜0.7

LCMS (m/z): 437.2 (M+H)⁺

¹H NMR (400 MHz, CDCl₃) δ: 8.49 (d, J=5.2 Hz, 1H), 8.03 (d, J=8.0 Hz, 1H), 7.95 (s, 1H), 7.91-7.89 (m, 1H), 7.73 (dd, J=14.8 & 2.4 Hz, 1H), 7.21-7.18 (m, 2H), 6.93 (t, J=9.2 Hz, 1H), 3.96-3.85 (m, 6H), 3.26-3.18 (m, 2H), 2.89-2.83 (m, 1H), 2.70 (s, 3H), 2.55-2.50 (m, 1H), 1.23 (d, J=6.0 Hz, 3H)

### Step 3: Synthesis of 4-(2-((3-fluoro-4-(2-methylmorpholino)phenyl)amino)pyrimidin-4-yl)-2-methylbenzoic acid

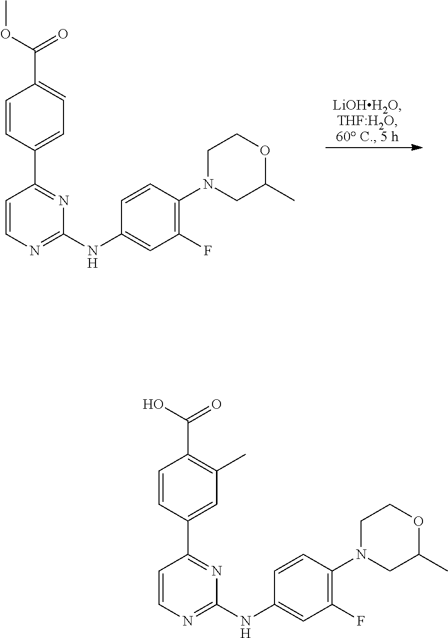 [SMILES: COC(=O)c1ccc(-c2ccnc(Nc3ccc(N4CCOC(C)C4)c(F)c3)n2)cc1 ; "LiOH·H2O,THF:H2O,60° C., 5 h" ; Cc1cc(-c2ccnc(Nc3ccc(N4CCOC(C)C4)c(F)c3)n2)ccc1C(=O)O]

To a stirred solution of methyl 4-(2-((3-fluoro-4-(2-methylmorpholino)phenyl)amino)pyrimidin-4-yl)-2-methylbenzoate (700 mg, 1.6 mmol, 1.0 eq) in THF:H₂O (2:1) (7 mL) at 0° C., was added LiOH·H₂O (269 mg, 6.4 mmol, 4.0 eq) and stirred at 60° C. for 5 h. After completion of reaction by TLC, reaction mixture was washed with EtOAc (50 mL). Aqueous layer was acidified with 2N HCl solution (10 mL), precipitated solid was filtered and dried under vacuum to afford 4-(2-((3-fluoro-4-(2-methylmorpholino)phenyl)amino)pyrimidin-4-yl)-2-methylbenzoic acid as yellow solid (500 mg, Crude).

TLC system: EtOAc/Hexane (70:30)

R_{f} value: ˜0.2

LCMS (m/z): 423.4 (M+H)⁺

¹H NMR (400 MHz, DMSO-d₆) δ: 13.10 (s, 1H), 9.78 (s, 1H), 8.59 (d, J=5.2 Hz, 1H), 8.09 (s, 1H), 8.05-8.03 (m, 1H), 7.97-7.94 (m, 1H), 7.81 (dd, J=13.2 Hz, J=2.4 Hz, 1H), 7.50-7.45 (m, 2H), 7.02 (t, J=9.6 Hz, 1H), 3.88-3.84 (m, 1H), 3.72-3.65 (m, 2H), 3.19-3.16 (m, 1H), 3.12-3.10 (m, 1H), 2.75-2.71 (m, 1H), 2.62 (s, 3H), 2.43-2.41 (m, 1H), 1.12 (d, J=6.4 Hz, 3H)

### Step 4: Synthesis of 4-(2-((3-fluoro-4-(2-methylmorpholino)phenyl)amino)pyrimidin-4-yl)-2-methyl-N-((tetrahydro-2H-pyran-2-yl)oxy)benzamide

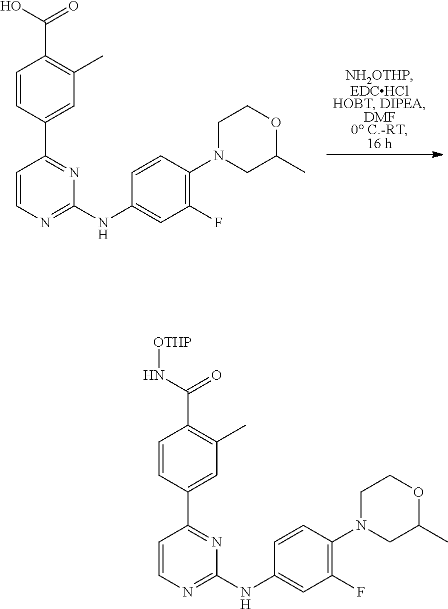

To a stirred solution of 4-(2-((3-fluoro-4-(2-methylmorpholino)phenyl)amino)pyrimidin-4-yl)-2-methylbenzoic acid (350 mg, 0.83 mmol, 1.0 eq) in DMF (4 mL) at 0° C., was added DIPEA (0.43 mL, 2.48 mmol, 3.0 eq), HOBt (112 mg, 0.83 mmol, 1.0 eq) followed by EDC-HCl (160 mg, 0.83 mmol, 1.0 eq) and stirred for 30 min. After that NH₂OTHP (116 mg, 0.99 mmol, 1.2 eq) was added and stirred at room temperature for 16 h. After completion of reaction by TLC, reaction mixture was quenched with ice cold water (20 mL) and extracted with EtOAc (2×40 mL). The combined organic layer was dried over Na₂SO₄, filtered and concentrated under reduced pressure to afford 4-(2-((3-fluoro-4-(2-methylmorpholino) phenyl)amino)pyrimidin-4-yl)-2-methyl-N-((tetrahydro-2H-pyran-2-yl)oxy)benzamide as yellow solid (300 mg, Crude).

TLC system: EtOAc/Hexane (50:50)

R_{f} value: ˜0.3

LCMS (m/z): 522.3 (M+H)⁺

¹H NMR (400 MHz, DMSO-d₆) δ: 9.25 (brs, 1H), 8.39 (d, J=5.2 Hz, 1H), 7.84 (s, 1H), 7.76-7.71 (m, 2H), 7.46 (brs, 1H), 7.38 (d, J=7.6 Hz, 1H), 7.16 (d, J=8.8 Hz, 1H), 7.08 (d, J=5.2 Hz, 1H), 6.93 (t, J=9.2 Hz, 1H), 5.22 (brs, 1H), 4.09-3.84 (m, 4H), 3.73-3.70 (m, 1H), 3.27-3.19 (m, 2H), 2.87-2.85 (m, 1H), 2.56-2.50 (m, 1H), 2.46 (s, 3H), 1.92-1.89 (m, 2H), 1.69-1.61 (m, 2H), 1.25-1.23 (m, 5H)

### Step 5: Synthesis of 4-(2-((3-fluoro-4-(2-methylmorpholino)phenyl)amino)pyrimidin-4-yl)-N-hydroxy-2-methylbenzamide

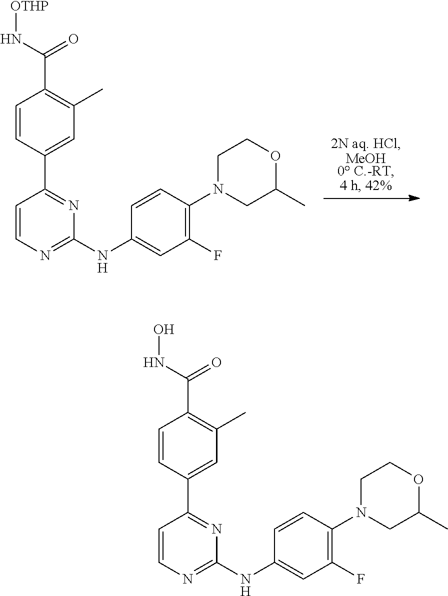

To a stirred solution of 4-(2-((3-fluoro-4-(2-methylmorpholino) phenyl)amino)pyrimidin-4-yl)-2-methyl-N-((tetrahydro-2H-pyran-2-yl)oxy)benzamide (300 mg, 0.57 mmol, 1 eq) in MeOH (3 mL) at 0° C., was added 2 N aq. HCl (1.5 mL, 5 Vol) and stirred at room temperature for 4 h. After completion of reaction by TLC, the reaction mixture was concentrated under reduced pressure and purified by reverse phase column chromatography (Grace) [elution with 45-50% acetonitrile in 0.1% formic acid in H₂O] and lyophilized to afford 4-(2-((3-fluoro-4-(2-methylmorpholino)phenyl)amino)pyrimidin-4-yl)-N-hydroxy-2-methylbenzamide as yellow solid (102 mg, yield: 40%).

TLC system: EtOAc (100)

R_{f} value: ˜0.1

LCMS (m/z): 438.3 (M+H)⁺

¹H NMR (400 MHz, DMSO-d₆) δ: 10.94 (s, 1H), 9.76 (s, 1H), 9.14 (s, 1H), 8.57 (d, J=5.2 Hz, 1H), 8.06 (s, 1H), 7.99 (d, J=8.0 Hz, 1H), 7.82 (dd, J=13.2 Hz, J=2.4 Hz, 1H), 7.49-7.42 (m, 3H), 7.01 (d, J=9.6 Hz, 1H), 3.88-3.85 (m, 1H), 3.72-3.65 (m, 2H), 3.17 (d, J=11.6 Hz, 1H), 3.10 (d, J=11.6 Hz, 1H), 2.75-2.67 (m, 1H), 2.49 (s, 3H), 2.44-2.39 (m, 1H), 1.22 (d, J=6.4 Hz, 3H)

### Synthetic Example 13

### Synthesis of Synthesis of 4-(2-((3-Fluoro-4-(1-(2-Methylmorpholino)Ethyl)Phenyl)Amino)Pyrimidin-4-Yl)-N-Hydroxybenzamide

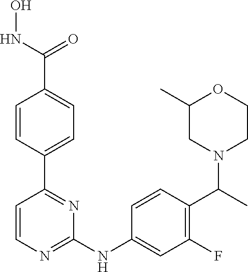 [SMILES: CC1CN(C(C)c2ccc(Nc3nccc(-c4ccc(C(=O)NO)cc4)n3)cc2F)CCO1]

### Step 1: Synthesis of 4-(1-(2-fluoro-4-nitrophenyl)ethyl)-2-methylmorpholine

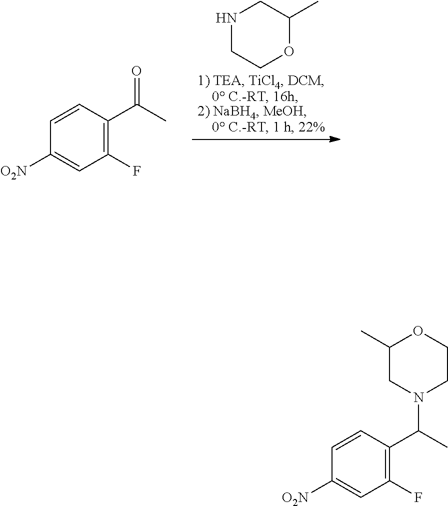

To a stirred solution of 1-(2-fluoro-4-nitrophenyl)ethan-1-one (2 g, 10.9 mmol, 1.0 eq) in dichloromethane (20 mL, 10 Vol), was added TEA (3.8 mL, 27.2 mmol, 2.5 eq) and 2-methylmorpholine. The reaction mixture was cooled to 0° C., and added TiCl₄ drop wise (0.6 mL, 5.4 mmol, 0.5 eq) and stirred at room temperature for 16 h. Later the reaction mixture was cooled to 0° C., added NaBH₄ (1.2 g, 32.7 mmol, 3 eq) and methanol (10 mL). The reaction mixture was allowed to stir at room temperature for 1 h. After completion of reaction by TLC, ice-cold water was added to the reaction mixture and extracted with dichloromethane (100 mL). The combined organic layer was dried over anhydrous Na₂SO₄, filtered, and concentrated under reduced pressure. Obtained crude compound was purified by reverse phase column chromatography (Grace) [elution with 20-25% of acetonitrile in 0.1% formic acid in H₂O] to afford 4-(1-(2-fluoro-4-nitrophenyl)ethyl)-2-methylmorpholine as white gummy liquid (650 mg, yield: 22%).

TLC system: EtOAc/Hexane (30:70)

R_{f} value: ˜0.1

LCMS (m/z): 268.9 (M+H)⁺

### Step 2: Synthesis of 3-fluoro-4-(1-(2-methylmorpholino)ethyl)aniline

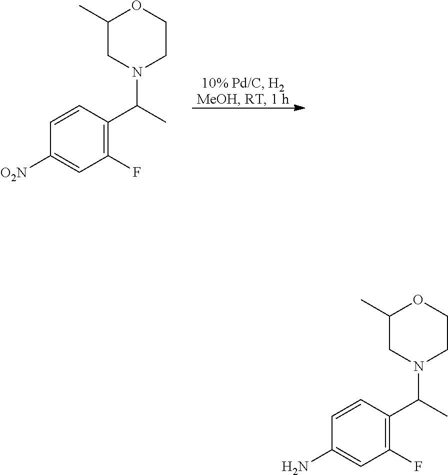 [SMILES: CC1CN(C(C)c2ccc([N+](=O)[O-])cc2F)CCO1 ; "10% Pd/C, H2MeOH, RT, 1 h" ; CC1CN(C(C)c2ccc(N)cc2F)CCO1]

To a stirred solution of 4-(1-(2-fluoro-4-nitrophenyl)ethyl)-2-methylmorpholine (700 mg, 1.86 mmol, 1.0 eq) in MeOH (7 mL), was add 10% Pd/C (210 mg, 30% w/w) and reaction mixture was stirred at room temperature for 1 h under H₂ balloon atmosphere. After completion of reaction by TLC, reaction mixture filtered through a pad of diatomaceous earth (e.g., Celite®) and washed with 10% dichloromethane/MeOH. Filtrate concentrated under reduced pressure to afford 3-fluoro-4-(1-(2-methylmorpholino)ethyl)aniline as brown gummy liquid (573 mg, Crude).

TLC system: EtOAc/Hexane (50:50)

R_{f} value: ˜0.15

LCMS (m/z): 239.2 (M+H)⁺

### Step 3: Synthesis of methyl 4-(2-((3-fluoro-4-(1-(2-methylmorpholino)ethyl)phenyl)amino)pyrimidin-4-yl)benzoate

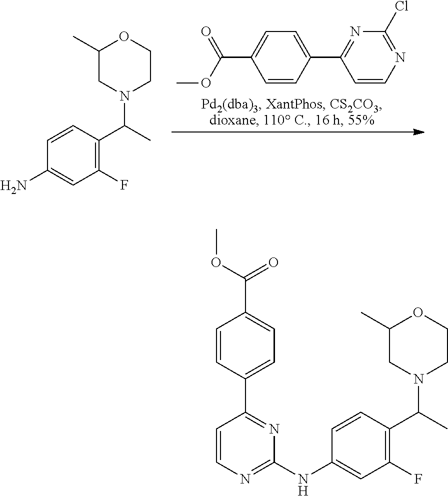

To a degassed stirred solution of methyl 4-(2-chloropyrimidin-4-yl)benzoate (600 mg, 2.41 mmol, 1 eq) and 3-fluoro-4-(1-(2-methylmorpholino)ethyl)aniline (Crude) (573 mg, 2.41 mmol, 1.0 eq) in 1,4-dioxane (6 mL) at room temperature, was added Cs₂CO₃ (1.5 g, 4.81 mmol, 2.0 eq), XanthPhos (14 mg, 0.24 mmol, 0.1 eq) followed by Pd₂(dba)₃ (110 mg, 0.12 mmol, 0.05 eq). The reaction mixture was stirred at 110° C. for 16 h. After completion of reaction by TLC, diluted with water (50 mL) and extracted with 10% MeOH in dichloromethane (2×50 mL). Combined organic layer was dried over Na₂SO₄, filtered, and concentrated under reduced pressure. Crude product was purified by silica gel (100-200) [elution with 40-50% EtOAc in pet ether] to afford methyl 4-(2-((3-fluoro-4-(1-(2-methylmorpholino)ethyl)phenyl)amino)pyrimidin-4-yl)benzoate as yellow solid (600 mg, yield: 55%).

TLC system: EtOAc/Hexane (50:50)

R_{f} value: ˜0.4

LCMS(m/z): 451.16 (M+H)⁺

### Step 4: Synthesis of 4-(2-((3-fluoro-4-(1-(2-methylmorpholino)ethyl)phenyl)amino)pyrimidin-4-yl)benzoic acid

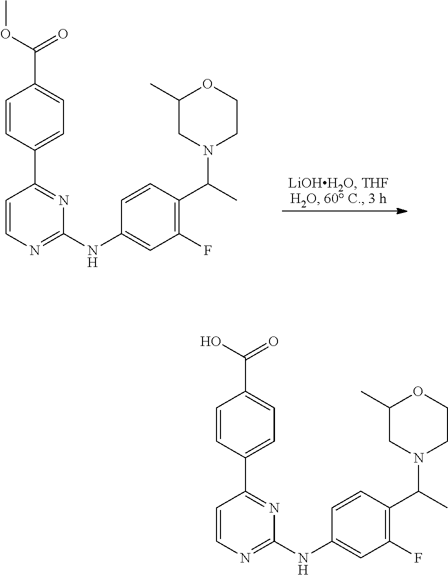 [SMILES: COC(=O)c1ccc(-c2ccnc(Nc3ccc(C(C)N4CCOC(C)C4)c(F)c3)n2)cc1 ; "LiOH·H2O, THFH2O, 60° C., 3 h" ; CC1CN(C(C)c2ccc(Nc3nccc(-c4ccc(C(=O)O)cc4)n3)cc2F)CCO1]

To a stirred solution of methyl 4-(2-((3-fluoro-4-(1-(2-methylmorpholino)ethyl)phenyl)amino)pyrimidin-4-yl)benzoate (600 mg, 1.33 mmol, 1.0 eq) in THF:H₂O (1:1) (10 mL) at 0° C., was added LiOH·H₂O (112 mg, 2.66 mmol, 2 eq) and stirred at 60° C. for 3 h. After completion of reaction by TLC, reaction mixture was washed with ethyl acetate (15 mL). Aqueous layer was acidified with 2N HCl solution (6 mL), precipitated solid was filtered and dried under vacuum to afford 4-(2-((3-fluoro-4-(1-(2-methylmorpholino)ethyl)phenyl)amino)pyrimidin-4-yl)benzoic acid as pale yellow solid (400 mg, Crude).

TLC system: EtOAc (100)

R_{f} value: ˜0.15

LCMS(m/z): 437.1 (M+H)⁺

### Step 5: Synthesis of 4-(2-((3-fluoro-4-(1-(2-methylmorpholino)ethyl)phenyl)amino)pyrimidin-4-yl)-N-((tetrahydro-2H-pyran-2-yl)oxy)benzamide

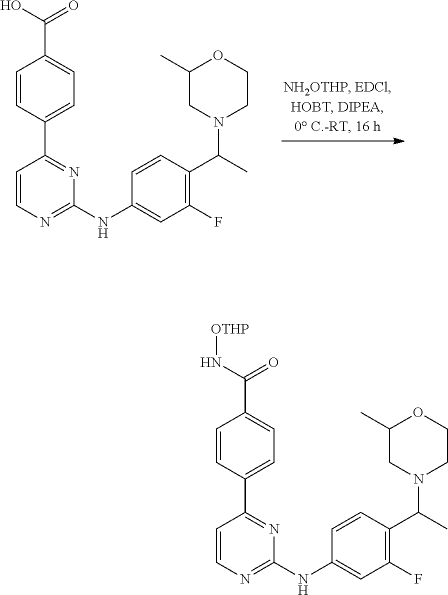

To a stirred solution of 4-(2-((3-fluoro-4-(1-(2-methylmorpholino)ethyl)phenyl)amino)pyrimidin-4-yl)benzoic acid (400 mg, 0.92 mmol, 1.0 eq) (Crude) in DMF (4 mL) at 0° C., was added DIPEA (0.5 mL, 2.75 mmol, 3.0 eq), HOBt (124 mg, 0.92 mmol, 1.0 eq) followed by EDC-HCl (176 mg, 0.92 mmol, 1.0 eq) and stirred for 30 min. After that NH₂OTHP (129 mg, 1.10 mmol, 1.2 eq) was added and stirred at room temperature for 16 h. After completion of reaction by TLC, reaction mixture was quenched with ice cold water (20 mL) and extracted with EtOAc (2×30 mL). Combined organic layer was dried over Na₂SO₄, filtered and concentrated under reduced pressure to afford 4-(2-((3-fluoro-4-(1-(2-methylmorpholino)ethyl)phenyl)amino)pyrimidin-4-yl)-N-((tetrahydro-2H-pyran-2-yl)oxy)benzamide as brown gummy solid (400 mg, crude—77% purity).

TLC system: EtOAc/Hexane (70:30)

R_{f} value: ˜0.4

LCMS (m/z): 536.5 (M+H)⁺

### Step 6: Synthesis of 4-(2-((3-fluoro-4-(1-(2-methylmorpholino)ethyl)phenyl)amino)pyrimidin-4-yl)-N-hydroxybenzamide

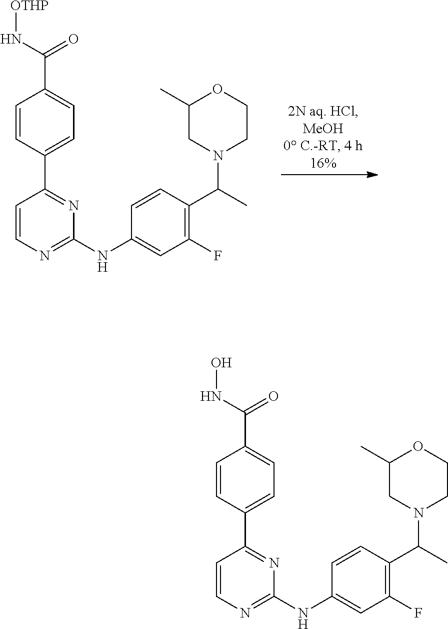

To a stirred solution of 4-(2-((3-fluoro-4-(1-(2-methylmorpholino)ethyl)phenyl)amino)pyrimidin-4-yl)-N-((tetrahydro-2H-pyran-2-yl)oxy)benzamide (200 mg, 0.37 mmol, 1 eq) (Crude) in MeOH (2 mL) at 0° C., was added 2 N aq. HCl (2 mL, 10 Vol) and stirred at room temperature for 4 h. After completion of reaction by TLC, reaction mixture was concentrated under reduced pressure and purified by reverse phase column chromatography (Grace) [elution with 25-30% acetonitrile in 0.1% formic acid in H₂O], lyophilized to afford 4-(2-((3-fluoro-4-(1-(2-methylmorpholino)ethyl)phenyl)amino)pyrimidin-4-yl)-N-hydroxybenzamide as off white solid (50 mg, yield: 16% in 3 steps).

TLC system: EtOAc (100)

R_{f} value: ˜0.1

LCMS (m/z): 452.3 (M+H)⁺

¹H NMR (400 MHz, DMSO-d₆) δ: 11.37 (s, 1H), 9.96 (s, 1H), 9.14 (s, 1H), 8.63 (d, J=5.2 Hz, 1H), 8.24 (d, J=8.4 Hz, 2H), 7.93 (d, J=8.4 Hz, 2H), 7.83 (d, J=13.6 Hz, 1H), 7.56 (d, J=8.4 Hz, 1H), 7.52 (d, J=5.2 Hz, 1H), 7.34 (t, J=8.2 Hz, 1H), 3.78-3.67 (m, 1H), 3.54-3.43 (m, 2H), 2.89-2.84 (br, 1H), 2.63-2.59 (br, 1H), 2.05-1.58 (br, 2H), 1.34-1.32 (d, 3H), 1.06-0.96 (2d, 3H)

### Synthetic Example 14

### Synthesis of N-Hydroxy-4-(2-(2-(2-Methylmorpholino)Acetamido)Pyrimidin-4-Yl)Benzamide

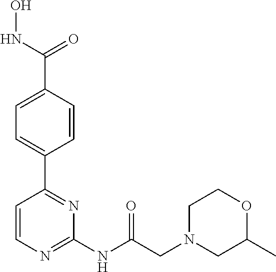 [SMILES: CC1CN(CC(=O)Nc2nccc(-c3ccc(C(=O)NO)cc3)n2)CCO1]

### Step 1: Synthesis of methyl 4-(2-((tert-butoxycarbonyl)amino)pyrimidin-4-yl)benzoate

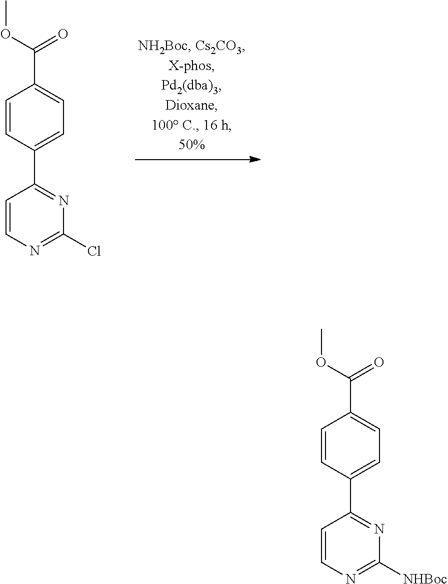 [SMILES: COC(=O)c1ccc(-c2ccnc(Cl)n2)cc1 ; COC(=O)c1ccc(-c2ccnc(NC(=O)OC(C)(C)C)n2)cc1]

To a degassed stirred solution of methyl 4-(2-chloropyrimidin-4-yl)benzoate (3 g, 12.04 mmol, 1 eq) and tert-butyl carbamate (1.7 g, 14.45 mmol, 1.0 eq) in 1,4-dioxane (30 mL) at room temperature, was added Cs₂CO₃ (9.7 g, 30.12 mmol, 2.5 eq), X-phos (1.14 g, 2.40 mmol, 0.2 eq) followed by Pd₂(dba)₃ (1.1 mg, 1.20 mmol, 0.1 eq). The reaction mixture was stirred at 100° C. for 16 h. After completion of reaction by TLC, diluted with water (50 mL) and extracted with Ethyl acetate (2×100 mL). The combined organic layer was dried over Na₂SO₄, filtered, and concentrated under reduced pressure. The crude product was purified by silica gel (100-200) [elution with 20-30% EtOAc/Hexane] to afford methyl 4-(2-((tert-butoxycarbonyl)amino)pyrimidin-4-yl)benzoate as off white solid (2 g, yield: 50%).

TLC system: EtOAc (100%)

R_{f} value: ˜0.3

LCMS(m/z): 229.8 (M+H−Boc)⁺

¹H NMR (400 MHz, CDCl₃) δ: 8.69 (d, J=5.2 Hz, 1H), 8.15 (s, 4H), 7.53 (brs, 1H), 7.41 (d, J=5.2 Hz, 1H), 3.96 (s, 3H), 1.57 (s, 9H)

### Step 2: Synthesis of methyl 4-(2-aminopyrimidin-4-yl)benzoate

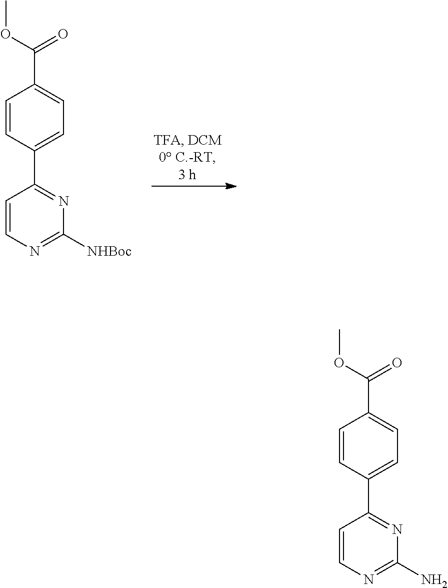 [SMILES: COC(=O)c1ccc(-c2ccnc(NC(=O)OC(C)(C)C)n2)cc1 ; COC(=O)c1ccc(-c2ccnc(N)n2)cc1]

To a stirred solution of methyl 4-(2-((tert-butoxycarbonyl)amino)pyrimidin-4-yl)benzoate (1.7 g, 5.16 mmol, 1 eq) in dichloromethane (17 mL) at 0° C., was added trifluoroacetic acid (17 mL, 10 Vol) and stirred at room temperature for 3 h. After completion of reaction by TLC, reaction mixture was concentrated under reduced pressure, diluted with water (50 mL), basified with aq. NaHCO₃ solution (30 mL) and extracted with 10% MeOH/dichloromethane (2×100 mL). The combined organic layer was dried over Na₂SO₄, filtered, concentrated under reduced pressure and triturated with diethyl ether (30 mL) to afford methyl 4-(2-aminopyrimidin-4-yl)benzoate as an off white solid (1.2 g, Crude).

TLC system: MeOH/dichloromethane (10:90)

R_{f} value: ˜0.2

LCMS (m/z): 230.26 (M+H)⁺

### Step 3: Synthesis of 4-(2-aminopyrimidin-4-yl)benzoic acid

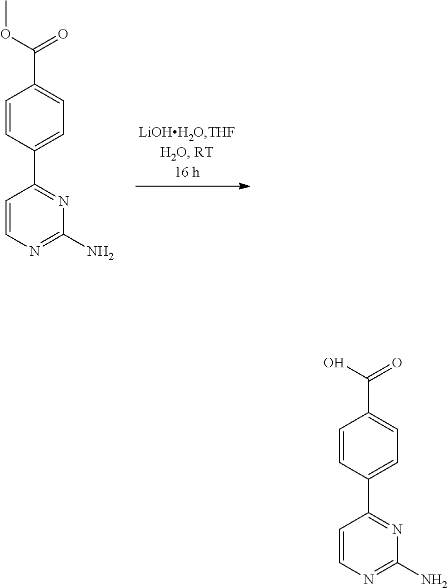 [SMILES: COC(=O)c1ccc(-c2ccnc(N)n2)cc1 ; Nc1nccc(-c2ccc(C(=O)O)cc2)n1]

To a stirred solution of methyl 4-(2-aminopyrimidin-4-yl)benzoate (1.2 g, 5.24 mmol, 1.0 eq)(crude) in THF:H₂O (1:1) (12 mL) at 0° C., was added LiOH·H₂O (440 mg, 10.48 mmol, 2 eq) and stirred at room temperature for 16 h. After completion of reaction by TLC, reaction mass was concentrated, diluted with water (10 mL) and acidified with 2N HCl solution (5 mL). Precipitated solid filtered and dried under vacuum to afford 4-(2-aminopyrimidin-4-yl)benzoic acid as off white solid (970 mg, Crude).

TLC system: EtOAc (100%)

R_{f} value: ˜0.05

LCMS (m/z): 316.2 (M+H)⁺

### Step 4: Synthesis of 4-(2-aminopyrimidin-4-yl)-N-((tetrahydro-2H-pyran-2-yl)oxy)benzamide

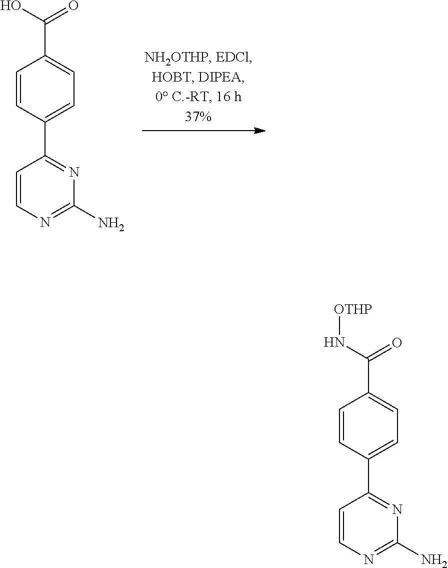

To a stirred solution of 4-(2-aminopyrimidin-4-yl)benzoic acid (970 mg, 4.51 mmol, 1.0 eq) (Crude) in DMF (10 mL) at 0° C., was added DIPEA (2.3 mL, 13.5 mmol, 3.0 eq), HOBt (609 mg, 4.51 mmol, 1.0 eq), followed by EDC-HCl (866 mg, 4.51 mmol, 1.0 eq) and stirred for 30 min. After that NH₂OTHP (633 mg, 5.41 mmol, 1.2 eq) was added and stirred at room temperature for 16 h. After completion of reaction by TLC, reaction mixture was quenched with ice cold water (40 mL) and extracted with 10% MeOH/dichloromethane (2×70 mL). The combined organic layer was dried over Na₂SO₄, filtered, and concentrated under reduced pressure. The crude product was purified by silica gel (100-200) [elution with 5-10% MeOH/dichloromethane] to afford 4-(2-aminopyrimidin-4-yl)-N-((tetrahydro-2H-pyran-2-yl)oxy)benzamide as off white solid (600 mg, Yield: 37% in 3 steps).

TLC system: EtOAc (100%)

R_{f} value: ˜0.2

LCMS (m/z): 315.2 (M+H)⁺

### Step 5: Synthesis of 4-(2-(2-chloroacetamido)pyrimidin-4-yl)-N-((tetrahydro-2H-pyran-2-yl)oxy)benzamide

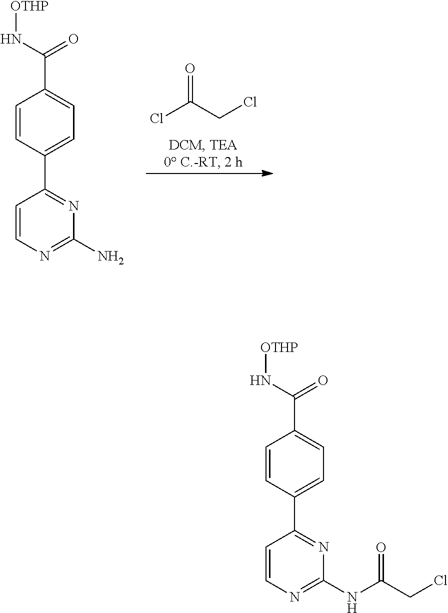

To a stirred solution of 4-(2-aminopyrimidin-4-yl)-N-((tetrahydro-2H-pyran-2-yl)oxy)benzamide (600 mg, 1.91 mmol, 1 eq) in dichloromethane (6 mL) at 0° C. was added TEA (0.8 mL, 5.73 mmol, 3 eq), followed by 2-chloroacetyl chloride (0.23 mL, 2.86 mmol, 1.5 eq) and stirred at room temperature for 3 h. After completion of reaction by TLC, reaction mixture was quenched with ice cold water (20 mL) and extracted with EtOAc (2×50 mL). The combined organic layer was dried over Na₂SO₄, filtered and concentrated under reduced pressure to afford 4-(2-(2-chloroacetamido)pyrimidin-4-yl)-N-((tetrahydro-2H-pyran-2-yl)oxy)benzamide as brown gummy liquid (700 mg, crude—43% purity). The crude product was used in the next step without further purification.

TLC system: EtOAc (100)

R_{f} value: ˜0.4

LCMS (m/z): 391.2 (M+H)⁺

### Step 6: Synthesis of 4-(2-(2-(2-methylmorpholino)acetamido)pyrimidin-4-yl)-N-((tetrahydro-2H-pyran-2-yl)oxy)benzamide

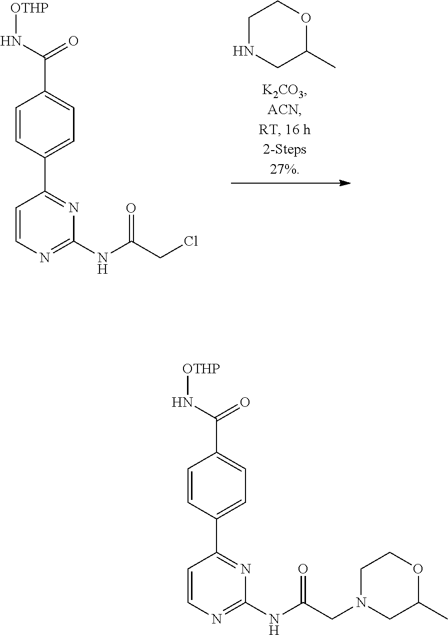

To a stirred solution of 4-(2-(2-chloroacetamido)pyrimidin-4-yl)-N-((tetrahydro-2H-pyran-2-yl)oxy)benzamide (700 mg, 1.79 mmol, 1.0 eq) in acetonitrile (7 mL) at room temperature, was added K₂CO₃ (743 mg, 5.38 mmol, 3.0 eq), followed by 2-methylmorpholine (0.14 mL, 1.25 mmol, 0.7 eq) and stirred at room temperature for 16 h. After completion of reaction by TLC, Filter through a pad of diatomaceous earth (e.g., Celite®) and washed with acetonitrile (20 mL). Filtrate was dried over Na₂SO₄, filtered, and concentrated under reduced pressure. The crude product was purified by reverse phase column chromatography [elution with 15-20% acetonitrile in 0.1% formic acid in H₂O] and concentrated to afford 4-(2-(2-(2-methylmorpholino)acetamido)pyrimidin-4-yl)-N-((tetrahydro-2H-pyran-2-yl)oxy)benzamide as yellow gummy liquid (240 mg, 2-Steps yield: 27%).

TLC system: MeOH/dichloromethane (5:95)

R_{f} value: ˜0.1

LCMS (m/z): 456.3 (M+H)⁺

### Step 7: Synthesis of N-hydroxy-4-(2-(2-(2-methylmorpholino)acetamido)pyrimidin-4-yl)benzamide

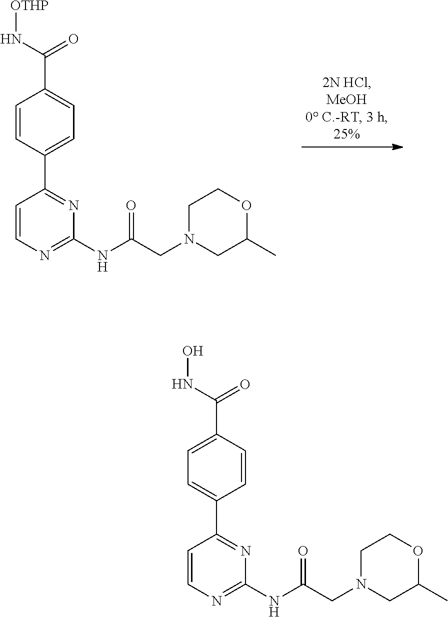

To a stirred solution of 4-(2-(2-(2-methylmorpholino)acetamido)pyrimidin-4-yl)-N-((tetrahydro-2H-pyran-2-yl)oxy)benzamide (230 mg, 0.50 mmol, 1 eq) in MeOH (2.5 mL) at 0° C., was added 2 N aq. HCl (1.15 mL, 5 Vol) and stirred at room temperature for 3 h. After completion of reaction by TLC, reaction mixture was concentrated under reduced pressure and purified by Prep-HPLC (formic acid buffer). The collected fractions were frozen and lyophilized to afford N-hydroxy-4-(2-(2-(2-methylmorpholino)acetamido)pyrimidin-4-yl)benzamide as pale yellow solid (46 mg, yield:25%).

TLC system: MeOH/dichloromethane (10:90)

R_{f} value: ˜0.15

LCMS (m/z): 372.2 (M+H)⁺

¹H NMR (400 MHz, DMSO-d₆) δ: 11.39 (s, 1H), 10.33 (s, 1H), 9.16 (s, 1H), 8.76 (d, J=5.2 Hz, 1H), 8.29 (d, J=8.4 Hz, 2H), 7.92 (d, J=8.4 Hz, 2H), 7.84 (d, J=5.2 Hz, 1H), 3.77-3.74 (m, 1H), 3.56-3.51 (m, 2H), 3.33-3.31 (m, 2H), 2.83-2.73 (m, 2H), 2.33-2.26 (m, 1H), 2.00-1.95 (m, 1H), 1.05 (d, J=6.0 Hz, 3H)

### Synthetic Example 15

### Synthesis of Synthesis of (S)-4-(2-((3-Fluoro-4-((2-Methylmorpholino)Methyl)Phenyl)Amino)Pyrimidin-4-Yl)-N-Hydroxybenzamide Formic Acid Salt

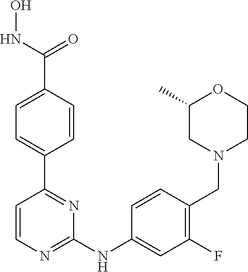 [SMILES: C[C@H]1CN(Cc2ccc(Nc3nccc(-c4ccc(C(=O)NO)cc4)n3)cc2F)CCO1]

### Step 1: Synthesis of (S)-(2-fluoro-4-nitrophenyl)(2-methylmorpholino)methanone

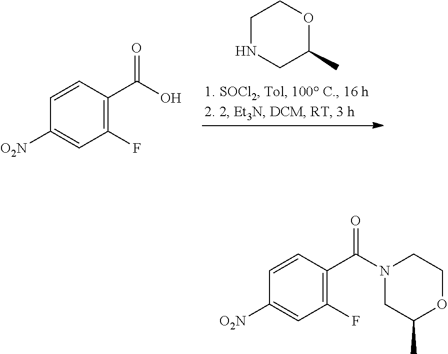

To a stirred solution of 2-fluoro-4-nitrobenzoic acid (1 g, 5.40 mmol, 1.0 eq) in Toluene (10 mL), at 0° C., was added SOCl₂ (1.1 mL, 16.2 mmol, 3.0 eq) and stirred at 100° C. for 16 h. After completion of reaction by TLC, reaction mixture was concentrated under reduced pressure to afford crude acid-chloride. The crude product was dissolved in dichloromethane (10 mL) at room temperature, added Et₃N (2.3 mL, 16.2 mmol, 3.0 eq) and (S)-2-methylmorpholine (0.65 g, 6.48 mmol, 1.2 eq) and stirred for additional 3 h. After completion of reaction by TLC, diluted with water (30 mL) and extracted with dichloromethane (3×50 mL). Combined organic layer was dried over Na₂SO₄, filtered and concentrated under reduced pressure to afford (S)-(2-fluoro-4-nitrophenyl)(2-methylmorpholino)methanone as off white solid (1.4 g, Crude).

TLC system: EtOAc/Hexane (30:70)

R_{f} value: ˜0.35

¹H NMR (400 MHz, CDCl₃) δ: 8.15-8.11 (m, 1H), 8.03-7.99 (m, 1H), 7.62-7.59 (m, 1H), 4.59-4.53 (m, 1H), 4.04-4.01 and 3.87-3.84 (m, 1H), 3.67-3.55 (m, 2H), 3.31 & 2.97 (br s, 1H), 3.24-3.20 (m, 1H), 3.07-3.00 & 2.73-2.67 (m, 1H), 1.27 & 1.13 (2d, 3H)

Splitting of aliphatic might be due to rotamers

### Step 2: Synthesis of (S)-(4-amino-2-fluorophenyl)(2-methylmorpholino)methanone

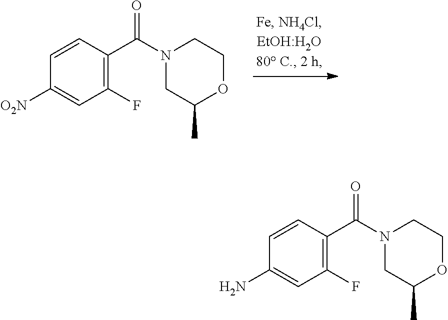 [SMILES: C[C@H]1CN(C(=O)c2ccc([N+](=O)[O-])cc2F)CCO1 ; C[C@H]1CN(C(=O)c2ccc(N)cc2F)CCO1]

To a stirred solution of (S)-(2-fluoro-4-nitrophenyl)(2-methylmorpholino)methanone (1.4 g, 5.22 mmol, 1 eq) in EtOH:H₂O (14 mL) at room temperature, was added Fe (1.4 g, 26.1 mmol, 5 eq) and NH₄Cl (1.39 g, 26.1 mmol, 5 eq) and reaction mixture was heated to 80° C., stirred for 2 h. After completion of reaction by TLC, reaction mixture was filtered through a pad of diatomaceous earth (e.g., Celite®) and washed with 10% MeOH/dichloromethane (40 mL). Filtrate was concentrated, diluted with water (80 mL) and extracted with 10% MeOH/dichloromethane (3×50 mL). Combined organic layer was dried over Na₂SO₄, filtered and concentrated under reduced pressure to afford (S)-(4-amino-2-fluorophenyl)(2-methylmorpholino)methanone as yellow gummy solid (1.25 g, Crude).

TLC system: EtOAc/Hexane (50:50)

R_{f} value: ˜0.25

LCMS(m/z): 239.1 (M+H)⁺

### Step 3: Synthesis of (S)-3-fluoro-4-((2-methylmorpholino)methyl)aniline

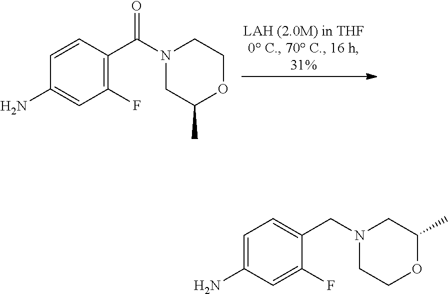 [SMILES: C[C@H]1CN(C(=O)c2ccc(N)cc2F)CCO1 ; C[C@H]1CN(Cc2ccc(N)cc2F)CCO1]

To a stirred solution of (S)-(4-amino-2-fluorophenyl)(2-methylmorpholino)methanone (1.2 g, 5.04 mmol, 1.0 eq) in THF (12 mL) at 0° C., was added LAH (2.0 M in THF) (7.5 mL, 15.2 mmol, 3.0 eq) and stirred at 70° C. for 16 h. After completion of reaction by TLC, the reaction mixture was quenched with NH₄Cl solution (50 mL) and extracted with 10% MeOH/dichloromethane (2×100 mL). The combined organic layer was dried over Na₂SO₄, filtered, concentrated under reduced pressure. The crude product was purified by silica gel column (100-200 mesh) [elution with 5-10% MeOH/dichloromethane] to afford (S)-3-fluoro-4-((2-methylmorpholino)methyl)aniline as pale yellow gummy (370 mg, Yield:31% in 3 steps).

TLC system: EtOAc/Hexane (100%)

R_{f} value: ˜0.12

LCMS(m/z): 225.2 (M+H)⁺

### Step 4: Synthesis of methyl (S)-4-(2-((3-fluoro-4-((2-methylmorpholino)methyl)phenyl)amino)pyrimidin-4-yl)benzoate

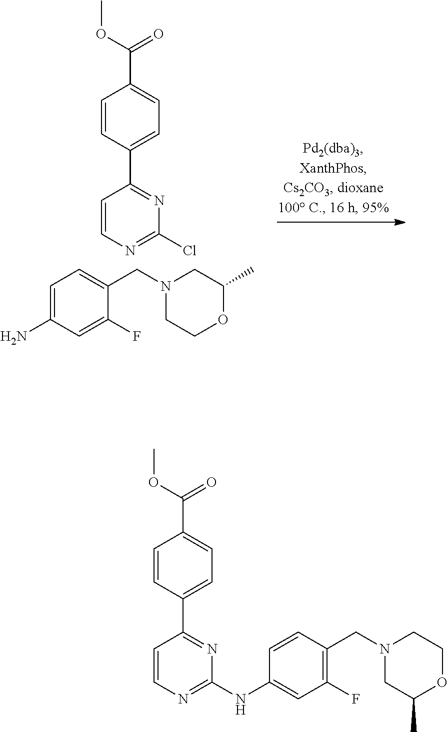

To a stirred solution of methyl 4-(2-chloropyrimidin-4-yl)benzoate (150 mg, 0.60 mmol, 1 eq) and (S)-3-fluoro-4-((2-methylmorpholino)methyl)aniline (135 mg, 0.60 mmol, 1 eq) in 1,4-dioxane (3 mL) at room temperature, was added Cs₂CO₃ (391 g, 1.20 mmol, 2.0 eq), XanthPhos (35 mg, 0.06 mmol, 0.1 eq) followed by Pd₂(dba)₃ (28 mg, 0.03 mmol, 0.05 eq). The reaction mixture was stirred at 100° C. for 16 h. After completion of reaction by TLC, diluted with water (50 mL) and extracted with 10% MeOH in dichloromethane (2×30 mL). The combined organic layer was dried over Na₂SO₄, filtered, and concentrated under reduced pressure. The crude product was purified by silica column (100-200 mesh) [elution with 70-90% EtOAc/Hexane] to afford methyl (S)-4-(2-((3-fluoro-4-((2-methylmorpholino)methyl)phenyl)amino)pyrimidin-4-yl)benzoate as yellow gummy liquid (250 mg, yield: 95%; purity: 83%).

TLC system: EtOAc/Hexane (50:50)

R_{f} value: ˜0.3

LCMS(m/z): 437.44 (M+H)⁺

### Step 5: Synthesis of (S)-4-(2-((3-fluoro-4-((2-methylmorpholino)methyl)phenyl)amino)pyrimidin-4-yl)benzoic acid

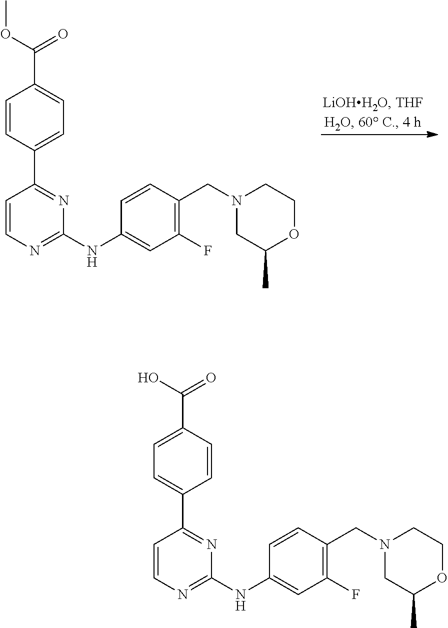 [SMILES: COC(=O)c1ccc(-c2ccnc(Nc3ccc(CN4CCO[C@@H](C)C4)c(F)c3)n2)cc1 ; "LiOH·H2O, THFH2O, 60° C., 4 h" ; C[C@H]1CN(Cc2ccc(Nc3nccc(-c4ccc(C(=O)O)cc4)n3)cc2F)CCO1]

To a stirred solution of methyl (S)-4-(2-((3-fluoro-4-((2-methylmorpholino)methyl)phenyl)amino)pyrimidin-4-yl)benzoate (250 mg, 0.57 mmol, 1.0 eq) in THF:H₂O (1:1) (2.5 mL) at 0° C., was added LiOH·H₂O (48 mg, 1.14 mmol, 2 eq) and stirred at 60° C. for 4 h. After completion of reaction by TLC, reaction mixture was washed with diethyl ether (10 mL). Aqueous layer was acidified with 2N HCl solution (2 mL), precipitated solid was filtered and dried under reduced pressure to afford (S)-4-(2-((3-fluoro-4-((2-methylmorpholino)methyl)phenyl)amino)pyrimidin-4-yl)benzoic acid as pale yellow solid (170 mg, Crude).

TLC system: MeOH/dichloromethane (5:95)

R_{f} value: ˜0.1

LCMS(m/z): 423.41 (M+H)⁺

### Step 6: Synthesis of 4-(2-((3-fluoro-4-(((S)-2-methylmorpholino)methyl)phenyl)amino)pyrimidin-4-yl)-N-((tetrahydro-2H-pyran-2-yl)oxy)benzamide

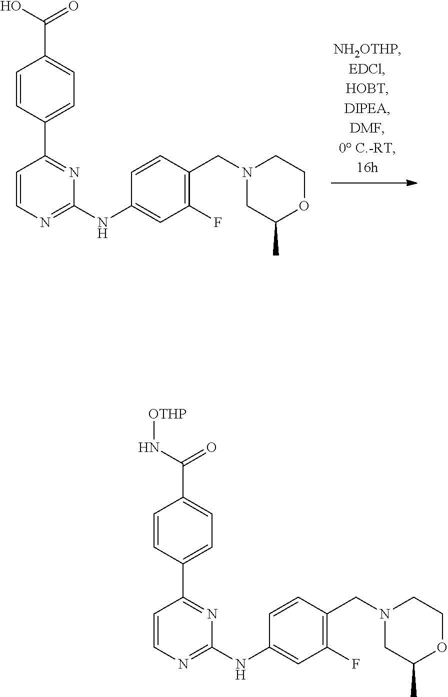

To a stirred solution of (S)-4-(2-((3-fluoro-4-((2-methylmorpholino)methyl)phenyl)amino)pyrimidin-4-yl)benzoic acid (170 mg, 0.40 mmol, 1 eq) in DMF (1.7 mL) at 0° C., was added DIPEA (0.2 mL, 1.20 mmol, 3.0 eq), HOBt (54 mg, 0.40 mmol, 1.0 eq) followed by EDC-HCl (77 mg, 0.40 mmol, 1.0 eq) and stirred for 15 min. Later, added NH₂OTHP (57 mg, 0.48 mmol, 1.2 eq) and stirred at room temperature for 16 h. After completion of reaction by TLC, the reaction mixture was quenched with ice cold water (10 mL) and extracted with EtOAc (2×20 mL). The combined organic layer was dried under reduced pressure to afford 4-(2-((3-fluoro-4-(((S)-2-methylmorpholino)methyl)phenyl)amino)pyrimidin-4-yl)-N-((tetrahydro-2H-pyran-2-yl)oxy)benzamide as yellow solid (140 mg, Crude).

TLC system: EtOAc (100%)

R_{f} value: ˜0.25

LCMS(m/z): 522.56 (M+H)⁺

### Step 7: Synthesis of (S)-4-(2-((3-fluoro-4-((2-methylmorpholino)methyl)phenyl)amino)pyrimidin-4-yl)-N-hydroxybenzamide formic acid salt

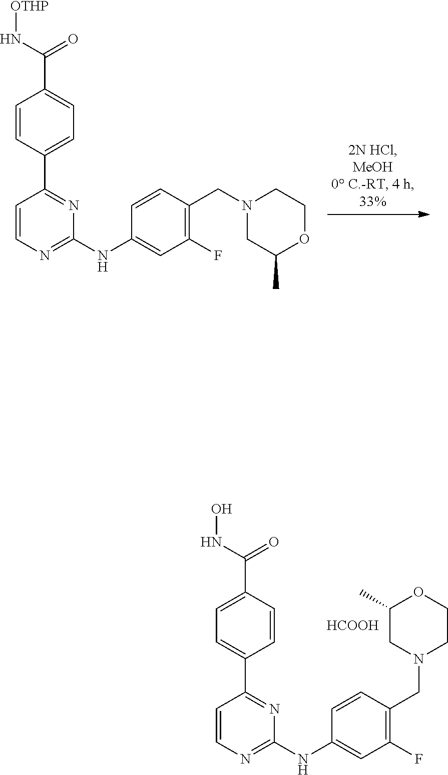

To a stirred solution of 4-(2-((3-fluoro-4-(((S)-2-methylmorpholino)methyl)phenyl)amino)pyrimidin-4-yl)-N-((tetrahydro-2H-pyran-2-yl)oxy)benzamide (140 mg, 0.26 mmol, 1 eq) in MeOH (1.4 mL) at 0° C., was added 2N aq. HCl (0.7 mL, 5 Vol) and stirred at room temperature for 4 h. After completion of reaction by TLC, reaction mixture was concentrated under reduced pressure and purified by reverse phase column chromatography [elution with 15-18% acetonitrile in 0.1% formic acid in H₂O] to afford (S)-4-(2-((3-fluoro-4-((2-methylmorpholino)methyl)phenyl)amino)pyrimidin-4-yl)-N-hydroxybenzamide as formic acid salt as off white solid (43 mg, yield: 33%).

TLC system: MeOH/dichloromethane (10:90)

R_{f} value: ˜0.1

LCMS (m/z): 438.2 (M+H)⁺

¹H NMR (400 MHz, DMSO-d₆) δ: 9.98 (s, 1H), 9.16 (s, 1H), 8.63 (d, J=5.2 Hz, 1H), 8.24 (d, J=8.4 Hz, 2H), 8.13 (s, 1H; formic acid), 7.92 (d, J=8.4 Hz, 2H), 7.85 (dd, J=1.6 Hz, 13.2 Hz, 1H), 7.56-7.52 (m, 2H), 7.30 (t, J=8.4 Hz, 1H), 3.74-3.71 (m, 1H), 3.50-3.44 (m, 4H), 2.72-2.62 (m, 2H), 2.07-2.05 (br, 1H), 1.75 (br, 1H), 1.02 (d, J=6.4 Hz, 3H)

### Synthetic Example 16

### Synthesis of Synthesis of (R)-4-(2-((3-Fluoro-4-((2-Methylmorpholino)Methyl)Phenyl)Amino)Pyrimidin-4-Yl)-N-Hydroxybenzamide Trifluoroacetic Acid Salt

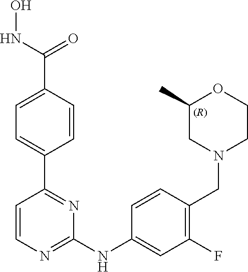

### Step 1: Synthesis of (R)-(2-fluoro-4-nitrophenyl)(2-methylmorpholino)methanone

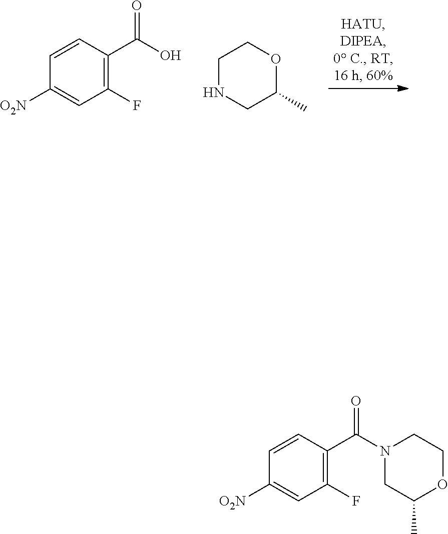

To a stirred solution of 2-fluoro-4-nitrobenzoic acid (1 g, 5.40 mmol, 1 eq) in DMF (10 mL) at 0° C., was added HATU (2.4 g, 6.50 mmol, 1.2 eq), DIPEA (2.3 mL, 13.5 mmol, 3.0 eq), and stirred for 10 min followed by addition of (R)-2-methylmorpholine (0.89 g, 6.50 mmol, 1.0 eq) and stirred at room temperature for 16 h. After completion of reaction by TLC, reaction mixture was quenched with ice cold water (20 mL) and extracted with EtOAc (3×50 mL). The combined organic layer was washed with brine solution (20 mL), dried over Na₂SO₄, filtered, and concentrated under reduced pressure to afford crude product. The crude product was purified by silica gel (100-200 mesh) column chromatography [elution with 25% EtOAc in pet ether] to afford (R)-(2-fluoro-4-nitrophenyl)(2-methylmorpholino)methanone as yellow solid (860 mg, Yield: 60%).

TLC system: EtOAc:Hexane (50:50)

R_{f} value: ˜0.5

LCMS(m/z): 269.1 (M+H)⁺

### Step 2: Synthesis of (R)-(4-amino-2-fluorophenyl)(2-methylmorpholino)methanone

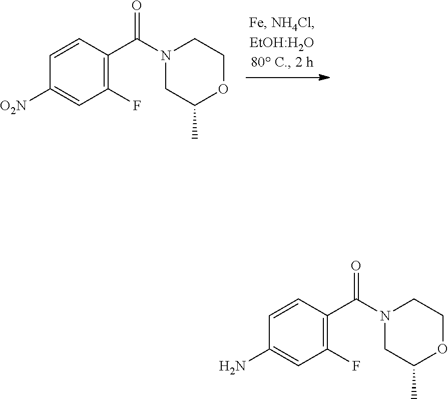 [SMILES: C[C@@H]1CN(C(=O)c2ccc([N+](=O)[O-])cc2F)CCO1 ; C[C@@H]1CN(C(=O)c2ccc(N)cc2F)CCO1]

To a stirred solution of (R)-(2-fluoro-4-nitrophenyl)(2-methylmorpholino)methanone (850 mg, 3.17 mmol, 1 eq) in EtOH:H₂O (2:1) (9 mL) at room temperature, was added Fe (888 mg, 15.8 mmol, 5 eq) and NH₄Cl (840 mg, 15.8 mmol, 5 eq) and reaction mixture was heated to 80° C., stirred for 2 h. After completion of reaction by TLC, reaction mixture was filtered through a pad of diatomaceous earth (e.g., Celite®) and washed with EtOAc (30 mL). Filtrate was concentrated, diluted with water (80 mL) and extracted with EtOAc (3×50 mL). Combined organic layer was dried over Na₂SO₄, filtered and concentrated under reduced pressure to afford (R)-(4-amino-2-fluorophenyl)(2-methylmorpholino)methanone as yellow solid (800 mg, crude). Crude product was used in the next step without further purification.

TLC system: EtOAc/Hexane (70:30)

R_{f} value: ˜0.4

### Step 3: Synthesis of (R)-3-fluoro-4-((2-methylmorpholino)methyl)aniline

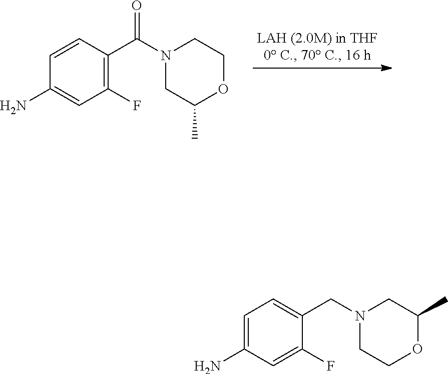 [SMILES: C[C@@H]1CN(C(=O)c2ccc(N)cc2F)CCO1 ; C[C@@H]1CN(Cc2ccc(N)cc2F)CCO1]

To a stirred solution of (R)-(4-amino-2-fluorophenyl)(2-methylmorpholino)methanone (800 mg, 3.36 mmol, 1.0 eq) in THF (8 mL) at 0° C., was added LAH (2.0 M in THF) (5 mL, 10.1 mmol, 3.0 eq) and stirred at 70° C. for 16 h. After completion of reaction by TLC, the reaction mixture was quenched with NH₄Cl solution (50 mL) and extracted with EtOAc (2×50 mL). The combined organic layer was dried over Na₂SO₄, filtered, concentrated under reduced pressure to afford (R)-3-fluoro-4-((2-methylmorpholino)methyl)aniline as pale yellow gummy (550 mg, Crude).

TLC system: EtOAc/Hexane (70:30)

R_{f} value: ˜0.3

LCMS(m/z): 225.3 (M+H)⁺

### Step 4: Synthesis of methyl (R)-4-(2-((3-fluoro-4-((2-methylmorpholino)methyl)phenyl)amino)pyrimidin-4-yl)benzoate

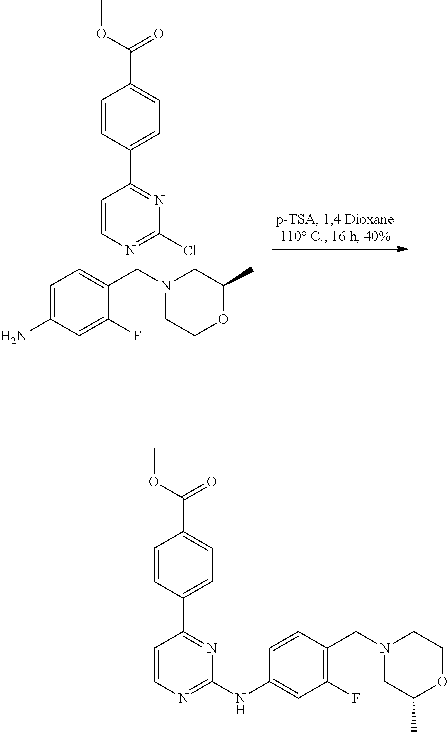

To a degassed stirred solution of methyl 4-(2-chloropyrimidin-4-yl)benzoate (600 mg, 2.41 mmol, 1 eq) and (R)-3-fluoro-4-((2-methylmorpholino)methyl)aniline (550 mg, 2.41 mmol, 1.0 eq) in 1,4-dioxane (12 mL) at room temperature, was added p-TSA (395 mg, 2.16 mmol, 0.9 eq) and stirred at 110° C. for 16 h. After completion of reaction by TLC, reaction mixture was diluted with water (50 mL) and extracted with 10% MeOH/dichloromethane (2×50 mL). The combined organic layer was dried over Na₂SO₄, filtered, concentrated and purified by reverse phase column chromatography (Grace) [elution with 25-30% acetonitrile in 0.1% formic acid in H₂O] to afford methyl (R)-4-(2-((3-fluoro-4-((2-methylmorpholino)methyl)phenyl)amino)pyrimidin-4-yl)benzoate as yellow gummy solid (420 mg, yield: 40%).

TLC system: EtOAc (100%)

R_{f} value: ˜0.9

LCMS(m/z): 437.47 (M+H)⁺

### Step 5: Synthesis of (R)-4-(2-((3-fluoro-4-((2-methylmorpholino)methyl)phenyl)amino)pyrimidin-4-yl)benzoic acid

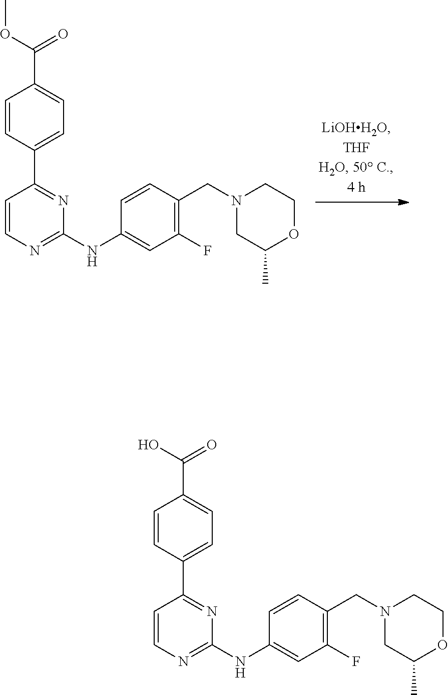 [SMILES: COC(=O)c1ccc(-c2ccnc(Nc3ccc(CN4CCO[C@H](C)C4)c(F)c3)n2)cc1 ; C[C@@H]1CN(Cc2ccc(Nc3nccc(-c4ccc(C(=O)O)cc4)n3)cc2F)CCO1]

To a stirred solution of methyl (R)-4-(2-((3-fluoro-4-((2-methylmorpholino)methyl)phenyl)amino)pyrimidin-4-yl)benzoate (420 mg, 0.96 mmol, 1.0 eq) in THF:H₂O (1:1) (4.5 mL) at 0° C., was added LiOH·H₂O (81 mg, 1.93 mmol, 2 eq) and stirred at 50° C. for 4 h. After completion of reaction by TLC, reaction mass was concentrated and washed with Et2O (30 mL). Aqueous layer was acidified with 2N HCl solution (10 mL), solidification observed, filtered and dried under vacuum to afford (R)-4-(2-((3-fluoro-4-((2-methylmorpholino)methyl)phenyl)amino)pyrimidin-4-yl)benzoic acid as brown solid (300 mg, Crude).

TLC system: MeOH/dichloromethane (10:90)

R_{f} value: ˜0.2

LCMS(m/z): 423.3 (M+H)⁺

### Step 6: Synthesis of 4-(2-((3-fluoro-4-(((R)-2-methylmorpholino)methyl)phenyl)amino)pyrimidin-4-yl)-N-((tetrahydro-2H-pyran-2-yl)oxy)benzamide

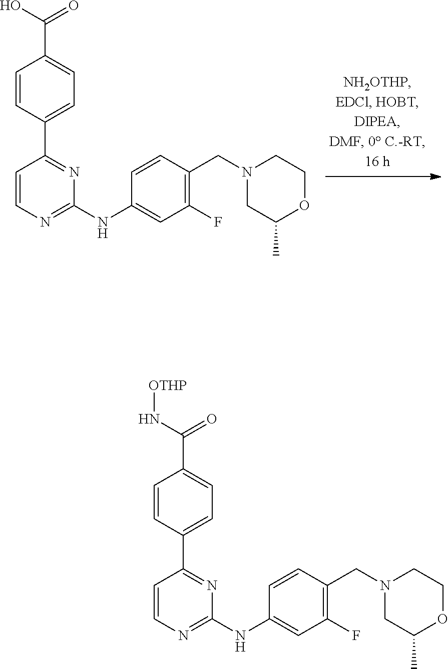

To a stirred solution of (R)-4-(2-((3-fluoro-4-((2-methylmorpholino)methyl)phenyl)amino)pyrimidin-4-yl)benzoic acid (200 mg, 0.47 mmol, 1 eq) in DMF (2 mL) at 0° C., was added DIPEA (0.25 mL, 1.42 mmol, 3.0 eq), HOBt (64 mg, 0.47 mmol, 1.0 eq) followed by EDC-HCl (91 mg, 0.47 mmol, 1.0 eq) and stirred for 30 min. Later, NH₂OTHP (64 mg, 0.57 mmol, 1.2 eq) was added and stirred at room temperature for 16 h. After completion of reaction by TLC, reaction mixture was quenched with ice cold water (10 mL) and extracted with EtOAc (2×25 mL). The combined organic layer was washed with brine solution (10 mL), dried over Na₂SO₄, filtered and concentrated under reduced pressure to afford 4-(2-((3-fluoro-4-(((R)-2-methylmorpholino)methyl)phenyl)amino)pyrimidin-4-yl)-N-((tetrahydro-2H-pyran-2-yl)oxy)benzamide as light brown gummy liquid (200 mg, 52% purity).

TLC system: EtOAc (100%)

R_{f} value: ˜0.5

LCMS(m/z): 522.23 (M+H)⁺

### Step 7: Synthesis of (R)-4-(2-((3-fluoro-4-((2-methylmorpholino)methyl)phenyl)amino)pyrimidin-4-yl)-N-hydroxybenzamide trifluoroacetic acid salt

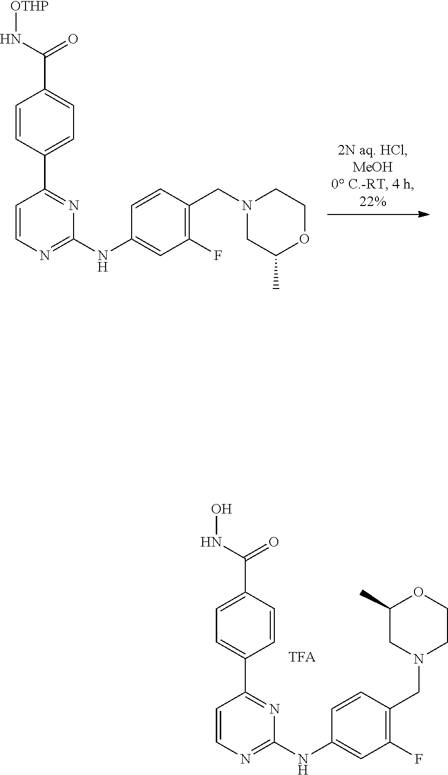

To a stirred solution of 4-(2-((3-fluoro-4-(((R)-2-methylmorpholino)methyl)phenyl)amino)pyrimidin-4-yl)-N-((tetrahydro-2H-pyran-2-yl)oxy)benzamide (200 mg, crude, 1 eq) in MeOH (2.0 mL) at 0° C., was added 2N aq. HCl (1 mL, 5 Vol) and stirred at room temperature for 4 h. After completion of reaction by TLC, reaction mixture was concentrated under reduced pressure and purified by Prep-HPLC (trifluoroacetic acid buffer). The collected fractions were frozen and lyophilized to afford (R)-4-(2-((3-fluoro-4-((2-methylmorpholino)methyl)phenyl)amino)pyrimidin-4-yl)-N-hydroxybenzamide trifluoroacetic acid salt as pale yellow solid (45 mg, yield: 22%).

TLC system: EtOAc (100%)

R_{f} value: ˜0.1

LCMS (m/z): 438.3 (M+H)⁺

¹H NMR (400 MHz, DMSO-d₆) δ: 11.39 (s, 1H), 10.24 (s, 1H), 9.93 (s, 1H), 9.17 (s, 1H), 8.68 (d, J=5.2 Hz, 1H), 8.25 (d, J=8.4 Hz, 2H), 8.02 (d, J=13.2 Hz, 1H), 7.93 (d, J=8.4 Hz, 2H), 7.67 (d, J=8.8 Hz, 1H), 7.60 (d, J=5.2 Hz, 1H), 7.50 (t, J=8.4 Hz, 1H), 4.34 (br s, 2H), 4.00-3.97 (m, 1H), 3.73-3.64 (m, 2H), 3.38-3.34 (m, 1H), 3.09-3.07 (m, 1H), 2.82-2.80 (m, 1H), 1.13 (d, J=6 Hz, 3H)

### Synthetic Example 17

### Synthesis of 4-(5-Chloro-2-((3-Fluoro-4-(Morpholine-4-Carbonyl) Phenyl)Amino)Pyrimidin-4-Yl)-N-Hydroxybenzamide

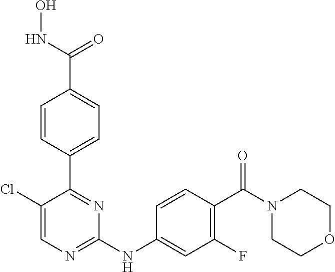 [SMILES: O=C(NO)c1ccc(-c2nc(Nc3ccc(C(=O)N4CCOCC4)c(F)c3)ncc2Cl)cc1]

### Step 1: Methyl 4-(2,5-dichloropyrimidin-4-yl)benzoate

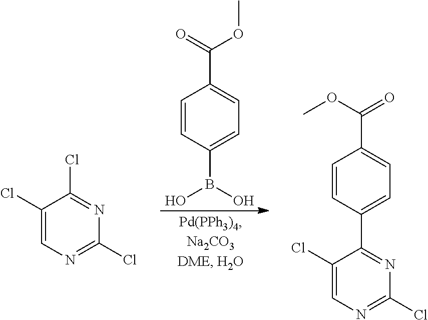 [SMILES: Clc1ncc(Cl)c(Cl)n1 ; COC(=O)c1ccc(B(O)O)cc1 ; COC(=O)c1ccc(-c2nc(Cl)ncc2Cl)cc1]

To a mixture of 2,4,5-trichloropyrimidine (3.0 g, 16.67 mmol) and (4-(methoxycarbonyl)phenyl)boronic acid (3.0 g, 16.67 mmol) in 1,2-dimethoxy-ethan (40 mL) and H₂O (4 mL) was added Na₂CO₃ (3.5 g, 33.36 mmol) followed by Pd(PPh₃)₄(1.9 g, 1.67 mmol). The reaction mixture was degassed under N₂ atmosphere for three times and stirred at 120° C. for 12 hrs. The mixture was diluted with EtOAc (40 mL), washed with water and brine, dried over anhydrous Na₂SO₄, filtered, and concentrated to dryness. The residue was purified by chromatography (silica gel, 0-5% EtOAc in PE) to give methyl 4-(2,5-dichloropyrimidin-4-yl)benzoate (2.2 g, 46.6% yield) as white solid.

LC/MS (ESI) m/z: 284 (M+H)⁺

### Step 2: Methyl-4-(5-chloro-2-((3-fluoro-4-(morpholine-4-carbonyl) phenyl)amino)pyrimidin-4-yl)benzoate

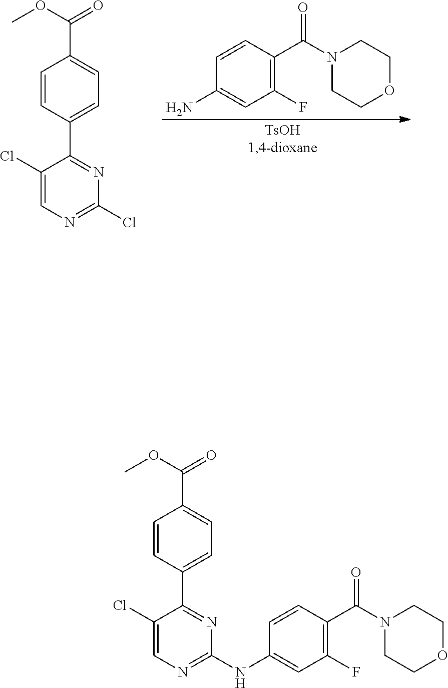

To a mixture of methyl 4-(2,5-dichloropyrimidin-4-yl)benzoate (1.0 g, 3.50 mmol) and (4-amino-2-fluorophenyl)(morpholino)methanone (875 mg, 3.90 mmol) in 1,4-dioxane (10 mL) was added 4-methylbenzenesulfonic acid (60 mg, 0.35 mmol). The reaction mixture was stirred at 120° C. for 12 hrs. The mixture was diluted with EtOAc (60 mL), washed with water and brine, dried over anhydrous Na₂SO₄, filtered, and concentrated to dryness. The residue was purified by chromatography (silica gel, 0-17% EtOAc in PE) to give tert-butyl (tert-butoxycarbonyl)(3-(3-((tert-butoxycarbonyl)amino)prop-1-yn-1-yl)pyridin-2-yl) carbamate (0.628 g, 38.2% yield) as yellow oil.

LC/MS (ESI) m/z: 471 (M+H)⁺

¹H NMR (400 MHz, DMSO-d₆) δ 10.45 (s, 1H), 8.77 (s, 1H), 8.14 (d, J=8.4 Hz, 2H), 7.96 (d, J=8.4 Hz, 2H), 7.85 (dd, J=12.8, 1.6 Hz, 1H), 7.56 (dd, J=8.4, 2.0 Hz, 1H), 7.35 (t, J=8.4 Hz, 1H), 3.91 (s, 3H), 3.63-3.54 (m, 8H)

### Step 3: 4-(5-Chloro-2-((3-fluoro-4-(morpholine-4-carbonyl)phenyl)amino) pyrimidin-4-yl)-N-hydroxybenzamide

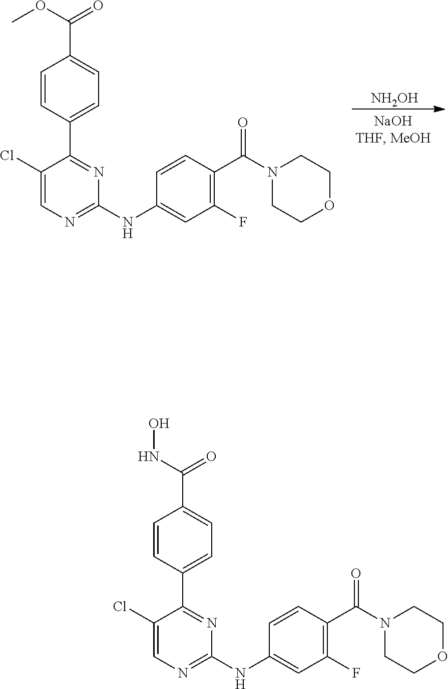 [SMILES: COC(=O)c1ccc(-c2nc(Nc3ccc(C(=O)N4CCOCC4)c(F)c3)ncc2Cl)cc1 ; "NH2OH" ; O=C(NO)c1ccc(-c2nc(Nc3ccc(C(=O)N4CCOCC4)c(F)c3)ncc2Cl)cc1]

To a solution of methyl-4-(5-chloro-2-((3-fluoro-4-(morpholine-4-carbonyl) phenyl)amino)pyrimidin-4-yl)benzoate (170 mg, 0.36 mmol) in MeOH (1.5 mL) and tetrahydrofuran (1.5 mL) was added NaOH (70 mg, 1.72 mmol) followed by hydroxylamine (24 mg, 0.36 mmol, 50% in water) was added dropwise under N₂ atmosphere at 0° C. The reaction was stirred at 25° C. for 12 hrs. The mixture was acidified with acetic acid to pH ˜3 and extracted with EtOAc twice. The combined organic layers were dried over anhydrous Na₂SO₄, filtered, and concentrated under reduced pressure. The residue was purified by perp-HPLC (C18, 0˜90% acetonitrile in H₂O with 0.1% formic acid) to give 4-(5-chloro-2-((3-fluoro-4-(morpholine-4-carbonyl)phenyl)amino) pyrimidin-4-yl)-N-hydroxybenzamide (74 mg, 43.6% yield) as white solid.

LC/MS (ESI) m/z: 472 (M+H)⁺

¹H NMR (400 MHz, DMSO-d₆) δ 11.35 (s, 1H), 10.41 (s, 1H), 9.15 (s, 1H), 8.75 (s, 1H), 7.93-7.84 (m, 5H), 7.57-7.53 (m, 1H), 7.34 (t, J=8.4 Hz, 1H), 3.68-3.60 (m, 4H), 3.56-3.51 (m, 2H), 3.29-3.23 (m, 2H)

### Step 4: (4-Amino-2-fluorophenyl)(morpholino)methanone

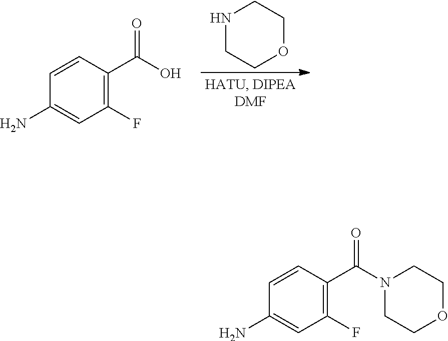 [SMILES: Nc1ccc(C(=O)O)c(F)c1 ; C1COCCN1 ; Nc1ccc(C(=O)N2CCOCC2)c(F)c1]

To a solution of 4-amino-2-fluorobenzoic acid (5.0 g, 32.2 mmol) in DMF (100 mL) was added DIPEA (9.6 g, 74.3 mmol) followed by HATU (15.95 g, 42.0 mmol) and morpholine (3.4 g, 38.7 mmol). The reaction was stirred at 25° C. for 3 hrs. The mixture was diluted with EtOAc (60 mL), washed with water and brine, dried over anhydrous Na₂SO₄, filtered, and concentrated to dryness. The residue was purified by chromatography (silica gel, 0-100% EtOAc in PE) to give (4-amino-2-fluorophenyl)(morpholino)methanone (6.5 g, 90% yield) as brown oil.

LC/MS (ESI) m/z: 225 (M+H)⁺

¹H NMR (400 MHz, CDCl₃) δ 7.21 (t, J=8.0 Hz, 1H), 6.47 (dd, J=8.0, 2.4 Hz, 1H), 6.33 (dd, J=12.0, 2.4 Hz, 1H), 3.76-3.65 (m, 6H), 3.35-3.43 (m, 2H)

### Synthetic Example 18

### Synthesis of N′-Ethyl-4-(2-((3-Fluoro-4-(Morpholine-4-Carbonyl)Phenyl)Amino)Pyrimidin-4-Yl)Benzohydrazide

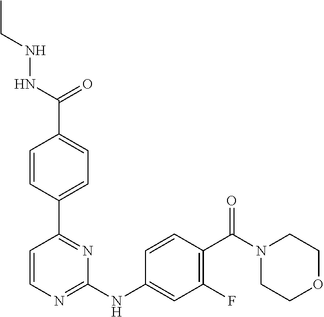 [SMILES: CCNNC(=O)c1ccc(-c2ccnc(Nc3ccc(C(=O)N4CCOCC4)c(F)c3)n2)cc1]

### Step 1: Methyl 4-(2-chloropyrimidin-4-yl)benzoate

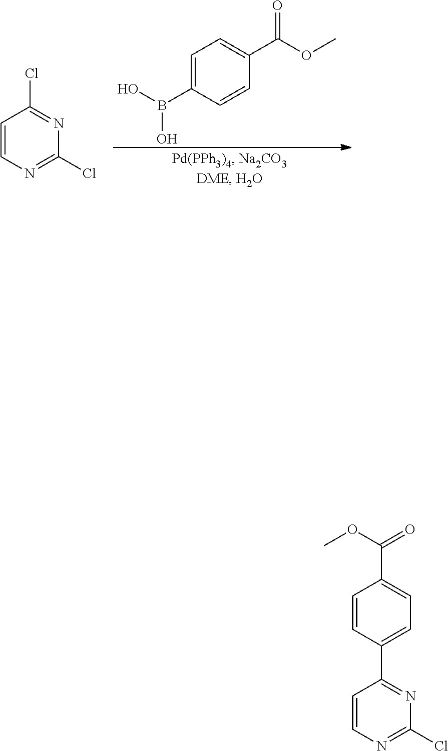 [SMILES: Clc1ccnc(Cl)n1 ; COC(=O)c1ccc(B(O)O)cc1 ; COC(=O)c1ccc(-c2ccnc(Cl)n2)cc1]

To a solution of 2,4-dichloropyrimidine (2.0 g, 13.43 mmol) in DME (25 mL) and H₂O (2.5 mL) was added (4-(methoxycarbonyl) phenyl) boronic acid (2.42 g, 13.43 mmol) followed by Na₂CO₃ (2.85 g, 26.85 mmol) and Pd(PPh₃)₄. The mixture was degassed under N₂ atmosphere for three times and stirred at 85° C. for 6 hrs. The mixture was concentrated under reduced pressure to give methyl 4-(2-chloropyrimidin-4-yl) benzoate (2.7 g, 80% yield) as white solid.

LC/MS (ESI) m/z: 249 (M+H)⁺

### Step 2: Methyl 4-(2-((3-fluoro-4-(morpholine-4-carbonyl)phenyl)amino)pyrimidin-4-yl)benzoate

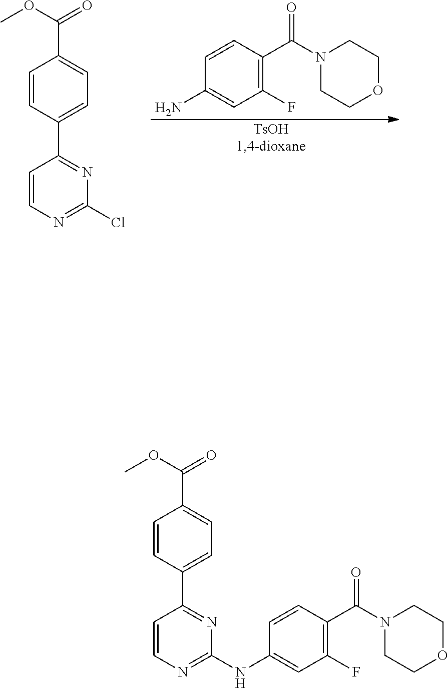

To a mixture of methyl 4-(2-chloropyrimidin-4-yl)benzoate (720 mg, 2.90 mmol) and (4-amino-2-fluorophenyl)(morpholino)methanone (500 mg, 2.23 mmol) in 1,4-dioxane (20 mL) was added p-toluenesulfonic acid (45 mg, 0.22 mmol). The reaction mixture was stirred at 120° C. for 16 hrs. The mixture was diluted with EtOAc (20 mL), washed with water and brine, dried over anhydrous Na₂SO₄, filtered, and concentrated to dryness. The residue was purified by chromatography (silica gel, 0-50% EtOAc in PE) to give methyl 4-(2-((3-fluoro-4-(morpholine-4-carbonyl)phenyl)amino)pyrimidin-4-yl)benzoate (500 mg, 39.5% yield) as yellow solid.

LC/MS (ESI) m/z: 437 (M+H)⁺

### Step 3: 4-(2-((3-Fluoro-4-(morpholine-4-carbonyl)phenyl)amino)pyrimidin-4-yl)benzoic acid

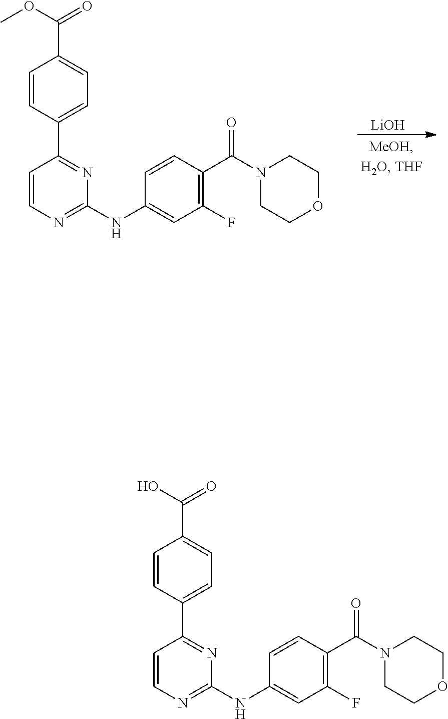 [SMILES: COC(=O)c1ccc(-c2ccnc(Nc3ccc(C(=O)N4CCOCC4)c(F)c3)n2)cc1 ; "LiOH" ; O=C(O)c1ccc(-c2ccnc(Nc3ccc(C(=O)N4CCOCC4)c(F)c3)n2)cc1]

To a solution of methyl 4-(2-((3-fluoro-4-(morpholine-4-carbonyl)phenyl)amino)pyrimidin-4-yl)benzoate (500 mg, 1.14 mmol) in MeOH (5 mL), THF (5 mL) and H₂O (5 mL) was added LiOH—H₂O (192 mg, 4.58 mmol) at 0° C. The reaction was stirred at room temperature for 16 hrs. The solvent was removed under vacuum and the residue was diluted with H₂O (30 mL). The mixture was adjusted with 1N aq. HCl to pH˜3. The mixture was extracted with EtOAc (3×30 mL) and the combined organic layers were washed with brine, dried over anhydrous Na₂SO₄, filtered and concentrated to give 4-(2-((3-fluoro-4-(morpholine-4-carbonyl)phenyl)amino)pyrimidin-4-yl)benzoic acid (360 mg, 74.1% yield) as yellow solid.

LC/MS (ESI) m/z: 423 (M+H)⁺

### Step 4: Tert-butyl 1-ethyl-2-(4-(2-((3-fluoro-4-(morpholine-4-carbonyl)phenyl)amino)pyrimidin-4-yl)benzoyl)hydrazine-1-carboxylate

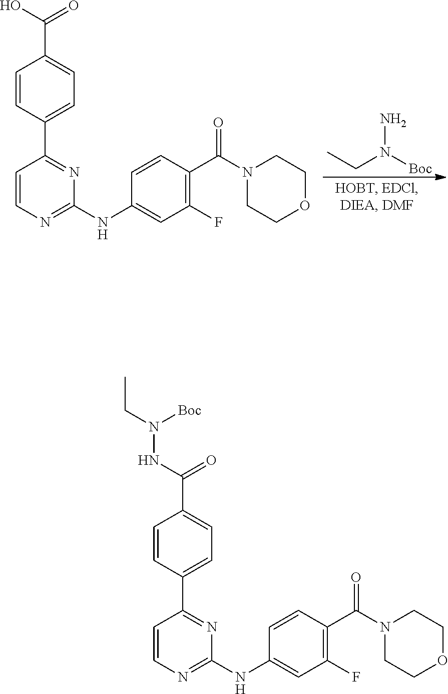

To a solution of 4-(2-((3-fluoro-4-(morpholine-4-carbonyl)phenyl)amino)pyrimidin-4-yl)benzoic acid (360 mg, 0.85 mmol) in DMF (6 mL) was added tert-butyl 1-ethylhydrazine-1-carboxylate (137 mg, 0.85 mmol) followed by DIPEA (222 mg, 1.70 mmol), EDC-HCl (140 mg, 1.02 mmol) and HOBt (197 mg, 1.02 mmol). The reaction mixture was stirred at room temperature for 16 hrs. The mixture was diluted EtOAc (20 mL), washed with water and brine, dried over anhydrous Na₂SO₄, filtered and concentrated to give tert-butyl 1-ethyl-2-(4-(2-((3-fluoro-4-(morpholine-4-carbonyl)phenyl)amino)pyrimidin-4-yl)benzoyl)hydrazine-1-carboxylate (450 mg, 75.4% yield) as yellow solid.

LC/MS (ESI) m/z: 565 (M+H)⁺

### Step 5: N-ethyl-4-(2-((3-fluoro-4-(morpholine-4-carbonyl)phenyl)amino)pyrimidin-4-yl)benzohydrazide

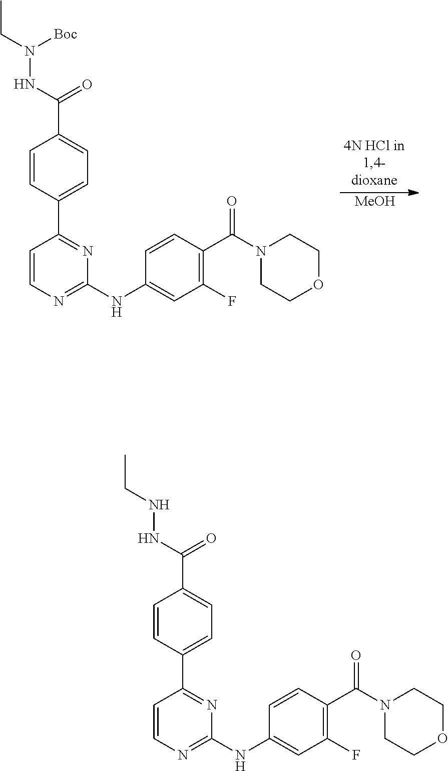 [SMILES: "MeOH" ; CCN(NC(=O)c1ccc(-c2ccnc(Nc3ccc(C(=O)N4CCOCC4)c(F)c3)n2)cc1)C(=O)OC(C)(C)C ; CCNNC(=O)c1ccc(-c2ccnc(Nc3ccc(C(=O)N4CCOCC4)c(F)c3)n2)cc1]

To a solution of tert-butyl 1-ethyl-2-(4-(2-((3-fluoro-4-(morpholine-4-carbonyl)phenyl)amino)pyrimidin-4-yl)benzoyl)hydrazine-1-carboxylate (450 mg, 0.80 mmol) in MeOH (6 mL) was added 4N HCl in 1,4-dioxane (6 mL, 24 mmol) at 0° C. The reaction was stirred at room temperature for 4 hrs. The solvent was removed under vacuum and the residue was diluted with H₂O (30 mL). The mixture was adjusted with saturated aq. NaHCO₃ to pH-9. The mixture was extracted with EtOAc (3×30 mL) and the combined organic layers were washed with brine, dried over anhydrous Na₂SO₄, filtered and concentrated. The residue was washed with MeOH to give N-ethyl-4-(2-((3-fluoro-4-(morpholine-4-carbonyl)phenyl)amino)pyrimidin-4-yl)benzohydrazide (110 mg, 29.6% yield) as yellow solid.

LC/MS (ESI) m/z: 465 (M+H)⁺

¹H NMR (400 MHz, DMSO-d₆) δ 11.51 (s, 1H), 8.42 (d, J=5.1 Hz, 1H), 8.19 (d, J=8.8 Hz, 2H), 7.85 (d, J=8.8 Hz, 2H), 7.17 (d, J=5.2 Hz, 1H), 4.37 (s, 4H), 3.95-3.64 (m, 4H), 1.98-1.69 (m, 4H)

### Synthetic Example 19

### Synthesis of N-Ethyl-4-(2-((4-(Morpholine-4-Carbonyl)Phenyl)Amino)Pyrimidin-4-Yl)Benzohydrazide

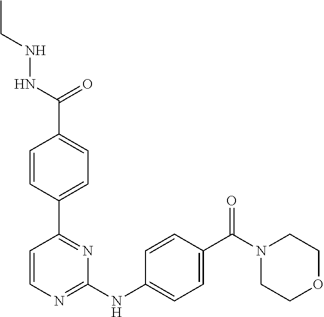 [SMILES: CCNNC(=O)c1ccc(-c2ccnc(Nc3ccc(C(=O)N4CCOCC4)cc3)n2)cc1]

### Step 1: Methyl 4-(2-((4-(morpholine-4-carbonyl)phenyl)amino)pyrimidin-4-yl)benzoate

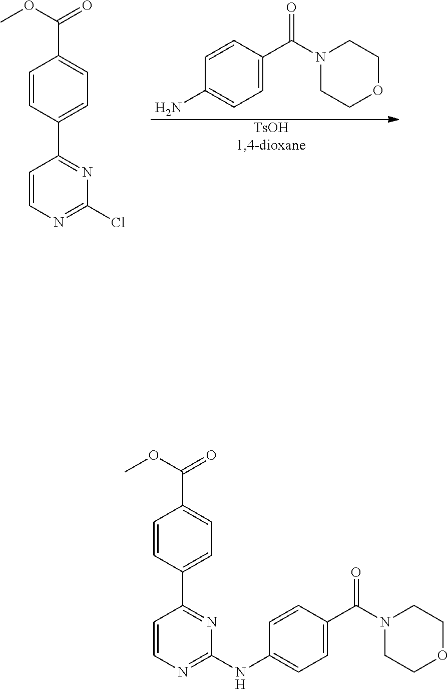 [SMILES: COC(=O)c1ccc(-c2ccnc(Cl)n2)cc1 ; Nc1ccc(C(=O)N2CCOCC2)cc1 ; "TsOH1,4-dioxane" ; COC(=O)c1ccc(-c2ccnc(Nc3ccc(C(=O)N4CCOCC4)cc3)n2)cc1]

To a mixture of methyl 4-(2-chloropyrimidin-4-yl)benzoate (723 mg, 2.91 mmol) and (4-aminophenyl)(morpholino)methanone (500 mg, 2.42 mmol) in 1,4-dioxane (10 mL) was added p-toluenesulfonic acid (41 mg, 0.24 mmol). The reaction mixture was stirred at 120° C. for 16 hrs. The mixture was concentrated under reduced pressure. The residue was purified by silica gel chromatography (0-100% EA in PE) to give methyl 4-(2-((4-(morpholine-4-carbonyl)phenyl)amino)pyrimidin-4-yl)benzoate (400 mg, 40.2% yield) as yellow solid.

LC/MS (ESI) m/z: 419 (M+H)⁺

### Step 2: 4-(2-((4-(Morpholine-4-carbonyl)phenyl)amino)pyrimidin-4-yl)benzoic acid

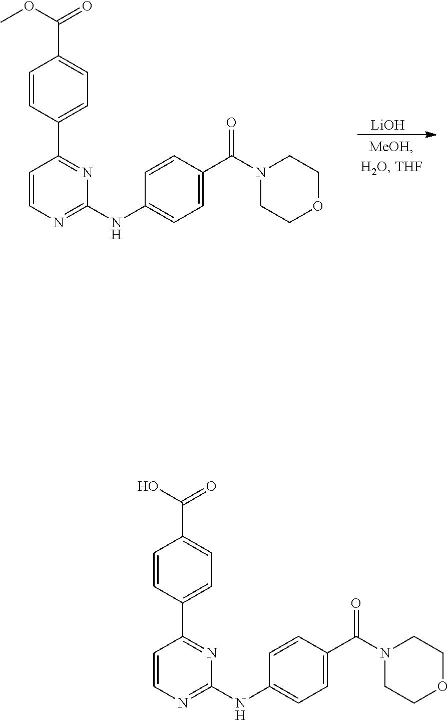 [SMILES: COC(=O)c1ccc(-c2ccnc(Nc3ccc(C(=O)N4CCOCC4)cc3)n2)cc1 ; "LiOH" ; O=C(O)c1ccc(-c2ccnc(Nc3ccc(C(=O)N4CCOCC4)cc3)n2)cc1]

To a solution of methyl 4-(2-((4-(morpholine-4-carbonyl)phenyl)amino)pyrimidin-4-yl)benzoate (360 mg, 0.86 mmol) in MeOH (4.5 mL), THF (4.5 mL) and H₂O (4.5 mL) was added LiOH—H₂O (82 mg, 3.44 mmol) at 0° C. The reaction mixture was stirred at room temperature for 16 hrs. The solvent was removed under vacuum and the residue was diluted with H₂O (20 mL). The mixture was adjusted with 1N aq. HCl to pH-3. The mixture was extracted with EtOAc (3×20 mL) and the combined organic layers were washed with brine, dried over anhydrous Na₂SO₄, filtered and concentrated to give 4-(2-((4-(morpholine-4-carbonyl)phenyl)amino)pyrimidin-4-yl)benzoic acid (210 mg, 60.2% yield) as yellow solid.

LC/MS (ESI) m/z: 405 (M+H)⁺

### Step 3: Tert-butyl-1-ethyl-2-(4-(2-((4-(morpholine-4-carbonyl)phenyl)amino)pyrimidin-4-yl)benzoyl)hydrazine-1-carboxylate

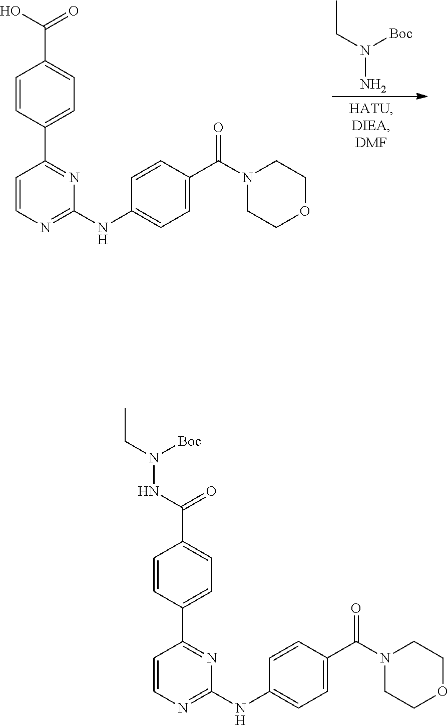 [SMILES: O=C(O)c1ccc(-c2ccnc(Nc3ccc(C(=O)N4CCOCC4)cc3)n2)cc1 ; CCN(N)C(=O)OC(C)(C)C ; CCN(NC(=O)c1ccc(-c2ccnc(Nc3ccc(C(=O)N4CCOCC4)cc3)n2)cc1)C(=O)OC(C)(C)C]

To a solution of 4-(2-((4-(morpholine-4-carbonyl)phenyl)amino)pyrimidin-4-yl)benzoic acid (200 mg, 0.49 mmol) in DMF (10 mL) was added tert-butyl 1-ethylhydrazine-1-carboxylate (95 mg, 0.59 mmol) followed by DIPEA (128 mg, 0.99 mmol) and HATU (226 mg, 0.59 mmol). The reaction was stirred at room temperature for 4 hrs. The mixture was diluted EtOAc (20 mL), washed with water and brine, dried over anhydrous Na₂SO₄, filtered and concentrated to give tert-butyl-1-ethyl-2-(4-(2-((4-(morpholine-4-carbonyl)phenyl)amino)pyrimidin-4-yl)benzoyl)hydrazine-1-carboxylate (288 mg crude) as yellow solid.

LC/MS (ESI) m/z: 547 (M+H)⁺

### Step 4: N′-ethyl-4-(2-((4-(morpholine-4-carbonyl)phenyl)amino)pyrimidin-4-yl)benzohydrazide

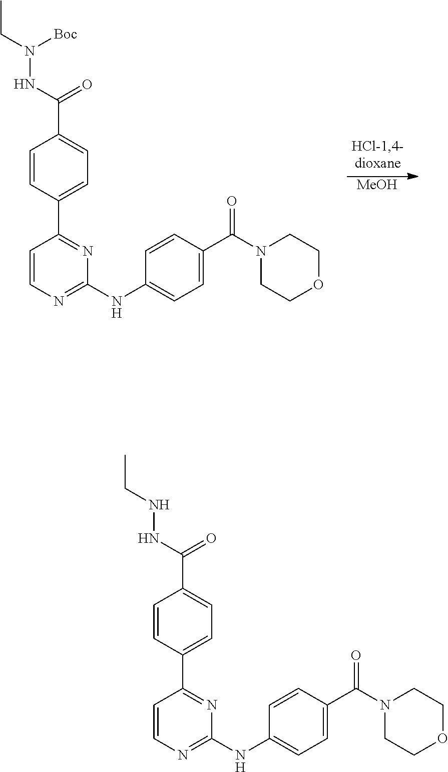 [SMILES: "MeOH" ; CCN(NC(=O)c1ccc(-c2ccnc(Nc3ccc(C(=O)N4CCOCC4)cc3)n2)cc1)C(=O)OC(C)(C)C ; CCNNC(=O)c1ccc(-c2ccnc(Nc3ccc(C(=O)N4CCOCC4)cc3)n2)cc1]

To a solution of tert-butyl-1-ethyl-2-(4-(2-((4-(morpholine-4-carbonyl)phenyl)amino)pyrimidin-4-yl)benzoyl)hydrazine-1-carboxylate (288 mg, 0.52 mmol) in MeOH (4 mL) was added 4 N HCl in 1,4-dioxane (4 mL) at 0° C. The reaction was stirred at room temperature for 16 hrs. The mixture was concentrated under reduced pressure and the residue was purified by prep-HPLC (C18, 0˜90% acetonitrile in H₂O with 0.1% formic acid) to give N-ethyl-4-(2-((4-(morpholine-4-carbonyl)phenyl)amino)pyrimidin-4-yl)benzohydrazide.

LC/MS (ESI) m/z: 447 (M+H)⁺

¹H NMR (400 MHz, DMSO-d₆) δ 10.16 (s, 1H), 9.98 (s, 1H), 8.63 (d, J=5.1 Hz, 1H), 8.26 (d, J=8.4 Hz, 2H), 8.00 (d, J=8.4 Hz, 2H), 7.92 (d, J=8.6 Hz, 2H), 7.53 (d, J=5.2 Hz, 1H), 7.41 (d, J=8.5 Hz, 2H), 3.64-3.59 (m, 4H), 3.56-3.48 (m, 4H), 2.85 (q, J=7.1 Hz, 2H), 1.06 (t, J=7.2 Hz, 3H).

### Synthetic Example 20

### Synthesis of N-(4-(4-(2-Ethylhydrazine-1-Carbonyl)Phenyl)Pyrimidin-2-Yl)Morpholine-4-Carboxamide

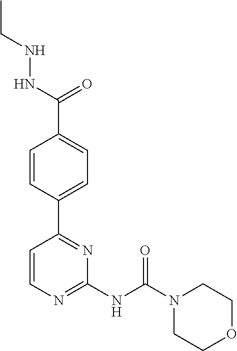 [SMILES: CCNNC(=O)c1ccc(-c2ccnc(NC(=O)N3CCOCC3)n2)cc1]

### Step 1: 4-Nitrophenyl morpholine-4-carboxylate

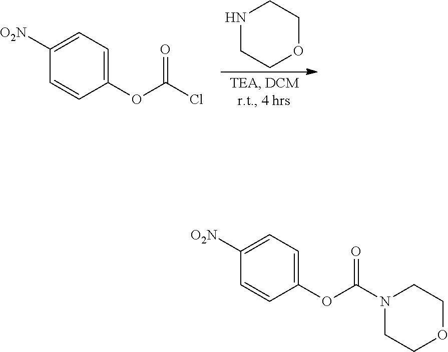 [SMILES: O=C(Cl)Oc1ccc([N+](=O)[O-])cc1 ; C1COCCN1 ; O=C(Oc1ccc([N+](=O)[O-])cc1)N1CCOCC1]

To a mixture of 4-nitrophenyl carbonochloridate (2.0 g, 9.92 mmol) and triethylamine (1.2 g, 11.91 mmol) in dichloromethane (20 mL) was added morpholine (0.86 g, 9.92 mmol) at 0° C. The reaction mixture was stirred at room temperature for 4 hrs. The mixture was diluted with dichloromethane (20 mL), washed with water and brine, dried over anhydrous Na₂SO₄, filtered, and concentrated to dryness. The residue was purified by chromatography (silica gel, 0-20% EtOAc in PE) to give 4-nitrophenyl morpholine-4-carboxylate (1.8 g, 72.1% yield) as white solid.

LC/MS (ESI) m/z: 253 (M+H)⁺

### Step 2: N-(4-chloropyrimidin-2-yl)morpholine-4-carboxamide

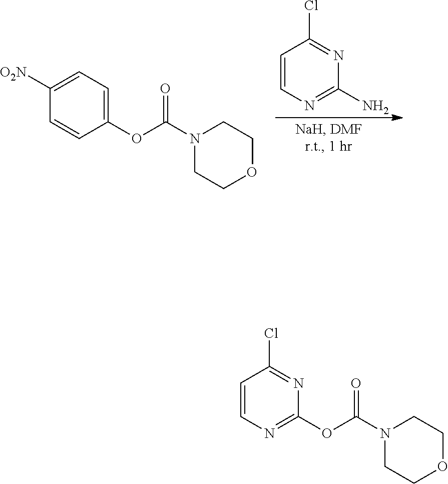 [SMILES: O=C(Oc1ccc([N+](=O)[O-])cc1)N1CCOCC1 ; Nc1nccc(Cl)n1 ; O=C(Oc1nccc(Cl)n1)N1CCOCC1]

To a mixture of 4-chloropyrimidin-2-amine (200 mg, 1.54 mmol) and 4-nitrophenyl morpholine-4-carboxylate (430 mg, 1.70 mmol) in DMF (4 mL) was added NaH (130 mg, 3.08 mmol, 60%, dispersion in paraffin liquid) at 0° C. The reaction mixture was stirred at room temperature for 1 hr. The mixture was quenched with H₂O (50 mL) and was diluted with EtOAc (30 mL), washed with brine, dried over anhydrous Na₂SO₄, filtered and concentrated to dryness give N-(4-chloropyrimidin-2-yl)morpholine-4-carboxamide (360 mg, 96.5% yield) as yellow oil.

LC/MS (ESI) m/z: 243 (M+H)⁺

### Step 3: Tert-butyl 1-ethyl-2-(4-(2-(morpholine-4-carboxamido)pyrimidin-4-yl)benzoyl)hydrazine-1-carboxylate

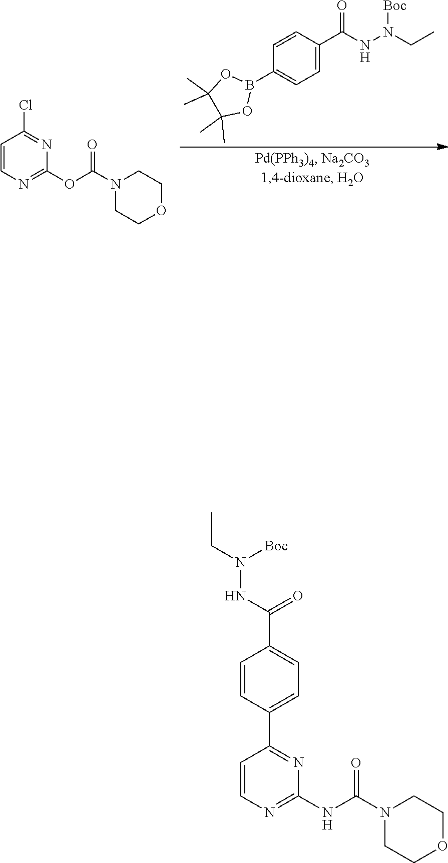 [SMILES: O=C(Oc1nccc(Cl)n1)N1CCOCC1 ; CCN(NC(=O)c1ccc(B2OC(C)(C)C(C)(C)O2)cc1)C(=O)OC(C)(C)C ; CCN(NC(=O)c1ccc(-c2ccnc(NC(=O)N3CCOCC3)n2)cc1)C(=O)OC(C)(C)C]

To a mixture of N-(4-chloropyrimidin-2-yl)morpholine-4-carboxamide (137 mg, 0.563 mmol) and tert-butyl 1-ethyl-2-(4-(4,4,5,5-tetramethyl-1,3,2-dioxaborolan-2-yl)benzoyl)hydrazine-1-carboxylate (200 mg, 0.512 mmol) in 1,4-dioxane (10 mL) and H₂O (2 mL) was added Na₂CO₃ (109 mg, 1.024 mmol) followed by Pd(PPh₃)₄ (59 mg, 0.051 mmol). The mixture was degassed under N₂ atmosphere for three times and stirred under N₂ atmosphere at 80° C. for 6 hr. The mixture was diluted with EtOAc (20 mL), washed with water and brine, dried over anhydrous Na₂SO₄, filtered, and concentrated to dryness. The residue was purified by chromatography (silica gel, 0-50% EtOAc in PE) to give tert-butyl 1-ethyl-2-(4-(2-(morpholine-4-carboxamido)pyrimidin-4-yl)benzoyl)hydrazine-1-carboxylate (120 mg, 49.8% yield) as yellow solid.

LC/MS (ESI) m/z: 471 (M+H)⁺

### Step 4: N-(4-(4-(2-ethylhydrazine-1-carbonyl)phenyl)pyrimidin-2-yl)morpholine-4-carboxamide

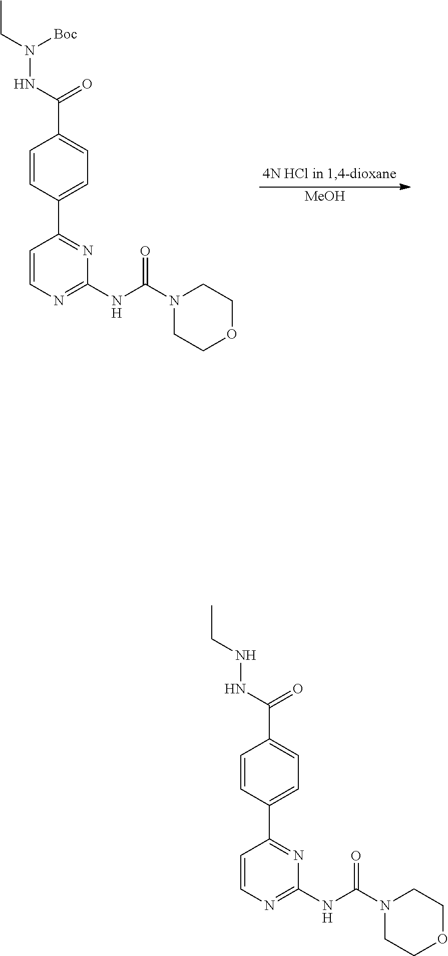 [SMILES: "MeOH" ; CCN(NC(=O)c1ccc(-c2ccnc(NC(=O)N3CCOCC3)n2)cc1)C(=O)OC(C)(C)C ; CCNNC(=O)c1ccc(-c2ccnc(NC(=O)N3CCOCC3)n2)cc1]

To a solution of tert-butyl 1-ethyl-2-(4-(2-(morpholine-4-carboxamido)pyrimidin-4-yl)benzoyl)hydrazine-1-carboxylate (120 mg, 0.255 mmol) in MeOH (5 mL) was added 4 N HCl in 1,4-dioxane (5 mL) at 0° C. The reaction mixture was stirred at room temperature for 5 hrs. The mixture was concentrated to dryness and the residue was dissolved in H₂O (5 mL), based with saturated aq. NaHCO₃ to pH ˜8. The mixture was extracted with EtOAc (3×20 mL) and the combined organic layers were washed with brine, dried over anhydrous Na₂SO₄, filtered and concentrated. The residue was purified by perp-HPLC (C18, 0˜90% acetonitrile in H₂O with 0.1% formic acid) to give N-(4-(4-(2-ethylhydrazine-1-carbonyl)phenyl)pyrimidin-2-yl)morpholine-4-carboxamide (35 mg, 53.2% yield) as white solid.

LC/MS (ESI) m/z: 371(M+H)⁺

¹H NMR (400 MHz, DMSO-d₆) δ 10.16 (s, 1H), 9.63 (s, 1H), 8.65 (d, J=5.2 Hz, 1H), 8.24 (d, J=8.4 Hz, 2H), 7.97 (d, J=8.5 Hz, 2H), 7.66 (d, J=5.2 Hz, 1H), 5.18-5.09 (m, 1H), 3.73-3.53 (m, 4H), 3.44 (dd, J=25.4, 20.9 Hz, 4H), 2.84 (d, J=6.7 Hz, 2H), 1.05 (t, J=7.2 Hz, 3H)

### Synthetic Example 21

### Synthesis of N′-Ethyl-4-(2-Morpholinopyrimidin-4-Yl)Benzohydrazide

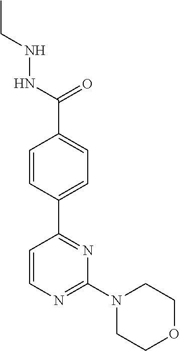 [SMILES: CCNNC(=O)c1ccc(-c2ccnc(N3CCOCC3)n2)cc1]

### Step 1: Tert-butyl 2-(4-(2-chloropyrimidin-4-yl)benzoyl)-1-ethylhydrazine-1-carboxylate

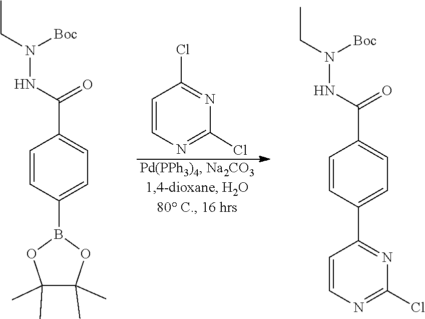

To a mixture of tert-butyl 1-ethyl-2-(4-(4,4,5,5-tetramethyl-1,3,2-dioxaborolan-2-yl)benzoyl)hydrazine-1-carboxylate (650 mg, 1.67 mmol) and 2,4-dichloropyrimidine (273 mg, 1.83 mmol) in 1,4-dioxane (15 mL) and H₂O (3 mL) was added Na₂CO₃ (106 mg, 2.5 mmol) followed by Pd(PPh₃)₄ (192 mg, 0.16 mmol). The reaction mixture was degassed under N₂ atmosphere for three times and was stirred at 80° C. for 16 hrs. The mixture was diluted with EtOAc (20 mL), washed with water and brine, dried over anhydrous Na₂SO₄, filtered, and concentrated to dryness. The residue was purified by chromatography (silica gel, 0-50% EtOAc in PE) to give tert-butyl 2-(4-(2-chloropyrimidin-4-yl)benzoyl)-1-ethylhydrazine-1-carboxylate (452 mg, 71.8% yield) as white solid.

LC/MS (ESI) m/z: 377 (M+H)⁺

### Step 2: Tert-butyl 1-ethyl-2-(4-(2-morpholinopyrimidin-4-yl)benzoyl)hydrazine-1-carboxylate

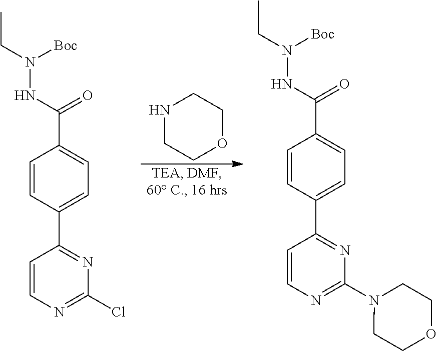

To a mixture of tert-butyl 2-(4-(2-chloropyrimidin-4-yl)benzoyl)-1-ethylhydrazine-1-carboxylate (100 mg, 0.27 mmol) and morpholine (25 mg, 0.29 mmol) in DMF (3 mL) was added triethylamine (40 mg, 0.40 mmol). The reaction was stirred at 60° C. for 16 hrs. The mixture was diluted with EtOAc (10 mL), washed with water and brine, dried over anhydrous Na₂SO₄, filtered, and concentrated to dryness. The residue was purified by chromatography (silica gel, 0-30% EtOAc in PE) to give tert-butyl 1-ethyl-2-(4-(2-morpholinopyrimidin-4-yl)benzoyl)hydrazine-1-carboxylate (88 mg, 77.6% yield) as yellow solid.

LC/MS (ESI) m/z: 428 (M+H)⁺

### Step 3: N′-ethyl-4-(2-morpholinopyrimidin-4-yl)benzohydrazide

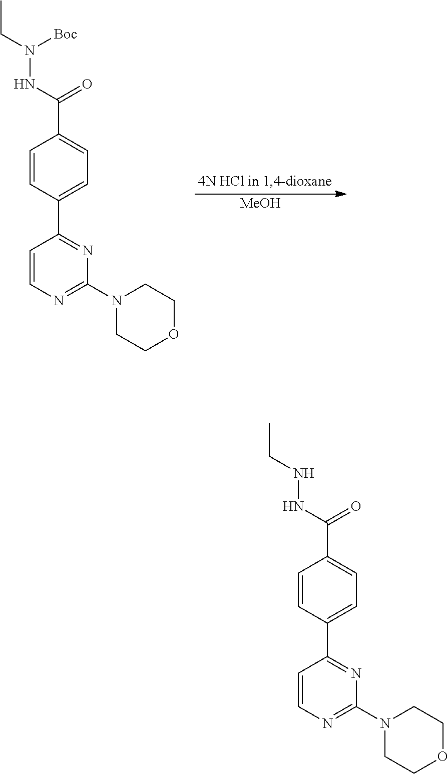 [SMILES: "MeOH" ; CCN(NC(=O)c1ccc(-c2ccnc(N3CCOCC3)n2)cc1)C(=O)OC(C)(C)C ; CCNNC(=O)c1ccc(-c2ccnc(N3CCOCC3)n2)cc1]

To a solution of tert-butyl 1-ethyl-2-(4-(2-morpholinopyrimidin-4-yl)benzoyl)hydrazine-1-carboxylate (88 mg, 0.20 mmol) in MeOH (3 mL) at 0° C. The reaction mixture was stirred at room temperature for 5 hrs. The mixture was concentrated to dryness and the residue was dissolved in H₂O (5 mL), based with saturated aq. NaHCO₃ to pH ˜8. The mixture was extracted with EtOAc (3×20 mL), and the combined organic layers were washed with brine, dried over anhydrous Na₂SO₄, filtered and concentrated. The residue was purified by perp-HPLC (C18, 0˜90% acetonitrile in H₂O with 0.1% formic acid) to give N′-ethyl-4-(2-morpholinopyrimidin-4-yl)benzohydrazide (20.3 mg, 30.3% yield) as white solid.

LC/MS (ESI) m/z: 328 (M+H)⁺

¹H NMR (400 MHz, DMSO-d₆) δ 10.16 (s, 1H), 8.50 (d, J=5.1 Hz, 1H), 8.22 (d, J=8.5 Hz, 2H), 7.94 (d, J=8.5 Hz, 2H), 7.32 (d, J=5.1 Hz, 1H), 5.14 (s, 1H), 3.80 (d, J=5.1 Hz, 4H), 3.71 (d, J=5.1 Hz, 4H), 2.84 (q, J=7.1 Hz, 2H), 1.05 (t, J=7.2 Hz, 3H)

### Synthetic Example 22

### Synthesis of 4-(5-Chloro-2-Morpholinopyrimidin-4-Yl)-N′-Ethylbenzohydrazide

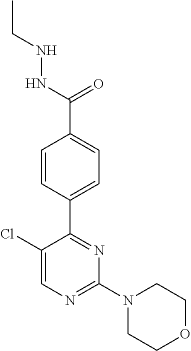 [SMILES: CCNNC(=O)c1ccc(-c2nc(N3CCOCC3)ncc2Cl)cc1]

### Step 1: Tert-butyl (1,3-dioxoisoindolin-2-yl)(ethyl)carbamate

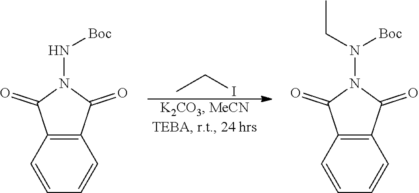

To a solution of tert-butyl (1,3-dioxoisoindolin-2-yl)carbamate (5.0 g, 19.06 mmol) in acetonitrile (50 mL) was added iodoethane (3.6 g, 22.88 mmol) followed by benzyltriethylammonium chloride (434 mg, 1.90 mmol) and K₂CO₃ (5.3 g, 38.13 mmol). The mixture was stirred at 55° C. for 12 hrs. The mixture was diluted with EtOAc (50 mL), washed with water and brine, dried over anhydrous Na₂SO₄, filtered, and concentrated to dryness. The residue was purified by chromatography (silica gel, 0-20% EtOAc in PE) to give tert-butyl (1,3-dioxoisoindolin-2-yl)(ethyl)carbamate (2.87 g, 51.9% yield) as white solid.

LC/MS (ESI) m/z: 291 (M+H)⁺

¹H NMR (400 MHz, CDCl₃) δ 8.02-7.85 (m, 2H), 7.79 (ddd, J=14.5, 5.4, 3.1 Hz, 2H), 3.72 (dq, J=14.1, 7.2 Hz, 2H), 1.55-1.30 (m, 9H), 1.21 (dt, J=12.3, 7.3 Hz, 3H)

### Step 2: Tert-butyl 1-ethylhydrazine-1-carboxylate

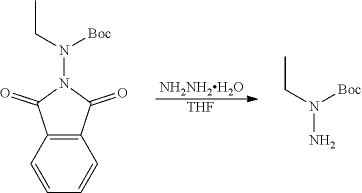

To a solution of tert-butyl (1,3-dioxoisoindolin-2-yl)(ethyl)carbamate (2.9 g, 9.89 mmol) in dichloromethane (20 mL) was added hydrazinium hydroxide solution (1.2 g, 19.77 mmol, 85% in water). The reaction mixture was stirred at 25° C. for 12 hrs. The mixture was filtered, and the filtrate was concentrated to dryness. The residue was purified by chromatography (silica gel, 0-20% EtOAc in PE) to give tert-butyl 1-ethylhydrazine-1-carboxylate (1.5 g, 95.3% yield) as yellow oil.

LC/MS (ESI) m/z: 161 (M+H)⁺

¹H NMR (400 MHz, CDCl₃) δ 3.99-3.55 (m, 2H), 3.33 (q, J=7.1 Hz, 2H), 1.40 (s, 9H), 1.05 (t, J=7.1 Hz, 3H)

### Step 3: Tert-butyl 1-ethyl-2-(4-(4,4,5,5-tetramethyl-1,3,2-dioxaborolan-2-yl) benzoyl) hydrazine-1-carboxylate

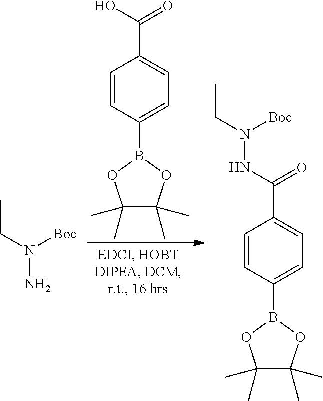

To a solution of 4-(4,4,5,5-tetramethyl-1,3,2-dioxaborolan-2-yl)benzoic acid (774 mg, 3.12 mmol) in dichloromethane (10 mL) was added EDC-HCl (718 mg, 3.74 mmol) followed by HOBt (506 mg, 3.74 mmol), DIPEA (806 mg, 6.24 mmol) and tert-butyl 1-ethylhydrazine-1-carboxylate (500 mg, 3.12 mmol). The mixture was stirred room temperature for 16 hrs. The mixture was diluted with dichloromethane (20 mL), washed with water and brine, dried over anhydrous Na₂SO₄, filtered, and concentrated to dryness. The residue was purified by chromatography (silica gel, 0-10% EtOAc in PE) to give tert-butyl 1-ethyl-2-(4-(4,4,5,5-tetramethyl-1,3,2-dioxaborolan-2-yl) benzoyl) hydrazine-1-carboxylate (480 mg, 39.4% yield) as white solid.

LC/MS (ESI) m/z: 391 (M+H)⁺

¹H NMR (400 MHz, CDCl₃) δ 7.88 (d, J=7.7 Hz, 2H), 7.77 (d, J=7.8 Hz, 2H), 3.67 (dt, J=21.5, 7.1 Hz, 2H), 1.67-1.41 (m, 9H), 1.36 (s, 12H), 1.20 (t, J=7.2 Hz, 3H)

### Step 4: Tert-butyl 2-(4-(2,5-dichloropyrimidin-4-yl) benzoyl)-1-ethylhydrazine-1-carboxylate

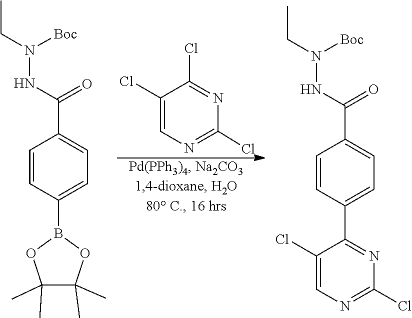

To a solution of tert-butyl 1-ethyl-2-(4-(4,4,5,5-tetramethyl-1,3,2-dioxaborolan-2-yl) benzoyl) hydrazine-1-carboxylate (200 mg, 0.51 mmol) in 1,4-dioxane (5 mL) and

H₂O (1 mL) was added 2,4,5-trichloropyrimidine (104 mg, 0.56 mmol) followed by Na₂CO₃ (108 mg, 0.10 mmol) and Pd (PPh₃)₄ (60 mg, 0.05 mmol). The reaction mixture was degassed under N₂ atmosphere for three times and was stirred at 80° C. for 16 hrs. The mixture was filtered, and the filtrate was concentrated under reduced pressure. The residue was purified by chromatography (silica gel, 0-50% EtOAc in PE) to give tert-butyl 2-(4-(2,5-dichloropyrimidin-4-yl) benzoyl)-1-ethylhydrazine-1-carboxylate (200 mg, 95.0% yield) as white solid.

LC/MS (ESI) m/z: 311 (M−100+H)⁺

¹H NMR (400 MHz, CDCl₃) δ 8.61 (s, 1H), 7.80 (d, J=17.3 Hz, 4H), 3.61-3.55 (m, 2H), 1.42 (d, J=11.8 Hz, 9H), 1.15-1.10 (m, 3H)

### Step 5: Methyl-4-(5-chloro-2-((3-fluoro-4-(morpholine-4-carbonyl) phenyl) amino) pyrimidin-4-yl) benzoate

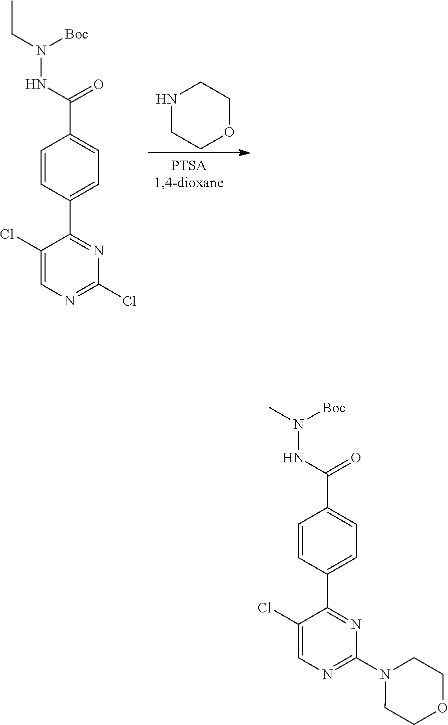 [SMILES: CCN(NC(=O)c1ccc(-c2nc(Cl)ncc2Cl)cc1)C(=O)OC(C)(C)C ; C1COCCN1 ; CN(NC(=O)c1ccc(-c2nc(N3CCOCC3)ncc2Cl)cc1)C(=O)OC(C)(C)C]

To a solution of tert-butyl 2-(4-(2,5-dichloropyrimidin-4-yl) benzoyl)-1-ethylhydrazine-1-carboxylate (200 mg, 0.49 mmol) in 1,4-dioxane (10 mL) was added morpholine (47 mg, 0.54 mmol) followed by p-toluenesulfonic acid (8 mg, 0.05 mmol). The reaction mixture was stirred at 100° C. for 16 hrs. The mixture was concentrated under reduced pressure to dryness. The residue was purified by chromatography (silica gel, 0-16% EtOAc in petroleum ether) to give methyl-4-(5-chloro-2-((3-fluoro-4-(morpholine-4-carbonyl) phenyl) amino) pyrimidin-4-yl) benzoate carbamate (160 mg, yield 71.2% yield) as yellow oil.

LC/MS (ESI) m/z: 462 (M+H)⁺

### Step 6: 4-(5-Chloro-2-morpholinopyrimidin-4-yl)-N′-ethylbenzohydrazide

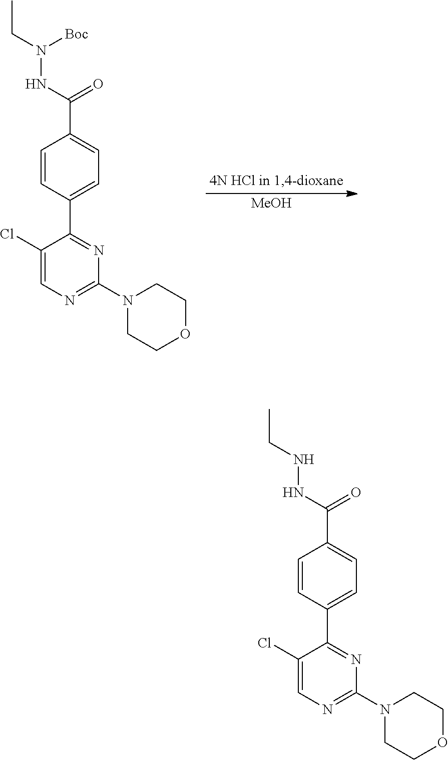 [SMILES: "MeOH" ; CCN(NC(=O)c1ccc(-c2nc(N3CCOCC3)ncc2Cl)cc1)C(=O)OC(C)(C)C ; CCNNC(=O)c1ccc(-c2nc(N3CCOCC3)ncc2Cl)cc1]

To a solution of methyl-4-(5-chloro-2-((3-fluoro-4-(morpholine-4-carbonyl) phenyl) amino) pyrimidin-4-yl) benzoate (160 mg, 0.35 mmol) in MeOH (10.0 mL) was added 4 N HCl in 1,4-dioxane (1.8 mL, 6.93 mmol) at 0° C. The reaction was stirred at 25° C. for 12 hrs. The solvent was removed under vacuum and the residue was diluted with H₂O (20 mL). The mixture was based with saturated aq. NaHCO₃ to pH-8. The mixture was extracted with EtOAc (3×20 mL) and the combined organic layers were washed with brine, dried over anhydrous Na₂SO₄, filtered and concentrated. The residue was washed with acetonitrile to give 4-(5-chloro-2-morpholinopyrimidin-4-yl)-N′-ethylbenzohydrazide (50 mg, 39.8% yield) as white solid.

LC/MS (ESI) m/z: 362 (M+H)⁺

¹H NMR (400 MHz, DMSO-d₆) δ 10.16 (s, 1H), 8.56 (s, 1H), 7.94 (d, J=8.5 Hz, 2H), 7.85 (d, J=8.5 Hz, 2H), 5.14 (s, 1H), 3.70 (dd, J=19.6, 5.2 Hz, 8H), 2.84 (d, J=6.9 Hz, 2H), 1.05 (t, J=7.1 Hz, 3H)

### Synthetic Example 23

### Synthesis of 4-(2-(2-Oxa-7-Azaspiro[3.5]Nonan-7-Yl)Pyrimidin-4-Yl)-N′-Ethylbenzohydrazide

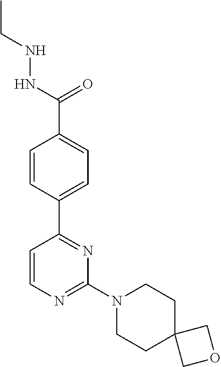 [SMILES: CCNNC(=O)c1ccc(-c2ccnc(N3CCC4(CC3)COC4)n2)cc1]

### Step 1: Tert-butyl-2-(4-(2-(2-oxa-7-azaspiro[3.5]nonan-7-yl)pyrimidin-4-yl)benzoyl)-1-ethylhydrazine-1-carboxylate

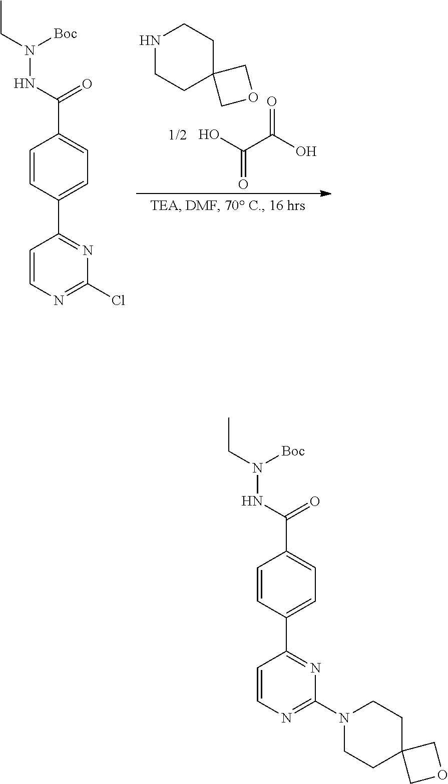

To a mixture of tert-butyl 2-(4-(2-chloropyrimidin-4-yl)benzoyl)-1-ethylhydrazine-1-carboxylate (160 mg, 0.42 mmol) and 2-oxa-7-azaspiro[3.5]nonane oxalate (81 mg, 0.49 mmol) in DMF (8 mL) was added TEA (87 mg, 0.85 mmol). The reaction mixture was stirred at 70° C. for 16 hrs. The mixture was diluted with EtOAc (30 mL), washed with water and brine, dried over anhydrous Na₂SO₄, filtered and concentrated under reduced pressure to give tert-butyl-2-(4-(2-(2-oxa-7-azaspiro[3.5]nonan-7-yl)pyrimidin-4-yl)benzoyl)-1-ethylhydrazine-1-carboxylate (210 mg, 100% yield) as yellow oil.

LC/MS (ESI) m/z: 468(M+H)⁺

### Step 2: 4-(2-(2-Oxa-7-azaspiro[3.5]nonan-7-yl)pyrimidin-4-yl)-N′-ethylbenzohydrazide

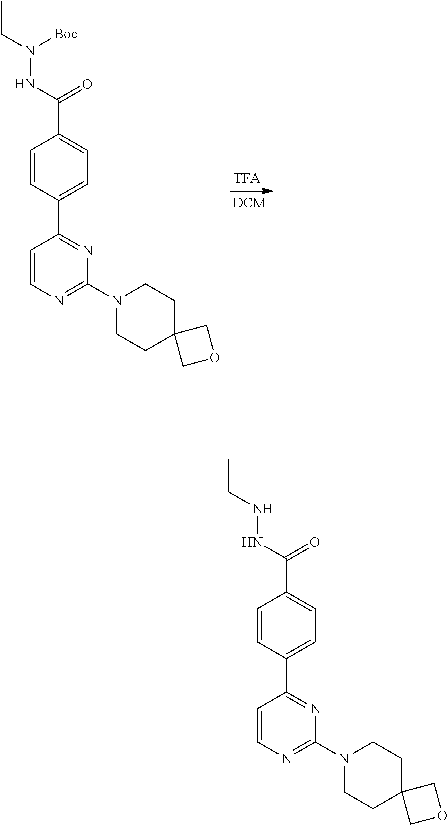 [SMILES: CCN(NC(=O)c1ccc(-c2ccnc(N3CCC4(CC3)COC4)n2)cc1)C(=O)OC(C)(C)C ; CCNNC(=O)c1ccc(-c2ccnc(N3CCC4(CC3)COC4)n2)cc1]

To a solution of tert-butyl-2-(4-(2-(2-oxa-7-azaspiro[3.5]nonan-7-yl)pyrimidin-4-yl)benzoyl)-1-ethylhydrazine-1-carboxylate (210 mg, 0.42 mmol) in dichloromethane (4 mL) was added 4N HCl in 1,4-dioxane (4 mL, 4 mmol) at 0° C. The mixture was stirred at room temperature for 2 hrs. The solvent was removed under vacuum and the residue was diluted with H₂O (10 mL). The mixture was adjusted with saturated aq. NaHCO₃ to pH˜9. The mixture was extracted with EtOAc (3×20 mL) and the combined organic layers were washed with brine, dried over anhydrous Na₂SO₄, filtered and concentrated. The residue was purified by prep-HPLC (C18, 0˜90% acetonitrile in H₂O with 0.1% formic acid) to give 4-(2-(2-oxa-7-azaspiro[3.5]nonan-7-yl)pyrimidin-4-yl)-N′-ethylbenzohydrazide (17.2 mg, 12.2% yield) as yellow solid.

LC/MS (ESI) m/z: 368(M+H)⁺

¹H NMR (400 MHz, DMSO-d₆) δ 11.37 (s, 1H), 8.49 (d, J=5.1 Hz, 1H), 8.28 (d, J=8.4 Hz, 2H), 8.00 (d, J=8.4 Hz, 2H), 7.26 (d, J=5.2 Hz, 1H), 4.41-4.34 (m, 4H), 3.81-3.78 (m, 4H), 3.21-3.14 (m, 2H), 1.91-1.78 (m, 4H), 1.20 (t, J=7.2 Hz, 3H)

### Synthetic Example 24

### Synthesis of 4-(5-Chloro-2-(2-Oxa-7-Azaspiro[3.5]Nonan-7-Yl)Pyrimidin-4-Yl)-N′-Ethylbenzohydrazide

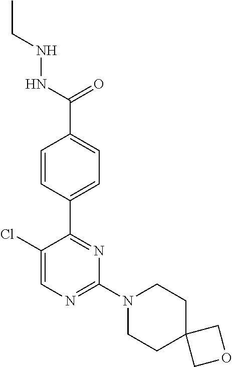 [SMILES: CCNNC(=O)c1ccc(-c2nc(N3CCC4(CC3)COC4)ncc2Cl)cc1]

### Step 1: Tert-butyl 2-(4-(5-chloro-2-(2-oxa-7-azaspiro[3.5]nonan-7-yl)pyrimidin-4-yl)benzoyl)-1-ethylhydrazine-1-carboxylate

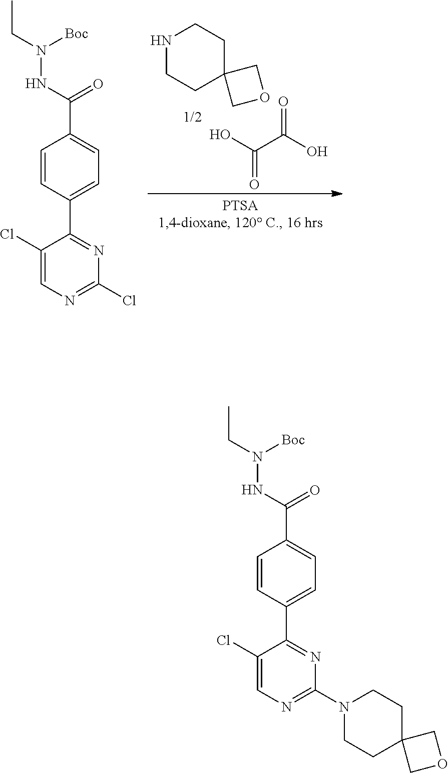

To a mixture of tert-butyl 2-(4-(2,5-dichloropyrimidin-4-yl)benzoyl)-1-ethylhydrazine-1-carboxylate (300 mg, 0.73 mmol) and 2-oxa-7-azaspiro[3.5]nonane oxalate (138 mg, 0.80 mmol) in 1,4-dioxane (3 mL) was added p-toluenesulfonic acid (12.6 mg, 0.07 mmol). The mixture was stirred at 120° C. for 16 hrs. The mixture was diluted with EtOAc (20 mL), washed with water and brine, dried over anhydrous Na₂SO₄, filtered, and concentrated under reduced pressure to give a residue. The residue was purified by chromatography (silica gel, 50%-100% EtOAc in PE) to give tert-butyl 2-(4-(5-chloro-2-(2-oxa-7-azaspiro[3.5]nonan-7-yl)pyrimidin-4-yl)benzoyl)-1-ethylhydrazine-1-carboxylate (174 mg, 47.5% yield) as yellow oil.

LC/MS (ESI) m/z: 502(M+H)⁺

### Step 2: 4-(5-Chloro-2-(2-oxa-7-azaspiro[3.5]nonan-7-yl)pyrimidin-4-yl)-N′-ethylbenzohydrazide

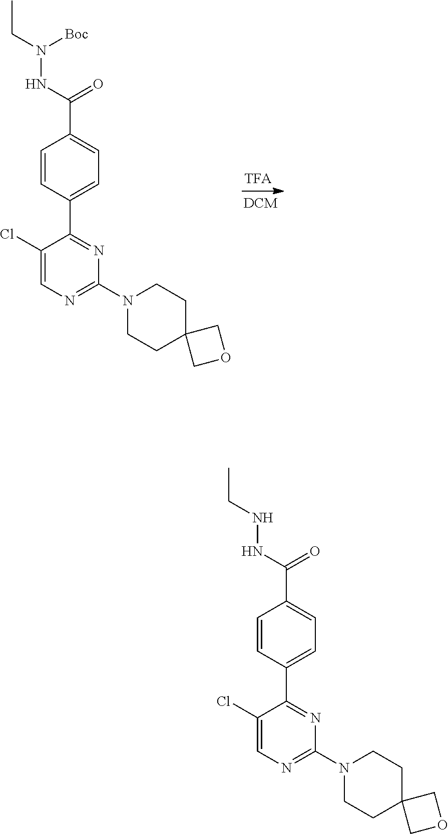 [SMILES: CCN(NC(=O)c1ccc(-c2nc(N3CCC4(CC3)COC4)ncc2Cl)cc1)C(=O)OC(C)(C)C ; CCNNC(=O)c1ccc(-c2nc(N3CCC4(CC3)COC4)ncc2Cl)cc1]

To a solution of tert-butyl 2-(4-(5-chloro-2-(2-oxa-7-azaspiro[3.5]nonan-7-yl)pyrimidin-4-yl)benzoyl)-1-ethylhydrazine-1-carboxylate (174 mg, 0.37 mmol) in dichloromethane (5 mL) was added 4N HCl in 1,4-dioxane (5 mL, 5 mmol) at 0° C. The mixture was at room temperature for 2 hrs. The solvent was removed under vacuum and the residue was diluted with H₂O (10 mL). The mixture was adjusted with saturated aq. NaHCO₃ to pH-9. The mixture was extracted with EtOAc (3×20 mL), and the combined organic layers were washed with brine, dried over anhydrous Na₂SO₄, filtered and concentrated. The residue was purified by prep-HPLC (C18, 0˜90% acetonitrile in H₂O with 0.1% formic acid) to give 4-(5-chloro-2-(2-oxa-7-azaspiro[3.5]nonan-7-yl)pyrimidin-4-yl)-N′-ethylbenzohydrazide (58 mg, 39.1% yield) as white solid.

LC/MS (ESI) m/z: 402(M+H)⁺

¹H NMR (400 MHz, CDCl₃) δ 8.32 (s, 1H), 7.91 (d, J=8.4 Hz, 2H), 7.84 (d, J=8.4 Hz, 2H), 7.67 (s, 1H), 4.50 (s, 4H), 3.81-3.74 (m, 4H), 3.02 (q, J=7.2 Hz, 2H), 1.94-1.90 (m, 4H), 1.18 (t, J=7.2 Hz, 3H)

### Synthetic Example 25

### Synthesis of 4-(2-(2-Oxa-7-Azaspiro[3.5]Nonan-7-Yl)Pyrimidin-4-Yl)-N-Hydroxybenzamide

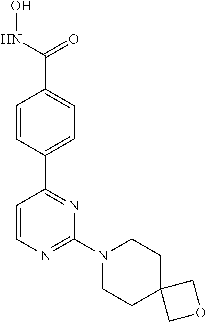 [SMILES: O=C(NO)c1ccc(-c2ccnc(N3CCC4(CC3)COC4)n2)cc1]

### Step 1: methyl 4-(2-chloropyrimidin-4-yl)benzoate

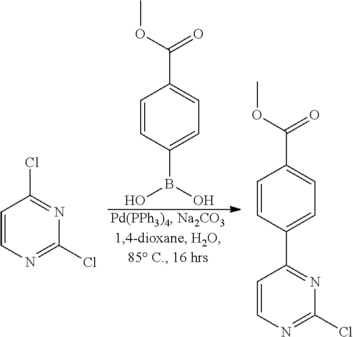 [SMILES: Clc1ccnc(Cl)n1 ; COC(=O)c1ccc(B(O)O)cc1 ; "Pd(PPh3)4, Na2CO31,4-dioxane, H2O,85° C., 16 hrs" ; COC(=O)c1ccc(-c2ccnc(Cl)n2)cc1]

To a solution of 2,4-dichloropyrimidine (3.0 g, 20.13 mmol) in 1,4-dioxane (40 mL) and H₂O (4 mL) was added (4-(methoxycarbonyl)phenyl)boronic acid (5.28 g, 20.13 mmol) followed by Pd(PPh₃)₄ (2.32 g, 2.13 mmol) and Na₂CO₃ (4.26 g, 40.26 mmol). The mixture was degassed under N₂ atmosphere for 3 times and stirred under N₂ atmosphere at 85° C. for 16 hrs. The mixture diluted with dichloromethane (60 mL), washed with water and brine, dried over anhydrous Na₂SO₄, filtered, and concentrated to dryness. The reside was purified by silica gel chromatography (dichloromethane:petroleum ether=5:1) to give methyl 4-(2-chloropyrimidin-4-yl)benzoate (2.5 g, 97.98% yield) as white solid.

LC/MS (ESI) m/z: 249.0 (M+H)⁺

### Step 2: methyl 4-(2-(2-oxa-7-azaspiro[3.5]nonan-7-yl)pyrimidin-4-yl)benzoate

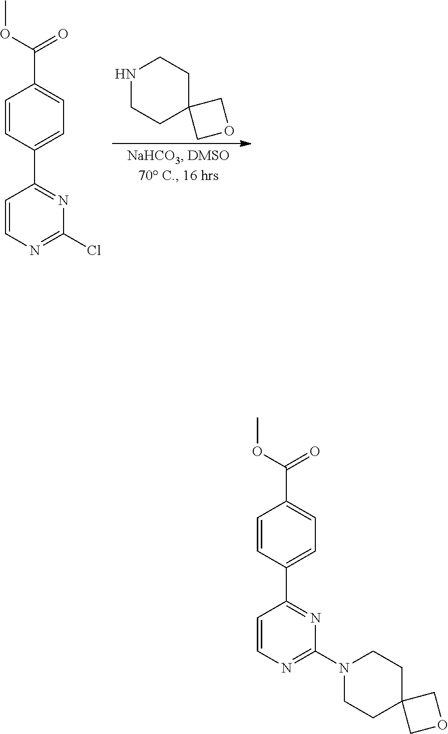 [SMILES: COC(=O)c1ccc(-c2ccnc(Cl)n2)cc1 ; C1CC2(CCN1)COC2 ; COC(=O)c1ccc(-c2ccnc(N3CCC4(CC3)COC4)n2)cc1]

To a mixture of methyl 4-(2-chloropyrimidin-4-yl)benzoate (300 mg, 1.21 mmol) and NaHCO₃ (305 mg, 3.63 mmol) in DMSO (3 mL) was added 2-oxa-7-azaspiro[3.5]nonane (229 mg, 1.33 mmol). The mixture was stirred at 25° C. for 16 hrs. The reaction mixture was triturated in H₂O (10 mL) and filtered to give methyl 4-(2-(2-oxa-7-azaspiro[3.5]nonan-7-yl)pyrimidin-4-yl)benzoate (400 mg, 98.67% yield) as white solid.

LC/MS (ESI) m/z: 340.1 (M+H)⁺

### Step 3: 4-(2-(2-oxa-7-azaspiro[3.5]nonan-7-yl)pyrimidin-4-yl)-N-hydroxybenzamide

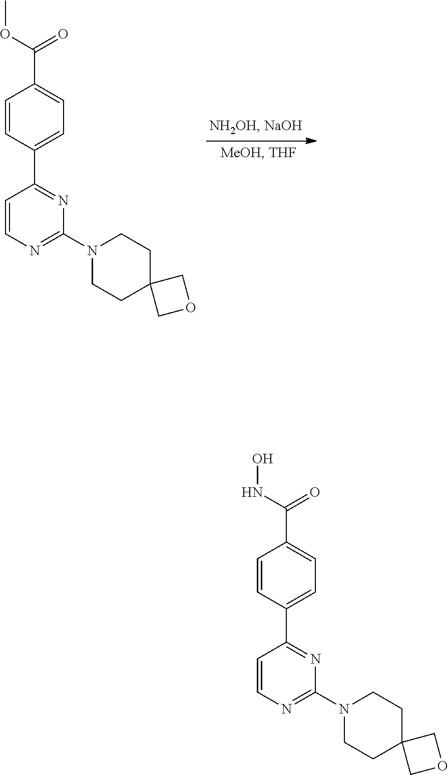 [SMILES: COC(=O)c1ccc(-c2ccnc(N3CCC4(CC3)COC4)n2)cc1 ; O=C(NO)c1ccc(-c2ccnc(N3CCC4(CC3)COC4)n2)cc1]

To a mixture of methyl 4-(2-(2-oxa-7-azaspiro[3.5]nonan-7-yl)pyrimidin-4-yl)benzoate (400 mg, 1.18 mmol) and Hydroxylamine aqueous solution (78 mg, 2.36 mmol) in MeOH (4 mL) and THF (4 mL) was added NaOH (236 mg, 5.9 mmol). The mixture was stirred at 25° C. for 16 hrs. The crude product was purified by Prep-HPLC (trifluoroacetic acid) to give 4-(2-(2-oxa-7-azaspiro[3.5]nonan-7-yl)pyrimidin-4-yl)-N-hydroxybenzamide (39.9 mg, 99.14% yield) as white powder.

LC/MS (ESI) m/z: 341.25 (M+H)⁺

¹H NMR (400 MHz, DMSO) δ 11.34 (s, 1H), 8.46 (d, J=5.1 Hz, 1H), 8.19 (d, J=8.4 Hz, 2H), 7.87 (d, J=8.4 Hz, 2H), 7.23 (d, J=5.2 Hz, 1H), 4.37 (s, 4H), 3.88-3.68 (m, 4H), 2.01-1.67 (m, 4H)

### Synthetic Example 26

### Synthesis of N-Hydroxy-4-(2-(2-Oxo-1-Oxa-3,8-Diazaspiro[4.5]Decan-8-Yl)Pyrimidin-4-Yl)Benzamide

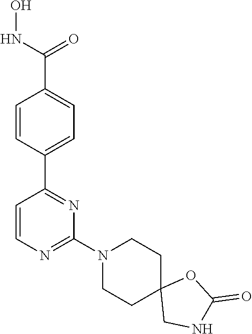 [SMILES: O=C1NCC2(CCN(c3nccc(-c4ccc(C(=O)NO)cc4)n3)CC2)O1]

### Step 1: methyl 4-(2-(4,4-difluoropiperidin-1-yl)pyrimidin-4-yl)benzoate

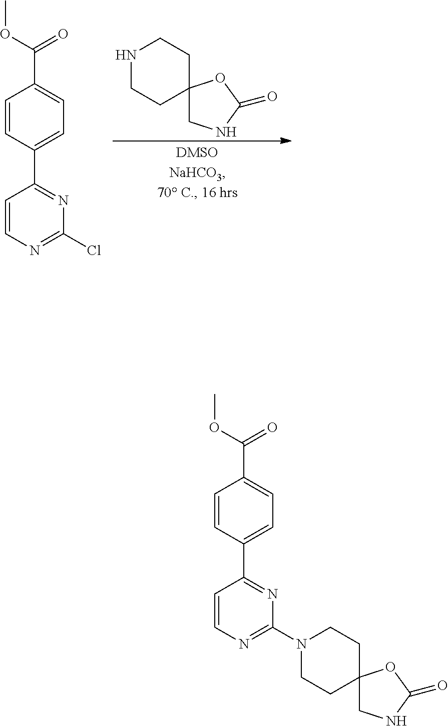 [SMILES: COC(=O)c1ccc(-c2ccnc(Cl)n2)cc1 ; O=C1NCC2(CCNCC2)O1 ; COC(=O)c1ccc(-c2ccnc(N3CCC4(CC3)CNC(=O)O4)n2)cc1]

To a mixture of methyl 4-(2-chloropyrimidin-4-yl)benzoate (350 mg, 1.41 mmol) and NaHCO₃ (355 mg, 4.22 mmol) in DMSO (4 mL) was added 1-oxa-3,8-diazaspiro[4.5]decan-2-one (242 mg, 2.82 mmol). The mixture was stirred at 70° C. for 16 hrs. The reaction mixture was triturated in H₂O (10 mL) and filtered to give methyl 4-(2-(2-oxo-1-oxa-3,8-diazaspiro[4.5]decan-8-yl)pyrimidin-4-yl)benzoate (300 mg, 57.85% yield) as white solid.

LC/MS (ESI) m/z: 369.0 (M+H)⁺

### Step 2: N-hydroxy-4-(2-(2-oxo-1-oxa-3,8-diazaspiro[4.5]decan-8-yl)pyrimidin-4-yl)benzamide

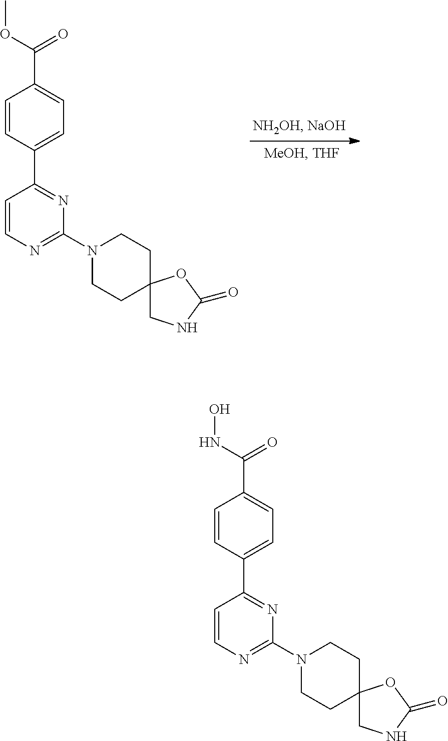 [SMILES: COC(=O)c1ccc(-c2ccnc(N3CCC4(CC3)CNC(=O)O4)n2)cc1 ; O=C1NCC2(CCN(c3nccc(-c4ccc(C(=O)NO)cc4)n3)CC2)O1]

To a mixture of methyl 4-(2-(2-oxo-1-oxa-3,8-diazaspiro[4.5]decan-8-yl)pyrimidin-4-yl)benzoate (300 mg, 0.81 mmol) and Hydroxylamine aqueous solution (79 mg, 2.40 mmol) in MeOH (4 mL) and THF (4 mL) was added NaOH (160 mg, 4.07 mmol). The mixture was stirred at 25° C. for 16 hrs. The crude product was purified by Prep-HPLC (formic acid) to give N-hydroxy-4-(2-(2-oxo-1-oxa-3,8-diazaspiro[4.5]decan-8-yl)pyrimidin-4-yl)benzamide (68 mg, 22.73% yield) as white powder.

LC/MS (ESI) m/z: 370.1 (M+H)⁺

¹H NMR (400 MHz, DMSO) δ 11.34 (s, 1H), 9.12 (d, J=1.5 Hz, 1H), 8.48 (d, J=5.1 Hz, 1H), 8.20 (d, J=8.4 Hz, 2H), 7.87 (d, J=8.4 Hz, 2H), 7.55 (s, 1H), 7.27 (d, J=5.1 Hz, 1H), 4.21 (d, J=13.7 Hz, 2H), 3.69 (dd, J=16.3, 6.4 Hz, 2H), 3.30 (s, 2H), 1.91-1.74 (m, 4H)

### Synthetic Example 27

### Synthesis of 4-(2-(4,4-Difluoropiperidin-1-Yl)Pyrimidin-4-Yl)-N-Hydroxybenzamide

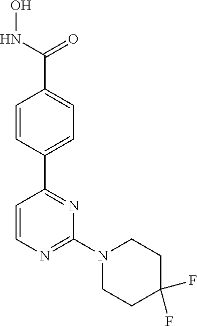 [SMILES: O=C(NO)c1ccc(-c2ccnc(N3CCC(F)(F)CC3)n2)cc1]

### Step 1: methyl 4-(2-(4,4-difluoropiperidin-1-yl)pyrimidin-4-yl)benzoate

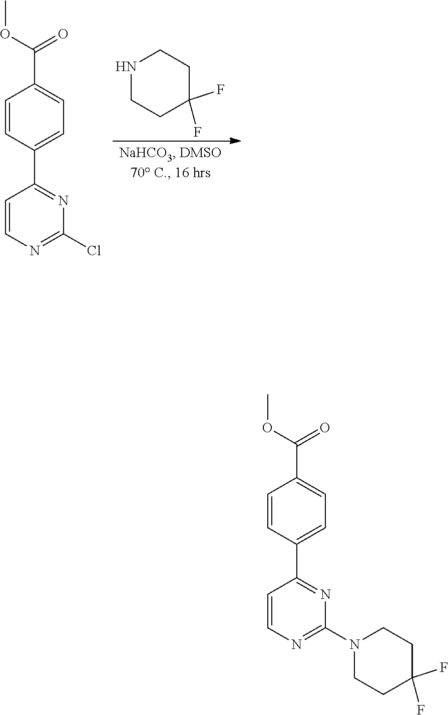 [SMILES: COC(=O)c1ccc(-c2ccnc(Cl)n2)cc1 ; FC1(F)CCNCC1 ; COC(=O)c1ccc(-c2ccnc(N3CCC(F)(F)CC3)n2)cc1]

To a mixture of methyl 4-(2-chloropyrimidin-4-yl)benzoate (300 mg, 1.21 mmol) and NaHCO₃ (305 mg, 3.63 mmol) in DMSO (3 mL) was added 4,4-difluoropiperidine (161 mg, 1.33 mmol). The mixture was stirred at 25° C. for 16 hrs. The reaction mixture was triturated in H₂O (10 mL) and filtered to give methyl 4-(2-(4,4-difluoropiperidin-1-yl)pyrimidin-4-yl)benzoate (400 mg, 98.81% yield) as white solid.

LC/MS (ESI) m/z: 334.1 (M+H)⁺

### Step 2: 4-(2-(4,4-difluoropiperidin-1-yl)pyrimidin-4-yl)-N-hydroxybenzamide

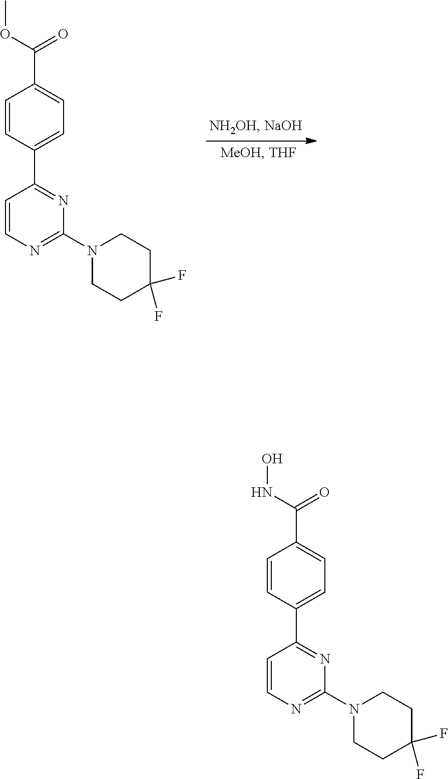 [SMILES: COC(=O)c1ccc(-c2ccnc(N3CCC(F)(F)CC3)n2)cc1 ; O=C(NO)c1ccc(-c2ccnc(N3CCC(F)(F)CC3)n2)cc1]

To a mixture of methyl 4-(2-(4,4-difluoropiperidin-1-yl)pyrimidin-4-yl)benzoate (400 mg, 1.20 mmol) and Hydroxylamine aqueous solution (79 mg, 2.40 mmol) in MeOH (4 mL) and THF (4 mL) was added NaOH (240 mg, 6.00 mmol). The mixture was stirred at 25° C. for 16 hrs. The crude product was purified by Prep-HPLC (trifluoroacetic acid) to give 4-(2-(4,4-difluoropiperidin-1-yl)pyrimidin-4-yl)-N-hydroxybenzamide (124.8 mg, 99.59% yield) as white powder.

LC/MS (ESI) m/z: 335.50 (M+H)⁺

¹H NMR (400 MHz, DMSO) δ 11.34 (s, 111), 8.46 (d, J=5.1 Hz, 1H), 8.19 (d, J=8.4 Hz, 2H), 7.87 (d, J=8.4 Hz, 2H), 7.23 (d, J=5.2 Hz, 1H), 4.37 (s, 4H), 3.88-3.68 (m, 4H), 2.01-1.67 (m, 4H)

### Biological Example 1

### Biochemical Assay Results

Representative compounds were tested for inhibitory activity against HDAC6 according to the following procedures.

TABLE 2
Activity of Representative Compounds

| Compound | HDAC6 | Compound | HDAC6 |
|---|---|---|---|
| No. | IC₅₀ (μM) | No. | IC₅₀ (μM) |
| I-1 | +++++ | I-2 | + |
| I-3 | +++++ | I-4 | +++++ |
| I-5 | +++++ | I-6 | +++++ |
| I-7 | +++++ | I-8 | +++++ |
| I-9 | +++++ | I-10 | +++++ |
| I-11 | +++++ | I-12 | ++++ |
| I-13 | +++++ | I-14 | +++++ |
| I-15 | +++++ | I-16 | +++++ |
| I-17 | +++++ | I-18 | +++++ |
| I-19 | +++++ | I-20 | ++++ |
| I-21 | +++++ | I-22 | + |
| I-23 | +++++ | I-24 | + |
| I-25 | +++++ | I-26 | +++++ |
| I-27 | +++++ | I-30 | ++++ |
| I-31 | +++++ | I-32 | ++++ |
| I-33 | +++++ | I-34 | ++++ |
| I-35 | +++ | I-36 | ++++ |
| I-38 | ++++ | I-39 | ++++ |
| I-40 | ++++ | − | − |

For HDAC6 IC₅₀ activity in Table 2:
+ IC₅₀ concentration greater than 100 μM
++ IC₅₀ concentration ranging from greater than 35 to 100 μM
+++ IC₅₀ concentration ranging from greater than 10 to 35 μM
++++ IC₅₀ concentration ranging from greater than 1 to 10 μM
+++++ IC₅₀ concentration of 1 μM or less
− no value/not tested

### Biological Example 2

### HDAC6 Selectivity

| | Compound I-8 | Control | |
|---|---|---|---|
| HDACs | IC₅₀ (nM) | Compound ID: | IC₅₀ (nM) |
| HDAC1 | 1178 | Trichostatin A | 3.11 |
| HDAC2 | 2799 | Trichostatin A | 7.05 |
| HDAC3 | 990 | Trichostatin A | 4.05 |
| HDAC4 | 11080 | TMP 269 | 114.99 |
| HDAC5 | 7775 | TMP 269 | 160.00 |
| HDAC6 | 7.32 | Trichostatin A | 1.64 |
| HDAC7 | 12400 | TMP 269 | 42.49 |
| HDAC8 | 101.7 | Trichostatin A | 316.00 |
| HDAC9 | 21970 | TMP 269 | 16.20 |
| HDAC10 | 9.15 | Quisinostat | 8.37 |
| HDAC11 | 416.20 | Trichostatin A | 1740 |

Compound I-8 was tested in 10-dose IC₅₀ mode, in singlet, with 3-fold serial dilution starting at 100 millimolar against 11 HDACs. Compound I-8 showed a marked selectivity for HDAC6 when compared to other HDACs.

| | Compound I-5 | Control | |
|---|---|---|---|
| HDACs | IC₅₀ (nM) | Compound ID: | IC₅₀ (nM) |
| HDAC1 | 1639 | Trichostatin A | 3.11 |
| HDAC3 | 1355 | Trichostatin A | 4.05 |
| HDAC8 | 162.10 | Trichostatin A | 316.00 |
| HDAC10 | 73.65 | Quisinostat | 8.37 |
| HDAC11 | 781.69 | Trichostatin A | 1740 |

Compound I-5 was tested as described for compound I-8 above.

### Biological Example 3

### Nanobret Assay for Target Engagement in HEK293 Cells Transiently Transfected With HDAC6

NanoLuc Fusion vector HEK293 cells were transfected with 1 μg of HDAC6-NanoLuc Fusion vector. The transfected cells were treated with compound I-8 starting at 100 μM, 10-dose with 3-fold dilution), SAHA (reference compounds starting at 10 μM, 10-dose with 3-fold dilution for 1 hour. Curve fits were performed only when % NanoBret signal at the highest concentration of compounds was less than 55%.

| | HDAC6 | HDAC6 |
|---|---|---|
| | IC₅₀ (nM) | IC₅₀ (uM) |
| Compound I-8 | 34.67 | 0.035 |
| SAHA | 493.70 | 0.494 |

Compound I-8 exhibited on target HDAC6 inhibition with an IC₅₀ of 34.67 nM.

### Biological Example 4

### Compound I-15 and Compound I-16 from Racemic Compound I-8 Show Potent HDAC6 Activity at 1 Micro Molar

| | Testing Conc. (μM) | IC₅₀ (nM) |
|---|---|---|
| Compound I-15 | 1 | 16 |
| Compound I-16 | 1 | 12.37 |
| Trichostatin A | | 876.20 |

Compound I-15 and compound I-16 were tested in 10-dose IC₅₀ mode, in singlet, with 3-fold serial dilution starting at 1 μM. Data include raw data, % enzyme activity and curve fits.

### Biological Example 5

### HDAC6 Inhibitors for their Ability to Increase Acetylated Alpha-Tubulin Dose-Dependently in SH-SY5Y Neuroblastoma Cells

SH-SY5Y neuroblastoma cells were seeded at 1,000,000 cells/well of three 6-well plates and incubated for 24 hours. Cells were treated with 2 control and experimental HDAC6 inhibitors at 1 μM and 10 μM for 24-hours. Following treatment, cells were lysed, and proteins extracted for western blot. Proteins were separated by gel electrophoresis and transferred to nitrocellulose membranes. Membranes were blocked and probed with primary Abs overnight.

acetyl-α-tubulin (Lys40)(D20G3); Cell Signaling Technologies, #5335S

GAPDH; Millipore Sigma, #MAB374

Membranes were probed with secondary Abs for 1 hour and then the blots were imaged using the iBright 1500 (ThermoFisher)

The Western Blot produced bands for acetyl-α-tubulin (52 kDa) and GAPDH (37 kDa) at the expected sizes in relation to the protein ladder. As a sample loading control, GAPDH indicates that samples had comparable levels of protein loaded in each lane. For all HDAC6 inhibitors, there was an increase in the expression of acetyl-α-tubulin upon treatment with 1 μM/4 μM compound and this was enhanced by treating with 10 μM/40 μM compounds. These results conclude that HDAC6 inhibitors induce acetylation of α-tubulin in SH-SY5Y neuroblastoma cells in a dose-dependent manner.

Samples that showed bands for acetylated alpha tubulin and GAPDH for doses of 1 micromolar and 10 micromolar include compound I-7, compound I-8, compound I-9, compound I-25, compound I-26, and compound I-27. In all, these HDAC6 inhibitors induce acetylation of α-tubulin in SH-SY5Y neuroblastoma cells in a dose-dependent manner.

### Biological Example 6

### HDAC6 Inhibitors in Dose Response Against Levels of Acetylated Alpha-Tubulin in SH-SY5Y Neuroblastoma Cells

### Cell Type: SH-SY5Y

Cell Seeding: Seeded at 2,000 cells/well in 384-well plates 24-hours prior to compound treatment

Compound Treatment: 24-hour

### Drug Concentrations & Replicates

HDAC6 inhibitor compounds tested (1 control and 6 experimental)

9-point dose-response curves for all compounds, starting at 30 μM with 3-fold serial dilutions

4 technical replicate wells per condition tested

Cells were seeded in 90 μL media. 10 μL of 10× compound solutions added to 90 μL media for 24-hours

### ICC Methods

Fixation: 4% PFA for 15 minutes (after removing all media and washing with DPBS+0.1% Tween-20)

Permeabilization: 0.1% Triton X-100 for 20 minutes

Primary antibody: acetyl-α-tubulin (Lys40/D20G3; Cell Signaling Technologies, #5335S) 1:1000 dilution in DPBS+5% goat serum+0.1% Tween-20, 1 hour at room temp on orbital shaker

Secondary antibody: Goat anti-Rabbit IgG (H+L) Highly Cross-Adsorbed Secondary Antibody, Alexa Fluor™ Plus 488, Catalog #A32731, 1:1000 dilution+Hoechst 33342, 1:10,000 dilution in DPBS+5% goat serum+0.1% Tween-20, 1 hour at room temp on orbital shaker

### Imaging Methods

High-content imaging system: Image Xpress Micro Confocal from Molecular Devices

Automated imaging of 4 regions per well (same regions in all wells) were obtained with a 20× objective lens

Max projection images were obtained from 5-image Z-stack in each well-region with confocal imaging

### Quantification Method

Hoechst stain used to create mask of nuclei

Integrated fluorescence intensity of acetyl-α-tubulin was calculated using an automated image-analysis algorithm in Meta Xpress software

Average intensity per cell was calculated by dividing the integrated fluorescence intensity by the number of nuclei.

Dose-response curves for the HDAC6 inhibitors were graphed in Prism.

### ICC Results

Secondary-only controls were clean and DMSO-treated controls exhibited low levels of acetyl-α-tubulin immunostaining, as expected.

Cytotoxicity was observed in most compounds at 30 μM concentration. Several compounds exhibited toxicity at 10 μM and 3 μM as well, as seen in the example images shown for each compound. All the HDAC6 inhibitor compounds induced increases in acetyl-α-tubulin, and IC₅₀ values ranged from 0.82 μM to 4.6 μM. The control compound run with this assay, ACY-1215 (ricolinostat), had IC₅₀ value of 1.78 μM. Technical replicates for each condition tested were in strong agreement and identify a clear rank-ordering of the most and least potent HDAC6 inhibitors. All compounds had IC₅₀ values <5 μM and few compounds had IC₅₀ values <1 μM. ACY-1215 (ricolinostat) has the following structure:

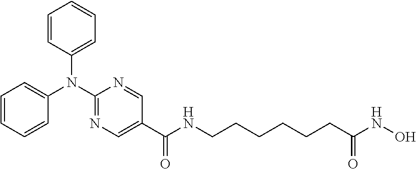 [SMILES: O=C(CCCCCCNC(=O)c1cnc(N(c2ccccc2)c2ccccc2)nc1)NO]

Immunocytochemistry (ICC) and Western Blotting was used as an experimental in vitro evaluation of dose response efficacy compound I-8 in SH-SY5Y neuroblastoma cells. The testing showed HDAC6 inhibitors induce acetylation of α-tubulin in SH-SY5Y neuroblastoma cells in a dose-dependent manner.

### Biological Example 7

### Oral Bioavailability and Brain/CSF Uptake Study of Compound I-8 in Male Swiss Albino Mice

Compound I-8 was evaluated for bioavailability, pharmacokinetics and brain/csf to plasma distribution in mouse plasma following oral and intravenous administration of compound I-8 in male Swiss albino mouse.

A total of 24 male Swiss albino mouse were allotted for the study, Animals were allotted to 2 groups containing 12 mice/group, on day of dosing all the animals were fasted 3 hours before dosing. The test formulation was administered as a single dose by intravenous and oral route via tail vein and oral gavage needle at a dose of 1 and 5 mg/kg with a dose volume of 5 and 10 mL/kg, respectively.

Approximately 0.1 mL of blood sample from each animal was collected via retro orbital at predetermined time points (3 animals/timepoint) in pre-labeled Eppendorf tubes containing anticoagulant. After blood collection at 2 h, 4 h and 6 h, CSF and whole brain was withdrawn, and brain tissue was homogenized as a terminal sampling. Blood samples were centrifuged at 10000 rpm for 5 min under refrigeration (2-4° C.) within 0.5 h to obtain plasma. The separated plasma samples were collected in pre labeled tubes within 60 min of separation and stored at −20° C. up to 24 h. Post 24 h sample collection, all plasma samples were transferred to deep freezer custodian to store below or at −70° C. until shipment.

Brain tissues were blotted and dried and then transferred to 15 mL falcon tubes. Initially all tissue samples were stored at −20±3° C. Later all brain tissue samples were homogenized with PBS at 1:4 ratio and the Brain homogenate was transferred to deep freezer (−70±10° C.) and stored until bioanalysis.

Mouse plasma, CSF and Brain homogenate concentrations were analyzed to quantify compound I-8 using a fit-for purpose LC-MS/MS method with LLOQ of 0.98 ng/mL. The PK parameters were evaluated using Phoenix WinNonlin® Ent-Version 8.3 by non-compartmental analysis.

Intravenous administration of compound I-8 at dose of 1 mg/kg to male Swiss Albino mice, revealed very high clearance ˜328.28 mL/min/kg compared with normal hepatic blood flow (i.e., 90 mL/min/kg) this abnormal high clearance could be one of the reasons of stability or partitioning to red blood cells or high tissue distribution. Steady state and central compartment volume of distribution was found to be very high suggesting high tissue distribution.

Oral administration of compound I-8 at dose of 5 mg/kg to male Swiss Albino mice, demonstrated no lag phase in absorption and time to reach peak concentration was 8 h. Compound I-8 exhibited moderate to high systemic availability. Oral bioavailability was found to be 53%.

| | | | AUCₗₐₛₜ | AUC_{inf} | Cl | | |
|---|---|---|---|---|---|---|---|
| Group | Tₘₐₓ | C₀/Cₘₐₓ | (ng · h/ | (ng · h/ | (mL/ | Vₛₛ | Vd |
| (Dose) | (h) | (ng/mL) | mL) | mL) | min/kg) | (L/kg) | (L/kg) |
| G1 | NA | 27.33 | 27.12 | 50.77 | 328.28 | 187.74 | 171.31 |
| (1 mg/kg) | | | | | | | |
| G2 | 8 | 18.32 | 72.31 | NR | NA | | |
| (5 mg/kg) | | | | | | | |

| | T_{1/2} | MRT | |
|---|---|---|---|
| Group (Dose) | (h) | (h) | % F |
| G1 (1 mg/kg) | 6.03 | 3.28 | — |
| G2 (5 mg/kg) | NR | 4.75 | 53 |

C₀: Back extrapolated concentration at time zero;
^{a} Nominal dose and AUC_{inf} of IV and PO were used to calculate bioavailability;
NA: Not applicable;
NR: not reportable due to improper elimination phase.

### Biological Example 8

### Pre-Clinical ALS Mouse Model Efficacy Results

Effect of test compound I-8 in Cyagen, male B6SJL.SOD1-G93A 9-week-old transgenic mouse model of amyotrophic lateral sclerosis

Compound I-8 effect was evaluated in Cyagen, Male B6SJL.SOD1-G93A 9-week-old mice, an animal model of amyotrophic lateral sclerosis. Animals in disease control group, demonstrated disease progression compared to vehicle control group and the same was evident from neurological scoring. Also, disease control group demonstrated significant impairment in motor strength, motor coordination and motor balance compared to vehicle control group at week 10, 12, 14 and 16.

Improvement was observed in motor behavior at both the doses of compound I-8 (G3 and G4) compared to disease control group from week 10 until week 16. Only significant improvement was observed in motor balance of G4 group at week 10 compared to G2, disease control group.

Future studies on compound I-8 which is a racemic was treated in these male B6SJL.SOD1-G93A mice was a racemic compound and was separated using chiral separation techniques in the compound I-15 and compound I-16 isomers. The mice PK studies show promising oral, and brain up take with compound I-16.

### Biological Example 9

### Effect of Test Compound I-16 in TG2576 Animal Model of Alzheimer'S Disease

Alzheimer's disease (AD) accounts for 60-80% of these cases. AD is an irreversible and progressive neurodegenerative disorder that presents with cognitive impairment and memory loss as the primary clinical symptoms. The disease is mainly caused by the accumulation of extracellular amyloid β (Aβ) and intracellular neurofibrillary tau tangles. Structural brain changes are thought to begin 20 years or more before the onset of clinical symptoms. The disease has several phases, which include a long preclinical phase with no clinical symptoms, a mild cognitive impairment phase, and a disease phase. The length of each phase is affected by the patient's age, genetics, gender, and other factors. Although AD has been studied over time, fundamental treatment strategies for the disease remain underdeveloped. It is therefore important to develop new therapeutic medications for AD patients that are more effective than the existing treatments.

30 (20 B6; SJL-Tg (APPSWE) 2576 Kha+10 wild type) 6 months old male mice are procured from Jackson laboratories. All animals quarantine for 7-14 days and acclimatize for at least one day prior to experiment initiation. During this period, the mice are observed daily for clinical signs.

B6; SJL-Tg (APPSWE) 2576 Kha mice are weighed and randomized into 2 different groups. Wild type animals are grouped as such. All the test compounds are freshly formulated and administered to respective animals of each group as per the body weight.

After randomization, basal behavior of the animals is recorded and analyzed in open field, Y-Maze and EPM. Post basal behavior, animals are treated with compound I-16 for the duration of 3 months (i.e., from 6 month till 9 month. During the treatment duration, animals are subjected to different behavioral tests such as OFT, Y-maze and EPM at 7, 8 and 9 months. At the end of the experiment (i.e., at 9 months), animals are subjected to Morris water maze test.

At termination, animals are dosed and then under mild anesthesia blood will be collected retro-orbitally and separated plasma is utilized for bioanalysis (G3) and ex vivo analysis (optional). Then animals are euthanized under CO₂ asphyxiation and brain is collected and divided into two equal hemispheres. One hemisphere is fixed in 10% NBF for immunohistochemistry and from another hemisphere, dissected cortex and hippocampus is utilized for ex vivo analysis.

### Summary of Observations:

Y Maze: G1, wild type age match control+vehicle and G3, Tg2576 AD disease mice+compound I-16 50 mg/kg did not show any significant difference in number of spontaneous alterations but G1, wild type age match control+vehicle showed significant increase in % spontaneous alteration compared to G2, Tg2576 AD disease mice+vehicle indicating the Alzheimer's kind of symptoms in G2 group.

Elevated Plus Maze: G1, wild type age match control+Vehicle and G3, Tg2576 AD disease mice+compound I-16 50 mg/kg did not show any significant difference in time spent in both open and closed arms compared to G2, Tg2576 AD disease mice+vehicle.

Open Field Test: G1, wild type age match control+vehicle and G3, Tg2576 AD disease mice+compound I-16 50 mg/kg did not show any significant difference in total distance travelled, mean velocity and in time spent in both central and peripheral zone compared to G2, Tg2576 AD disease mice+vehicle.

### Biological Example 10

### In Vivo Pharmacology Study Parameters: Compound I-16

Compound I-16 (free base; purity of 98.77%) was tested in a vehicle of PEG 400 (30%), Tween 80 (2%), and water (68%) at a concentration of 1 mg/mL. Dose formulations were prepared every day prior to dose administration. Oral dosages of 50 mg/kg were used unless otherwise indicated. 80 mg/kg doses were similarly prepared at a concentration of 8 mg/mL Male mice aged 6 months were used. 30 total animals were used with 10 animals in each group and 3 groups total. Groups were separated according to the following parameters.

| | | Dose; Route of | Dose | Concen- |
|---|---|---|---|---|
| | | administration. | volume | tration |
| Group | Treatment | Regimen | (mL/kg) | (mg/mL) |
| G1 | Wild type age match | NA | 10 | NA |
| | control + Vehicle | | | |
| G2 | Tg2576 AD Disease | NA | 10 | NA |
| | Mice + Vehicle | | | |
| G3 | Tg2576 AD Disease | 50 mg/kg | 10 | 5 |
| | Mice + compound I-16 | 80 mg/Kg, p.o.* | | |

*80 mg/Kg has been administered from day 36

Mice were weighed and randomized into groups. All the test formulations were freshly administered to respective animals of each group as per body weight. Basal behavior of the animals was recorded and analyzed in open field, Y-Maze, and EPM. Post basal behavior, animals were treated with compound I-16 for the duration of 3 months (i.e., from 6 months until 9 months). During the treatment duration, animals were subjected to different behavioral tests such as OFT, Y-maze, and EPM at 7, 8 and 9 months. At the end of experiments (i.e., at 9 months) animals were subjected to Morris water maze test.

At termination, animals were given a final dose, and under mild anesthesia, blood was collected retro-orbitally and separated plasma were utilized for bio analysis (G3) and ex vivo analysis. Then animals were euthanized under CO₂ asphyxiation and brain tissue was collected and divided into two equal hemispheres. One hemisphere was fixed in 10% NBF for immunohistochemistry and from another hemisphere, dissected cortex and hippocampus was utilized for ex vivo analysis.

### Biological Example 11

### Pharmacology Study Observations and Parameters: Compound I-16

### Clinical Sign & Mortality

Animals were observed for clinical signs to treatment throughout the study. Cage-side observations were made to detect any changes and general activity of the animals. After dose administration, all the animals were observed carefully for treatment related clinical signs, including morbidity and mortality.

### Open Field Test

Prior to the treatment initiation at 7, 8 and 9 months, animals were subjected to open field test. Locomotor activities were observed in a squared open field arena (plastic; 42(L)×42(W)×36(H) cm) with a light intensity of approximately 200 lux. The total distance travelled, and velocity were quantified using an automated video tracking software Ethovision XT for the duration of 10 minutes. A camera, located directly above the centre of the open field, records the session. Locomotor activities were expressed as the total distance travelled and velocity.

### Y-Maze Test

To test spatial working memory, animals were subjected to spontaneous alteration test in Y-maze (Arm length—57 cm). During the test, the animal was placed at the end of one arm and allowed to explore the arena for a duration of 10 minutes. During the testing session, the number of entries in each arm were recorded and % alterations were calculated.

### Elevated Plus Maze

Animals were subjected to elevated plus maze to evaluate anxiety-like behavior. Animals were placed in the centre of the arena with two open and two closed arms facing towards closed arms and allowed to explore the arena for a duration of 5 minutes. Animal behavior was video recorded using overhead video recording camera. Total time spent in the closed and open arms were quantified using automated animal tracking software Ethovision XT.

### Morris Water Maze Test

Animals were subjected to MWM to test spatial working memory. Animals were subjected to habituation, acquisition, and probe trial phase. On day 0, animals were habituated to the Morris water maze tank filled with water for a duration of 90 seconds. During the acquisition phase from day 1 to 4, animals were allowed to swim or search the hidden platform for a duration of 60 seconds. Animals were subjected to a total of 4 trials from 4 different positions each day. On the day of the probe test, animals were allowed to swim for a duration of 60 seconds without platform and time spent in the target quadrant was recorded. During the acquisition phase, latency to reach the platform was calculated.

### Bioanalysis

At the end of the experiment, animals in group G3 were dosed with the test compound, blood was collected under mild anesthesia, and separated plasma was utilized for drug bioanalysis by LC-MS/MS.

All data were expressed as the mean±SEM and presented in tabular and/or graphical form. Statistical analysis was conducted using a one-way ANOVA/Two-way ANOVA followed by Dunnett's/Bonferroni's Multiple Comparison Test. Data were considered statistically significant if the P value was less than 0.05. In the Figures and experimental examples related to this data, the following marks have the following indications:
* Denotes a significant difference as compared to G2.
# Denotes a significant difference as compared to G1.

Based on a two-way ANOVA followed by Bonferroni's Multiple Comparison Test:
#/*P<0.05
##/**P<0.01
####/***P<0.001
####/****P<0.0001

### Results and Discussion

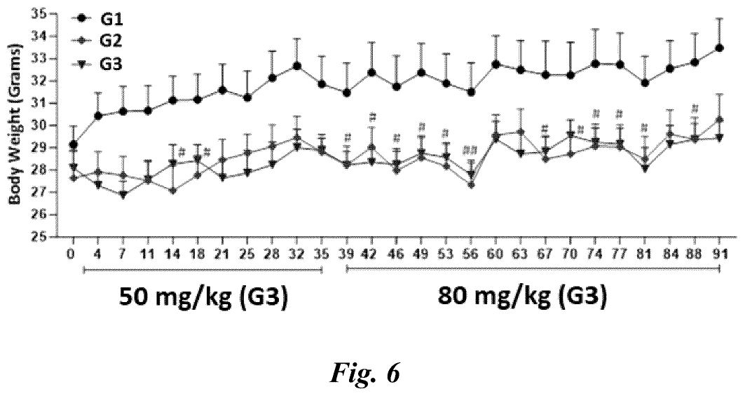

No mortality was observed during the study and all the animals were clinically performed well during the entire study. Body weight data was collected and selected data is shown in the table below and in FIG. 6

| Day | G1 | G2 | G3 |
|---|---|---|---|
| Day 0 | 29.14 ± 0.83 | 27.64 ± 1.18 | 28.08 ± 0.84 |
| Day 4 | 30.42 ± 1.03 | 27.91 ± 0.91 | 27.31 ± 0.52 |
| Day 7 | 30.63 ± 1.12 | 27.76 ± 0.86 | 26.85 ± 0.66 |
| Day 14 | 31.13 ± 1.08 | 27.07 ± 1.10^{#} | 28.27 ± 0.87 |
| Day 21 | 31.58 ± 1.16 | 28.45 ± 0.92 | 27.65 ± 0.86 |
| Day 28 | 32.13 ± 1.20 | 29.06 ± 0.97 | 28.23 ± 1.02 |
| Day 35 | 31.85 ± 1.25 | 28.81 ± 0.78 | 28.88 ± 0.55 |
| Day 42 | 32.38 ± 1.34 | 29.03 ± 0.88^{#} | 28.35 ± 0.63 |
| Day 49 | 32.37 ± 1.31 | 28.55 ± 0.90^{#} | 28.76 ± 0.76 |
| Day 56 | 31.50 ± 1.30 | 27.33 ± 0.99^{##} | 27.77 ± 0.67 |
| Day 63 | 32.49 ± 1.32 | 29.71 ± 1.03^{#} | 28.72 ± 1.03 |
| Day 70 | 32.26 ± 1.47 | 28.72 ± 0.92^{#} | 29.54 ± 0.72 |
| Day 77 | 32.73 ± 1.40 | 29.03 ± 1.00^{#} | 29.16 ± 0.71 |
| Day 84 | 32.55 ± 1.26 | 29.61 ± 1.08 | 29.14 ± 0.86 |
| Day 91 | 33.48 ± 1.32 | 30.27 ± 1.12 | 29.41 ± 0.78 |

### Y-Maze Test

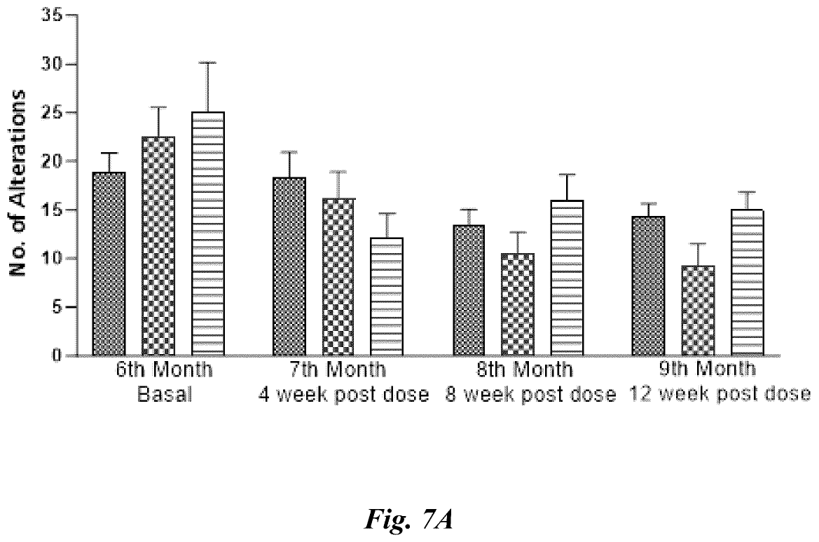

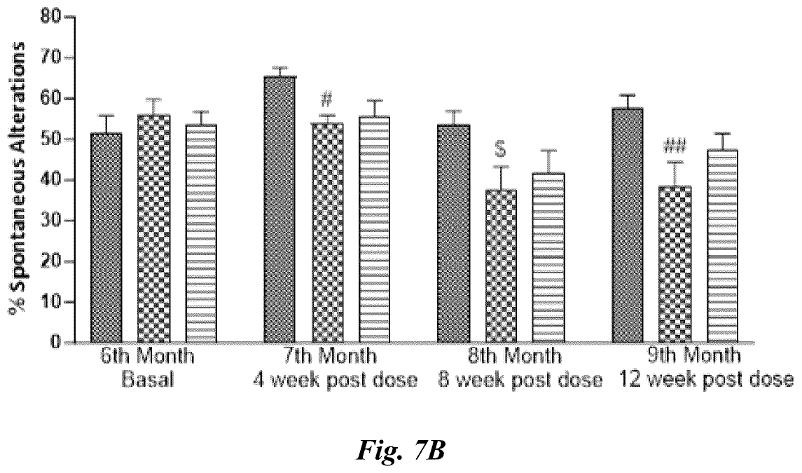

The effect of compound I-16 on Y-Maze is shown in the tables below and in FIGS. 7A and 7B. Percentage of spontaneous alteration is calculated according to the following equation:

% Spontaneous alteration=(Spontaneous alteration/Total Arm entries−2)×100

G1 and G3 did not show any significant difference in the number of spontaneous alterations but G1 showed significant increase in % spontaneous alteration compared to G2 indicating the Alzheimer's kind of symptoms in G2 group.

| | | |
|---|---|---|
| Treatment | # of Spontaneous | % Spontaneous |
| Group | Alterations | Alterations |

6th months (Basal)

| | | |
|---|---|---|
| G1 | 18.80 ± 2.06 | 51.36 ± 4.36 |
| G2 | 22.60 ± 2.92 | 56.03 ± 3.72 |
| G3 | 25.00 ± 5.19 | 53.60 ± 3.25 |

7th months

| | | |
|---|---|---|
| G1 | 17.20 ± 2.59 | 62.48 ± 3.56 |
| G2 | 15.10 ± 2.66 | 56.81 ± 3.47^{#} |
| G3 | 18.70 ± 6.96 | 57.59 ± 3.85 |

8th months

| | | |
|---|---|---|
| G1 | 13.50 ± 1.59 | 53.66 ± 3.25 |
| G2 | 10.50 ± 2.19^{$} | 37.74 ± 5.56 |
| G3 | 16.00 ± 2.59 | 41.65 ± 5.52 |

9th months

| | | |
|---|---|---|
| G1 | 14.40 ± 1.28 | 57.77 ± 3.00 |
| G2 | 9.30 ± 2.19^{##} | 32.34 ± 4.02 |
| G3 | 16.00 ± 2.48 | 43.66 ± 4.55 |

### Elevated Plus Maze

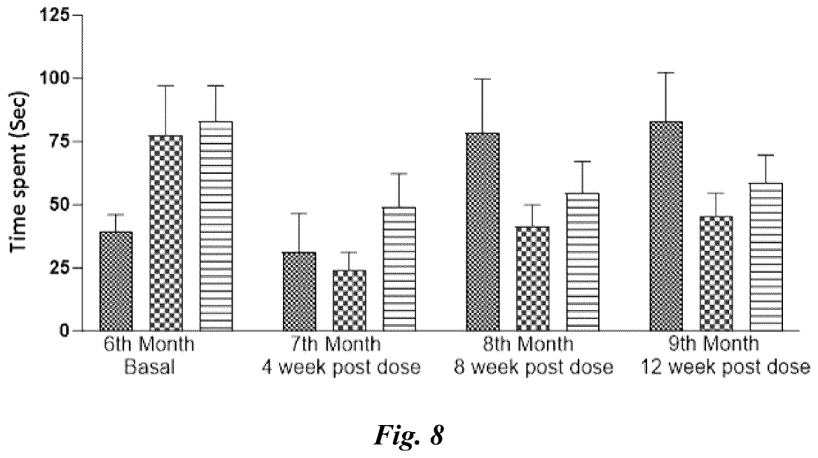

The effect of compound I-16 on Elevated Plus Maze is shown in the tables below and and FIG. 8. G1 and G3 did not show any significant difference in time spent in both open and closed arms compared to G2.

| | | |
|---|---|---|
| Treatment Group | Open arms | Closed arms |

6th months (Basal)

| | | |
|---|---|---|
| G1 | 39.58 ± 6.47 | 191.70 ± 19.04 |
| G2 | 72.20 ± 18.39 | 155.34 ± 25.27 |
| G3 | 82.94 ± 14.23 | 110.86 ± 16.69 |

7th months

| | | |
|---|---|---|
| G1 | 31.25 ± 15.32 | 235.52 ± 20.08 |
| G2 | 30.22 ± 10.63 | 232.68 ± 15.26 |
| G3 | 73.82 ± 27.58 | 165.48 ± 28.89 |

8th months

| | | |
|---|---|---|
| G1 | 78.54 ± 21.24 | 221.46 ± 21.24 |
| G2 | 41.52 ± 8.57 | 258.48 ± 8.57 |
| G3 | 54.73 ± 12.41 | 245.27 ± 12.41 |

9th months

| | | |
|---|---|---|
| G1 | 83.30 ± 19.06 | 216.70 ± 19.06 |
| G2 | 45.80 ± 8.83 | 254.20 ± 8.83 |
| G3 | 58.55 ± 11.16 | 241.46 ± 11.16 |

### Open Field Test

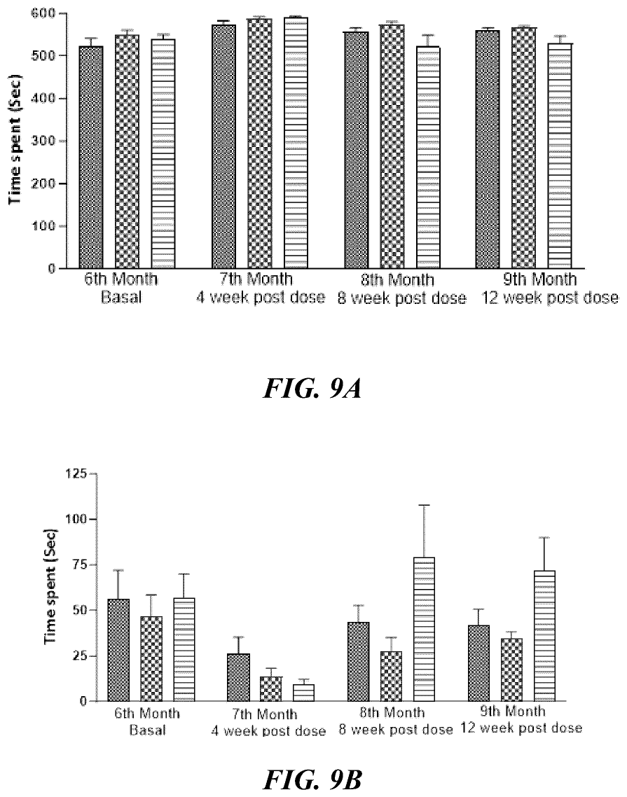

The effect of compound I-16 on Open field Test is shown in the tables below and FIGS. 9A and 9B. G1 and G3 did not show any significant difference in total distance, velocity, time spent in center & peripheral zones compared to G2.

| | | |
|---|---|---|
| Treatment Group | Central zone | Peripheral zone |

6th months (Basal)

| | | |
|---|---|---|
| G1 | 56.22 ± 15.78 | 522.92 ± 17.81 |
| G2 | 46.37 ± 12.10 | 549.51 ± 10.98 |
| G3 | 57.11 ± 12.95 | 537.81 ± 12.63 |

7th months

| | | |
|---|---|---|
| G1 | 26.38 ± 9.12 | 573.62 ± 9.12 |
| G2 | 7.51 ± 3.09 | 592.49 ± 3.09 |
| G3 | 25.12 ± 12.39 | 574.88 ± 12.39 |

8th months

| | | |
|---|---|---|
| G1 | 43.44 ± 9.34 | 556.56 ± 9.34 |
| G2 | 50.10 ± 23.71 | 549.90 ± 23.71 |
| G3 | 79.09 ± 28.86 | 520.91 ± 28.86 |

9th months

| | | |
|---|---|---|
| G1 | 42.09 ± 8.56 | 557.91 ± 8.56 |
| G2 | 34.39 ± 4.10 | 566.61 ± 3.60 |
| G3 | 61.69 ± 12.36 | 538.31 ± 12.36 |

### Morris Water Maze

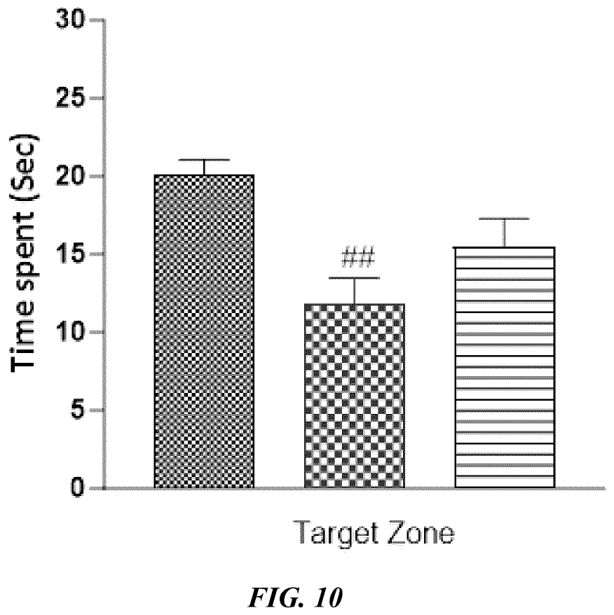

The effect of Compound I-16 on Morris Water Maze is shown in the table below and FIG. 10. G1 showed a significant increase in the time spent in target zone compared to G2 indicating the Alzheimer's symptoms in G2 group.

| Treatment Group | NW (Target Quadrant) |
|---|---|
| G1 | 18.66 ± 1.50 |
| G2 | 11.84 ± 1.65^{##} |
| G3 | 15.49 ± 1.80 |

### Bioanalysis in Plasma

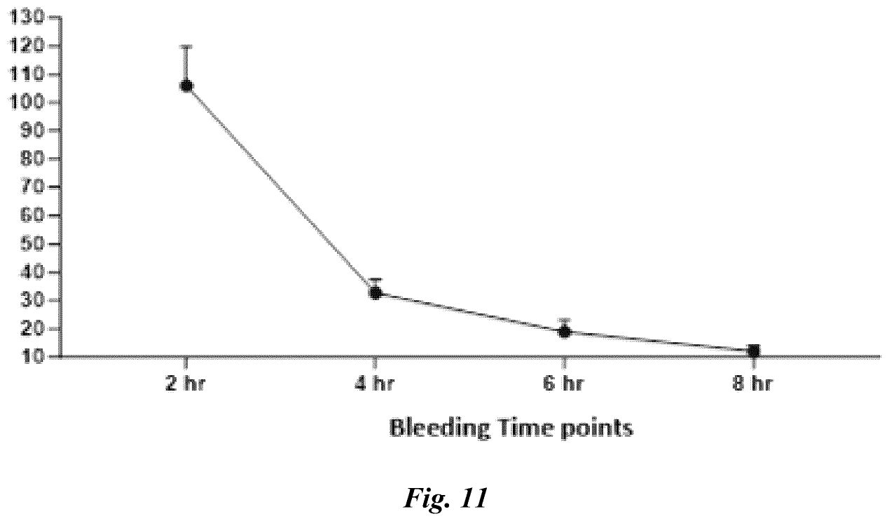

The drug concentration analysis of compound I-16 is shown in the table below and in FIG. 11.

| | | | | Brain Conc |
|---|---|---|---|---|
| Plasma Concentrations (ng/mL) | | | | (ng/mL) |
| 2 hr post | 4 hr post | 6 hr post | 8 hr post | 9 hr post |
| dose | dose | dose | dose | dose |
| 105.86 ± 13.76 | 32.64 ± 4.75 | 19.04 ± 4.16 | 12.13 ± 2.10 | No Peak |

### Summary of Results & Discussion

Alzheimer's disease research employed a Tg2576 mouse model to evaluate potential treatments for the disease. The APPK670/671L mutant type of amyloid precursor protein (APP) linked to early-onset familial Alzheimer's disease (AD), associated with amyloid plaque development, progressive cognitive impairments, are overexpressed in Tg2576 mice.

B6; SJL-Tg (APPSWE) 2576 Kha mice and age matched wild type mice at the age of 6 months were involved in the study. At this age animals were subjected to different behavioral tests like Y maze for cognitive impairment, Elevated Plus Maze (EPM) and Open filed test (OFT) for anxiety, which is a comorbidity of Alzheimer's disease, and measured their basal scoring.

The dosing was initiated in the animals of G3 initially for one month. Then at the age of 7 months all the animals were subjected to all the behavioral tests again.

In the Y maze investigation, the G2 group at this age had a significant decline in the percentage of spontaneous alterations compared to G1 age matched wild type mice, a test of mice spatial working memory that indicates the cognitive impairment which is an onset of disease.

From the start of 8^{th} month age, the dose of compound I-16 in G3 increased to 80 mg/kg. After a month of treatment, G2 group showed significant decrease in percentage of spontaneous alterations compared to G1, supporting the constant disease progression in Tg2576 AD Disease Mice.

In the test of EPM at the 8^{th} and 9^{th} months, G1 showed some trend in increase of time spent in open arm compared to G2, which may have been related to increase of anxiety in G2, but it was not significant. However, in OFT there was no significant difference in time spent at the center zone among all the groups.

In the 9^{th} month all the mice were subjected to Morris Water Maze (MWM) test, a behavioral test used to assess spatial learning and memory, where the time spent by mice in target zone was measured. G2 group showed a significant decrease in the time spent in target zone compared to G1, which correlated the impairment in the cognition of G2 mice (i.e., a symptom of Alzheimer's disease).

After the water maze test, G3 mice were bled for plasma drug concentration analysis at different time points, and there was a time course dependent decrease in compound I-16 concentrations.

### Biological Example 12

### Effect of Compound I-8 on ALS Mouse Model

The test compound used in these studies was compound I-8. Vehicle for test compound formulations can be found below:

| | |
|---|---|
| Vehicle | PEG-400 (30%) + Tween 20 (2%) + Milli Q |
| | water (68%) − until day 6 of dosing; |
| | Milli Q water − was used from day 7 onward. |
| Dose | 15 mg/kg and 30 mg/kg, p.o. and QD |
| Formulation Type | Solution |
| Dose Volume | 10 mL/kg |

Experimental design is further detailed in the table below:

| | | Dose; Route of administration; | | |
|---|---|---|---|---|
| Group | Treatment | Regimen, Dosage volume | Dose/volume | |
| G1 | Vehicle | 10 mL/kg, p.o., Q.D | 10 | mL/kg |
| | Control | | | |
| G2 | Disease | 10 mL/kg, p.o., Q.D. | 10 | mL/kg |
| | control | | | |
| G3 | Compound | 15 mg/kg, p.o., Q.D., | 15 | mg/kg, |
| | I-8 | 10 mL/kg. | | |
| G4 | Compound | 30 mg/kg later 50 mg/kg, | 30 | mg/kg |
| | I-8 | p.o., Q.D., 10 mL/kg | | |

Note:
G4, animals were administered 30 mg/kg dose until day 33 and 50 mg/kg from day 34 onwards
Unless indicated otherwise, data is presented as follows:
Data is shown as Mean ± S.E.M (n = 8-12)
*Significant difference as compared to G2
#Significant difference as compared to G1
Two- way ANOVA followed by Bonferroni's Multiple Comparison test.
*/#P < 0.05, **/##P < 0.01, ***/###P < 0.001, ****/####P < 0.0001.

### Experimental Protocol and Dose Administration

Male mice and age-matched control mice were procured. All animals were quarantine for 7 days and acclimatized for one day prior to experiment initiation. During this period, the animals were observed daily for clinical signs.

Mice were weighed and randomized into different groups and age matched control groups were kept as such. Test compound I-8 was freshly formulated and administered to the respective animals of each group as per body weight. Animals in G1 and G2 were administered with vehicle. G3 and G4 group animals were dosed with compound I-8 at the dose of 15 mg/kg and 30 mg/kg respectively, per orally; Q.D. G4 animals were administered 30 mg/kg dose until day 34 and 50 mg/kg from day 35 onwards.

At week 9, animals were treated either with vehicle or compound I-8 per orally Q.D. Animals were scored (twice in a week until week 12 and every day thereafter until termination) for motor impairment using Vercelli neurological scoring system at the scale of 4-0. Animals were subjected to different tests for motor coordination and muscle strength assessment once in two weeks.

At 16 weeks (9+7-week behavioral assessment), animals were dosed either with the vehicle or test compound and 4 hours post dosing, CSF, blood, and brain were collected for bioanalysis. Muscles (tibialis, soleus and gastrocnemius) and spinal cord were collected for histology (optional).

### Clinical Signs & Mortality

Animals were observed for any clinical signs to treatment throughout the study. Cage-side observations were made to detect any changes and general activity of the animals. After dose administration, all the animals were observed carefully for treatment related clinical signs, including morbidity and mortality.

### Body Weight

The body weight of each animal was recorded at the time of randomization and once in 2 days during the study period.

### Neurological Scoring

Animals were scored for motor deficits using the scoring system:
4—Normal/no sign of motor dysfunction
3—Presence of hindlimb tremors when suspended by tail
2—Presence of gait abnormalities
3—Dragging at least one hindlimb
Absence of right reflex within 30 seconds

Animals were scored twice a week from initiation of treatment until week 12 and everyday thereafter until termination.

### Wire Suspension Test (Inverted Screen Test)

Wire hang test was used to measure the muscle strength of animals. During the test, the animals were placed on the top of a wire lid of a housing cage. Then the lid was inverted and slightly shaken to ensure the grip of animal on the lid. Latency to fall(s) off the lid was recorded. A total of 3 trials were recorded and averaged to analyze the data. A cutoff time of 90 s was used to end the trial.

### Beam Walk Test

Beam walk test was used for the assessment of motor coordination or balance of animals. The beam apparatus consisted of 1-meter beams with a flat surface of 6 mm width resting 50 cm above the tabletop on two poles. Animals were placed at one end of the beam, allowed to traverse the beam and reach another end with a cage filled with the home cage bedding. Animals were trained on beams for 2 consecutive days, 3 trials per day with 30 s inter-trial interval. On day 3 (day of test), animals were tested on the beam. A total 3 trials were performed, and time taken to traverse the beam & number of slips were recorded, average data was analyzed. For animals unable to traverse the beam, a cutoff time of 60 s was assigned.

Note: Number of slips were recorded until week 12 due to disease progression after that.

### Rotarod Test

The Rotarod test was used for motor coordination assessment in mice. Animals were accustomed to the rotarod at 10 rpm for 300 s followed by training for 2 days at accelerated rpm (4-40 for 300 s) with 15 minutes inter trial interval. On the day of the test, animals were subjected to rotarod test at 4-40 rpm for 300 s with 15 minutes inter trial interval. During the training and test, the latency to fall off the rod was recorded. A total of 3 trials were recorded, data was averaged and analyzed.

Note: Behavioral tests were performed once in two weeks, i.e. at week 10, 12, 14 and 16.

### Bioanalysis

After completion of week 16 (9+7-week behavioral assessment), animals were dosed with the vehicle or test compound. 4 hours post dosing, CSF and blood (retro-orbitally) were collected under mild isoflurane anesthesia. Blood was centrifuged at 5000 rpm for 10 minutes at 4° C. and separated plasma was utilized for plasma drug concentration analysis. Then animals were euthanized, and brain tissue was collected. CSF, plasma and brain tissue samples from G3 and G4 were prepared for bioanalysis and compounds were extracted, and the supernatant was injected for LC-MS/MS bioanalysis. Drug concentration of compound I-8 was calculated from respective calibration curve for G3 and G4.

### Evaluation

All data was expressed as the mean±SEM. Data on each parameter was summarized in tabular form. Appropriate graphical representation was done using suitable methods. Statistical analysis was done with Graph Pad Prism-9 using one-way ANOVA followed by Bonferroni's post hoc test. Data were considered statistically significant if P value is less than 0.05.

### Clinical Signs & Mortality

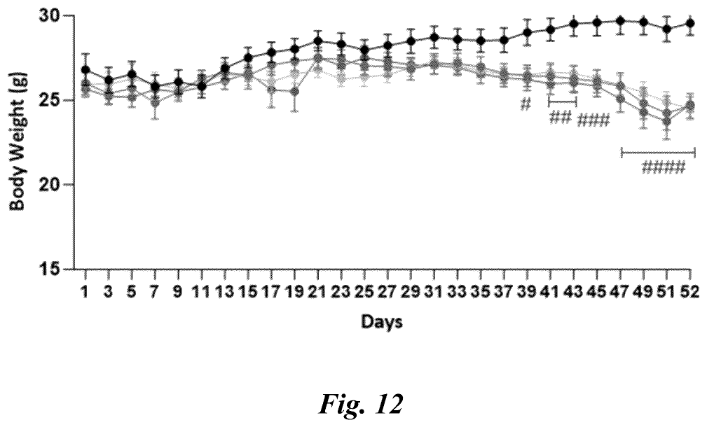

One mortality was observed in G2 and two mortalities in G4 were reported during the study. The effect of compound I-8 on body weight is shown in the table below (representative data) and FIG. 12. G2 demonstrated significant reduction in body weight vs. G1 from day 39 until day 52.

| Day | G1 | G2 | G3 | G4 |
|---|---|---|---|---|
| Day 1 | 26.81 ± 0.92 | 26.01 ± 0.67 | 25.99 ± 0.73 | 25.66 ± 0.48 |
| Day 7 | 25.82 ± 0.66 | 24.84 ± 0.95 | 25.97 ± 0.70 | 25.64 ± 0.40 |
| Day 13 | 26.89 ± 0.62 | 26.64 ± 0.57 | 26.54 ± 0.52 | 26.14 ± 0.52 |
| Day 21 | 28.50 ± 0.60 | 27.47 ± 0.63 | 26.81 ± 0.45 | 27.53 ± 0.60 |
| Day 27 | 28.23 ± 0.67 | 26.99 ± 0.56 | 26.51 ± 0.46 | 27.29 ± 0.52 |
| Day 35 | 28.52 ± 0.68 | 26.97 ± 0.60 | 26.76 ± 0.43 | 26.55 ± 0.57 |
| Day 43 | 29.52 ± 0.73 | 26.27 ± 0.76^{##} | 26.60 ± 0.50 | 26.04 ± 0.60 |
| Day 49 | 29.62 ± 0.76 | 24.82 ± 0.92^{####} | 25.47 ± 0.62 | 24.30 ± 0.96 |
| Day 52^{$} | 29.56 ± 0.71 | 24.66 ± 0.73^{####} | 24.52 ± 0.68 | 24.75 ± 0.45 |

Note:
^{$}- n = 6-8 at day 52 due to mortalities in G2 and G4 groups
Note:
n = 6-8 at day 52 due to mortalities in G2 and G4 groups

### Neurological Scoring

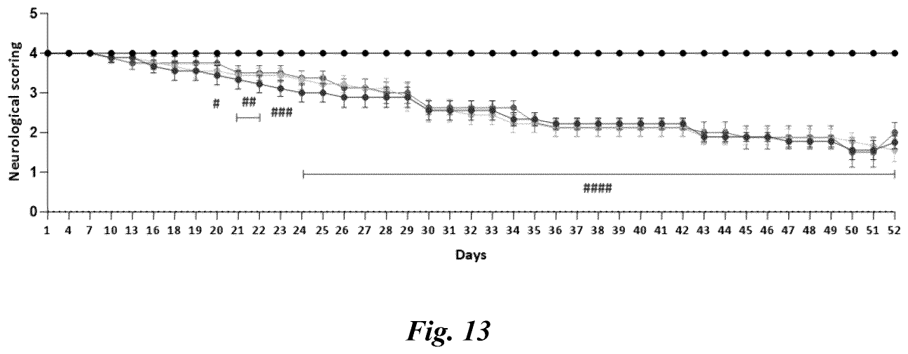

The effect of compound I-8 on neurological scoring is shown in the table below (representative data) and FIG. 13. G2 group demonstrated significant motor deficit vs. G1 from day 20 until day 52.

| Day | G1 | G2 | G3 | G4 |
|---|---|---|---|---|
| Day 1 | 4 ± 0 | 4 ± 0 | 4 ± 0 | 4 ± 0 |
| Day 7 | 4 ± 0 | 4 ± 0 | 4 ± 0 | 4 ± 0 |
| Day 13 | 4 ± 0 | 3.89 ± 0.11 | 3.89 ± 0.11 | 3.75 ± 0.16 |
| Day 21 | 4 ± 0 | 3.33 ± 0.24^{##} | 3.44 ± 0.18 | 3.5 ± 0.19 |
| Day 28 | 4 ± 0 | 2.89 ± 0.26^{####} | 3.11 ± 0.26 | 3 ± 0.27 |
| Day 35 | 4 ± 0 | 2.33 ± 0.17^{####} | 2.22 ± 0.22 | 2.25 ± 0.25 |
| Day 42 | 4 ± 0 | 2.22 ± 0.15^{####} | 2.11 ± 0.20 | 2.13 ± 0.23 |
| Day 49 | 4 ± 0 | 1.78 ± 0.15^{####} | 1.89 ± 0.20 | 1.88 ± 0.30 |
| Day 52^{$} | 4 ± 0 | 1.75 ± 0.16^{####} | 1.56 ± 0.29 | 2 ± 0.26 |

Note:
^{$}- n = 6-8 at day 52 due to mortalities in G2 and G4 groups

### Beam Walk Test

The effect of compound I-8 on Beam Walk Test is shown in the table below and FIG. 14. G2 demonstrated significant increase in latency to traverse the beam vs. G1 on week 10, week 14 and week 16. G4 demonstrated significant reduction in latency to traverse the beam vs. G2 on week 10. G2 demonstrated significant reduction in number of slips vs. G1 on week 10.

Beam Walk Test- Latency to traverse the beam(s)

| Treatment | | | | |
|---|---|---|---|---|
| Group | Week 10 | Week 12 | Week 14 | Week 16 |
| G1 | 19.5 ± 2.51 | 14.89 ± 3.09 | 7.58 ± 0.60 | 9 ± 1.36 |
| G2 | 34.93 ± 6.92^{#} | 27.56 ± 3.97 | 33.30 ± 7.01^{###} | 60 ± 0^{####} |
| G3 | 21.37 ± 5.99 | 25.96 ± 5.59 | 20.52 ± 6.85 | 58.21 ± 1.79 |
| G4 | 17.04 ± 5.71* | 29.83 ± 6.82 | 25.29 ± 7.64 | 60 ± 0 |

Beam Walk Test: Number of Slips

| Treatment | | |
|---|---|---|
| Group | Week 10 | Week 12 |
| G1 | 4.19 ± 0.50 | 0.47 ± 0.11 |
| G2 | 1.29 ± 0.17^{####} | 0.89 ± 0.18 |
| G3 | 1.42 ± 0.14 | 0.93 ± 0.19 |
| G4 | 1.33 ± 0.40 | 1.38 ± 0.34 |

### Rotarod Test

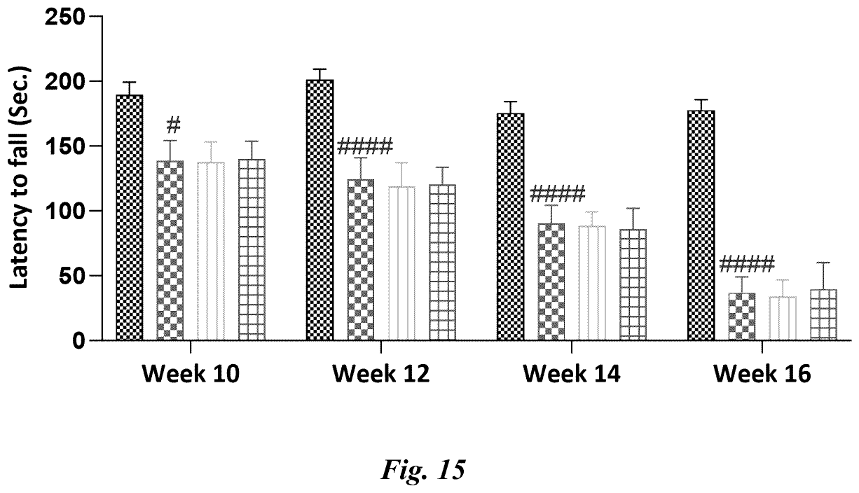

The effect of compound I-8 on Rotarod Test is shown in the table below and FIG. 15. G2 demonstrated significant reduction in latency to fall of the rod vs. G1 on week 10, week 12, week 14 and week 16.

| Treatment | | | | |
|---|---|---|---|---|
| Group | Week 10 | Week 12 | Week 14 | Week 16 |
| G1 | 189.5 ± 9.75 | 201.44 ± 7.85 | 175.47 ± 8.89 | 177.81 ± 8.04 |
| G2 | 138.59 ± 15.71# | 124.37 ± 16.55^{####} | 90.15 ± 14.06^{####} | 37.04 ± 12.06^{####} |
| G3 | 137.59 ± 15.62 | 119.19 ± 18.07 | 88.63 ± 10.57 | 34.04 ± 12.74 |
| G4 | 140 ± 13.47 | 120.58 ± 13.17 | 86 ± 16.17 | 39.79 ± 20.43 |

### Wire Suspension Test

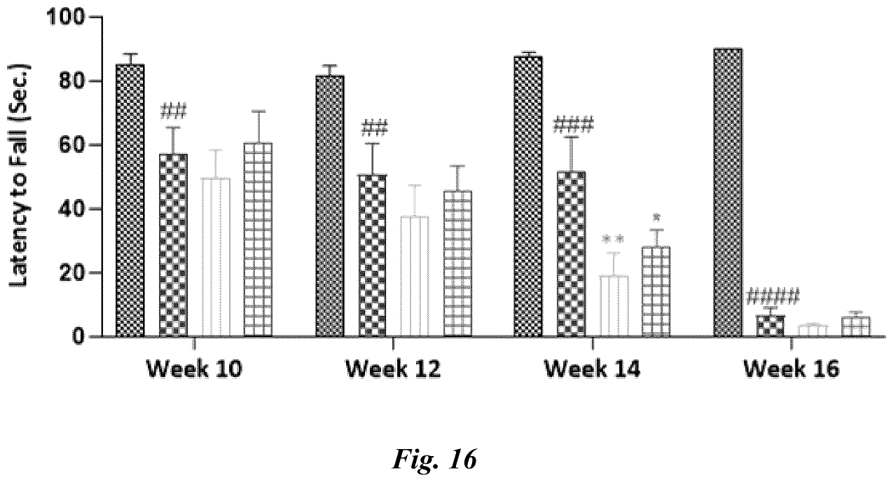

The effect of compound I-8 on Wire Suspension test is shown in the table below and FIG. 16. G2 demonstrated significant reduction in latency to fall of the wire vs. G1 on week 10, week 12, week 14 and week 16. G3 demonstrated a significant reduction in latency to fall of the wire vs. G2 on week 14. G4 demonstrated a significant reduction in latency to fall of the wire vs. G2 on week 14.

| Treatment | | | | |
|---|---|---|---|---|
| Group | Week 10 | Week 12 | Week 14 | Week 16 |
| G1 | 85.44 ± 3.05 | 81.79 ± 2.87 | 87.58 ± 1.29 | 90 ± 0 |
| G2 | 57.07 ± 8.38^{#} | 50.81 ± 9.51^{##} | 51.78 ± 10.85^{###} | 6.88 ± 2.423^{####} |
| G3 | 49.70 ± 8.667 | 37.48 ± 9.92 | 19.22 ± 7.18** | 3.71 ± 0.73 |
| G4 | 60.38 ± 10.00 | 45.46 ± 7.98 | 28.13 ± 5.38* | 6.46 ± 1.58 |

### Bioanalysis Results—4-Hour Timepoint

| | | Plasma | Brain | CSF |
|---|---|---|---|---|
| G3 | Mean | 12.35 | 2.34 | — |
| | SD | 4.35 | 0.19 | — |
| | % CV | 35.25 | 8.15 | — |
| G4 | Mean | 136.15 | 3.87 | 2.70 |
| | SD | 137.97 | — | 0.34 |
| | % CV | 101.33 | — | 12.57 |

### SUMMARY

The effect of compound I-8 was evaluated in mice using an animal model of amyotrophic lateral sclerosis (ALS). Animals in the disease control group demonstrated disease progression compared to vehicle control group and the same was evident from neurological scoring. Also, the disease control group demonstrated significant impairment in motor strength, motor coordination and motor balance compared to vehicle control group at week 10, 12, 14 and 16. A significant improvement was observed in motor balance of G4 group at week 10 compared to G2.

The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having the following Structure (I): 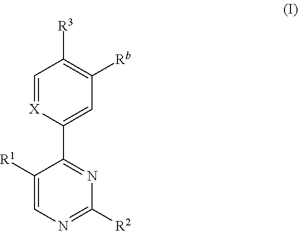
or a pharmaceutically acceptable salt, stereoisomer, or tautomer thereof, wherein:
X is N or CR^{a};
R^{a} is hydrogen or halo;
R^{b} is hydrogen, halo, or C₁-C₆ alkyl;
R¹ is hydrogen or halo;
R² has one of the following structures: 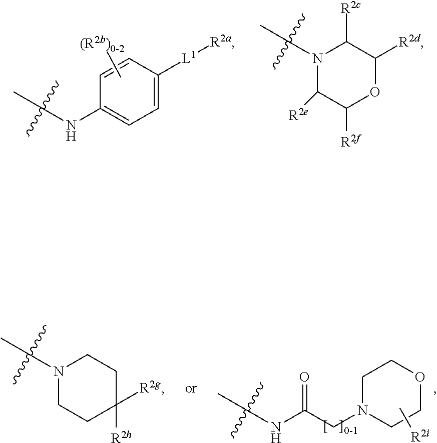
wherein:
L¹ is a direct bond, —CH₂—, —C(CH₃)H— or —C(═O)—;
R^{2a} has one of the following structures: 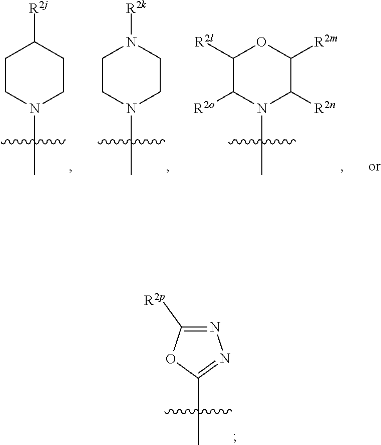
R^{2b} is halo;
R^{2c}, R^{2d}, R^{2e}, and R^{2f} are each independently hydrogen, C₁-C₆ alkyl, or
R^{2c} and R^{2f} join to form a C₁-C₆ alkylene bridge between the carbons to which they are attached, or
R^{2d} and R^{2e} join to form a C₁-C₆ alkylene bridge between the carbons to which they are attached;
R^{2g} and R^{2h} are each independently hydrogen, halo, or
R^{2g} and R^{2h}, together with the carbon to which they are attached join to form a 3-8 membered heterocyclyl;
R²ⁱ is hydrogen or C₁-C₆ alkyl;
R^{2j} is hydrogen, halo, or C₁-C₆ alkyl;
R^{2k} is hydrogen, C₁-C₆ alkyl, or 3 to 8 membered heterocyclyl;
R^{2l}, R^{2m}, R²ⁿ, and R^{2o} are each independently hydrogen, C₁-C₆ alkyl, or
R^{2l} and R²ⁿ join to form a C₁-C₆ alkylene bridge between the carbons to which they are attached, or
R^{2m} and R^{2o} join to form a C₁-C₆ alkylene bridge between the carbons to which they are attached;
R^{2p} is hydrogen, C₁-C₆ alkyl, or C₁-C₆ haloalkyl;
R³ has one of the following structures: 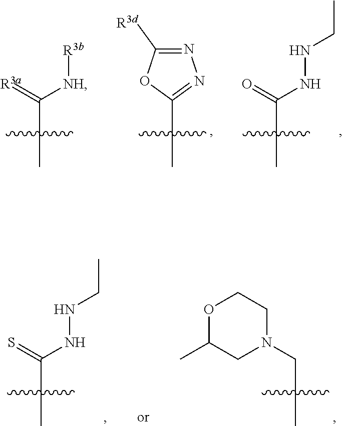
wherein:
R^{3a} is O or S;
R^{3b} is —OH, —CN, or —R^{3e};
R^{3d} is C₁-C₆ haloalkyl; and
R^{3e} is C₁-C₆ alkyl;
provided that when L¹ is a direct bond, R^{2b} is halo.

2. The compound of claim 1, or a pharmaceutically acceptable salt, stereoisomer, or tautomer thereof, wherein X is CR^{a} and R^{a} is hydrogen.

3. (canceled)

4. The compound of claim 1, or a pharmaceutically acceptable salt, stereoisomer, or tautomer thereof, wherein X is CR^{a} and R^{a} is fluoro.

5. The compound of claim 1, or a pharmaceutically acceptable salt, stereoisomer, or tautomer thereof, wherein X is N.

6. The compound of claim 1, or a pharmaceutically acceptable salt, stereoisomer, or tautomer thereof, wherein R^{b} is hydrogen.

7. The compound of claim 1, or a pharmaceutically acceptable salt, stereoisomer, or tautomer thereof, wherein R^{b} is methyl.

8. The compound of claim 1, or a pharmaceutically acceptable salt, stereoisomer, or tautomer thereof, wherein R¹ is hydrogen.

9. The compound of claim 1, or a pharmaceutically acceptable salt, stereoisomer, or tautomer thereof, wherein R¹ is chloro.

10. The compound of claim 1, or a pharmaceutically acceptable salt, stereoisomer, or tautomer thereof, wherein R² has the following structure:

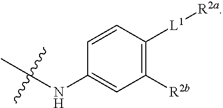

11. The compound of claim 1, or a pharmaceutically acceptable salt, stereoisomer, or tautomer thereof, wherein R² has the following structure:

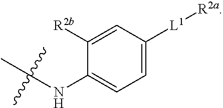

12-14. (canceled)

15. The compound of claim 1, or a pharmaceutically acceptable salt, stereoisomer, or tautomer thereof, wherein R² has one of the following structures:

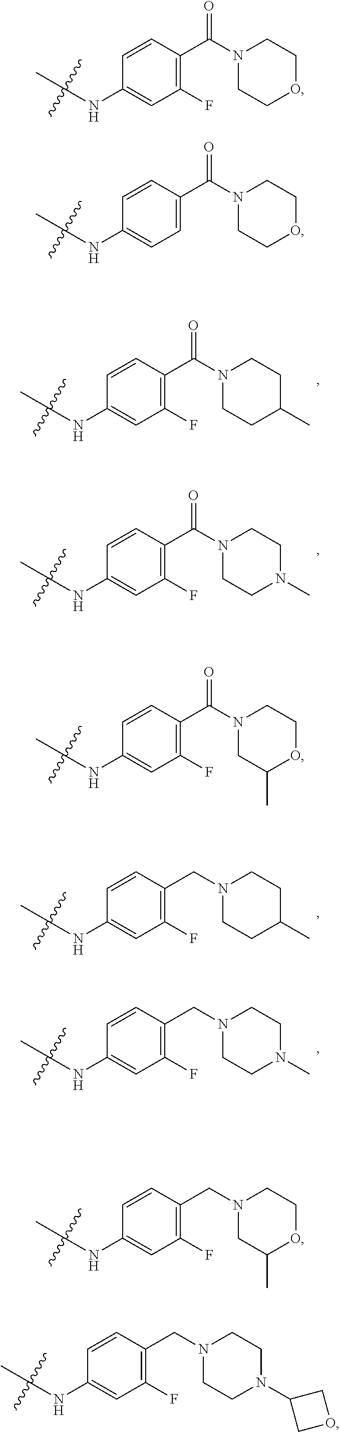

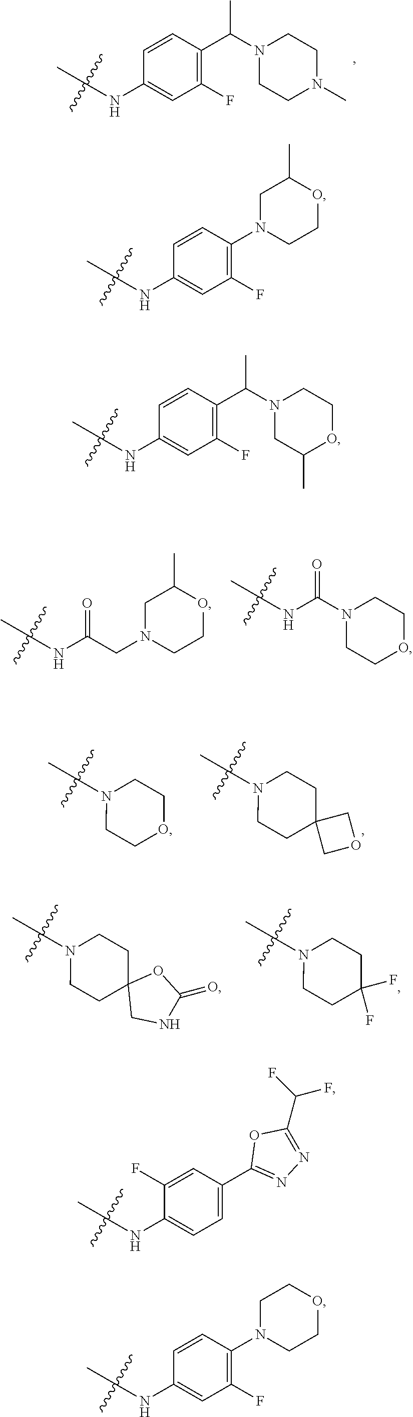

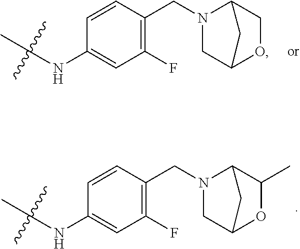 [SMILES: *Nc1ccc(CN2CC3CC2CO3)c(F)c1 ; *Nc1ccc(CN2CC3CC2C(C)O3)c(F)c1]

16. The compound of claim 1, or a pharmaceutically acceptable salt, stereoisomer, or tautomer thereof, wherein R² has one of the following structures:

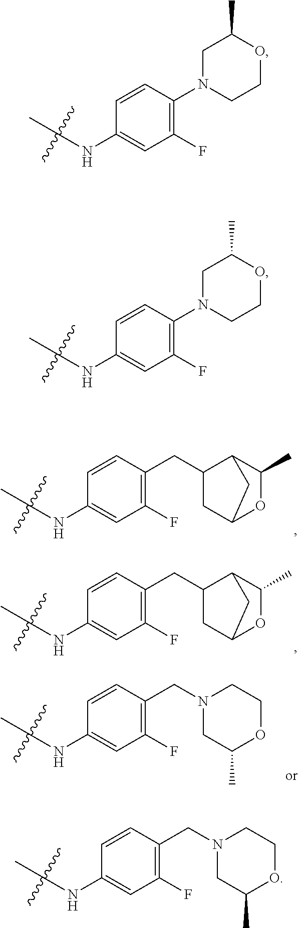

17-19. (canceled)

20. The compound of claim 1, or a pharmaceutically acceptable salt, stereoisomer, or tautomer thereof, wherein R³ has one of the following structures:

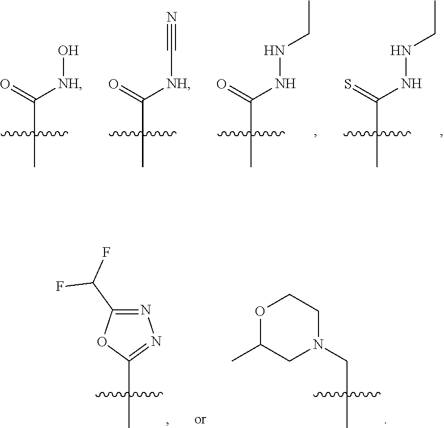

21. The compound of claim 1, or a pharmaceutically acceptable salt, stereoisomer, or tautomer thereof, wherein R³ has one of the following structures:

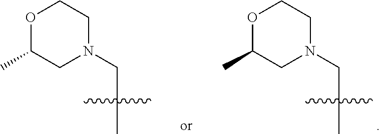

22. The compound of claim 1, or a pharmaceutically acceptable salt, stereoisomer, or tautomer thereof, wherein the compound is selected from Table 1.

23. (canceled)

24. A pharmaceutical composition comprising the compound of claim 1, or a pharmaceutically acceptable salt, stereoisomer, or tautomer thereof, and a pharmaceutically acceptable excipient.

25. A method of treating a HDAC6 mediated disease, the method comprising administering the compound of claim 1, or a pharmaceutically acceptable salt, stereoisomer, or tautomer thereof or the pharmaceutical composition of claim 24 to a subject in need thereof.

26. The method of claim 25, wherein the HDAC6 mediated disease is cancer, a neurodegenerative disease, a neurological disorder, Alzheimer's disease, amyotrophic lateral sclerosis (ALS), Rett syndrome, idiopathic pulmonary fibrosis, non-small cell lung cancer, chronic lymphocytic leukemia, multiple myeloma, or an autoimmune disease.

27-28. (canceled)

29. A pharmaceutical composition comprising the compound of claim 22 or a pharmaceutically acceptable salt, stereoisomer, or tautomer thereof, and a pharmaceutically acceptable excipient.
